(19) United States
(12) Patent Application Publication
(10) Pub. No.: US 2025/0144232 A1
(43) Pub. Date: May 8, 2025

(54) PROGRAMMABLE LIPID NANOPARTICLE DELIVERY VIA CORONA PROTEIN ENGINEERING

(71) Applicants: Massachusetts Institute of Technology, Cambridge, MA (US); Brigham and Women's Hospital, Boston, MA (US); Beth Israel Deaconess Medical Center, Boston, MA (US)

(72) Inventors: Omar Abudayyeh, Cambridge, MA (US); Jonathan Gootenberg, Cambridge, MA (US); Cian Schmitt-Ulms, Cambridge, MA (US); Alisan Kayabolen, Cambridge, MA (US)

(21) Appl. No.: 18/902,341

(22) Filed: Sep. 30, 2024

Related U.S. Application Data
(60) Provisional application No. 63/690,138, filed on Sep. 3, 2024.
(60) Provisional application No. 63/541,094, filed on Sep. 28, 2023.

(51) Int. Cl. A61K 48/00; A61K 9/51; A61K 47/64

(52) U.S. Cl. CPC A61K 48/0033; A61K 9/5123; A61K 47/64

(57) ABSTRACT

Disclosed are compositions comprising a lipid nanoparticle and a modified biomolecular corona. In some embodiments, the modified biomolecular corona comprises a fused cell-specific binding domain. In some embodiments, the modified biomolecular corona protein has been additionally modified such that it does not bind substantially to its natural receptor. In some embodiments, the fused cell-specific binding domain binds to a target cell.

### RELATED APPLICATIONS

This application claims the benefit of priority to U.S. Provisional Patent Application Ser. No. 63/541,094, filed Sep. 28, 2023; and U.S. Provisional Patent Application Ser. No. 63/690,138, filed Sep. 3, 2024; each of which is hereby incorporated by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Dec. 19, 2024, is named MTV-20701_SL.xml and is 295,422 bytes in size.

### BACKGROUND

Significant progress has been made in the development of new nucleic acid-based therapeutic technologies. These technologies include CRISPR tools for genome editing, modified mRNA for gene and protein replacement, and oligo-based therapies for gene silencing. While these technologies have been transformative, they have failed to live up to their full potential due to shortcomings of delivery technologies, preventing most human organs from being addressable via systemic intravenous infusions. Accordingly, there is a great need to identify the novel delivery technologies of nucleic acids.

### SUMMARY OF THE INVENTION

In one aspect the present disclosure provides a composition, comprising a lipid nanoparticle and a modified lipid binding protein. In some embodiments, the modified lipid binding protein comprises a fused cell-specific binding domain. In some embodiments, the modified lipid binding protein has been additionally modified such that it does not bind substantially to its natural receptor. In some embodiments, the fused cell-specific binding domain binds to a target cell.

In some embodiments, the lipid binding protein is a biomolecular corona protein. In some embodiments, lipid binding protein is an Apolipoprotein, such as ApoA-I, ApoA-II, ApoA-IV, ApoA-V, ApoB, ApoB-48 and ApoB-100, ApoC-I, ApoC-II, ApoC-III, ApoD, ApoE, ApoF, ApoH (Beta-2-glycoprotein I), ApoJ (Clusterin), ApoL, ApoM, ApoO (Apolipoprotein O), or Apo (a)-component of Lipoprotein (a). In some embodiments, the Apolipoprotein is ApoE. In some embodiments, the lipid binding protein is a Fatty Acid Binding Protein (FABP), such as L-FABP, I-FABP, or H-FABP. In some embodiments, the lipid binding protein is β2-GPI, Vtn5, a cellular retinol-binding protein (CRBP), a cellular retinoic acid-binding protein (CRABP), a sterol carrier protein (SCP), NPC1, NPC2, a StarD protein (like StarD1, StarD2), a phosphatidylserine-binding protein, an oxysterol-binding protein (OSBP), a Acyl-CoA-binding protein (ACBD), a PLIN protein, or perilipin.

In some embodiments, the lipid binding protein is an engineered lipid binding protein. In some embodiments, the lipid binding protein has mutation in its receptor binding domain. In some embodiments, ApoE has a mutation in its low-density lipoprotein receptor (LDLR) binding domain. In some embodiments, the mutation in ApoE is R142A, R142C, R143S, R142A/K143S, R142C/K143S, R145C, R172, I250S/L252S, A237R, or A241R.

In some embodiments, the modified lipid binding protein prevents off-target delivery. In some embodiments, the cell-specific binding domain binds to a cell-specific surface protein. In some embodiments, the cell-specific binding domain allows for localization to a specific cell membrane, and/or uptake into a specific cell. In some embodiments, the cell-specific binding domain is fused to the amino terminus, the carboxy terminus, or the flexible solvent-exposed loop of the lipid binding protein. In some embodiments, the cell-specific binding domain is fused to the lipid binding protein via a flexible linker, such as GGGS (SEQ ID NO: 267), (GGS) 5 (SEQ ID NO: 268), or XTEN. In some embodiments, the cell-specific binding domain is vitronectin, EGFR DARPin, an scFv, myomixer, an integrin-binding peptide, or (KKEEE) 3K binder (SEQ ID NO: 269). In some embodiments, the scFv binds to cubilin, Alpha-v beta-3, EGFR, CD34, CD90, CD117, CD19, CD20, CD3, CD5, CD4, myomaker, Megalin, or Cubilin.

In some embodiments, the lipid binding protein is further fused to a lipid-binding peptide. In some embodiments, the lipid-binding peptide is 18A, 4F, or ETC-642. In some embodiments, the lipid-binding peptide is fused to the amino terminus, the carboxy terminus, or the flexible solvent-exposed loop of the lipid binding protein. In some embodiments, the lipid-binding peptide is fused to the lipid binding protein via a flexible linker. In some embodiments, the flexible linker is GGGS (SEQ ID NO: 267), (GGS) 5 (SEQ ID NO: 268), or XTEN.

In some embodiments, the lipid binding protein comprises a mutation in its lipid-binding domain and/or its amino terminal fragment. In some embodiments, the composition further comprises a serum-stabilizing tag. In some embodiments, the serum-stabilizing tag is XTEN. In some embodiments, the lipid nanoparticle and the modified lipid binding protein are mixed in a ratio of about 1:1, 1:2, 1:3, 1:4, 1:5, 1:6, 1:7, 1:8, 1:9, or 1:10. In some embodiments, the modified lipid binding protein is in the form of conditioned media (CM). In some embodiments, the composition further comprises a nucleic acid. In some embodiments, the nucleic acid is a DNA, an RNA or combination thereof. In some embodiments, the RNA is a messenger RNA (mRNA), a non-coding RNA (ncRNA) or a combination thereof. In some embodiments, the non-coding RNA (ncRNA) is long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA) or ribosomal RNA (rRNA) or combination thereof. In some embodiments, the nucleic acid comprises at least one chemical modification.

In one aspect the present disclosure provides a composition, comprising a lipid nanoparticle and a modified lipid binding protein. In some embodiments, the modified lipid binding protein comprises a conjugated cell-specific binding domain. In some embodiments, the modified lipid binding protein has been additionally modified such that it does not bind substantially to its natural receptor. In some embodiments, the conjugated cell-specific binding domain binds to a target cell. In some embodiments, the cell-specific binding domain is conjugated to the modified lipid binding protein via a protein A/G.

In one aspect the present disclosure provides a composition, comprising a lipid nanoparticle and a modified lipid binding protein. In some embodiments, the lipid nanoparticle comprises a conjugated cell-specific binding domain. In some embodiments, the modified lipid binding protein has been additionally modified such that it does not bind substantially to its natural receptor. In some embodiments, the conjugated cell-specific binding domain binds to a target cell. In some embodiments, the cell-specific binding domain is conjugated to the lipid nanoparticle via a covalent bond.

In some embodiments, the lipid binding protein is a biomolecular corona protein. In some embodiments, lipid binding protein is an Apolipoprotein, such as ApoA-I, ApoA-II, ApoA-IV, ApoA-V, ApoB, ApoB-48 and ApoB-100, ApoC-I, ApoC-II, ApoC-III, ApoD, ApoE, ApoF, ApoH (Beta-2-glycoprotein I), ApoJ (Clusterin), ApoL, ApoM, ApoO (Apolipoprotein O), or Apo (a)-component of Lipoprotein (a). In some embodiments, the Apolipoprotein is ApoE. In some embodiments, the lipid binding protein is a Fatty Acid Binding Protein (FABP), such as L-FABP, I-FABP, or H-FABP. In some embodiments, the lipid binding protein is β2-GPI, Vtn5, a cellular retinol-binding protein (CRBP), a cellular retinoic acid-binding protein (CRABP), a sterol carrier protein (SCP), NPC1, NPC2, a StarD protein (like StarD1, StarD2), a phosphatidylserine-binding protein, an oxysterol-binding protein (OSBP), a Acyl-CoA-binding protein (ACBD), a PLIN protein, or perilipin.

In some embodiments, the lipid binding protein is an engineered lipid binding protein. In some embodiments, the lipid binding protein has mutation in its receptor binding domain. In some embodiments, ApoE has a mutation in its low-density lipoprotein receptor (LDLR) binding domain. In some embodiments, the mutation in ApoE is R142A, R142C, R143S, R142A/K143S, R142C/K143S, R145C, R172, 1250S/L252S, A237R, or A241R.

In some embodiments, the modified lipid binding protein prevents off-target delivery. In some embodiments, the cell-specific binding domain binds to a cell-specific surface protein. In some embodiments, the cell-specific binding domain allows for localization to a specific cell membrane, and/or uptake into a specific cell. In some embodiments, the cell-specific binding domain is fused to the amino terminus, the carboxy terminus, or the flexible solvent-exposed loop of the lipid binding protein. In some embodiments, the cell-specific binding domain is fused to the lipid binding protein via a flexible linker, such as GGGS (SEQ ID NO: 267), (GGS) 5 (SEQ ID NO: 268), or XTEN. In some embodiments, the cell-specific binding domain is vitronectin, EGFR DARPin, an scFv, myomixer, an integrin-binding peptide, or (KKEEE) 3K binder (SEQ ID NO: 269). In some embodiments, the scFv binds to cubilin, Alpha-v beta-3, EGFR, CD34, CD90, CD117, CD19, CD20, CD3, CD5, CD4, myomaker, Megalin, or Cubilin.

In some embodiments, the lipid binding protein is further fused to a lipid-binding peptide. In some embodiments, the lipid-binding peptide is 18A, 4F, or ETC-642. In some embodiments, the lipid-binding peptide is fused to the amino terminus, the carboxy terminus, or the flexible solvent-exposed loop of the lipid binding protein. In some embodiments, the lipid-binding peptide is fused to the lipid binding protein via a flexible linker. In some embodiments, the flexible linker is GGGS (SEQ ID NO: 267), (GGS) 5 (SEQ ID NO: 268), or XTEN.

In some embodiments, the lipid binding protein comprises a mutation in its lipid-binding domain and/or its amino terminal fragment. In some embodiments, the composition further comprises a serum-stabilizing tag. In some embodiments, the serum-stabilizing tag is XTEN. In some embodiments, the lipid nanoparticle and the modified lipid binding protein are mixed in a ratio of about 1:1, 1:2, 1:3, 1:4, 1:5, 1:6, 1:7, 1:8, 1:9, or 1:10. In some embodiments, the modified lipid binding protein is in the form of conditioned media (CM). In some embodiments, the composition further comprises a nucleic acid. In some embodiments, the nucleic acid is a DNA, an RNA or combination thereof. In some embodiments, the RNA is a messenger RNA (mRNA), a non-coding RNA (ncRNA) or a combination thereof. In some embodiments, the non-coding RNA (ncRNA) is long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA) or ribosomal RNA (rRNA) or combination thereof. In some embodiments, the nucleic acid comprises at least one chemical modification.

In one aspect the present disclosure provides a method of treating or preventing a disease, comprising administering to a subject in need thereof an effective amount of the composition described herein. In some embodiments, the disease is cancer, an infectious disease, a muscular disease, a neurodegenerative disease, or a disease and/or disorder ameliorated by humoral and/or cellular immune response. In some embodiments, the disease is associated with a genetic mutation. In some embodiments, the composition corrects the genetic mutation.

### BRIEF DESCRIPTION OF THE DRAWINGS

FIG. 1 shows an LNP synthesis protocol.

FIG. 2 shows a set of images taken using a fluorescent microscope of LNPs packaging a GFP cargo. LNPs are mixed with human or mouse serum at each of the concentrations listed (100% to 0.78%).

FIG. 3 shows a set of images taken using a fluorescent microscope of LNPs packaging one of two GFP cargos. Two amounts of RNA are used (25 ng and 75 ng) as well as modified or unmodified GFP RNA. LNPs are transduced alone or after incubation with wild-type or mutant APOE CM.

FIG. 4 shows a DLS curve for several LNP samples incubated with different constructs. LNPs are either mixed with PBS, recombinant protein, or condition media. This plot shows the approximate distribution of sizes of the particles in the incubated mixture in nanometers. ApoE3 conditions only show modest size increase with ApoE3 recombinant, mock (pDY0088) CM. Larger increase with ApoE3 conditioned media (also best performing transduction).

FIG. 5 shows representative EM images from LNPs incubated with the indicated mixtures.

FIG. 6 shows the ImageJ sizing strategy for approximating LNP size as wells as the quantification from applying this sizing strategy across a set of representative images for each sample. All sizes measured are plotted on the grouped chart. Sizes are expressed in nm.

FIG. 7 shows a denaturing stain-free gel run on a protein purified from either the secreted fraction or cell lysate from HEK293s transfected with a construct expressing His-tagged APOE3. The conditioned labeled “elutions” are the output of the purification process, while the media/lysate lanes are the inputs. “FT” represents the flowthrough from the initial binding and washing stage of the Ni-NTA column used for purification here. Protein inputs are not normalized before loading on the gel to show relative abundances.

FIG. 8 shows the raw output and purified protein concentrations from a BCA run on the outputs of Ni-NTA affinity purification of the wildtype APOE from cell lysate or from media. Elution volume is 3 mL here—can potentially reduce this volume or add more input. Concentration from each fraction as follows: Past recombinant protein amounts for transduction were: 1.5 μg from 1 mg/mL stock.

FIG. 9 shows the relative transduction efficiencies measured as %-GFP-positive cells from HEK293FT cells transduced with either LNP only or LNP mixed with purified His-tagged APOE wildtype protein purified from cell lysate or conditioned media. Several protein amounts are used for each of the above conditions. An unpurified CM control and unpurified APOE mutant control are transduced on the left as controls.

FIG. 10 shows AlphaFold predictions of structures of wild-type and mutant or tagged APOE constructs, showing a relatively conserved core structure that matches the wildtype construct. FIG. 10 discloses SEQ ID NOs 2, 2 and 2, respectively, in order of appearance.

FIG. 11 shows a denaturing stain-free gel run on a protein purified from either the secreted fraction or cell lysate from HEK293s transfected with a construct expressing various types of ligand-tagged APOE3. Constructs shown left to right are: APOE3-wt, APOE3_R142A_K143S, EE_GS10_APOE3_R142A_K143S, MoonTag_GS10_APOE3_R142A_K143S, SunTag_GS10_APOE3_R142A_K143S, ALFA_GS10_APOE3_R142A_K143S, MyoAAV-1A_GS10_APOE3_R142A_K143S, AAV-PHP-eB_GS10_APOE3_R142A_K143S, GYR_GS10_APOE3_R142A_K143S, MockTfx, scFv-CD19_GS10_APOE3-trunc191, APOE4, and scFv-CD19_GS10_18A. In all cases, constructs are generated by inserting the tag or ligand in question between the signal peptide and APOE protein, with a training GS10 linker (SP-ligand-GS10-APOE).

FIG. 12 shows a denaturing stain-free gel run on a protein purified from either the secreted fraction or cell lysate from HEK293s transfected with a construct expressing various types of ligand-tagged APOE3. In all cases, constructs are generated by inserting the tag or ligand in question between the signal peptide and APOE protein, with a training GS10 linker (SP-ligand-GS10-APOE).

FIG. 13 shows a set of images taken using a fluorescent microscope of LNPs packaging a GFP cargo. LNPs are either naked, coated with wildtype APOE CM, coated with mutant CM (top row) or incubated with varying amounts (listed) of purified wildtype APOE, mutant APOE, or N-terminal flag-GS10-mutant APOE constructs.

FIG. 14 shows relative transduction efficiencies of several core LNP conditions (naked LNP, coating with FBS (fetal bovine serum) or coating with recombinant lipoprotein.

FIG. 15 shows transduction efficiencies of several core LNP conditions (naked LNP, coating with FBS (fetal bovine serum) or coating with recombinant lipoprotein expressed as a % of GFP-positive cells. These efficiencies are the quantifications of the images in FIG. 14.

FIG. 16 shows a set of images taken using a fluorescent microscope of lipid nanoparticles (LNPs) packaging a GFP cargo. LNPs are either naked, coated with wildtype APOE CM, coated with mutant CM (top row) or incubated with varying amounts (listed) of purified mutant APOE or N-terminal flag-GS10-mutant APOE constructs. LNPs are incubated and dosed on either normal HEK293FT cells (top row) or HEK293FT cells transfected 24h prior with an anti-FLAG ScFv construct.

FIG. 17 shows a set of images taken using a fluorescent microscope of LNPs packaging a GFP cargo. Ligands are incubated with CM from cells transfected with either the wildtype APOE, mutant APOE, or N-terminally conjugated ligand APOE constructs. These LNPs are dosed on HEK cells transfected with one of several ScFvs against different ligands.

FIG. 18 shows a quantification (in % GFP positive cells) from flow cytometry of cells from the FIG. 17 crossing different ligand-APOE conjugated constructs with target cells expressing different synthetic receptors.

FIG. 19 shows a set of images taken using a fluorescent microscope of LNPs packaging a GFP cargo. LNPs are incubated with regular APOE, mutant “dead” APOE, or APOE conjugated to a CD19-targeting ScFv, and transduced either on normal HEK293FTs or HEK293FTs transfected with a CD19 receptor expression vector. The panel on the right shows a quantification by flow of GFP-positive cells from the same experiment.

FIG. 20 shows a quantification by flow of GFP-positive cells from LNPs packaging a GFP cargo. LNPs are incubated with regular APOE, mutant “dead” APOE, or APOE conjugated to a CD4-targeting ScFv, and transduced either on normal HEK293FTs or HEK293FTs transfected with a CD4 receptor expression vector.

FIG. 21 shows a subset of images taken using a fluorescent microscope of LNPs packaging a GFP cargo. LNPs are incubated with regular APOE, mutant “dead” APOE, or APOE conjugated to a “minibinder” (see Cao L., et al. Nature. 2022 May; 605 (7910): 551-560) for the transferrin receptor and transduced either on normal HEK293FTs or HEK293FTs transfected with a Transferin (TFRC) receptor expression vector. The panel on the right shows a quantification by flow of GFP-positive cells from the same experiment.

FIG. 22 shows a subset of images taken using a fluorescent microscope of LNPs packaging a GFP cargo. LNPs are incubated with regular APOE, mutant “dead” APOE, or APOE conjugated to an ScFv for CD90 or CD30 and transduced either on normal HEK293FTs or HEK293FTs transfected with a CD90 receptor expression vector. The panel on the right shows a quantification by flow of GFP-positive cells from the same experiment,

FIG. 23 shows a subset of images taken using a fluorescent microscope of LNPs packaging a GFP cargo. LNPs are incubated with regular APOE, mutant “dead” APOE, or APOE with the “knob” domain from an adenovirus inserted and transduced either on HEK293FTs or A549s,

FIG. 24 shows the quantification by flow of cells transduced in FIG. 23. LNPs are incubated with regular APOE, mutant “dead” APOE, or APOE with the “knob” domain from an adenovirus inserted and transduced either on HEK293FTs or A549s.

FIG. 25 shows a set of images taken using a fluorescent microscope of LNPs packaging a GFP cargo, transduced on Hepg2s. LNPs are either naked, coated with wildtype APOE CM, or coated with mutant CM (top row). Controls for mock transfection (no plasmid) and messengerMax lipofection are shown alongside.

FIG. 26 shows that homemade LNPs transduce well, have apparent tropism to liver. Left Figure shows quantification of luminescence from an initial set of LNP injections, testing two different quantities of LNP produced by two different methods. PBS control is indicated as PBS, LNP amounts are designated 1× (higher dose) and dilute (lower dose). Right Figure shows images with IVIS for the mice in question, measuring photons/s.

FIG. 27 shows last minute QC: Competition against purified protein shows only modest transduction block in vitro. Left Figure shows luminescence measurements from LNPs coated with one condition, then mixed with a second condition (ex. Initial incubation with mutant APOE, then incubation with 1% serum in PBS. Right Figure shows luminescence values for LNPs incubated with mutant APOE, then incubated with 1% serum normalized to luminescence of LNPs directly incubated with 1% serum.

FIG. 28 shows round 2 LNPs with F.luc efficiently transduce following retro-orbital injection. Right Figure show images taken with IVIS from mice injected with PBS or control “naked” LNPs.

FIG. 29 shows images taken with IVIS from mice injected with LNPs coated with purified mutant (MUT) or wildtype (APOE) APOE protein. Imaging for luciferase signal post luciferin injection.

FIG. 30 shows Mutant APOE-coated LNPs have Fluc signal equivalent to PBS injection. Left Figure shows a quantification of the luminescence signal from images also described in FIGS. 28 and 29. Right Figure show what images quantification is based on, also shown in FIGS. 28 and 29.

FIG. 31 shows that mmmAPOE coated constructs traffic only to spleen. Top Figure, right shows images of organs harvests from mice injected with PBS, normal LNPs, or LNPs coated with either wildtype or mutant APOE purified protein. These are the same mice pictured in FIGS. 13 and 14. Organs are labeled on the images-“LNP” represents LNP only, “MUT” represents purified mutant APOE incubation, “WT” represents incubations with purified wildtype APOE. Bottom Figure shows quantification of luminescence values from ROIs on each of the organs.

FIG. 32 shows that LNPs coated with wildtype APOE show weaker transduction of the liver, and LNPs coated with mutant APOE appear to be completely de-targeted. Left Figure shows subset of images show on FIGS. 28 and 29, showing only normal LNPs and mutant APOE incubated “de-targeted” LNP conditions. Right Figure shows quantification of relative luminescence values in the liver and spleen between these conditions.

FIG. 33 shows a competition assay for binding of mutant ApoE to LNPs in the presence of wild type ApoE, or mouse serum (MS). 1× or 4× concentrated conditioned media for mutant or wild type ApoE and 1% or 10% of MS were used for this assay.

FIG. 34 shows ligand fusion strategy to different position on ApoE protein. The figure shows loop structures on ApoE structure where ligand insertions were planned, and gel images showing expressions of these fused proteins.

FIG. 35 shows ligand fusion strategy to different position on ApoE protein. The figure shows the results for transduction of HEK cells overexpressing Anti-Flag or Anti-MoonTag receptors, with LNPs coated ApoE fused to Flag or MoonTag peptides.

FIG. 36 shows the effects of a double point mutation (I250S_L252S) in lipid binding domain (LBD) of ApoE, or a lipid binding peptide (18A) fused to ApoE, on LNP retargeting efficiency. This figure shows fluorescent microscope images of transductions using LNPs coated with different ApoE constructs,

FIG. 37 shows the effects of a double point mutation (1250S_L252S) in lipid binding domain (LBD) of ApoE, or a lipid binding peptide (18A) fused to ApoE, on LNP retargeting efficiency. This figure shows flow cytometry results for a first independent experiments.

FIG. 38 shows the effects of a double point mutation (I250S_L252S) in lipid binding domain (LBD) of ApoE, or a lipid binding peptide (18A) fused to ApoE, on LNP retargeting efficiency. This figure shows flow cytometry results for a second independent experiments.

FIG. 39 shows the effects of various point mutations in lipid binding domain (LBD) of ApoE on its competition efficiency for LNP coating. The figure shows competition of MoonTag-ApoE with EE-ApoE or 10% mouse serum (MS),

FIG. 40 shows the effects of various point mutations in lipid binding domain (LBD) of ApoE on its competition efficiency for LNP coating. The figure shows competition of EE-ApoE with Moon Tag-ApoE or 10% MS.

FIG. 41 shows the effects of various point mutations in lipid binding domain (LBD) of ApoE on its competition efficiency for LNP coating. The figure shows results of another set of mutations on EE-ApoE.

FIG. 42 shows the effects of various point mutations in lipid binding domain (LBD) of ApoE on its competition efficiency for LNP coating. The figure shows validation experiments for potentially working mutations for EE- and MoonTag-ApoE with 1% MS.

FIG. 43 shows the effects of leucine zipper peptides or several new lipid binding peptides on the competition efficiency of ApoE for LNP coating. This figure shows expression gels for these constructs.

FIG. 44 shows the effects of leucine zipper peptides or several new lipid binding peptides on the competition efficiency of ApoE for LNP coating. This figure shows expression gels for these constructs.

FIG. 45 shows the effects of leucine zipper peptides or several new lipid binding peptides on the competition efficiency of ApoE for LNP coating. This figure shows fluorescent images (RNA is labeled with Cy5 dye) of competition assay of these constructs with 1% mouse serum (MS).

FIG. 46 shows the effects of leucine zipper peptides or several new lipid binding peptides on the competition efficiency of ApoE for LNP coating. This figure shows luminescence readout of the same competition assay.

FIG. 47 shows retargeting efficacies with LNPs coated with CD4-binder on either plain HEK293FT cells or expressing corresponding receptors.

FIG. 48 shows retargeting efficacies with LNPs coated with TFRC-binder on either plain HEK293FT cells or expressing corresponding receptors.

FIG. 49 shows retargeting efficacies with LNPs coated with CD90-binder on either plain HEK293FT cells or expressing corresponding receptors.

FIG. 50 shows transduction level of HEK293T or A549 cells, which is a human lung cancer cell line, with LNPs coated with mutant ApoE fused to EGFR or TFRC minibinders. It is clear that EGFR-minibinders induce transduction on A549 cells as they overexpress EGFR naturally, but not on HEK293FT cells. On the other hand, TFRC minibinders didn't induce retargeting in any of these cell lines as they are not overexpressing transferrin receptor.

FIG. 51 shows transduction level of HEK293T or A549 cells, which is a human lung cancer cell line, with LNPs coated with mutant ApoE fused to EGFR or TFRC minibinders. It is clear that EGFR-minibinders induce transduction on A549 cells as they overexpress EGFR naturally, but not on HEK293FT cells. On the other hand, TFRC minibinders didn't induce retargeting in any of these cell lines as they are not overexpressing transferrin receptor.

FIG. 52 shows transduction level of A549 cells with LNPs coated with mutant ApoE fused to short peptide binders, shown to target different mouse tissues (e.g. heart, muscle, lung, etc.) in the literature. One of the lung targeting peptides induce slightly higher transduction than the others. Transduction efficiency with a kidney targeting peptide was even higher than the one with EGFR minibinder. TPM values from Protein Atlas database show that A549 cells express higher level of LGALS3 which is the target receptor of this peptide, than EGFR. FIG. 52 discloses SEQ ID NOs 271-276 and 276, respectively, in order of appearance.

FIG. 53 (top) shows transduction level of MLE-12 cells, which are mouse lung epithelial cells, with LNPs coated with mutant ApoE fused to CD51 or CD146 targeting scFvs. Different protein to RNA (encapsulated in LNP) ratios, 40×, 10×, 2.5×, 0.625× were tested. MS-10 is 10% mouse serum and used as positive control. FIG. 53 (bottom) shows competition assay of proteins with 10% mouse serum for binding to LNPs.

FIG. 54 (top) shows images with IVIS for the mice in question, measuring photons/s. FIG. 54 (bottom) shows quantification of luminescence from a set of LNP or control injections. From left to right; PBS, uncoated LNP, and LNPs coated with mutant ApoE fused to CD63-, A7B1- or CD117-targeting scFvs, respectively,

FIG. 55 shows images of various organs ex vivo with IVIS for the mice in question, measuring photons/s and quantification of luminescence from a set of LNP or control injections. This figure shows LNP only.

FIG. 56 shows images of various organs ex vivo with IVIS for the mice in question, measuring photons/s and quantification of luminescence from a set of LNP or control injections. This figure shows LNP coated with mutant ApoE fused to CD63-targeting scFvs.

FIG. 57 shows images of various organs ex vivo with IVIS for the mice in question, measuring photons/s and quantification of luminescence from a set of LNP or control injections. This figure shows LNP coated with mutant ApoE fused to A7B1-targeting scFvs.

FIG. 58 shows images of various organs ex vivo with IVIS for the mice in question, measuring photons/s and quantification of luminescence from a set of LNP or control injections. This figure shows LNP coated with mutant ApoE fused to CD117-targeting scFvs.

FIG. 59 shows images of various organs ex vivo with IVIS for the mice in question, measuring photons/s and quantification of luminescence from a set of LNP or control injections. This figure shows summary plot showing quantification of organs from mice treated with each particle,

FIG. 60 shows luminescence images with IVIS for the mice in question, measuring photons/s. From left to right; PBS, uncoated LNP, LNP coated with mutant ApoE fused to CD146-targeting scFv, and LNP coated with mutant ApoE, respectively.

FIG. 61 shows transduction level of MLE-12 and HEK293FT cells with LNPs coated with mutant ApoB either alone or fused to EGFR minibinder, RGD/PK7 peptide, or TFRC minibinder. Different protein to RNA (encapsulated in LNP) ratios, 40×, 10×, 2.5× were tested. MS-10 is 10% mouse serum and used as positive control.

FIG. 62 shows competition assay of proteins with 10% mouse serum for binding to LNPs.

FIG. 63 (top) shows luminescence images with IVIS for the mice in question, measuring photons/s. From left to right; PBS, uncoated LNP, LNP coated with mutant ApoE, and LNP coated with mutant ApoE fused to RGD/PK7 peptide, respectively. FIG. 63 (bottom) shows quantification of luminescence from the whole-body images of the same mice.

FIG. 64 shows: top images of various organs ex vivo with IVIS for the mice in question, measuring photons/s. Bottom shows quantification of luminescence from a set of LNP or control injections. This figure shows LNP only.

FIG. 65 shows: top images of various organs ex vivo with IVIS for the mice in question, measuring photons/s. Bottom shows quantification of luminescence from a set of LNP or control injections. This figure shows LNP coated with mutant ApoE fused to RGD/PK7.

FIG. 66 shows: top images of various organs ex vivo with IVIS for the mice in question, measuring photons/s. Bottom shows quantification of luminescence from a set of LNP or control injections. This figure shows LNP coated with mutant ApoE.

FIG. 67 shows summary plot showing quantification of organs from mice treated with each particle.

FIG. 68 (top) shows luminescence level on either plain HEK293FT or CD5-overexpressing cells, transduced with different doses of LNPs coated with mutant ApoE fused to Protein A/G and incubated with CD5. FIG. 68 (bottom) shows the results for the same experiment by using plain or CD117-overexpressing cells and LNPs coated with Protein A/G+CD117 antibody.

FIG. 69 shows luminescence level on primary mouse T cells, transduced with different doses of LNPs (100 or 375 μg of RNA) coated with mutant ApoE fused to Protein A/G or Streptavidin and incubated with CD3, CD4, CD5, or CD8 antibodies either as unconjugated or biotin conjugated.

FIG. 70 (left) shows fluorescence images of livers and spleens ex vivo with IVIS for the mice in question, measuring photons/s. FIG. 70 (right) shows quantification of fluorescence from a set of LNP or control injections. Injected samples are PBS (1), LNP only (2), LNPs coated with mutant ApoE fused to Protein A/G and incubated with PBS (3), CD4 antibody (4) or CD5 (5) antibody, respectively.

FIG. 71 shows luminescence level on HEK293FT controls and primary mouse T cells, transduced with different doses of LNPs (100 or 375 μg of RNA) conjugated with CD4 or CD5 antibodies.

FIGS. 72A-72C shows that compared to LNP only conditions, the addition of the mutant Apo-E protein produces a two-fold effect of reducing (off-target) liver signal and increase on-target T cell signal. FIG. 72A shows images of organs showing radiance in photons/s. Spleen (top) and liver (bottom) are shown for 2 mice injected with cd5 LNPs alone or LNPs mixed with protein. FIG. 72B shows radiance of spleens and livers in photons/s. FIG. 72C shows luminescence from purified T cells. ALC_Cd5_camp and ALC_Cd5_camp_mmmApoE (2 ug=˜0.075 mg/kg) Conditions 1,2.

FIGS. 73A-73B show that compared to LNP only conditions, the addition of the mutant Apo-E protein reduces liver background by ˜80% without affecting on-target signal. FIG. 73A shows images of organs showing radiance in photons/s. Spleen (top) and liver (bottom) are shown for 9 mice injected with cd5 LNPs alone or LNPs mixed with protein. FIG. 73A left shows results for ALC_Cd5_chol, ALC_Cd5_camp, ALC_Cd5_chol+mmmApoE (90×), and ALC_Cd5_DOPE_camp+mmmApoE (90×)-(1 ug=˜0.05 mg/kg), Conditions 1,2,5,8. FIG. 73A rights shows ALC_Cd5_DOPE, ALC_Cd5_DOPE_camp, ALC_Cd5_camp+mmmApoE (90×), ALC_Cd5_DOPE+mmmApoE (90×)−PBS (1 ug=˜0.05 mg/kg), Conditions 3,4,6,7. FIG. 73B shows liver radiance with and without mutant ApoE at 90× dose from all mice. FIG. 73C shows T cell luminescence post purification from each mouse.

FIGS. 74A-74C show that the luminescence signal in the organ images is almost entirely contributed by the T cells, which the CD5 antibody targets. FIG. 74A shows images of various organs ex vivo with IVIS, measuring photons/s or luminescence from a set of LNP or control injections. ROIs calculated per organ to quantify the signal. FIG. 74B shows a list of conditions and doses for LNP injections. FIG. 74C shows quantification of luminescence signals from T cells isolated from mice injected with CD5-conjugated fluc LNPs, unconjugated LNPs or PBS.

FIGS. 75A-75B show that the luminescence signal in the organ images is contributed by the T cells, which the CD5 antibody targets. FIG. 75A shows images of organs showing radiance in photons/s. Spleen (top) and liver (bottom) are shown for 5 mice. FIG. 75B shows luminescence of T cells extracted from spleens for PBS and ALC mice.

FIGS. 76A-76B show hat compared to LNP only conditions, the addition of the mutant Apo-E protein produces a two-fold effect of reducing (off-target) liver signal and increase on-target T cell signal. FIG. 76A shows images of organs showing radiance in photons/s. Spleen (top), liver (bottom), and bone marrow (top right). Images are shown for 5 mice. FIG. 76B shows radiance measurements for livers and spleens in pictured organs.

FIGS. 77A-77B show that that antibodies conjugated with CD5 using SATA or TCEP chemistry cause strong luminescence in both the liver (bottom organ in arrangements) and spleen (top left organ). FIG. 77A shows images of various organs ex vivo with IVIS, measuring photons/s or luminescence from a set of LNP or control injections. FIG. 77B shows same image but with ROIs calculated per organ to quantify the signal.

FIGS. 78A-78B show that the luminescence signal in the organ images on the preceding slide is almost entirely contributed by the T cells, which the CD5 antibody targets. FIG. 78A shows quantification of luminescence signals from T cells isolated from mice injected with CD5-conjugated fluc LNPs or PBS. FIG. 78B shows same data but acquired with higher gain on a Biotek spectrophotometer.

FIG. 79 shows images of T cells isolated from loxP-tdTomato mic injected with CD5-conjugated LNPs carrying Cre mRNA showing positive fluorescence. Day 2-Cd5 conj Cre-LNP on T cells isolated from loxP-tdTomato mice.

FIG. 80 shows luminescence of T cells and HSCs purified from mice shown in the preceding drawing.

### DETAILED DESCRIPTION OF THE INVENTION

Lipid nanoparticles (LNPs) have emerged as a preferred delivery modality, especially for first small interfering RNA (siRNA) and mRNA delivery, for a number of reasons: 1) Ease of manufacturing, 2) Reduced immunogenicity and redosability, and 3) Clinically mature development, including for the first small siRNA drug, Onpattro, and the mRNA vaccines used for SARS-COV-2 vaccines. Despite these advantages, LNPs suffer from a critical shortcoming: as they are primarily absorbed by hepatocytes when systemically injected, LNPs have been difficult to retarget to extrahepatic tissues. LNP engineering has been attempted for new organs through a variety of methods including: 1) LNP component screening to identify formulations that target new tissues, 2) supplementation of LNP formulations with additional components to target new tissues, and 3) conjugation of binding domains, like antibody fragments, to LNPs for retargeting. While these approaches have expanded the targeting scope of LNP to new tissues including lung, spleen, T cells, and endothelial cells, they suffer from multiple shortcomings: 1) insufficient de-targeting of the liver to enable therapeutic utility, 2) difficulties in scale up and manufacturing, and 3) inability to rationally program tropism to new tissues and targets. These limitations substantially reduce the broad applicability of these novel LNP formulations for in-human therapeutic use.

LNP interaction with human biology underlies the mechanism of targeting: adsorption of blood proteins to the surface of the particle form a protein corona, influencing the stability, uptake, and clearance of the particles. Binding of these corona proteins to cell surface receptors mediates the uptake of LNPs and release of cargo, as was found for ApoE-mediated uptake of commonly used LNP formulations, or for β2-GPI or Vtn-mediated uptake of retargeted spleen- or lung-targeting LNPs, respectively. These instances support that indirect manipulation of the LNP corona can be tailored to deliver nucleic acids to specific sites in live animals. We explored and leveraged this biological mechanism to directly engineer a synthetic protein corona, creating a new method for delivery, protein corona LNPs (pcLNPs).

To engineer pcLNPs, we explored the natural protein corona of a clinically validated LNP formulation, MC38, to determine if corona components can be engineered as a general lipid-binding scaffold for presentation of receptor binding domains to retarget delivery. This process of binder presentation is directly analogous to viral capsid engineering for the presentation of binding peptides, which has been successful for redirection of adeno-associated virus (AAV) vectors to novel tissues. We determined suitable regions of these nominated lipid binding domains for presentation of binders, including known receptor-targeting single-chain variable fragment (scFv) binders and peptide binders. Furthermore, we used engineering of lipid binding domains to improve stability of pcLNP candidates. We developed novel pcLNP formulations towards new cell types, tested these pcLNPs in vitro and in vivo, and showcased pcLNP with genome editing and mRNA delivery applications. The simplicity of the pcLNP formulation process makes it compatible with existing LNP manufacturing processes, allowing for a scalable and cost effective method that is compatible with future manufacturing. pcLNPs convert LNP design into a programmable protein problem, generating many new formulations for desirable tissue targets and creating many new therapies for patients.

The optimal delivery modality is reprogrammable and scalable: scalable—easy to produce and does not rely on cellular manufacturing, reprogrammable—can be easy targeted to specific tissues or cell types, orthogonal—does not have natural off target tissue uptake (e.g., liver), flexible—compatible with multiple payloads including DNA, mRNA, siRNA, small molecules, and redosable—does not provoke immune response.

Proposed herein is a revolutionary approach in LNP design, focusing on the exploration and engineering of natural binding proteins within its protein corona to achieve targeted delivery. Unlike conventional strategies which attempt to identify new tissue targets through phospholipid and cholesterol component screening or adding new components to LNP formulations, this proposal leverages the inherent lipid binding proteins of an extensively used LNP formulation with clinical validation, MC3. The idea of engineering these proteins as a general scaffold for ligand presentation for cell-specific targeting presents an innovative avenue that expands upon state-of-the-art LNP formulation and design approaches, and offers a more direct approach to influencing nanoparticle behavior and targeting in a programmable fashion.

This approach focuses on various protein engineering strategies, with cell-specific binders that de-target the liver. The flexibility of the lipid-binding protein scaffold allows use of a variety of known protein binding ligands to provide extra-hepatic tropism. Moreover, because we are mutagenizing the corona scaffold protein ApoE to eliminate natural receptor binding, we are able to detarget pcLNPs from their native tissue targeting, such as liver, offering an LNP platform that can be targeted to specific tissues. The innovative use of protein engineering in this context promises to improve precision and control over the targeting capabilities of LNPs, a critical factor in enhancing therapeutic efficacy and reducing off-target effects.

In addition, this approach demonstrates applications such as mRNA delivery and genome editing, using the newly developed pcLNP formulations, showcase the broad applicability of the platform. This innovative approach generates a plethora of novel formulations aimed at desirable tissue targets, thereby creating numerous therapeutic opportunities. Whereas other approaches to engineering LNPs with protein ligands has required costly chemical manufacturing workflows that were not amenable to large scale manufacturing that is needed for human therapeutics, our pcLNP formulation strategy is simple, requiring only the addition of a single engineered corona protein to be included during the LNP mixing step. This paves the way for future scalable manufacturing in a cost effective manner, making our technology readily translatable for nucleic acid therapeutics. This is a leap forward in the field, with the potential to revolutionize targeted therapy and provide novel treatment options for patients.

### Developing of Programmable LNP Targeting Via Protein Corona Engineering

We explored and engineered the protein corona that forms on the MC3-based LNP, a key component of Onpattro, the first United States Food and Drug Administration (FDA)-approved siRNA drug. This MC3 formulation is highly versatile, can be repurposed for mRNA delivery, and has been used for a variety of gene replacement and antiviral therapies. Because of the universality of the MC3 nanoparticles, we focused on this formulation as our lipid chassis for protein corona engineering to create retargeted pcLNPs. To develop the protein component of pcLNP, we profile the lipid binding proteins that form the MC3 corona and nominate the strongest binding proteins as chassis for engineering. After producing pcLNP with these purified components, we mutagenize any natural receptor binding domains of the lipid binding proteins to detarget them from native interactions, effectively creating modular platforms for presentation of binding partners. We then explore engineering of these natural receptors for optimal location of binders, including the N-terminus, C-terminus, and flexible solvent-exposed loops. We initially demonstrate the feasibility of binder presentation with synthetic binding pairs, before applying known tissue-specific binders for cell line-specific targeting. This produce a diverse array of pcLNPs, suitable for subsequent refinement and evaluation in vivo.

### (a) Enhancing and Detargeting LNP Uptake by Canonical Receptors Via Manipulation of the Protein Corona

Despite the widespread use of MC3-based lipid nanoparticles, there are no comprehensive analyses of the MC3 LNP-associated protein corona. We explore the MC3 corona in an unbiased fashion, using a combination of SDS-PAGE and mass spectrometry. LNPs are generated with either microfluidic or vortexing, and all nanoparticle formulations are incubated either with human or mouse plasma ex vivo prior to purification via centrifugation to isolate protein bound LNPs. Corona components are denatured and visualized with Coomasie staining on SDS-PAGE gels, providing high level profiling of the corona components, which we compare with other commonly used formulations, such as four-component dendrimer LNPs and LP01. Once we have validated isolation of the corona, we perform unbiased mass spectrometry to develop a quantitative sense of the proteins bound. Using silver staining, we isolate bands from gel electrophoresis separated samples, prepare them for discovery proteomics via trypsin digest, and profile tryptic peptides on a tandem mass spectrometer. To identify putative lipid binding scaffolds, we manually cluster identified proteins by function, including apolipoproteins, coagulation proteins, complement proteins, and immune proteins, and sort the relative abundances to find the top 10 lipid binding proteins. To ensure our findings are not mouse strain or human donor specific, we evaluate serum from multiple different mouse strains, such as C57BL/6J, SJL/J, and BALB/cByJ strains of both sexes, and multiple human donors of both sexes. Top protein candidates that are reproducible across different samples and contexts are selected as putative chassis for engineering.

Proteins are evaluated for both endogenous binding capacity and stability of corona formation. The top 10 candidates are recombinantly expressed and individually incubated with MC3 LNPs, harboring EGFP mRNA, to generate engineered coronas around these LNPs, forming pcLNPs. To preliminarily assess the stability of these interactions, we incubate LNP complexes at 37 C for 12 hours to ensure that particles do not degrade or aggregate at physiological temperatures. We also confirm corona formation via pcLNP purification, western blotting, and mass spectrometry. We evaluate pcLNPs for cellular uptake and mRNA expression in common cell lines, such as HEK293FT, HepG2, Huh-7, and A549 cells, via flow cytometry. Binders that provide the best improvement of transfection efficiency versus uncoated LNPs and show stability are nominated for further development for detargeting from their natural receptors via mutagenesis of receptor binding domains. To mitigate risk of finding novel binders, we also explore the known LNP binders ApoE, β2-GPI, or Vtn5, with demonstrated hepatocyte, spleen, and lung targeting, respectively. We take these proteins through similar corona formation assays to evaluate their resulting pcLNPs.

Initially, we focus on ApoE development: once we verify ApoE pcLNPs are functional, we engineer ApoE to serve as a generic scaffold by mutagenizing the low density lipoprotein receptor (LDLR) binding domain, residues 134-150. Our mutagenesis campaign involve alanine scanning mutagenesis across this entire window, as well as evaluation of mutants with demonstrated disruption of LDLR binding, such as R142A, R142C, R143S, R142A/K143S, R142C/K143S, R145C, and R172A15. Receptor binding is evaluated by measuring the uptake of the LNPs using the EGFP mRNA cellular assay on the HEK293FT and Huh-7 cell lines, which express LDLR. Top mutants are combined to determine if there are synergies and to develop the optimal inactivated scaffold. We scalably screen variants via production and secretion from HEK293FT cells using the natural ApoE secretion signal, allowing for purification-free isolation of ApoE variants by harvesting of cell media. Harvested media is not supplemented with serum, allowing for direct incubation with MC3 LNPs to form pcLNP. Top candidates selected in this way are compared with affinity purified versions to ensure performance is similar. After we have tested and found the optimal ApoE mutant that is capable of binding the LNPs, but that is receptor binding dead (dApoE), we progress this scaffold for engineering and retargeting.

Testing ApoE to derisk the discovery of novel lipid binding proteins, we have established expression and secretion of ApoE from HEK293FT cells, and validated that serum-free media containing wild-type ApoE could be incubated with naked MC3 LNPs to form pcLNPs. We found that for both microfluidic-based and microfluidic-free methods, proteins derived from the corona were detectable by SDS-PAGE. When these pcLNPs containing EGFP mRNA were incubated on HEK293FT cells, which express LDLR, enhanced uptake was observed that was ˜2-4× more efficient by EGFP fluorescence than MC3 LNPs alone. Because the two methods of formulation were similar, we proceeded with microfluidic-free production given the lack of instrumentation requirements.

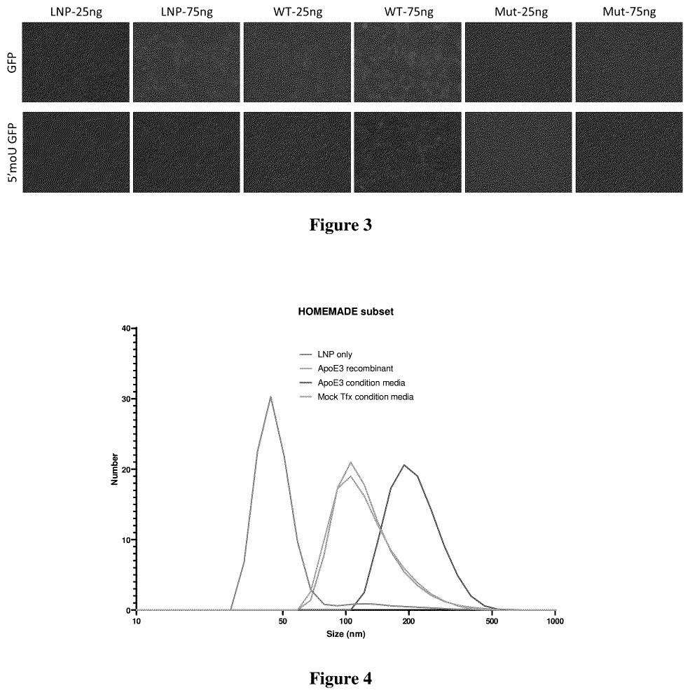

Having confirmed that MC3 LNPs can be converted into pcLNPs with robust corona formation and enhanced cellular uptake, we engineered them by mutating the ApoE receptor binding domain region. Informed by previous mutagenesis of ApoE, we cloned and expressed a specific ApoE mutant, ApoER142A/K143S, formed EGFP mRNA-containing pcLNPs with this candidate, and evaluated cellular uptake on HEK293FT cells. This ApoER142A/K143S mutant completely eliminated cellular uptake, with even lower transfection efficiency than MC3-only LNPs (FIG. 3c-d). We confirmed protein corona formation of ApoER142A/K143S by SDS-PAGE, strongly suggesting that this mutant was blocking cellular binding and uptake. This key finding confirms that the protein corona can be used for engineered modulation of LNP uptake and specifically that the ApoER142A/K143S can serve as a dApoE scaffold for presentation of binders.

(b) Retargeting LNP Uptake with Synthetic Binders

After demonstrating the feasibility of engineering the protein corona of MC3 LNP in vitro to detarget the natural receptor, we use the pcLNP platform to direct uptake of LNP and their associated cargos to arbitrary receptors and cell types. We engineer pcLNPs by determining optimal locations, orientations, and fusions for presentation of binding domains, allowing for arbitrary binders to be displayed on the surface of the pcLNP via fusion with dApoE. These binders will interact with cell- and tissue-specific surface proteins, allowing for localization to specific cell membranes, uptake, and endocytic escape.

First, we engineer pcLNPs using the dApoE scaffold, that can effectively associate with the lipid surface of MC3 LNPs for corona formation but lacks affinity for LDLR. Dead binding scaffolds prevent off-target delivery, allowing for specific binding to other surface receptors. If more suitable dead binding scaffolds are generated, we additionally evaluate those scaffolds. We initially explore fusions of binding domains to the N- and C-termini of dead binding scaffolds to determine the optimal locations for presentation of ligands. As these locations may be sterically inaccessible for binding, we assess a panel of flexible linkers with various lengths to aid in presentation, including GGGS (SEQ ID NO: 267), (GGS) 5 (SEQ ID NO: 268), and XTEN linkers. We initially present a Flag ligand, which will pair with a cell surface-presented anti-Flag antibody to promote pcLNP uptake in an orthogonal synthetic system. To understand if different fusion architectures affect protein expression and stability, we screen different variants via transfection of coding plasmids into HEK293FT cells and subsequent harvesting of the media for protein isolation. Protein expression and stability are evaluated via western blotting of the harvested media and the data informs if there are dApoE locations more or less amenable for ligand insertion.

Once we have linker architectures that allow for stable expression of N- and C-terminally tagged dApoE, we test these constructs for cell transduction by co-incubation of Flag-presenting dApoE with GFP mRNA-containing LNPs, and then apply the corresponding pcLNP-mRNA formulation to HEK293FT cells expressing anti-Flag antibody receptors. We assess the efficiency of transfection over the course of multiple days via fluorescence microscopy and flow cytometry. Importantly, as we have information on expression of these constructs, we determine correlations between transfection efficiency and dApoE-Flag expression. For constructs that have exceptionally high transfection efficiency in spite of low production yield, we purify these constructs from the cellular media to enable higher amounts of protein during the incubation. To confirm that the findings from linker engineering and the N- and C-termini are not ligand dependent, we also test production yield and transfection efficiency with an HA tag-dApoE construct and a corresponding anti-HA antibody expressed on the HEK239FT cell surface.

In addition to N- and C-terminal locations, we explore flexible loops for suitable presentation of binding domains. Insertion of binding peptides or antibodies for presentation has effectively augmented delivery approaches in AAV, and we use similar methods to screen dApoE regions for effective protein expression and subsequent activity of the pcLNPs. Using multiple structures of ApoE, both in lipid-bound and unbound conformations, we identify putative regions for insertion of binding domains based on regions that appear to be exposed or flexible, such as numerous loops between the alpha helices in the first 200 amino acids of the protein. We test insertion of both HA and Flag ligands, using a variety of linker choices, including GGGS (SEQ ID NO: 267), (GGS) 5 (SEQ ID NO: 268), and XTEN linkers, for both yield when expressed via crude secreted protein into media and for effective delivery of mRNA during pcLNP formulation. As accessibility in these regions may be more dependent on binder choice, we evaluate a whole set of binders, including Sun tag, Moon tag, EE tag, ALFA tag, and Myc tag synthetic ligands. We test this expanded set of tagged-dApoE constructs for purification yield and transfection efficiency as pcLNPs when dosed on HEK293FT cells expressing receptors that bind these epitope tags. We further test these binders for orthogonality, assessing their interactions with the other receptors to determine any potential off-target binding pairs due to residual activity from dApoE itself or cross-binding of the epitopes. By evaluating the crosses between these binders and their cognate receptors, we determine the extent of ligand-receptor driven orthogonality.

Once we have dApoE locations for optimizing the synthetic ligand binders, we assess the effects dApoE concentration on pcLNP production and potency. As over-supplementation with the tagged dApoE may yield to 1) aggregation of the pcLNP, 2) excessive binding to cell surfaces precluding endosomal escape and mRNA release, and 3) excessive immune responses in vivo, we perform titrations of engineered dApoE binders when formulating the pcLNPs, both with and without additional untagged dApoE supplementation to occupy additional binding sites. In parallel, we evaluate this pcLNP concentration panel over different expression levels of the receptors in HEK293FT cells, to model different densities of the cell surface receptors and the resulting effects on pcLNP uptake and delivery efficiency.

We have evaluated an initial set of linkers for presentation of ligands on the N-terminus of dApoE, finding that the expression yields of engineered binder-dApoE proteins are substantially ligand dependent. This dependency suggests that there is some steric hindrance or disruption of the dApoE structure, leading to destabilization of the construct. We have evaluated a panel of these synthetic ligand binders, produced as crude supernatants from HEK293FT cells, for production of pcLNPs and subsequent transfection of HEK293FT cells, with and without the corresponding receptors, finding that uptake was receptor dependent. To compensate for low yields of dApoE fusions, we also tested insertion of binders into various flexible loops, finding that these engineered dApoE constructs could rescue expression. To account for the low yield of poorly expressing dApoE constructs, we purified these engineered dApoE constructs via Ni-NTA pulldown of incorporated 6×His-tag tags (SEQ ID NO: 270) and doses these pcLNPs on HEK293FT cells, finding that purified dApoE binders effectively de-targeted natural binding.

After establishing preliminary sites for insertion, we tested an initial panel of tagged dApoE constructs (EE-tag, MoonTag, and SunTag) carrying EGFP mRNA for orthogonality, finding that these tags were completely selective for their cognate receptors with only minimal off-targets. Moreover, when matched with the correct receptor, these engineered pcLNPs had high transfection efficiency in the range of 7-9%. For the best performing binder-cognate pairs, we tested a titration of dApoE concentrations, finding that intermediate levels of binder-dApoE were most effective at aiding in pcLNP transfection.

### (c) Targeting LNPs Towards Tissue-Relevant Receptors

Building upon our demonstration of programmable pcLNP uptake by synthetic receptors, we leverage natural ligand-receptor interactions to achieve cell-specific targeting. We adapt the optimal dApoE insertion position to engineer pcLNPs with ligands that target specific receptors on desired cell types. First, we explore fusion of a variety of binding domains at optimized binding locations to test retargeting towards cell-specific receptors. Our panel of cell-specific binders include vitronectin, EGFR DARPin, various anti-CD scFvs, myomixer, integrin-binding peptides, (KKEEE) 3K binder (SEQ ID NO: 269), and cubilin scFv, to target the specific receptors aVbeta3 (lung epithelial cells), EGFR (lung epithelial cells), CD34 (hematopoietic stem cells), CD90 (hematopoietic stem cells), CD117 (hematopoietic stem cells), CD19 (B cells), CD20 (B cells), CD3 (T cells), CD5 (T cells), CD4 (T cells), myomaker (myocytes), Megalin (kidney epithelial cells), and Cubilin (kidney epithelial cells), respectively. Given the success of the N-terminal region and the receptor binding domain for dApoE expression and cell re-targeting, we include those regions for testing.

Given the potential for steric hindrance affecting the binding, especially with more complex binders such as scFvs, we employ a panel of linkers, including GGGS (SEQ ID NO: 267), (GGS) 5 (SEQ ID NO: 268), and XTEN linkers, to facilitate ligand presentation. To evaluate the effect of different fusion architectures on protein stability and expression, we screen variants for expression in HEK293FT cells using our secretion system, and expression in harvested media is measured via western blotting. If we encounter binder-specific issues with expression, we screen additional for refining any specific ligands.

Upon determining linker architectures for stable expression of ligand-tagged dApoE pcLNPs, we proceed to test these constructs for cell delivery. EGFP mRNA is packaged into pcLNP with engineered dApoE variants, and these pcLNP are dosed on HEK293FT cells overexpressing the desired receptors described above. The efficiency of transfection are monitored over multiple days using fluorescence microscopy and flow cytometry. We also test all pairings of ligand pcLNPs with receptors to evaluate orthogonality and specificity of these constructs, as well as ensuring that LDLR detargeting is maintained. As a stringent demonstration of retargeting, we apply these pcLNP to cell lines with endogenous expression of these receptors, allowing for evaluation of pcLNP retargeting against physiological levels of receptors. The cell lines we use for these tests, categorized by receptor type, include: 1) Lung receptors: A549, A-498, and 16HBE cells; 2) Kidney receptors: RPTEC, HEK293FT, and Caco-2 cells; 3) hematopoietic stem cell (HSC) receptors: HUVEC, HEL cells, and primary human HSCs; 4) T cell receptors: Jurkat, HEL cells, and primary human T cells; 5) B cell receptors: REH cells and primary human B cells; and 6) Muscle receptors: Rh30 cells and primary muscle cells. By using these diverse cell lines, each naturally expressing the desired target receptors, we accurately and robustly validate our constructs prior to in vivo development. These results corroborate our prior findings with the HEK293FT overexpression system and provide valuable information on our constructs' functionality in a broader physiological context.

To pilot binder presentation prior to identifying the optimal ApoE insertion site, we fused an scFv at the N-terminus of dApoE. We chose the CD19/CD19-scFv receptor/ligand pair, which has relevance for B cell targeting in vivo, and found that CD19scFv-dApoE protein could be expressed and readily secreted in HEK239FT cells. CD19scFv-dApoE was incubated with MC3 to generate pcLNP, which were tested on HEK293FT cells expressing the CD19 receptor. CD19-tagged corona pcLNPs could readily transfect and express EGFP in these cells, as measured by flow cytometry, and that untagged dApoE corona pcLNPs had no detectable transfection on the CD19 receptor expressing HEK293FT cells. These results derisk the concept that larger binders can be fused to dApoE and used for programmable targeting of cell-specific surface receptors.

While we focused on ApoE on engineered LNP coronas, other lipid binding proteins can serve as better corona scaffolds for ligand fusion and presentation to receptors. In addition to relying on the mass spectrometry data to guide corona scaffold choice, we also explore other known LNP corona proteins, such as B2-GPI, or Vtn, which have been shown to be useful for natural spleen and lung targeting respectively, for their binding to MC3 and reprogramming potential. We explore mutating the receptor binding domain of these proteins to detarget them from their natural receptors and engineer ligand fusions at different sites in the proteins to evaluate the stability and expression yield of different engineered constructs. We then compare these pcLNPs to the dApoE pcLNPs for overall retargeting efficiency on different receptors and cell types. There is a large space of potential LNP corona binders to choose from and an even larger engineering space, which provides multiple avenues for exploration and many chances to succeed with pcLNP retargeting.

Characterization and Engineering of pcLNP Stability

The successful application of pcLNPs for targeted nucleic acid delivery is contingent upon the stability of the protein corona in vivo. Degradation of the engineered corona, particularly when challenged with serum proteins, could lead to both reduced potency to novel tissues and regression of targeting back to the liver. We focus on characterizing and optimizing the stability of engineered pcLNPs under physiological conditions. First, we conduct a rigorous characterization of pcLNP stability via challenge with serum proteins and subsequent evaluation of the integrity of the engineered corona. Data such as dynamic light scattering, western blotting, and retargeting efficiency provide valuable insights into the stability of the pcLNPs under these physiological conditions. Once we have profiled stability, we use this data to improve the stability of pcLNPs, by introducing lipid-binding and serum stability tags to improve the corona stability and strength. In addition to engineering for stability, we also explore the potential benefits of co-binders to allow for AND-gate based targeting of two receptors on a cell. This provides critical insights into how corona modifications can improve pcLNP stability and expression in vivo and how we further improve the pcLNP targeting capabilities.

### (a) Characterization of Protein Corona Stability

We comprehensively characterize protein corona stability of pcLNPs, with a particular focus on whether the corona can survive challenge by serum proteins that would be a risk in vivo. To quantify stability, we perform crosses, challenging wild-type ApoE pcLNPs with dApoE protein at varying concentrations and ApoE pcLNPs with wild-type ApoE. We evaluate the competition assay at various timepoints of competition, including 30 minutes, 1 hour, 2 hours, 6 hours, 12 hours, and 24 hours, by dosing the post-challenged pcLNPs on cells and evaluating transfection efficiency. This approach allows us to observe any potential changes in transfection efficiency, offering insights into the robustness of the protein corona and susceptibility to swapping with exposed lipid-binding proteins. In the case of the wild-type ApoE pcLNPs, if they are susceptible to swapping and corona instability, we find decreased transfection efficiency of HEK293FT cells due to swapping with dApoE. Conversely, in the case of the dApoE pcLNPs, we expect increased transfection efficiency of HEK293FT cells due to swapping with wild-type ApoE proteins.

We also subject pcLNPs to human and mouse serum challenges, aiming to detect alterations in retargeting efficiency due to swapping of our engineered corona with different lipid binding proteins in either human or mouse serum. Given that serum is composed of a complex mixture of proteins, these tests simulate the in vivo conditions our pcLNPs would encounter, thus providing a real-world measure of corona stability. We incubate our Flag-tag dApoE pcLNPs with different concentrations of mouse or human serum and evaluate whether targeting of anti-Flag receptor expressing HEK293FT cells is maintained or decreased due to dissolution of the pcLNP corona. Incubations are also performed for different amounts of time as described above to simulate the amount of time the pcLNPs would be exposed to blood. To confirm the stability of the pcLNPs after serum treatment, we purify pcLNPs and profile the change in corona composition by SDS-PAGE and mass spectrometry.

We also evaluate if temperature can affect the stability of the corona. We conduct challenges at various temperatures to ascertain any heat stability issues, including room temperature, 37° C., 45° C., and 60° C. The hotter temperatures demonstrate the overall thermostability of the pcLNP corona and inform which storage conditions would be most appropriate for these particles. By systematically evaluating these factors, we gain a deep understanding of pcLNP stability, which inform subsequent engineering efforts.

We evaluated pcLNP corona stability. We have challenged wild-type ApoE pcLNPs with an excess of free dApoE for 0, 1, or 2 hours and tested the resulting impact on pcLNP transfection efficiency. We found minimal reduction in pcLNP transfection efficiency, suggesting that the corona layer around the LNP is stable and undergoes minimal exchange with free lipid binding proteins. Similarly, attempting to rescue dApoE coated pcLNPs via challenge with wild-type ApoE proteins for 0, 1, or 2 hours did not increase transfection efficiency, suggesting that the dApoE corona was refractory to replacement by exogenous wild-type ApoE. While these results support the conclusion that the pcLNP corona is stable enough to remain intact when used in vivo, we further evaluated stability using human serum as a challenger to corona stability to control for other lipid-binding proteins that could displace the ApoE corona. Incubating Flag-tag dApoE pcLNPs with human serum for 0, 1, or 2 hours and testing delivery efficiency on HEK293FT cells expressing the anti-Flag receptor, we found there were minimal effects on transfection efficiency. This suggests that the corona is indeed stable and that in vivo it should remain intact and guide the pcLNPs to their appropriate cell target.

(b) Engineering Improved pcLNP Stability

Once we have characterized the stability of our initial dApoE formulations, we use this information to improve the strength of the lipid-dApoE interaction and the stability of the particles in serum. To augment the natural interaction of ApoE with the LNP interface, we explore fusing a panel of lipid-binding peptides, including 18A, 4F, and ETC-642, to either the N- or C-terminus of the dApoE protein. We initially fuse 1, 2, or 3 repeats of these peptides at either termini, and test for stability of the resulting pcLNP using the panel of assays. Furthermore, we mutagenize the ApoE protein, in both the lipid binding region and in the N-terminal fragment to improve affinity for the LNP complex. It is known that natural ApoE variants, including ApoE3 and ApoE4, have different binding affinities for lipids, and we screen mutations in the lipid binding domain of ApoE to determine if they maintain or improve transfection of a Flag-fused dApoE. We also examine displacement of these peptide- or mutation-enhanced dApoE corona pcLNPs via challenge with unmodified dApoE, as well as serum incubation, and assess over time the relative transfection efficiencies of the resulting pcLNPs. This engineering yield improved LNP corona stability and prevent dissolution of the pcLNPs in later in vivo applications. Importantly, we assess this dApoE stability assay with both Flag-tagged dApoE, evaluated on anti-Flag presenting HEK293FT cells, as well as EGFR-DARPin-dApoE, evaluated on A549 lung cell lines, to determine the generalizability of this engineered interaction.

As protein stability in vivo is potentially compromised by binding with additional serum proteins, including serum proteases, we explore methods to protect pcLNP from degradation, or clearance. We use serum-stabilizing tags, including XTEN, to aid in steric shielding of the pcLNP and extend circulation time for the constructs. These constructs are evaluated for improved stability in the presence of serum proteases by incubation with serum over 72 hours and subsequent assessment of protein purity by western blot. Top stabilizing tags are tested for stability in vivo. We inject mice with pcLNP variants engineered for improved serum stability and measure the half-life of these candidates by harvesting blood and quantifying both the mRNA payload and Flag-dApoE levels with qPCR and ELISA, respectively. As the dApoE protein does not have uptake to tissues due to its detargeting, loss of circulating pcLNP is indicative of either off-target uptake or degradation. We select dApoE variants with improved pcLNP persistence in the serum, and use these in subsequent aims for higher efficiency delivery.

We have performed initial tests with a panel of lipid binding proteins fused to ApoE to enhance the stability of binding. By tethering 4F or 18A peptides at the C-terminus, replacing the lipid binding region, we can improve transduction of GFP mRNA into HEK293FT cells relative to wild type ApoE coronas. Furthermore, introduction of point mutations into the lipid binding region of ApoE substantially increases the transduction efficiency of these particles, demonstrating that multiple engineering approaches can generate improved pcLNPs, as measured by HEK293FT transduction.

(c) Enhancing pcLNP Targeting with Soluble Co-Binders

The ability to deliver to cells based on a logical operation of receptors, rather than on the presence of a single receptor, greatly increases the specificity of pcLNP-mediated delivery. We develop pcLNPs that can require two receptors on a cell for delivery, rather than one. In many instances, two receptors may be needed to properly target a cell type and avoid off-target delivery to undesired cell types. Recently, a technology called Co-localization-dependent Latching Orthogonal Cage/Key protein (Co-LOCKR) was developed, which allows protein dependent recognition of two receptors and actuation based off of that recognition. The system relies on two synthetic proteins, referred to as the “Cage” and “Key”, which can be designed to recognize specific and distinct cell surface markers. These proteins only activate when they simultaneously bind to designated markers on the cell surface in close proximity, an event that triggers a conformational change in the Co-LOCKR protein structure and reveals a specific ligand that can be targeted by other biologic moieties. We develop the Co-LOCKR technology in tandem with pcLNPs to allow for targeted delivery to cells expressing two receptors.

Previously, Co-LOCKR constructs were designed to recognize cells expressing both HER2 and EGFR and to expose the Bim antigen for recognition. As a proof of principle for pcLNP targeting, we employ these same constructs and use Bcl2, which specifically binds Bim, as the ligand presented on dApoE. Using either the N-terminus of dApoE or the best dApoE insertion position, we engineer Bcl2-dApoE constructs for formulating pcLNPs. We then evaluate their delivery efficiency on HEK293FT cells expressing both EGFR and HER2. The two Co-LOCKR proteins are supplied in tandem with the Bcl2-dApoE pcLNPs and then fluorescence sorting is used to quantify transfection efficiency. We compare this to cells not expressing the receptors or only expressing one of the two receptors to understand the specificity towards both receptors. Overall, the integration of the Co-LOCKR system with pcLNPs aims to create a powerful tool for targeted delivery. By identifying cells with specific combinations of surface markers and using a tailored delivery system, we increase the efficacy and safety of targeted therapeutics.

We also use our described engineering approaches to improve stability. We use alternative lipid-binding proteins, including other Apo proteins like ApoB, ApoC1, ApoC3, ApoD, and ApoJ, as well as B2-GPI or Vtn5.

In Vivo Re-Targeting of LNPs with Engineered Protein Coronas

After engineering pcLNPs for specific delivery and stability, we validate the functionality of our pcLNPs in vivo, using mouse models as a physiologically relevant platform. Given the importance of effective cellular retargeting and liver de-targeting for efficient drug delivery and reduced off-target expression and toxicity, we focus on demonstrating these capabilities within a living animal. HSCs and muscle cells serve as our target cell types, acting as proof-of-concept models that support the versatility of our approach. Importantly, the retargeting of these cells holds great therapeutic potential. Additionally, we investigate the redosing potential of our pcLNPs to understand their effectiveness and tolerance for both chronic treatments as well as to improve the genome editing rates in tissues. This also addresses the critical question of immunogenicity, ensuring our pcLNPs do not evoke an undesirable immune response that could compromise their safety and effectiveness, as well as their ability to be redosed. Finally, in vivo evaluation provides crucial insights into the stability of our protein corona in the presence of complex serum proteins. By integrating these key aspects into a comprehensive in vivo investigation, we strengthen our understanding of pcLNPs' behavior in a physiological context, thereby accelerating the translation of our platform into potential therapeutic applications.

### (a) In Situ Correction of Hematopoietic Disorders Via LNPs for HSC Targeting

HSCs are an important target for nucleic acid therapies and genome editing given the many diseases that originate from this cell type. HSCs serve as the foundation for the entire hematopoietic system, and their intrinsic self-renewal and multilineage differentiation capabilities make HSCs a compelling target for therapeutic strategies aimed at treating various hematopoietic disorders, such as thalassemia, sickle cell anemia, and certain types of leukemia. Specifically, the ability to deliver corrective genes or gene editing tools directly into HSCs in situ, using pcLNPs, could revolutionize the treatment of these conditions.

Current methods for HSC-based therapies often require ex vivo manipulation, where HSCs are extracted, genetically altered, expanded, and then reinfused into patients. While these approaches have shown promise, they also come with substantial challenges. Maintaining the stemness and potential of HSCs outside of their natural environment can be difficult, and there is a risk of off-target effects due to uncontrolled gene editing and DNA damage response pathways affecting their re-engraftment. Clinically, ex vivo therapies are invasive, time-consuming, costly, and may cause clinical complications related to the high-dose chemotherapy needed for condition prior to HSC transplantation. In contrast, in vivo HSC editing offers a more direct, less invasive, and potentially more efficient approach. If successful, this method could preserve the natural stem cell niche, avoid the potential risks associated with ex vivo manipulation, and significantly reduce treatment-related morbidity. Furthermore, it could also democratize access to gene therapies by reducing the cost and logistical burden.

To realize this potential, we use our engineered pcLNPs to deliver gene editing tools directly to HSCs in vivo. We first evaluate the efficiency of HSC targeting in mouse models using a Cre-loxP reporter strain, B6.Cg-Gt (ROSA) 26Sortm9 (CAG-tdTomato) Hze/J (The Jackson Laboratory, Strain. No. 007909), referred to as Ai9 mice. The Ai9 mouse is genetically engineered with a Cre reporter locus, which allows for CAG promoter driven tdTomato expression upon Cre-mediated recombination. Using the Ai9 mouse as a model system allows for a sensitive readout for pcLNP transduction: small amounts of Cre expression, delivered via mRNA with pcLNP, can be detected via flow cytometry of tdTomato+ cells. Having developed HSC-targeting pcLNPs, we employ the HSC-targeting dApoE pcLNP constructs for our in vivo studies, which includes pcLNPs containing dApoE tagged with scFvs against CD34, CD90, or CD117. For improved specificity, we additionally leverage the Co-LOCKR system to target the CD34/CD90 or CD34/CD117 pairs using pcLNPs with dApoE tagged with Bcl2. We track Cre-edited HSCs and their progeny using flow cytometry at different time points post pcLNP systemic delivery via tail vein injection. We also analyze other organs for off-target delivery, including liver, lung, muscle, and spleen. As part of the optimization process, we evaluate pcLNP dose, mRNA to LNP ratios, and animal age with regards to HSC dosing efficacy. Studies incorporate both male and female mice to control for any gender-specific differences.

We test delivery of adenine base editors to correct the sickle cell disease mutation. We employ base editing constructs previously shown to correct the disease like adenine base editors (ABE8e-NRCH) that can convert the SCD allele (HBBS) into Makassar β-globin (HBBG), a non-pathogenic variant. We co-package the ABE8e-NRCH mRNA with the guide RNA targeting the wild-type HBB sequence near the SCD mutation to show we can make efficient edits in the gene in vivo. We explore optimal ratios of these components and test base editing in the Ai9 mice. Furthermore, we investigate the impact of gene editing on the HSCs' ability to self-renew and differentiate into multiple lineages via flow cytometry. Throughout the dosing and readouts, mice are monitored for any signs of adverse effects or immune responses.

### (b) Genome Editing for Genetic Disorders in Skeletal Muscle Via LNP Delivery

The delivery of genetic payloads into skeletal muscle is crucial for the development of gene therapies against muscular diseases, such as Duchenne muscular dystrophy (DMD). This need has led to the development of a range of different muscle-targeting delivery approaches, including retargeted AAV34 and VLP technologies. However, current methods for muscle delivery face significant shortcomings, including potential immunogenicity, production difficulties, and limited payload capacities. We adapt pcLNP-based approaches for skeletal muscle delivery by using optimized muscle ligands, including myomixer and integrin binding peptides. We test skeletal muscle targeting with Ai9 mice, evaluating male and female mice to control for gender-specific differences, and dosing different quantities of pcLNP to determine the efficiency of transduction.

We test a model of restoration of DMD. Studies are conducted using adult mdx mice, an established model for DMD. We inject these mice with either pcLNP or AAV9, both carrying constructs encoding SaCas9 and guide RNAs designed for editing of the DMD gene via exon 23 removal, facilitating the production of a functional version of dystrophin protein. Using RT-PCR and western blotting for the DMD transcript and protein, we then quantify the efficiency of exon 23-deleted DMD mRNA production, as well as the expression of SaCas9 and guide RNAs. We measure phenotypic impact of editing by assessing specific force output of tibialis anterior muscles by the pcLNP-dosed mdx mice relative to both AAV9- and vehicle-dosed mice.

Further, we explore the efficacy of pcLNP in a mouse model of X-linked myotubular myopathy (XLMTM), a condition characterized by severe muscle wasting. For this purpose, we employ Mtm1 knockout (KO) mice that are known to exhibit a dramatic loss of muscle mass and reduced lifespans. These mice are injected with either AAV9 or pcLNP encoding human MTM1, controlled by the MHCK7 promoter. Notably, the dose is substantially lower than those being employed in current clinical trials for XLMTM. We monitor changes in body weight, activity, and survival over an extended period post-injection. Moreover, we quantify vector genome biodistribution, transgene mRNA, and protein expression in the tissues of the Mtm1 KO mice using qPCR and immunofluorescence.

### (c) Characterization of LNP-Protein Corona Immunogenicity and Redosing

One significant advantage of LNP-based approaches as compared to viral delivery methods is the capacity to redose, enabling a broader therapeutic window or the reversible delivery of transient mRNA therapies. Moreover, repeated dosing of genome editing constructs in LNPs has allowed for increasing the efficiency of gene editing in vivo. The engineered protein corona of pcLNPs prompts an assessment of the degree of immune responses triggered by the engineered dApoE and the associated binders. As dApoE proteins are similar to endogenous ApoE, there is no substantial immune responses after dosing, and that multiple administrations are feasible. Nevertheless, we evaluate repeat dosing of animals to measure both repeated efficacy as well as any adverse immune responses.

We repeatedly dose C57BL/6J mice, testing both male and female mice to control for gender-specific differences, with pcLNPs containing firefly luciferase mRNA. For evaluation, we use HSC or skeletal muscle targeting pcLNPs. We inject at day 1, day 15, day 30, and day 45, testing for production of luminescent signal in the intended tissues at each timepoint. Humoral immune responses are evaluated by quantifying dApoE-specific antibody titers in the serum using enzyme-linked immunosorbent assay (ELISA), which will provide insights into the B-cell mediated immune response. Cell-mediated immune responses are assessed through enzyme-linked immune absorbent spot (ELISPOT) assays and intracellular cytokine staining followed by flow cytometry, focusing on T-cell activation, proliferation, and cytokine production postdosing.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

As used herein, the singular forms “a”, “an,” and “the” include both singular and plural referents unless the context clearly dictates otherwise.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given ligand) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

“Immunotherapy” is treatment that uses a subject's immune system to treat cancer and includes, for example, checkpoint inhibitors, cancer vaccines, cytokines, cell therapy, CAR-T cells, and dendritic cell therapy.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, “circular RNA” or “circRNA” means a circular polynucleotide construct that encodes a peptide or protein as defined herein. Preferably, such a circRNA is a single stranded RNA molecule.

The term “replicon RNA” will be recognized and understood by the person of ordinary skill in the art to refer to an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding a peptide or protein as defined herein). Alternatively, the replicase may be provided on an independent coding RNA construct or a coding DNA construct. Downstream of the replicase may be a sub-genomic promoter that controls replication of the replicon RNA.

The terms “RNA” and “mRNA” mean a ribonucleic acid molecule, i.e., a polymer consisting of ribonucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e., ribose, of a first and a phosphate moiety of a second, adjacent monomer. The specific succession of the monomers is called the RNA-sequence. The mRNA (messenger RNA) provides the nucleotide coding sequence that may be translated into an amino-acid sequence of a particular peptide or protein.

The terms “antibody” and “antibodies” have been used interchangeably herein and means any antibody or antibody fragment (whether produced naturally or recombinantly) which retains antigen binding activity. This includes a monoclonal or polyclonal antibody, a single chain antibody, a Fab fragment of a monoclonal or polyclonal antibody, a chimeric antibody, a humanized antibody, a human antibody, a bispecific antibody, a multispecific antibody, or a nanobody.

The term “disease” as used herein, means an interruption, cessation, or disorder of body function, system, or organ. Non limiting examples of disease include malignant diseases, autoimmune diseases, inherited diseases, metabolic disorders, or infectious diseases.

The term “protein A/G” as used herein, means a fusion protein that combines IgG binding domains of both protein A and protein G. The fusion protein may be recombinant. Protein A/G may bind to one or all subclasses of human IgG.

### Nucleic Acid

The term “nucleic acid” as used herein means a polymer comprising two or more nucleotides for example, deoxyribonucleotides or ribonucleotides, either in an unmodified or modified form. The nucleic acid may be either single stranded or double stranded, linear or circular.

The term “nucleotide” as used herein means a ribonucleotide or deoxyribonucleotide. If the term nucleotide is used in the context of RNA, it refers to ribonucleotide, and if it is used in the context of DNA, it refers to deoxyribonucleotide.

The term “ribonucleic acid” or “RNA” has been used interchangeably herein and means a polymer of ribonucleotides. The RNA may be either single stranded or double stranded, linear or circular. The term RNA also includes messenger RNA (mRNA), and non-coding RNA (ncRNA).

The term “deoxyribonucleic acid” or “DNA” has been used interchangeably herein and means a polymer of deoxyribonucleotides. The DNA may be either single stranded or double stranded, linear or circular.

In some embodiments, the nucleic acid encodes an antigen such as, but not limited to: those derived from Cholera toxoid, tetanus toxoid, diphtheria toxoid, hepatitis B surface antigen, hemagglutinin, neuraminidase, influenza M protein, PfHRP2, pLDH, aldolase, MSP1, MSP2, AMA1, Der-p-1, Der-f-1, Adipophilin, AFP, AIM-2, ART-4, BAGE, alpha-fetoprotein, BCL-2, Bcr-Abl, BING-4, CEA, CPSF, CT, cyclin DIEp-CAM, EphA2, EphA3, ELF-2, FGF-5, G250, Gonadotropin Releasing Hormone, HER-2, intestinal carboxyl esterase (iCE), IL13Ralpha2, MAGE-1, MAGE-2, MAGE-3, MART-1, MART-2, M-CSF, MDM-2, MMP-2, MUC-1, NY-EOS-1, MUM-1, MUM-2, MUM-3, p53, PBF, PRAME, PSA, PSMA, RAGE-1, RNF43, RU1, RU2AS, SART-1, SART-2, SART-3, SAGE-1, SCRN 1, SOX2, SOXIO, STEAP1, survivin (BIRC5), Telomerase, TGFbetaR11, TRAG-3, TRP-1, TRP-2, TERT, or WT1; those derived from a virus, such as Cowpoxvirus, Vaccinia virus, Pseudocowpox virus, Human herpesvirus 1, Human herpesvirus 2, Cytomegalovirus, Human adenovirus A-F, Polyomavirus, Human papillomavirus, Parvovirus, Hepatitis A virus, Hepatitis B virus, Hepatitis C virus, Human immunodeficiency virus, Orthoreo virus, Rotavirus, Ebolavirus, parainfluenza virus, influenza virus (e.g., H5N1 influenza virus, influenza A virus, influenza B virus, influenza C virus), Measles virus, Mumps virus, Rubella virus, Pneumovirus, Human respiratory syncytial virus, Rabies virus, California encephalitis virus, Japanese encephalitis virus, Hantaan virus, Lymphocytic choriomeningitis virus, Coronavirus (e.g., SARS-COV-2), Enterovirus, Rhino virus, Poliovirus, Norovirus, Flavivirus, Dengue virus, West Nile virus, Yellow fever virus and varicella; those derived from a bacterium, such as Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli 0151: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica,′ or those derived from a protozoa, e.g., of the genus Plasmodium (Plasmodium falciparum, Plasmodium malariae, Plasmodium vivax, Plasmodium ovale or Plasmodium knowlesi.

The antigen may be an allergen derived from, without limitation, cells, cell extracts, proteins, polypeptides, peptides, peptide mimics of polysaccharides and other molecules, such as small molecules, lipids, glycolipids, and carbohydrates of plants, animals, fungi, insects, food, drugs, dust, and mites. Allergens include but are not limited to environmental aeroallergens; plant pollens (e.g., ragweed/hayfever); weed pollen allergens; grass pollen allergens; Johnson grass; tree pollen allergens; ryegrass; arachnid allergens (e.g., house dust mite allergens); storage mite allergens; Japanese cedar pollen/hay fever; mold/fungal spore allergens; animal allergens (e.g., dog, guinea pig, hamster, gerbil, rat, mouse, etc., allergens); food allergens (e.g., crustaceans; nuts; citrus fruits; flour; coffee); insect allergens (e.g., fleas, cockroach); venoms: (Hymenoptera, yellow jacket, honey bee, wasp, hornet, fire ant); bacterial allergens (e.g., streptococcal antigens; parasite allergens such as Ascaris antigen); viral antigens; drug allergens; hormones (e.g., insulin); enzymes (e.g., streptokinase); and drugs or chemicals capable of acting as incomplete antigens or haptens (e.g., the acid anhydrides and the isocyanates). Where a hapten is used in a composition of the disclosure, it may be attached to a carrier to form a hapten-carrier adduct. The hapten-carrier adduct is capable of initiating a humoral immune response, whereas the hapten itself would not elicit antibody production. Non-limiting examples of haptens are aniline, urushiol (a toxin in poison ivy), hydralazine, fluorescein, biotin, digoxigenin and dinitrophenol.

In other embodiments, the antigen is an antigen associated with a disease where it is desirable to sequester the antigen in circulation, such as for example an amyloid protein (e.g., Alzheimer's disease).

In some embodiments, the nucleic acid regulates or modulates cellular functions.

The term “cellular functions” as used herein means various cellular or biological processes such as, but not limited to, biosynthesis, cell division, cell cycle regulation, cellular metabolism, ion transport, absorption, secretion, homeostasis, replication, transcription, translation, cell signalling, endocytosis, exocytosis, phagocytosis, apoptosis, DNA replication, DNA repair, protein synthesis, gene regulation, cell repair, cell growth, cell differentiation, cellular trafficking, cell proliferation, metabolic pathways etc.

The terms “regulate” or “modulate” or “regulation” or “modulation” have been used interchangeably herein and means an act of controlling a cellular or biological process or to exert a modifying or controlling influence on cellular or biological process.

Messenger RNA (mRNA)

Messenger RNA (mRNA) is a polymer of ribonucleotides that encodes at least a protein or polypeptide or peptide. Typically, an mRNA includes at least a coding region, a 5′ UTR, a 3′ UTR, a 5′ cap and a poly(A) tail. UTR (untranslated regions) flanks the coding region or open reading frame (ORF). The 5′ UTR and the 3′ UTR are sections of the mRNA before the start codon and after the stop codon respectively. The 5′ UTR has a cap (5′ cap) consisting of altered nucleotides. mRNA also contains a polyadenylated region at its 3′ end having adenine nucleotides called poly(A) tail.

In some embodiments, the mRNA may be unmodified or modified or combination of both. The modification may be in the nucleobase of the nucleotide, or sugar moiety of the nucleotide, or the phosphate of the nucleotide. In some embodiments, unmodified mRNA may comprise naturally occurring nucleosides, for example, adenosine, guanosine, cytidine, and uridine. mRNA may comprise one or more modified nucleosides, for example, adenosine analog, guanosine analog, cytidine analog, or uridine analog.

In some embodiments, the one or more modified nucleosides is a nucleoside analog selected from 2-aminoadenosine, 3-methyl adenosine, 7-deazaadenosine, 7-deazaguanosine, 8-oxoadenosine, or 8-oxoguanosine.

In some embodiments, the one or more modified nucleosides is a uridine analog selected from propynyl-uridine, pseudouridine, C5-bromouridine, C5-fluorouridine, C5-iodouridine, C5-propynyl-uridine, 5-aza-uridine, 2-thio-5-aza-uridine, 2-thio-uridine, 4-thio-pseudouridine, 2-thio-pseudouridine, 5-hydroxy-uridine, 3-methyl-uridine, 5-carboxymethyl-uridine, 1-carboxymethyl-pseudouridine, 1-methyl-3-(3-amino-3-carboxypropyl) pseudouridine, 2-thio-2′-O-methyl-uridine, 5-methoxycarbonylmethyl-2′-O-methyl-uridine, 5-carboxymethylaminomethyl-2′-O-methyl-uridine, 3,2′-O-dimethyl-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurino-4-thio-pseudouridine, 1-methyl-pseudouridine, 4-thio-1-methyl-pseudouridine, 2-thio-1-methyl-pseudouridine, 1-methyl-1 deaza-pseudouridine, 2-thio-1-methyl-1-deaza-pseudouridine, dihydro-uridine, dihydro-pseudouridine, 2-thio-dihydro-uridine, 2-thio-dihydro-pseudouridine, 2-methoxy-uridine, 2-methoxy-4-thio-uridine, 4-methoxy-pseudouridine, or 4-methoxy-2-thio-pseudouridine.

In some embodiments, the one or more modified nucleosides is a cytidine analog selected from 5-methylcytidine, C5-propynyl-cytidine, C5-methylcytidine, pseudoisocytidine, 1-methyl-pseudoisocytidine, pyrrolo-pseudoisocytidine, 4-thio-pseudoisocytidine, 4-thio-1-methyl-pseudoisocytidine, 4-thio-1-methyl-1-deaza-pseudoisocytidine, 1-methyl-1-1 deaza-pseudoisocytidine, 4-methoxy-1-methyl-pseudoisocytidine or combinations thereof.

Methods for making modified nucleosides are well known in the art (WO 2020168466, U.S. Pat. Nos. 8,278,036; 8,691,966; 8,748,089; 8,835,108; 9,750,824; 10,232,055; WO2007024708; WO2012135805; WO2013052523; WO2011012316); all of which are incorporated by reference.

In some embodiments, the modified nucleoside is pseudouridine, for example, 1-methyl-pseudouridine, 1-propynyl-pseudouridine, 1-carboxymethyl-pseudouridine, 1-methyl-3-(3-amino-3-carboxypropyl) pseudouridine, 4-methoxy-pseudouridine, or 4-methoxy-2-thio-pseudouridine 4-thio-pseudouridine, 2-thio-pseudouridine, 4-thio-1-methyl-pseudouridine, 2-thio-1-methyl-pseudouridine, dihydro-pseudouridine or combination thereof.

In some embodiments, mRNA is obtained from natural sources (i.e., isolated from the cells), produced using recombinant expression system, or chemically synthesized.

mRNAs according to the present disclosure may be synthesized via in vitro transcription (IVT). Briefly, IVT is typically performed with a DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions may vary according to the specific application. Methods of making mRNA through IVT reaction is well known in the art (see for example, Beckert, Bertrand and Masquida, Benoit Methods in Molecular Biology (2011) 703, 29-41; Brunelle, Julie L. and Green Rachel Methods in Enzymology (2013) 530, 101-114; Kamakaka, Rohinton T. and Kraus W. Lee Current Protocols in Cell Biology (1999) 11.6.1-11.6.17; Kanwal, Fariha et al. Cellular Physiology and Biochemistry (2018) 48:1915-1927; WO2018157153; WO2020185811; WO2022082001)

In some embodiments, the in vitro transcription occurs in a single batch. In some embodiments, IVT reaction includes capping and tailing reactions either co-transcriptionally or separately. A cap analog is added to the in vitro transcription reaction and will be incorporated at the 5′ end of the mRNA during the reaction. Alternative method of capping involves adding the cap post-transcriptionally through an enzymatic reaction. The poly(A) tail can be incorporated into the DNA template sequence, and thus the poly (A) tail will be incorporated into the mRNA by T7 RNA polymerase during the in vitro transcription. Alternative method of tailing involves adding the poly (A) tail post-transcriptionally through an enzymatic reaction. In some embodiments, capping and tailing reactions are performed co-transcriptionally i.e., during the IVT reaction. In some embodiments, capping and tailing reactions are performed separately from IVT reaction.

mRNA produced as a result of IVT reaction may be purified using techniques well known in the art, such as, centrifugation, filtration and/or chromatographic techniques. The purification of mRNA may be accomplished before capping and tailing steps are performed or after capping and tailing. The synthesized mRNA may be purified by ethanol precipitation or filtration or chromatography methods. In some embodiments, tangential flow filtration is used to purify mRNA. In some embodiments, mRNA is purified by chromatographic step. In other embodiments, mRNA is purified by a combination of filtration and chromatography steps.

In some embodiments, a suitable mRNA sequence is an mRNA sequence encoding a protein, peptide, polypeptide or an antibody. In some embodiments, a suitable mRNA sequence is codon optimized for efficient expression in a host cell or organism. Codon optimization typically includes modifying a naturally-occurring or wild-type nucleic acid sequence encoding a peptide, polypeptide or protein to achieve the highest possible expression of peptide, polypeptide, protein or an antibody without altering the amino acid sequence.

Any length of mRNA can be encapsulated in the lipid nanoparticles of the present disclosure. The length of the mRNA used in the lipid nanoparticle of the present disclosure depends on the gene product or protein or protein fragment to be incorporated in the lipid nanoparticle. Thus, mRNA can be very short extending to about a few hundred nucleotides in length or very long extending to about several thousand nucleotides in length. In some embodiments, mRNA is about 0.5 kb, 1 kb, 1.5 kb, 2 kb, 2.5 kb, 3 kb, 3.5 kb, 4 kb, 4.5 kb, 5.5 kb 6 kb, 6.5 kb, 7 kb, 7.5 kb, 8 kb, 8.5 kb, 9 kb, 9.5 kb, 10 kb, 11 kb, 12 kb, 13, kb, 14, kb, 15 kb, 16 kb, 17 kb, 18 kb, 19 kb, 20 kb, 22 kb, 24, kb, 26 kb, 28 kb, or 30 kb in length. In other embodiments, mRNA is about 0.5 to 30 kb, 0.5 to 25 kb, 0.5 to 20 kb in length. In still other embodiments, mRNA is about 1 to 20 kb, 1 to 15 kb, or 1 to 10 kb in length.

In some embodiments, the mRNA is circular. In other embodiments, the mRNA is linear.

In some embodiments, the mRNA is self-amplifying or self-replicating. Self-amplifying or self-replicating mRNA as used herein means an mRNA that self-replicate upon delivery into the cells. Such mRNAs typically contain a replicase, usually derived from an alphavirus, which enables amplification of the original strand of mRNA encoding the protein of interest upon delivery into the cells (Beissert, Tim et al. Molecular Therapy 2020 28:119-128).

mRNA present in the lipid nanoparticle composition may be present in a biologically effective amount or therapeutically effective amount. In some embodiments, the biologically effective amount of mRNA present in the lipid nanoparticle composition is between 0.1 μg to 1000 μg, 0.1 μg to 950 μg, 0.1 μg to 900 μg, 0.1 μg to 850 μg, 0.1 μg to 800 μg, 0.1 μg to 750 μg, 0.1 μg to 700 μg, 0.1 μg to 650, 0.1 μg to 600, 0.1 μg to 550, 0.1 μg to 500 μg, 0.1 to 450 μg, 0.1 μg to 400 μg, 0.1 μg to 350 μg, 0.1 to 300 μg, 0.1 to 200 μg or any range therein. In some embodiments, the biologically effective amount of mRNA present in the lipid nanoparticle composition is from about 0.1 μg to 1000 μg, 0.1 μg to 950 μg, 0.1 μg to 900 μg, 0.1 μg to 850 μg, 0.1 μg to 800 μg, 0.1 μg to 750 μg, 0.1 μg to 700 μg, 0.1 μg to 650, 0.1 μg to 600, 0.1 μg to 550, 0.1 μg to 500 μg or any range therein.

In some embodiments, the biologically effective amount of mRNA present in the lipid nanoparticle composition is 0.1 μg, 0.2 μg, 0.3 μg, 0.4 μg, 0.5 μg, 0.6 μg, 0.7, ug, 0.8 μg, 0.9 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 100 μg, 110 μg, 120 μg, 130 μg, 140 μg, 150 μg, 160 μg, 170 μg, 180 μg, 190 μg, 200 μg, 220 μg, 240 μg, 250 μg, 260 μg, 280 μg, 300 μg, 350 μg, 400 μg, 450 μg, 500 μg, 600 μg, 700 μg, 800 μg, 900 μg, 1000 μg or any portion or fraction thereof.

In some embodiments, the mRNA encodes one or more proteins, one or more antibodies, or combination thereof. The mRNA encoding one or more proteins, or one or more antibodies may belong to any organism such as a prokaryote or a eukaryote, a unicellular organism, a multicellular organism, a virus, a bacterium, a mycoplasma, a protozoan, an animal or a human.

Non-Coding RNA (ncRNA)

The term “non-coding RNA” or “ncRNA” has been used interchangeably to mean any RNA molecule that is not generally translated, but sometimes can, into a polypeptide or protein and includes, long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA) and ribosomal RNA (rRNA). The term ncRNA also includes such RNAs that encode small peptides such as lncRNA.

In some embodiments, the nucleic acid component of the lipid nanoparticle compositions comprises ncRNA. The ncRNA may be naturally occurring or wild type. In other embodiments, the ncRNA may be synthetically produced.

In some embodiments, the ncRNA is single stranded or double stranded.

In some embodiments, the ncRNA is a few nucleotides long to several thousand nucleotides long.

In some embodiments, the ncRNA comprises long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (tRNA), ribosomal RNA (rRNA) or combination thereof.

In some embodiments, the long non-coding RNA (lncRNA) is more than 200 nucleotide long.

### Deoxyribonucleic Acid (DNA)

Any DNA molecule capable of transferring a gene into a cell, for example to express a transcript, can be incorporated into the lipid nanoparticle compositions described herein.

The term “DNA sequence” or “DNA segment” or “gene” has been used interchangeably and mean a segment or sequence of DNA capable of being used to produce a transcript which may either be a messenger RNA (mRNA) or non-coding RNAs (ncRNAs) such as long non-coding RNA (lncRNA), micro RNA (miRNA), small interfering RNA (siRNA), small nucleolar RNA (snoRNA), small nuclear RNA (snRNA), and PIWI-interacting RNA (piRNA), transfer RNA (RNA) or ribosomal RNA (rRNA).

In some embodiments, the DNA molecule is obtained form natural sources. In other embodiments, the DNA molecule is recombinantly or synthetically produced.

In some embodiments, the DNA molecule is modified or unmodified, linear or circular.

In some embodiments, the DNA molecule is double stranded or single stranded.

In some embodiments, the DNA molecule includes a coding sequence or a non-coding sequence.

In some embodiments, the DNA molecule is a few nucleotides long to several thousand nucleotides long.

### Methods of Treatment

Disclosed herein are methods of treating or preventing a disease. The method may comprise administering to a subject in need thereof the composition disclosed herein. The disease may be cancer, an infectious disease or a disease and/or disorder ameliorated by humoral and/or cellular immune response.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

“Humoral immune response” as referred to herein relates to antibody production and the accessory processes that accompany it, such as for example T-helper 2 (Th2) cell activation and cytokine production, isotype switching, affinity maturation and memory cell activation. It also refers to the effector functions of an antibody, such as for example toxin neutralization, classical complement activation, and promotion of phagocytosis and pathogen elimination. The humoral immune response is aided by CD4+Th2 cells and therefore the activation or generation of this cell type is also indicative of a humoral immune response as referred to herein.

A “humoral immune response” as referred to herein may also encompass the generation and/or activation of T-helper 17 (Thl7) cells. Thl7 cells are a subset of helper effector T-lymphocytes characterized by the secretion of host defense cytokines such as IL-17, IL-17F, IL-21, and IL-22. Th 17 cells are considered developmentally distinct from Thl and Th2 cells, and which have been postulated to facilitate the humoral immune response, such as for example, providing an important function in anti-microbial immunity and protecting against infections. Their production of IL-22 is thought to stimulate epithelial cells to produce antimicrobial proteins and production of IL-17 may be involved in the recruitment, activation and migration of neutrophils to protect against host infection by various bacterial and fungal species.

In some embodiments, the antigen encoded by the nucleic acid in the composition of the disclosure may be a cancer or tumor-associated protein, such as for example, a membrane surface-bound cancer antigen which is capable of being recognized by an antibody.

Cancers that may be treated and/or prevented by the use or administration of a composition of the disclosure include, without limitation, carcinoma, adenocarcinoma, lymphoma, leukemia, sarcoma, blastoma, myeloma, and germ cell tumors. In one embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukemia virus-1. A composition of the disclosure may be used for either the treatment or prophylaxis of cancer, for example, in the reduction of the severity of cancer or the prevention of cancer recurrences. Cancers that may benefit from the compositions of the disclosure include any malignant cell that expresses one or more tumor specific antigens.

In some embodiments, the antigen may be a toxin or an allergen that is capable of being neutralized by an antibody.

In some embodiments, the antigen may be an antigen associated with a disease where it is desirable to sequester the antigen in circulation, such as for example an amyloid protein (e.g., Alzheimer's disease). Thus, a composition of the disclosure may be suitable for use in the treatment and/or prevention of a neurodegenerative disease in a subject in need thereof, wherein the neurodegenerative disease is associated with the expression of an antigen. The subject may have a neurodegenerative disease or may be at risk of developing a neurodegenerative disease. Neurodegenerative diseases that may be treated and/or prevented by the use or administration of a composition of the disclosure include, without limitation, Alzheimer's disease, Parkinson's disease, Huntington's disease, and amyotrophic lateral sclerosis (ALS). For example, Alzheimer's disease is characterized by the association of B-amyloid plaques and/or tau proteins in the brains of patients with Alzheimer's disease (see, for example, Goedert and Spillantini, Science, 314:777-781, 2006). Herpes simplex virus type 1 has also been proposed to play a causative role in people carrying the susceptible versions of the apoE gene (Itzhaki and Wozniak, J Alzheimer's Dis 13:393-405, 2008).

In some embodiments, the composition may comprise a mixture of B cell epitopes as antigens for inducing a humoral immune response. The B cell epitopes may be linked to form a single polypeptide.

In some embodiments, the antigen may be any peptide or polypeptide that is capable of inducing a specific humoral immune response to a specific conformation on targeted tumor cells.

In some embodiments, the compositions of the present disclosure may be used to induce humoral and/or cellular immune response in a subject. Accordingly, compositions as described herein may be useful for treating or preventing diseases and/or disorders ameliorated by humoral immune responses (e.g., involving B-cells and antibody production). The compositions may find application in any instance in which it is desired to administer an antigen to a subject to induce a humoral immune response or antibody production.

A humoral immune response, as opposed to cell-mediated immunity, is mediated by secreted antibodies which are produced in the cells of the B lymphocyte lineage (B cells). Such secreted antibodies bind to antigens, such as for example those on the surfaces of foreign substances and/or pathogens (e.g., viruses, bacteria, etc.) and flag them for destruction.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigen to T cells, such as T helper CD4 or cytotoxic CD8, thus propagating the immune response. B cells, as well as T cells, are part of the adaptive immune response which is essential for vaccine efficacy. During an active immune response, induced either by vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the toll-like receptors (TLRs).

Antigen presenting cells, such as dendritic cells, macrophages and B cells, are drawn to vaccination sites and can interact with antigens and adjuvants contained in the vaccine. The adjuvant stimulates the cells to become activated and the antigen provides the blueprint for the target. Different types of adjuvants provide different stimulation signals to cells. For example, Poly I: C (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle et al., Curr Med Chem, 2008; So., J Immunol, 2012, which are incorporated hereby by reference in their entireties).

The compositions of the present disclosure, by stimulating strong antibody responses, may be capable of protecting a subject from a disease, disorder or ailment associated with an antigen capable of inducing a humoral immune response.

Without limitation, this includes for example, infectious diseases, cancers involving a membrane surface-bound cancer antigen which is recognized by an antibody, diseases where it is desirable to sequester antigen in circulation, like amyloid protein (e.g., Alzheimer's disease); neutralizing toxins with an antibody; neutralizing viruses or bacteria with an antibody; or neutralizing allergens (e.g., pollen) for the treatment of allergies.

In some embodiments, the composition may be administered via oral, nasal, rectal or parenteral administration. Parenteral administration includes intravenous, intraperitoneal, intradermal, subcutaneous, intramuscular, transepithelial, intrapulmonary, intrathecal, and topical modes of administration. In some embodiments, the composition is administered via intramuscular, subcutaneous or intradermal injection.

The amount of composition used in a single treatment may vary depending on factor such as the nature of negatively charged molecule to be delivered, the type of formulation, and the size of the subject. One skilled in the art will be able to determine, without undue experimentation, the effective amount of composition to use in a particular application.

The skilled artisan can determine suitable treatment regimes, routes of administration, dosages, etc., for any particular application in order to achieve the desired result. Factors that may be taken into account include, e.g., the nature of a polypeptide to be expressed; the disease state to be prevented or treated; the age, physical condition, body weight, sex and diet of the subject; and other clinical factors.

The subject to be treated may be any vertebrate, preferably a mammal, more preferably a human.

### Sequences

Table 1 shows sequences of constructs used herein.

| Name | DNA sequence |
|---|---|
| hsApoE-wt | ATGAAGGTTCTGTGGGCTGCGTTGCTGGTCACATTCCTGGCAGGATGCCAGGCCAA |
| | GGTGGAGCAAGCGGTGGAGACAGAGCCGGAGCCCGAGCTGCGCCAGCAGACCG |
| | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGCGCAAGCTGCGT |
| | AAGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGATACGC |
| | CTGCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACTAG (SEQ ID NO: 1) |
| hsApoE-wt | MKVLWAALLVTFLAGCQAKVEQAVETEPEPELRQQTEWQSGQRWELALGRFWDYLRW |
| | VQTLSEQVQEELLSSQVTQELRALMDETMKELKAYKSELEEQLTPVAEETRARLSKELQAA |
| | QARLGADMEDVCGRLVQYRGEVQAMLGQSTEELRVRLASHLRKLRKRLLRDADDLQKR |
| | LAVYQAGAREGAERGLSAIRERLGPLVEQGRVRAATVGSLAGQPLQERAQAWGERLRAR |
| | MEEMGSRTRDRLDEVKEQVAEVRAKLEEQAQQIRLQAEAFQARLKSWFEPLVEDMQRQ |
| | WAGLVEKVQAAVGTSAAPVPSDNH* (SEQ ID NO: 2) |
| hsApoE- | ATGAAGGTTCTGTGGGCTGCGTTGCTGGTCACATTCCTGGCAGGATGCCAGGCCAA |
| mut_R142A_ | GGTGGAGCAAGCGGTGGAGACAGAGCCGGAGCCCGAGCTGCGCCAGCAGACCG |
| K143S | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGATACGC |
| | CTGCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACTAG (SEQ ID NO: 3) |
| hsApoE- | MKVLWAALLVTFLAGCQAKVEQAVETEPEPELRQQTEWQSGQRWELALGRFWDYLRW |
| mut_R142A_ | VQTLSEQVQEELLSSQVTQELRALMDETMKELKAYKSELEEQLTPVAEETRARLSKELQAA |
| K143S | QARLGADMEDVCGRLVQYRGEVQAMLGQSTEELRVRLASHLASLRKRLLRDADDLQKR |
| | LAVYQAGAREGAERGLSAIRERLGPLVEQGRVRAATVGSLAGQPLQERAQAWGERLRAR |
| | MEEMGSRTRDRLDEVKEQVAEVRAKLEEQAQQIRLQAEAFQARLKSWFEPLVEDMQRQ |
| | WAGLVEKVQAAVGTSAAPVPSDNH* (SEQ ID NO: 4) |
| Flag_GS10 | ATGAAGGTTCTGTGGGCTGCGTTGCTGGTCACATTCCTGGCAGGATGCCAGGCCgac |
| APOE3_R142A_ | tacaaggacgacgatgacaagggatccggtgggtccggtagtggtggttccAAGGTGGAGCAAGCGGT |
| K143S | GGAGACAGAGCCGGAGCCCGAGCTGCGCCAGCAGACCGAGTGGCAGAGCGGCC |
| | AGCGCTGGGAACTGGCACTGGGTCGCTTTTGGGATTACCTGCGCTGGGTGCAGACA |
| | CTGTCTGAGCAGGTGCAGGAGGAGCTGCTCAGCTCCCAGGTCACCCAGGAACTGA |
| | GGGCGCTGATGGACGAGACCATGAAGGAGTTGAAGGCCTACAAATCGGAACTGGA |
| | GGAACAACTGACCCCGGTGGCGGAGGAGACGCGGGCACGGCTGTCCAAGGAGCT |
| | GCAGGCGGCGCAGGCCCGGCTGGGCGCGGACATGGAGGACGTGTGCGGCCGC |
| | CTGGTGCAGTACCGCGGCGAGGTGCAGGCCATGCTCGGCCAGAGCACCGAGGAG |
| | CTGCGGGTGCGCCTCGCCTCCCACCTGgccagcCTGCGTAAGCGGCTCCTCCGCG |
| | ATGCCGATGACCTGCAGAAGCGCCTGGCAGTGTACCAGGCCGGGGCCCGCGAGG |
| | GCGCCGAGCGCGGCCTCAGCGCCATCCGCGAGCGCCTGGGGCCCCTGGTGGAA |
| | CAGGGCCGCGTGCGGGCCGCCACTGTGGGCTCCCTGGCCGGCCAGCCGCTACA |
| | GGAGCGGGCCCAGGCCTGGGGCGAGCGGCTGCGCGCGCGGATGGAGGAGATGG |
| | GCAGCCGGACCCGCGACCGCCTGGACGAGGTGAAGGAGCAGGTGGCGGAGGTG |
| | CGCGCCAAGCTGGAGGAGCAGGCCCAGCAGATACGCCTGCAGGCCGAGGCCTTC |
| | CAGGCCCGCCTCAAGAGCTGGTTCGAGCCCCTGGTGGAAGACATGCAGCGCCAGT |
| | GGGCCGGGCTGGTGGAGAAGGTGCAGGCTGCCGTGGGCACCAGCGCCGCCCCT |
| | GTGCCCAGCGACAATCACTAG (SEQ ID NO: 5) |
| Flag_GS10_ | MKVLWAALLVTFLAGCQADYKDDDDKGSGGSGSGGSKVEQAVETEPEPELRQQTEWQ |
| APOE3_R142A_ | SGQRWELALGRFWDYLRWVQTLSEQVQEELLSSQVTQELRALMDETMKELKAYKSELEE |
| K143S | QLTPVAEETRARLSKELQAAQARLGADMEDVCGRLVQYRGEVQAMLGQSTEELRVRLAS |
| | HLASLRKRLLRDADDLQKRLAVYQAGAREGAERGLSAIRERLGPLVEQGRVRAATVGSLA |
| | GQPLQERAQAWGERLRARMEEMGSRTRDRLDEVKEQVAEVRAKLEEQAQQIRLQAEAF |
| | QARLKSWFEPLVEDMQRQWAGLVEKVQAAVGTSAAPVPSDNH* (SEQ ID NO: 6) |
| MoonTag_G | ATGAAGGTTCTGTGGGCTGCGTTGCTGGTCACATTCCTGGCAGGATGCCAGGCCaag |
| S10_APOE3_ | aacgagcaggagctgctggagctggacaagtgggccagcctgggatccggtgggtccggtagtggtggttccAA |
| R142A_K143S | GGTGGAGCAAGCGGTGGAGACAGAGCCGGAGCCCGAGCTGCGCCAGCAGACCG |
| | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGATACGC |
| | CTGCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACTAG (SEQ ID NO: 7) |
| MoonTag_ | MKVLWAALLVTFLAGCQAKNEQELLELDKWASLGSGGSGSGGSKVEQAVETEPEPELR |
| GS10_APOE3_ | QQTEWQSGQRWELALGRFWDYLRWVQTLSEQVQEELLSSQVTQELRALMDETMKELK |
| R142A_K143S | AYKSELEEQLTPVAEETRARLSKELQAAQARLGADMEDVCGRLVQYRGEVQAMLGQSTE |
| | ELRVRLASHLASLRKRLLRDADDLQKRLAVYQAGAREGAERGLSAIRERLGPLVEQGRVR |
| | AATVGSLAGQPLQERAQAWGERLRARMEEMGSRTRDRLDEVKEQVAEVRAKLEEQAQ |
| | QIRLQAEAFQARLKSWFEPLVEDMQRQWAGLVEKVQAAVGTSAAPVPSDNH* (SEQ ID |
| | NO: 8) |
| GSG_Flag_ | ATGAAGGTTCTGTGGGCTGCGTTGCTGGTCACATTCCTGGCAGGATGCCAGGCCAA |
| GSG_to_G127_ | GGTGGAGCAAGCGGTGGAGACAGAGCCGGAGCCCGAGCTGCGCCAGCAGACCG |
| APOE3_R142A_ | AGTGGCAGAGCGGCCAGCGCTGGGAACTGGCACTGGGTCGCTTTTGGGATTACCT |
| K143S | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | ggatccggtgactacaaggacgacgatgacaagggttccggaCAGAGCACCGAGGAGCTGCGGG |
| | TGCGCCTCGCCTCCCACCTGgccagcCTGCGTAAGCGGCTCCTCCGCGATGCCGAT |
| | GACCTGCAGAAGCGCCTGGCAGTGTACCAGGCCGGGGCCCGCGAGGGCGCCGA |
| | GCGCGGCCTCAGCGCCATCCGCGAGCGCCTGGGGCCCCTGGTGGAACAGGGCC |
| | GCGTGCGGGCCGCCACTGTGGGCTCCCTGGCCGGCCAGCCGCTACAGGAGCGG |
| | GCCCAGGCCTGGGGCGAGCGGCTGCGCGCGCGGATGGAGGAGATGGGCAGCCG |
| | GACCCGCGACCGCCTGGACGAGGTGAAGGAGCAGGTGGCGGAGGTGCGCGCCA |
| | AGCTGGAGGAGCAGGCCCAGCAGATACGCCTGCAGGCCGAGGCCTTCCAGGCCC |
| | GCCTCAAGAGCTGGTTCGAGCCCCTGGTGGAAGACATGCAGCGCCAGTGGGCCG |
| | GGCTGGTGGAGAAGGTGCAGGCTGCCGTGGGCACCAGCGCCGCCCCTGTGCCCA |
| | GCGACAATCACTAG (SEQ ID NO: 9) |
| GSG_Flag_ | MKVLWAALLVTFLAGCQAKVEQAVETEPEPELRQQTEWQSGQRWELALGRFWDYLRW |
| GSG_to_G127_ | VQTLSEQVQEELLSSQVTQELRALMDETMKELKAYKSELEEQLTPVAEETRARLSKELQAA |
| APOE3_R142A_ | QARLGADMEDVCGRLVQYRGEVQAMLGGSGDYKDDDDKGSGQSTEELRVRLASHLAS |
| K143S | LRKRLLRDADDLQKRLAVYQAGAREGAERGLSAIRERLGPLVEQGRVRAATVGSLAGQP |
| | LQERAQAWGERLRARMEEMGSRTRDRLDEVKEQVAEVRAKLEEQAQQIRLQAEAFQAR |
| | LKSWFEPLVEDMQRQWAGLVEKVQAAVGTSAAPVPSDNH* (SEQ ID NO: 10) |
| GSG_Flag_ | ATGAAGGTTCTGTGGGCTGCGTTGCTGGTCACATTCCTGGCAGGATGCCAGGCCAA |
| GSG_to_M125_ | GGTGGAGCAAGCGGTGGAGACAGAGCCGGAGCCCGAGCTGCGCCAGCAGACCG |
| APOE3_R142A_ | AGTGGCAGAGCGGCCAGCGCTGGGAACTGGCACTGGGTCGCTTTTGGGATTACCT |
| K143S | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGggatccgg |
| | tgactacaaggacgacgatgacaagggttccggaCTCGGCCAGAGCACCGAGGAGCTGCGGG |
| | TGCGCCTCGCCTCCCACCTGgccagcCTGCGTAAGCGGCTCCTCCGCGATGCCGAT |
| | GACCTGCAGAAGCGCCTGGCAGTGTACCAGGCCGGGGCCCGCGAGGGCGCCGA |
| | GCGCGGCCTCAGCGCCATCCGCGAGCGCCTGGGGCCCCTGGTGGAACAGGGCC |
| | GCGTGCGGGCCGCCACTGTGGGCTCCCTGGCCGGCCAGCCGCTACAGGAGCGG |
| | GCCCAGGCCTGGGGCGAGCGGCTGCGCGCGCGGATGGAGGAGATGGGCAGCCG |
| | GACCCGCGACCGCCTGGACGAGGTGAAGGAGCAGGTGGCGGAGGTGCGCGCCA |
| | AGCTGGAGGAGCAGGCCCAGCAGATACGCCTGCAGGCCGAGGCCTTCCAGGCCC |
| | GCCTCAAGAGCTGGTTCGAGCCCCTGGTGGAAGACATGCAGCGCCAGTGGGCCG |
| | GGCTGGTGGAGAAGGTGCAGGCTGCCGTGGGCACCAGCGCCGCCCCTGTGCCCA |
| | GCGACAATCACTAG (SEQ ID NO: 11) |
| GSG_Flag_ | MKVLWAALLVTFLAGCQAKVEQAVETEPEPELRQQTEWQSGQRWELALGRFWDYLRW |
| GSG_to_M125_ | VQTLSEQVQEELLSSQVTQELRALMDETMKELKAYKSELEEQLTPVAEETRARLSKELQAA |
| APOE3_R142A_ | QARLGADMEDVCGRLVQYRGEVQAMGSGDYKDDDDKGSGLGQSTEELRVRLASHLAS |
| K143S | LRKRLLRDADDLQKRLAVYQAGAREGAERGLSAIRERLGPLVEQGRVRAATVGSLAGQP |
| | LQERAQAWGERLRARMEEMGSRTRDRLDEVKEQVAEVRAKLEEQAQQIRLQAEAFQAR |
| | LKSWFEPLVEDMQRQWAGLVEKVQAAVGTSAAPVPSDNH* (SEQ ID NO: 12) |
| GS10_Flag_ | ATGAAGGTTCTGTGGGCTGCGTTGCTGGTCACATTCCTGGCAGGATGCCAGGCCAA |
| to_T83_APOE3_ | GGTGGAGCAAGCGGTGGAGACAGAGCCGGAGCCCGAGCTGCGCCAGCAGACCG |
| R142A_K143S | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCggatccggtgggtccggtagtggtggttccgactaca |
| | aggacgacgatgacaagGGATCCGGAGGGTCAGGATCCGGCGGGAGCCCGGTGGCGG |
| | AGGAGACGCGGGCACGGCTGTCCAAGGAGCTGCAGGCGGCGCAGGCCCGGCTG |
| | GGCGCGGACATGGAGGACGTGTGCGGCCGCCTGGTGCAGTACCGCGGCGAGGTG |
| | CAGGCCATGCTCGGCCAGAGCACCGAGGAGCTGCGGGTGCGCCTCGCCTCCCAC |
| | CTGgccagcCTGCGTAAGCGGCTCCTCCGCGATGCCGATGACCTGCAGAAGCGCCT |
| | GGCAGTGTACCAGGCCGGGGCCCGCGAGGGCGCCGAGCGCGGCCTCAGCGCC |
| | ATCCGCGAGCGCCTGGGGCCCCTGGTGGAACAGGGCCGCGTGCGGGCCGCCAC |
| | TGTGGGCTCCCTGGCCGGCCAGCCGCTACAGGAGCGGGCCCAGGCCTGGGGCG |
| | AGCGGCTGCGCGCGCGGATGGAGGAGATGGGCAGCCGGACCCGCGACCGCCTG |
| | GACGAGGTGAAGGAGCAGGTGGCGGAGGTGCGCGCCAAGCTGGAGGAGCAGGC |
| | CCAGCAGATACGCCTGCAGGCCGAGGCCTTCCAGGCCCGCCTCAAGAGCTGGTTC |
| | GAGCCCCTGGTGGAAGACATGCAGCGCCAGTGGGCCGGGCTGGTGGAGAAGGTG |
| | CAGGCTGCCGTGGGCACCAGCGCCGCCCCTGTGCCCAGCGACAATCACTAG |
| | (SEQ ID NO: 13) |
| GS10_Flag_ | MKVLWAALLVTFLAGCQAKVEQAVETEPEPELRQQTEWQSGQRWELALGRFWDYLRW |
| to_T83_APOE3_ | VQTLSEQVQEELLSSQVTQELRALMDETMKELKAYKSELEEQLTGSGGSGSGGSDYKDD |
| R142A_K143S | DDKGSGGSGSGGSPVAEETRARLSKELQAAQARLGADMEDVCGRLVQYRGEVQAMLG |
| | QSTEELRVRLASHLASLRKRLLRDADDLQKRLAVYQAGAREGAERGLSAIRERLGPLVEQ |
| | GRVRAATVGSLAGQPLQERAQAWGERLRARMEEMGSRTRDRLDEVKEQVAEVRAKLEE |
| | QAQQIRLQAEAFQARLKSWFEPLVEDMQRQWAGLVEKVQAAVGTSAAPVPSDNH* |
| | (SEQ ID NO: 14) |
| GS10_Flag_ | ATGAAGGTTCTGTGGGCTGCGTTGCTGGTCACATTCCTGGCAGGATGCCAGGCCAA |
| to_P84_APOE3_ | GGTGGAGCAAGCGGTGGAGACAGAGCCGGAGCCCGAGCTGCGCCAGCAGACCG |
| R142A_K143S | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGggatccggtgggtccggtagtggtggttccga |
| | ctacaaggacgacgatgacaagGGATCCGGAGGGTCAGGATCCGGCGGGAGCGTGGCG |
| | GAGGAGACGCGGGCACGGCTGTCCAAGGAGCTGCAGGCGGCGCAGGCCCGGCT |
| | GGGCGCGGACATGGAGGACGTGTGCGGCCGCCTGGTGCAGTACCGCGGCGAGGT |
| | GCAGGCCATGCTCGGCCAGAGCACCGAGGAGCTGCGGGTGCGCCTCGCCTCCCA |
| | CCTGgccagcCTGCGTAAGCGGCTCCTCCGCGATGCCGATGACCTGCAGAAGCGCC |
| | TGGCAGTGTACCAGGCCGGGGCCCGCGAGGGCGCCGAGCGCGGCCTCAGCGC |
| | CATCCGCGAGCGCCTGGGGCCCCTGGTGGAACAGGGCCGCGTGCGGGCCGCCA |
| | CTGTGGGCTCCCTGGCCGGCCAGCCGCTACAGGAGCGGGCCCAGGCCTGGGGC |
| | GAGCGGCTGCGCGCGCGGATGGAGGAGATGGGCAGCCGGACCCGCGACCGCCT |
| | GGACGAGGTGAAGGAGCAGGTGGCGGAGGTGCGCGCCAAGCTGGAGGAGCAGG |
| | CCCAGCAGATACGCCTGCAGGCCGAGGCCTTCCAGGCCCGCCTCAAGAGCTGGTT |
| | CGAGCCCCTGGTGGAAGACATGCAGCGCCAGTGGGCCGGGCTGGTGGAGAAGGT |
| | GCAGGCTGCCGTGGGCACCAGCGCCGCCCCTGTGCCCAGCGACAATCACTAG |
| | (SEQ ID NO: 15) |
| GS10_Flag_ | MKVLWAALLVTFLAGCQAKVEQAVETEPEPELRQQTEWQSGQRWELALGRFWDYLRW |
| to_P84_APOE3_ | VQTLSEQVQEELLSSQVTQELRALMDETMKELKAYKSELEEQLTPGSGGSGSGGSDYKD |
| R142A_K143S | DDDKGSGGSGSGGSVAEETRARLSKELQAAQARLGADMEDVCGRLVQYRGEVQAMLG |
| | QSTEELRVRLASHLASLRKRLLRDADDLQKRLAVYQAGAREGAERGLSAIRERLGPLVEQ |
| | GRVRAATVGSLAGQPLQERAQAWGERLRARMEEMGSRTRDRLDEVKEQVAEVRAKLEE |
| | QAQQIRLQAEAFQARLKSWFEPLVEDMQRQWAGLVEKVQAAVGTSAAPVPSDNH* |
| | (SEQ ID NO: 16) |
| GS10_Flag_ | ATGAAGGTTCTGTGGGCTGCGTTGCTGGTCACATTCCTGGCAGGATGCCAGGCCAA |
| to_M125_APOE3_ | GGTGGAGCAAGCGGTGGAGACAGAGCCGGAGCCCGAGCTGCGCCAGCAGACCG |
| R142A_K143S | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGggatccgg |
| | tgggtccggtagtggtggttccgactacaaggacgacgatgacaagGGATCCGGAGGGTCAGGATCC |
| | GGCGGGAGCCTCGGCCAGAGCACCGAGGAGCTGCGGGTGCGCCTCGCCTCCCA |
| | CCTGgccagcCTGCGTAAGCGGCTCCTCCGCGATGCCGATGACCTGCAGAAGCGCC |
| | TGGCAGTGTACCAGGCCGGGGCCCGCGAGGGCGCCGAGCGCGGCCTCAGCGC |
| | CATCCGCGAGCGCCTGGGGCCCCTGGTGGAACAGGGCCGCGTGCGGGCCGCCA |
| | CTGTGGGCTCCCTGGCCGGCCAGCCGCTACAGGAGCGGGCCCAGGCCTGGGGC |
| | GAGCGGCTGCGCGCGCGGATGGAGGAGATGGGCAGCCGGACCCGCGACCGCCT |
| | GGACGAGGTGAAGGAGCAGGTGGCGGAGGTGCGCGCCAAGCTGGAGGAGCAGG |
| | CCCAGCAGATACGCCTGCAGGCCGAGGCCTTCCAGGCCCGCCTCAAGAGCTGGTT |
| | CGAGCCCCTGGTGGAAGACATGCAGCGCCAGTGGGCCGGGCTGGTGGAGAAGGT |
| | GCAGGCTGCCGTGGGCACCAGCGCCGCCCCTGTGCCCAGCGACAATCACTAG |
| | (SEQ ID NO: 17) |
| GS10_Flag_ | MKVLWAALLVTFLAGCQAKVEQAVETEPEPELRQQTEWQSGQRWELALGRFWDYLRW |
| to_M125_APOE3_ | VQTLSEQVQEELLSSQVTQELRALMDETMKELKAYKSELEEQLTPVAEETRARLSKELQAA |
| R142A_K143S | QARLGADMEDVCGRLVQYRGEVQAMGSGGSGSGGSDYKDDDDKGSGGSGSGGSLG |
| | QSTEELRVRLASHLASLRKRLLRDADDLQKRLAVYQAGAREGAERGLSAIRERLGPLVEQ |
| | GRVRAATVGSLAGQPLQERAQAWGERLRARMEEMGSRTRDRLDEVKEQVAEVRAKLEE |
| | QAQQIRLQAEAFQARLKSWFEPLVEDMQRQWAGLVEKVQAAVGTSAAPVPSDNH* |
| | (SEQ ID NO: 18) |
| GS10_Flag_ | ATGAAGGTTCTGTGGGCTGCGTTGCTGGTCACATTCCTGGCAGGATGCCAGGCCAA |
| to_G127_APOE3_ | GGTGGAGCAAGCGGTGGAGACAGAGCCGGAGCCCGAGCTGCGCCAGCAGACCG |
| R142A_K143S | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | ggatccggtgggtccggtagtggtggttccgactacaaggacgacgatgacaagGGATCCGGAGGGTCA |
| | GGATCCGGCGGGAGCCAGAGCACCGAGGAGCTGCGGGTGCGCCTCGCCTCCCA |
| | CCTGgccagcCTGCGTAAGCGGCTCCTCCGCGATGCCGATGACCTGCAGAAGCGCC |
| | TGGCAGTGTACCAGGCCGGGGCCCGCGAGGGCGCCGAGCGCGGCCTCAGCGC |
| | CATCCGCGAGCGCCTGGGGCCCCTGGTGGAACAGGGCCGCGTGCGGGCCGCCA |
| | CTGTGGGCTCCCTGGCCGGCCAGCCGCTACAGGAGCGGGCCCAGGCCTGGGGC |
| | GAGCGGCTGCGCGCGCGGATGGAGGAGATGGGCAGCCGGACCCGCGACCGCCT |
| | GGACGAGGTGAAGGAGCAGGTGGCGGAGGTGCGCGCCAAGCTGGAGGAGCAGG |
| | CCCAGCAGATACGCCTGCAGGCCGAGGCCTTCCAGGCCCGCCTCAAGAGCTGGTT |
| | CGAGCCCCTGGTGGAAGACATGCAGCGCCAGTGGGCCGGGCTGGTGGAGAAGGT |
| | GCAGGCTGCCGTGGGCACCAGCGCCGCCCCTGTGCCCAGCGACAATCACTAG |
| | (SEQ ID NO: 19) |
| GS10_Flag_ | MKVLWAALLVTFLAGCQAKVEQAVETEPEPELRQQTEWQSGQRWELALGRFWDYLRW |
| to_G127_APOE3_ | VQTLSEQVQEELLSSQVTQELRALMDETMKELKAYKSELEEQLTPVAEETRARLSKELQAA |
| R142A_K143S | QARLGADMEDVCGRLVQYRGEVQAMLGGSGGSGSGGSDYKDDDDKGSGGSGSGGS |
| | QSTEELRVRLASHLASLRKRLLRDADDLQKRLAVYQAGAREGAERGLSAIRERLGPLVEQ |
| | GRVRAATVGSLAGQPLQERAQAWGERLRARMEEMGSRTRDRLDEVKEQVAEVRAKLEE |
| | QAQQIRLQAEAFQARLKSWFEPLVEDMQRQWAGLVEKVQAAVGTSAAPVPSDNH* |
| | (SEQ ID NO: 20) |
| GS10_Flag_ | ATGAAGGTTCTGTGGGCTGCGTTGCTGGTCACATTCCTGGCAGGATGCCAGGCCAA |
| to_G200_APOE3_ | GGTGGAGCAAGCGGTGGAGACAGAGCCGGAGCCCGAGCTGCGCCAGCAGACCG |
| R142A_K143S | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCggatccggtgggtccggtagtggtggttccgactacaaggacgacgatgacaagGGATCCGGA |
| | GGGTCAGGATCCGGCGGGAGCCAGCCGCTACAGGAGCGGGCCCAGGCCTGGGG |
| | CGAGCGGCTGCGCGCGCGGATGGAGGAGATGGGCAGCCGGACCCGCGACCGC |
| | CTGGACGAGGTGAAGGAGCAGGTGGCGGAGGTGCGCGCCAAGCTGGAGGAGCAG |
| | GCCCAGCAGATACGCCTGCAGGCCGAGGCCTTCCAGGCCCGCCTCAAGAGCTGG |
| | TTCGAGCCCCTGGTGGAAGACATGCAGCGCCAGTGGGCCGGGCTGGTGGAGAAG |
| | GTGCAGGCTGCCGTGGGCACCAGCGCCGCCCCTGTGCCCAGCGACAATCACTAG |
| | (SEQ ID NO: 21) |
| GS10_Flag_ | MKVLWAALLVTFLAGCQAKVEQAVETEPEPELRQQTEWQSGQRWELALGRFWDYLRW |
| to_G200_APOE3_ | VQTLSEQVQEELLSSQVTQELRALMDETMKELKAYKSELEEQLTPVAEETRARLSKELQAA |
| R142A_K143S | QARLGADMEDVCGRLVQYRGEVQAMLGQSTEELRVRLASHLASLRKRLLRDADDLQKR |
| | LAVYQAGAREGAERGLSAIRERLGPLVEQGRVRAATVGSLAGGSGGSGSGGSDYKDDD |
| | DKGSGGSGSGGSQPLQERAQAWGERLRARMEEMGSRTRDRLDEVKEQVAEVRAKLEE |
| | QAQQIRLQAEAFQARLKSWFEPLVEDMQRQWAGLVEKVQAAVGTSAAPVPSDNH* |
| | (SEQ ID NO: 22) |
| GS10_MoonTag_ | ATGAAGGTTCTGTGGGCTGCGTTGCTGGTCACATTCCTGGCAGGATGCCAGGCCAA |
| to_T83_ | GGTGGAGCAAGCGGTGGAGACAGAGCCGGAGCCCGAGCTGCGCCAGCAGACCG |
| APOE3_R142A_ | AGTGGCAGAGCGGCCAGCGCTGGGAACTGGCACTGGGTCGCTTTTGGGATTACCT |
| K143S | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCggatccggtgggtccggtagtggtggttccaagaac |
| | gagcaggagctgctggagctggacaagtgggccagcctgGGATCCGGAGGGTCAGGATCCGGCG |
| | GGAGCCCGGTGGCGGAGGAGACGCGGGCACGGCTGTCCAAGGAGCTGCAGGCG |
| | GCGCAGGCCCGGCTGGGCGCGGACATGGAGGACGTGTGCGGCCGCCTGGTGCA |
| | GTACCGCGGCGAGGTGCAGGCCATGCTCGGCCAGAGCACCGAGGAGCTGCGGGT |
| | GCGCCTCGCCTCCCACCTGgccagcCTGCGTAAGCGGCTCCTCCGCGATGCCGATG |
| | ACCTGCAGAAGCGCCTGGCAGTGTACCAGGCCGGGGCCCGCGAGGGCGCCGAG |
| | CGCGGCCTCAGCGCCATCCGCGAGCGCCTGGGGCCCCTGGTGGAACAGGGCCG |
| | CGTGCGGGCCGCCACTGTGGGCTCCCTGGCCGGCCAGCCGCTACAGGAGCGGG |
| | CCCAGGCCTGGGGCGAGCGGCTGCGCGCGCGGATGGAGGAGATGGGCAGCCGG |
| | ACCCGCGACCGCCTGGACGAGGTGAAGGAGCAGGTGGCGGAGGTGCGCGCCAA |
| | GCTGGAGGAGCAGGCCCAGCAGATACGCCTGCAGGCCGAGGCCTTCCAGGCCC |
| | GCCTCAAGAGCTGGTTCGAGCCCCTGGTGGAAGACATGCAGCGCCAGTGGGCCG |
| | GGCTGGTGGAGAAGGTGCAGGCTGCCGTGGGCACCAGCGCCGCCCCTGTGCCCA |
| | GCGACAATCACTAG (SEQ ID NO: 23) |
| GS10_MoonTag_ | MKVLWAALLVTFLAGCQAKVEQAVETEPEPELRQQTEWQSGQRWELALGRFWDYLRW |
| to_T83_ | VQTLSEQVQEELLSSQVTQELRALMDETMKELKAYKSELEEQLTGSGGSGSGGSKNEQE |
| APOE3_R142A_ | LLELDKWASLGSGGSGSGGSPVAEETRARLSKELQAAQARLGADMEDVCGRLVQYRGE |
| K143S | VQAMLGQSTEELRVRLASHLASLRKRLLRDADDLQKRLAVYQAGAREGAERGLSAIRERL |
| | GPLVEQGRVRAATVGSLAGQPLQERAQAWGERLRARMEEMGSRTRDRLDEVKEQVAE |
| | VRAKLEEQAQQIRLQAEAFQARLKSWFEPLVEDMQRQWAGLVEKVQAAVGTSAAPVPS |
| | DNH* (SEQ ID NO: 24) |
| GS10_MoonTag_ | ATGAAGGTTCTGTGGGCTGCGTTGCTGGTCACATTCCTGGCAGGATGCCAGGCCAA |
| to_M125_ | GGTGGAGCAAGCGGTGGAGACAGAGCCGGAGCCCGAGCTGCGCCAGCAGACCG |
| APOE3_ | AGTGGCAGAGCGGCCAGCGCTGGGAACTGGCACTGGGTCGCTTTTGGGATTACCT |
| R142A_K143S | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGggatccgg |
| | tgggtccggtagtggtggttccaagaacgagcaggagctgctggagctggacaagtgggccagcctgGGATC |
| | CGGAGGGTCAGGATCCGGCGGGAGCCTCGGCCAGAGCACCGAGGAGCTGCGGG |
| | TGCGCCTCGCCTCCCACCTGgccagcCTGCGTAAGCGGCTCCTCCGCGATGCCGAT |
| | GACCTGCAGAAGCGCCTGGCAGTGTACCAGGCCGGGGCCCGCGAGGGCGCCGA |
| | GCGCGGCCTCAGCGCCATCCGCGAGCGCCTGGGGCCCCTGGTGGAACAGGGCC |
| | GCGTGCGGGCCGCCACTGTGGGCTCCCTGGCCGGCCAGCCGCTACAGGAGCGG |
| | GCCCAGGCCTGGGGCGAGCGGCTGCGCGCGCGGATGGAGGAGATGGGCAGCCG |
| | GACCCGCGACCGCCTGGACGAGGTGAAGGAGCAGGTGGCGGAGGTGCGCGCCA |
| | AGCTGGAGGAGCAGGCCCAGCAGATACGCCTGCAGGCCGAGGCCTTCCAGGCCC |
| | GCCTCAAGAGCTGGTTCGAGCCCCTGGTGGAAGACATGCAGCGCCAGTGGGCCG |
| | GGCTGGTGGAGAAGGTGCAGGCTGCCGTGGGCACCAGCGCCGCCCCTGTGCCCA |
| | GCGACAATCACTAG (SEQ ID NO: 25) |
| GS10_MoonTag_ | MKVLWAALLVTFLAGCQAKVEQAVETEPEPELRQQTEWQSGQRWELALGRFWDYLRW |
| to_M125_ | VQTLSEQVQEELLSSQVTQELRALMDETMKELKAYKSELEEQLTPVAEETRARLSKELQAA |
| APOE3_ | QARLGADMEDVCGRLVQYRGEVQAMGSGGSGSGGSKNEQELLELDKWASLGSGGSG |
| R142A_K143S | SGGSLGQSTEELRVRLASHLASLRKRLLRDADDLQKRLAVYQAGAREGAERGLSAIRERL |
| | GPLVEQGRVRAATVGSLAGQPLQERAQAWGERLRARMEEMGSRTRDRLDEVKEQVAE |
| | VRAKLEEQAQQIRLQAEAFQARLKSWFEPLVEDMQRQWAGLVEKVQAAVGTSAAPVPS |
| | DNH* (SEQ ID NO: 26) |
| GS10_MoonTag_ | ATGAAGGTTCTGTGGGCTGCGTTGCTGGTCACATTCCTGGCAGGATGCCAGGCCAA |
| to_G127_ | GGTGGAGCAAGCGGTGGAGACAGAGCCGGAGCCCGAGCTGCGCCAGCAGACCG |
| APOE3_R142A_ | AGTGGCAGAGCGGCCAGCGCTGGGAACTGGCACTGGGTCGCTTTTGGGATTACCT |
| K143S | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | ggatccggtgggtccggtagtggtggttccaagaacgagcaggagctgctggagctggacaagtgggccagcct |
| | gGGATCCGGAGGGTCAGGATCCGGCGGGAGCCAGAGCACCGAGGAGCTGCGGGT |
| | GCGCCTCGCCTCCCACCTGgccagcCTGCGTAAGCGGCTCCTCCGCGATGCCGATG |
| | ACCTGCAGAAGCGCCTGGCAGTGTACCAGGCCGGGGCCCGCGAGGGCGCCGAG |
| | CGCGGCCTCAGCGCCATCCGCGAGCGCCTGGGGCCCCTGGTGGAACAGGGCCG |
| | CGTGCGGGCCGCCACTGTGGGCTCCCTGGCCGGCCAGCCGCTACAGGAGCGGG |
| | CCCAGGCCTGGGGCGAGCGGCTGCGCGCGCGGATGGAGGAGATGGGCAGCCGG |
| | ACCCGCGACCGCCTGGACGAGGTGAAGGAGCAGGTGGCGGAGGTGCGCGCCAA |
| | GCTGGAGGAGCAGGCCCAGCAGATACGCCTGCAGGCCGAGGCCTTCCAGGCCC |
| | GCCTCAAGAGCTGGTTCGAGCCCCTGGTGGAAGACATGCAGCGCCAGTGGGCCG |
| | GGCTGGTGGAGAAGGTGCAGGCTGCCGTGGGCACCAGCGCCGCCCCTGTGCCCA |
| | GCGACAATCACTAG (SEQ ID NO: 27) |
| GS10_MoonTag_ | MKVLWAALLVTFLAGCQAKVEQAVETEPEPELRQQTEWQSGQRWELALGRFWDYLRW |
| to_G127_ | VQTLSEQVQEELLSSQVTQELRALMDETMKELKAYKSELEEQLTPVAEETRARLSKELQAA |
| APOE3_R142A_ | QARLGADMEDVCGRLVQYRGEVQAMLGGSGGSGSGGSKNEQELLELDKWASLGSGG |
| K143S | SGSGGSQSTEELRVRLASHLASLRKRLLRDADDLQKRLAVYQAGAREGAERGLSAIRERL |
| | GPLVEQGRVRAATVGSLAGQPLQERAQAWGERLRARMEEMGSRTRDRLDEVKEQVAE |
| | VRAKLEEQAQQIRLQAEAFQARLKSWFEPLVEDMQRQWAGLVEKVQAAVGTSAAPVPS |
| | DNH* (SEQ ID NO: 28) |
| XTEN16_Flag_ | ATGAAGGTTCTGTGGGCTGCGTTGCTGGTCACATTCCTGGCAGGATGCCAGGCCAG |
| to_SP_APOE3_ | CGGCAGCGAGACCCCCGGCACCAGCGAGAGCGCCACCCCCGAGAGCgactacaag |
| R142A_K143S | gacgacgatgacaagTCCGGCTCAGAGACCCCAGGCACCTCTGAGTCTGCAACTCCTGA |
| | GTCAAAGGTGGAGCAAGCGGTGGAGACAGAGCCGGAGCCCGAGCTGCGCCAGCA |
| | GACCGAGTGGCAGAGCGGCCAGCGCTGGGAACTGGCACTGGGTCGCTTTTGGGAT |
| | TACCTGCGCTGGGTGCAGACACTGTCTGAGCAGGTGCAGGAGGAGCTGCTCAGCT |
| | CCCAGGTCACCCAGGAACTGAGGGCGCTGATGGACGAGACCATGAAGGAGTTGAA |
| | GGCCTACAAATCGGAACTGGAGGAACAACTGACCCCGGTGGCGGAGGAGACGCG |
| | GGCACGGCTGTCCAAGGAGCTGCAGGCGGCGCAGGCCCGGCTGGGCGCGGACA |
| | TGGAGGACGTGTGCGGCCGCCTGGTGCAGTACCGCGGCGAGGTGCAGGCCATGC |
| | TCGGCCAGAGCACCGAGGAGCTGCGGGTGCGCCTCGCCTCCCACCTGgccagcCT |
| | GCGTAAGCGGCTCCTCCGCGATGCCGATGACCTGCAGAAGCGCCTGGCAGTGTAC |
| | CAGGCCGGGGCCCGCGAGGGCGCCGAGCGCGGCCTCAGCGCCATCCGCGAGC |
| | GCCTGGGGCCCCTGGTGGAACAGGGCCGCGTGCGGGCCGCCACTGTGGGCTCC |
| | CTGGCCGGCCAGCCGCTACAGGAGCGGGCCCAGGCCTGGGGCGAGCGGCTGC |
| | GCGCGCGGATGGAGGAGATGGGCAGCCGGACCCGCGACCGCCTGGACGAGGTG |
| | AAGGAGCAGGTGGCGGAGGTGCGCGCCAAGCTGGAGGAGCAGGCCCAGCAGATA |
| | CGCCTGCAGGCCGAGGCCTTCCAGGCCCGCCTCAAGAGCTGGTTCGAGCCCCTG |
| | GTGGAAGACATGCAGCGCCAGTGGGCCGGGCTGGTGGAGAAGGTGCAGGCTGCC |
| | GTGGGCACCAGCGCCGCCCCTGTGCCCAGCGACAATCACTAG (SEQ ID NO: 29) |
| XTEN16_Flag_ | MKVLWAALLVTFLAGCQASGSETPGTSESATPESDYKDDDDKSGSETPGTSESATPESKV |
| to_SP_APOE3_ | EQAVETEPEPELRQQTEWQSGQRWELALGRFWDYLRWVQTLSEQVQEELLSSQVTQEL |
| R142A_K143S | RALMDETMKELKAYKSELEEQLTPVAEETRARLSKELQAAQARLGADMEDVCGRLVQYR |
| | GEVQAMLGQSTEELRVRLASHLASLRKRLLRDADDLQKRLAVYQAGAREGAERGLSAIR |
| | ERLGPLVEQGRVRAATVGSLAGQPLQERAQAWGERLRARMEEMGSRTRDRLDEVKEQV |
| | AEVRAKLEEQAQQIRLQAEAFQARLKSWFEPLVEDMQRQWAGLVEKVQAAVGTSAAPV |
| | PSDNH* (SEQ ID NO: 30) |
| XTEN16_Flag_ | ATGAAGGTTCTGTGGGCTGCGTTGCTGGTCACATTCCTGGCAGGATGCCAGGCCAA |
| to_T83_APOE3_ | GGTGGAGCAAGCGGTGGAGACAGAGCCGGAGCCCGAGCTGCGCCAGCAGACCG |
| R142A_K143S | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCAGCGGCAGCGAGACCCCCGGCACCA |
| | GCGAGAGCGCCACCCCCGAGAGCgactacaaggacgacgatgacaagTCCGGCTCAGAG |
| | ACCCCAGGCACCTCTGAGTCTGCAACTCCTGAGTCACCGGTGGCGGAGGAGACGC |
| | GGGCACGGCTGTCCAAGGAGCTGCAGGCGGCGCAGGCCCGGCTGGGCGCGGAC |
| | ATGGAGGACGTGTGCGGCCGCCTGGTGCAGTACCGCGGCGAGGTGCAGGCCATG |
| | CTCGGCCAGAGCACCGAGGAGCTGCGGGTGCGCCTCGCCTCCCACCTGgccagcC |
| | TGCGTAAGCGGCTCCTCCGCGATGCCGATGACCTGCAGAAGCGCCTGGCAGTGTA |
| | CCAGGCCGGGGCCCGCGAGGGCGCCGAGCGCGGCCTCAGCGCCATCCGCGAG |
| | CGCCTGGGGCCCCTGGTGGAACAGGGCCGCGTGCGGGCCGCCACTGTGGGCTC |
| | CCTGGCCGGCCAGCCGCTACAGGAGCGGGCCCAGGCCTGGGGCGAGCGGCTG |
| | CGCGCGCGGATGGAGGAGATGGGCAGCCGGACCCGCGACCGCCTGGACGAGGT |
| | GAAGGAGCAGGTGGCGGAGGTGCGCGCCAAGCTGGAGGAGCAGGCCCAGCAGAT |
| | ACGCCTGCAGGCCGAGGCCTTCCAGGCCCGCCTCAAGAGCTGGTTCGAGCCCCT |
| | GGTGGAAGACATGCAGCGCCAGTGGGCCGGGCTGGTGGAGAAGGTGCAGGCTGC |
| | CGTGGGCACCAGCGCCGCCCCTGTGCCCAGCGACAATCACTAG (SEQ ID NO: 31) |
| XTEN16_Flag_ | MKVLWAALLVTFLAGCQAKVEQAVETEPEPELRQQTEWQSGQRWELALGRFWDYLRW |
| to_T83_APOE3_ | VQTLSEQVQEELLSSQVTQELRALMDETMKELKAYKSELEEQLTSGSETPGTSESATPESD |
| R142A_K143S | YKDDDDKSGSETPGTSESATPESPVAEETRARLSKELQAAQARLGADMEDVCGRLVQYR |
| | GEVQAMLGQSTEELRVRLASHLASLRKRLLRDADDLQKRLAVYQAGAREGAERGLSAIR |
| | ERLGPLVEQGRVRAATVGSLAGQPLQERAQAWGERLRARMEEMGSRTRDRLDEVKEQV |
| | AEVRAKLEEQAQQIRLQAEAFQARLKSWFEPLVEDMQRQWAGLVEKVQAAVGTSAAPV |
| | PSDNH* (SEQ ID NO: 32) |
| XTEN16_Mo | ATGAAGGTTCTGTGGGCTGCGTTGCTGGTCACATTCCTGGCAGGATGCCAGGCCAA |
| onTag_to_T | GGTGGAGCAAGCGGTGGAGACAGAGCCGGAGCCCGAGCTGCGCCAGCAGACCG |
| 83_APOE3 | AGTGGCAGAGCGGCCAGCGCTGGGAACTGGCACTGGGTCGCTTTTGGGATTACCT |
| R142A_K14 | GCGCTGGGTGCAGACACTGTCTGAGCAGGTGCAGGAGGAGCTGCTCAGCTCCCAG |
| 3S | GTCACCCAGGAACTGAGGGCGCTGATGGACGAGACCATGAAGGAGTTGAAGGCCT |
| | ACAAATCGGAACTGGAGGAACAACTGACCAGCGGCAGCGAGACCCCCGGCACCA |
| | GCGAGAGCGCCACCCCCGAGAGCaagaacgagcaggagctgctggagctggacaagtgggcca |
| | gcctgTCCGGCTCAGAGACCCCAGGCACCTCTGAGTCTGCAACTCCTGAGTCACCG |
| | GTGGCGGAGGAGACGCGGGCACGGCTGTCCAAGGAGCTGCAGGCGGCGCAGGC |
| | CCGGCTGGGCGCGGACATGGAGGACGTGTGCGGCCGCCTGGTGCAGTACCGCG |
| | GCGAGGTGCAGGCCATGCTCGGCCAGAGCACCGAGGAGCTGCGGGTGCGCCTC |
| | GCCTCCCACCTGgccagcCTGCGTAAGCGGCTCCTCCGCGATGCCGATGACCTGCA |
| | GAAGCGCCTGGCAGTGTACCAGGCCGGGGCCCGCGAGGGCGCCGAGCGCGGC |
| | CTCAGCGCCATCCGCGAGCGCCTGGGGCCCCTGGTGGAACAGGGCCGCGTGCG |
| | GGCCGCCACTGTGGGCTCCCTGGCCGGCCAGCCGCTACAGGAGCGGGCCCAGG |
| | CCTGGGGCGAGCGGCTGCGCGCGCGGATGGAGGAGATGGGCAGCCGGACCCGC |
| | GACCGCCTGGACGAGGTGAAGGAGCAGGTGGCGGAGGTGCGCGCCAAGCTGGAG |
| | GAGCAGGCCCAGCAGATACGCCTGCAGGCCGAGGCCTTCCAGGCCCGCCTCAAG |
| | AGCTGGTTCGAGCCCCTGGTGGAAGACATGCAGCGCCAGTGGGCCGGGCTGGTG |
| | GAGAAGGTGCAGGCTGCCGTGGGCACCAGCGCCGCCCCTGTGCCCAGCGACAAT |
| | CACTAG (SEQ ID NO: 33) |
| XTEN16_ | MKVLWAALLVTFLAGCQAKVEQAVETEPEPELRQQTEWQSGQRWELALGRFWDYLRW |
| MoonTag_to_ | VQTLSEQVQEELLSSQVTQELRALMDETMKELKAYKSELEEQLTSGSETPGTSESATPESK |
| T83_APOE3_ | NEQELLELDKWASLSGSETPGTSESATPESPVAEETRARLSKELQAAQARLGADMEDVC |
| R142A_K143S | GRLVQYRGEVQAMLGQSTEELRVRLASHLASLRKRLLRDADDLQKRLAVYQAGAREGAE |
| | RGLSAIRERLGPLVEQGRVRAATVGSLAGQPLQERAQAWGERLRARMEEMGSRTRDRL |
| | DEVKEQVAEVRAKLEEQAQQIRLQAEAFQARLKSWFEPLVEDMQRQWAGLVEKVQAAV |
| | GTSAAPVPSDNH* (SEQ ID NO: 34) |
| XTEN16_Flag_ | ATGAAGGTTCTGTGGGCTGCGTTGCTGGTCACATTCCTGGCAGGATGCCAGGCCAA |
| to_L198_ | GGTGGAGCAAGCGGTGGAGACAGAGCCGGAGCCCGAGCTGCGCCAGCAGACCG |
| APOE3_ | AGTGGCAGAGCGGCCAGCGCTGGGAACTGGCACTGGGTCGCTTTTGGGATTACCT |
| R142A_K143S | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGA |
| | GCGGCAGCGAGACCCCCGGCACCAGCGAGAGCGCCACCCCCGAGAGCaagaacg |
| | agcaggagctgctggagctggacaagtgggccagcctgTCCGGCTCAGAGACCCCAGGCACCTC |
| | TGAGTCTGCAACTCCTGAGTCAGCCGGCCAGCCGCTACAGGAGCGGGCCCAGGC |
| | CTGGGGCGAGCGGCTGCGCGCGCGGATGGAGGAGATGGGCAGCCGGACCCGCG |
| | ACCGCCTGGACGAGGTGAAGGAGCAGGTGGCGGAGGTGCGCGCCAAGCTGGAGG |
| | AGCAGGCCCAGCAGATACGCCTGCAGGCCGAGGCCTTCCAGGCCCGCCTCAAGA |
| | GCTGGTTCGAGCCCCTGGTGGAAGACATGCAGCGCCAGTGGGCCGGGCTGGTGG |
| | AGAAGGTGCAGGCTGCCGTGGGCACCAGCGCCGCCCCTGTGCCCAGCGACAATC |
| | ACTAG (SEQ ID NO: 35) |
| XTEN16_Flag_ | MKVLWAALLVTFLAGCQAKVEQAVETEPEPELRQQTEWQSGQRWELALGRFWDYLRW |
| to_L198_ | VQTLSEQVQEELLSSQVTQELRALMDETMKELKAYKSELEEQLTPVAEETRARLSKELQAA |
| APOE3_R142A_ | QARLGADMEDVCGRLVQYRGEVQAMLGQSTEELRVRLASHLASLRKRLLRDADDLQKR |
| K143S | LAVYQAGAREGAERGLSAIRERLGPLVEQGRVRAATVGSLSGSETPGTSESATPESKNEQ |
| | ELLELDKWASLSGSETPGTSESATPESAGQPLQERAQAWGERLRARMEEMGSRTRDRL |
| | DEVKEQVAEVRAKLEEQAQQIRLQAEAFQARLKSWFEPLVEDMQRQWAGLVEKVQAAV |
| | GTSAAPVPSDNH* (SEQ ID NO: 36) |
| XTEN16_ | ATGAAGGTTCTGTGGGCTGCGTTGCTGGTCACATTCCTGGCAGGATGCCAGGCCAA |
| MoonTag_to_ | GGTGGAGCAAGCGGTGGAGACAGAGCCGGAGCCCGAGCTGCGCCAGCAGACCG |
| L198_APOE3_ | AGTGGCAGAGCGGCCAGCGCTGGGAACTGGCACTGGGTCGCTTTTGGGATTACCT |
| R142A_K143S | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGA |
| | GCGGCAGCGAGACCCCCGGCACCAGCGAGAGCGCCACCCCCGAGAGCaagaacg |
| | agcaggagctgctggagctggacaagtgggccagcctgTCCGGCTCAGAGACCCCAGGCACCTC |
| | TGAGTCTGCAACTCCTGAGTCAGCCGGCCAGCCGCTACAGGAGCGGGCCCAGGC |
| | CTGGGGCGAGCGGCTGCGCGCGCGGATGGAGGAGATGGGCAGCCGGACCCGCG |
| | ACCGCCTGGACGAGGTGAAGGAGCAGGTGGCGGAGGTGCGCGCCAAGCTGGAGG |
| | AGCAGGCCCAGCAGATACGCCTGCAGGCCGAGGCCTTCCAGGCCCGCCTCAAGA |
| | GCTGGTTCGAGCCCCTGGTGGAAGACATGCAGCGCCAGTGGGCCGGGCTGGTGG |
| | AGAAGGTGCAGGCTGCCGTGGGCACCAGCGCCGCCCCTGTGCCCAGCGACAATC |
| | ACTAG (SEQ ID NO: 37) |
| XTEN16_ | MKVLWAALLVTFLAGCQAKVEQAVETEPEPELRQQTEWQSGQRWELALGRFWDYLRW |
| MoonTag_to_ | VQTLSEQVQEELLSSQVTQELRALMDETMKELKAYKSELEEQLTPVAEETRARLSKELQAA |
| L198_APOE3_ | QARLGADMEDVCGRLVQYRGEVQAMLGQSTEELRVRLASHLASLRKRLLRDADDLQKR |
| R142A_K143S | LAVYQAGAREGAERGLSAIRERLGPLVEQGRVRAATVGSLSGSETPGTSESATPESKNEQ |
| | ELLELDKWASLSGSETPGTSESATPESAGQPLQERAQAWGERLRARMEEMGSRTRDRL |
| | DEVKEQVAEVRAKLEEQAQQIRLQAEAFQARLKSWFEPLVEDMQRQWAGLVEKVQAAV |
| | GTSAAPVPSDNH* (SEQ ID NO: 38) |
| CD4- | ATGAAGGTTCTGTGGGCTGCGTTGCTGGTCACATTCCTGGCAGGATGCCAGGCCGA |
| DARPin_APOE3_ | CCTGGGCAAGAGGCTGCTGGAGGCCGCCAGGGCCGGCCAGGACGACGAGGTGA |
| R142A_K143S | GGATCCTGATGGCCAACGGCGCCGACGTGAACGCCAAGGACGACGAGGGCAGGA |
| | CCAGCCTGCACCTGGCCGCCAGGGAGGGCCACCTGGAAATCGTGGAGGTGCTGC |
| | TGAAACACGGCGCCGACGTGAACGCCCAGGACTGGTACGGTAGTACTCCCCTCCA |
| | CCTGGCCGCTGCCTGGGGACATCTGGAGATTGTGGAAGTGCTGCTGAAGAACGTGG |
| | CCGACGTGAACGCCATGGACGGCGACGGCAGCACCCCCCTGCACCTGGCCGCC |
| | CACTACGCCCACCTGGAGGTGGTGGAGGTGCTGCTGAAGAACGGCGCGGACGTCA |
| | ATGCCCAGGATAAGTTCGGCAAGACCGCCTTCGACATCAGCATCGACAACGGCAAC |
| | GAGGACCTGGCCGAGATCCTGCAGAAGCTGAACggatccggtgggtccggtagtggtggttccA |
| | AGGTGGAGCAAGCGGTGGAGACAGAGCCGGAGCCCGAGCTGCGCCAGCAGACC |
| | GAGTGGCAGAGCGGCCAGCGCTGGGAACTGGCACTGGGTCGCTTTTGGGATTACC |
| | TGCGCTGGGTGCAGACACTGTCTGAGCAGGTGCAGGAGGAGCTGCTCAGCTCCCA |
| | GGTCACCCAGGAACTGAGGGCGCTGATGGACGAGACCATGAAGGAGTTGAAGGCC |
| | TACAAATCGGAACTGGAGGAACAACTGACCCCGGTGGCGGAGGAGACGCGGGCA |
| | CGGCTGTCCAAGGAGCTGCAGGCGGCGCAGGCCCGGCTGGGCGCGGACATGGA |
| | GGACGTGTGCGGCCGCCTGGTGCAGTACCGCGGCGAGGTGCAGGCCATGCTCGG |
| | CCAGAGCACCGAGGAGCTGCGGGTGCGCCTCGCCTCCCACCTGgccagcCTGCGT |
| | AAGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGATACGC |
| | CTGCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACTAG (SEQ ID NO: 39) |
| CD4- | MKVLWAALLVTFLAGCQADLGKRLLEAARAGQDDEVRILMANGADVNAKDDEGRTSLH |
| DARPin_APOE3_ | LAAREGHLEIVEVLLKHGADVNAQDWYGSTPLHLAAAWGHLEIVEVLLKNVADVNAMD |
| R142A_K143S | GDGSTPLHLAAHYAHLEVVEVLLKNGADVNAQDKFGKTAFDISIDNGNEDLAEILQKLNG |
| | SGGSGSGGSKVEQAVETEPEPELRQQTEWQSGQRWELALGRFWDYLRWVQTLSEQV |
| | QEELLSSQVTQELRALMDETMKELKAYKSELEEQLTPVAEETRARLSKELQAAQARLGAD |
| | MEDVCGRLVQYRGEVQAMLGQSTEELRVRLASHLASLRKRLLRDADDLQKRLAVYQAG |
| | AREGAERGLSAIRERLGPLVEQGRVRAATVGSLAGQPLQERAQAWGERLRARMEEMGS |
| | RTRDRLDEVKEQVAEVRAKLEEQAQQIRLQAEAFQARLKSWFEPLVEDMQRQWAGLVE |
| | KVQAAVGTSAAPVPSDNH* (SEQ ID NO: 40) |
| EGFR- | ATGAAGGTTCTGTGGGCTGCGTTGCTGGTCACATTCCTGGCAGGATGCCAGGCCGA |
| DARPin_APOE3_ | CCTGGGCAAGAAGCTGCTGGAGGCCGCCAGGGCCGGCCAGGACGACGAGGTGA |
| R142A_K143S | GGATCCTGATGGCCAACGGCGCCGACGTGAACGCCGACGACACCTGGGGCTGGA |
| | CCCCCCTGCACCTGGCCGCCTACCAGGGTCATCTTGAGATTGTTGAAGTGTTGTTGA |
| | AAAATGGTGCCGATGTGAACGCCTACGACTACATCGGGTGGACGCCGCTACATCTA |
| | GCAGCTGACGGCCACCTGGAGATCGTGGAGGTGCTGCTGAAGAATGGCGCCGATG |
| | TTAACGCGAGCGACTACATCGGCGACACCCCTCTGCATCTCGCGGCACACAACGG |
| | CCACCTTGAGATCGTCGAGGTGCTCTTAAAGCACGGCGCCGACGTGAACGCCCAG |
| | GACAAGTTCGGCAAGACCGCCTTCGACATCAGCATCGACAACGGCAACGAGGACC |
| | TGGCCGAGATCCTGCAGggatccggtgggtccggtagtggtggttccAAGGTGGAGCAAGCGGT |
| | GGAGACAGAGCCGGAGCCCGAGCTGCGCCAGCAGACCGAGTGGCAGAGCGGCC |
| | AGCGCTGGGAACTGGCACTGGGTCGCTTTTGGGATTACCTGCGCTGGGTGCAGACA |
| | CTGTCTGAGCAGGTGCAGGAGGAGCTGCTCAGCTCCCAGGTCACCCAGGAACTGA |
| | GGGCGCTGATGGACGAGACCATGAAGGAGTTGAAGGCCTACAAATCGGAACTGGA |
| | GGAACAACTGACCCCGGTGGCGGAGGAGACGCGGGCACGGCTGTCCAAGGAGCT |
| | GCAGGCGGCGCAGGCCCGGCTGGGCGCGGACATGGAGGACGTGTGCGGCCGC |
| | CTGGTGCAGTACCGCGGCGAGGTGCAGGCCATGCTCGGCCAGAGCACCGAGGAG |
| | CTGCGGGTGCGCCTCGCCTCCCACCTGgccagcCTGCGTAAGCGGCTCCTCCGCG |
| | ATGCCGATGACCTGCAGAAGCGCCTGGCAGTGTACCAGGCCGGGGCCCGCGAGG |
| | GCGCCGAGCGCGGCCTCAGCGCCATCCGCGAGCGCCTGGGGCCCCTGGTGGAA |
| | CAGGGCCGCGTGCGGGCCGCCACTGTGGGCTCCCTGGCCGGCCAGCCGCTACA |
| | GGAGCGGGCCCAGGCCTGGGGCGAGCGGCTGCGCGCGCGGATGGAGGAGATGG |
| | GCAGCCGGACCCGCGACCGCCTGGACGAGGTGAAGGAGCAGGTGGCGGAGGTG |
| | CGCGCCAAGCTGGAGGAGCAGGCCCAGCAGATACGCCTGCAGGCCGAGGCCTTC |
| | CAGGCCCGCCTCAAGAGCTGGTTCGAGCCCCTGGTGGAAGACATGCAGCGCCAGT |
| | GGGCCGGGCTGGTGGAGAAGGTGCAGGCTGCCGTGGGCACCAGCGCCGCCCCT |
| | GTGCCCAGCGACAATCACTAG (SEQ ID NO: 41) |
| EGFR- | MKVLWAALLVTFLAGCQADLGKKLLEAARAGQDDEVRILMANGADVNADDTWGWTPL |
| DARPin_APOE3_ | HLAAYQGHLEIVEVLLKNGADVNAYDYIGWTPLHLAADGHLEIVEVLLKNGADVNASDYI |
| R142A_K143S | GDTPLHLAAHNGHLEIVEVLLKHGADVNAQDKFGKTAFDISIDNGNEDLAEILQGSGGS |
| | GSGGSKVEQAVETEPEPELRQQTEWQSGQRWELALGRFWDYLRWVQTLSEQVQEELL |
| | SSQVTQELRALMDETMKELKAYKSELEEQLTPVAEETRARLSKELQAAQARLGADMEDVC |
| | GRLVQYRGEVQAMLGQSTEELRVRLASHLASLRKRLLRDADDLQKRLAVYQAGAREGAE |
| | RGLSAIRERLGPLVEQGRVRAATVGSLAGQPLQERAQAWGERLRARMEEMGSRTRDRL |
| | DEVKEQVAEVRAKLEEQAQQIRLQAEAFQARLKSWFEPLVEDMQRQWAGLVEKVQAAV |
| | GTSAAPVPSDNH* (SEQ ID NO: 42) |
| EE_GS10_ | ATGAAGGTTCTGTGGGCTGCGTTGCTGGTCACATTCCTGGCAGGATGCCAGGCCgag |
| APOE_mut_ | tacatgcccatggagggatccggtgggtccggtagtggtggttccAAGGTGGAGCAAGCGGTGGAGA |
| R142A_K143S | CAGAGCCGGAGCCCGAGCTGCGCCAGCAGACCGAGTGGCAGAGCGGCCAGCGC |
| | TGGGAACTGGCACTGGGTCGCTTTTGGGATTACCTGCGCTGGGTGCAGACACTGTCT |
| | GAGCAGGTGCAGGAGGAGCTGCTCAGCTCCCAGGTCACCCAGGAACTGAGGGCG |
| | CTGATGGACGAGACCATGAAGGAGTTGAAGGCCTACAAATCGGAACTGGAGGAACA |
| | ACTGACCCCGGTGGCGGAGGAGACGCGGGCACGGCTGTCCAAGGAGCTGCAGG |
| | CGGCGCAGGCCCGGCTGGGCGCGGACATGGAGGACGTGTGCGGCCGCCTGGTG |
| | CAGTACCGCGGCGAGGTGCAGGCCATGCTCGGCCAGAGCACCGAGGAGCTGCG |
| | GGTGCGCCTCGCCTCCCACCTGgccagcCTGCGTAAGCGGCTCCTCCGCGATGCC |
| | GATGACCTGCAGAAGCGCCTGGCAGTGTACCAGGCCGGGGCCCGCGAGGGCGC |
| | CGAGCGCGGCCTCAGCGCCATCCGCGAGCGCCTGGGGCCCCTGGTGGAACAGG |
| | GCCGCGTGCGGGCCGCCACTGTGGGCTCCCTGGCCGGCCAGCCGCTACAGGAG |
| | CGGGCCCAGGCCTGGGGCGAGCGGCTGCGCGCGCGGATGGAGGAGATGGGCAG |
| | CCGGACCCGCGACCGCCTGGACGAGGTGAAGGAGCAGGTGGCGGAGGTGCGCG |
| | CCAAGCTGGAGGAGCAGGCCCAGCAGATACGCCTGCAGGCCGAGGCCTTCCAGG |
| | CCCGCCTCAAGAGCTGGTTCGAGCCCCTGGTGGAAGACATGCAGCGCCAGTGGG |
| | CCGGGCTGGTGGAGAAGGTGCAGGCTGCCGTGGGCACCAGCGCCGCCCCTGTG |
| | CCCAGCGACAATCACTAG (SEQ ID NO: 43) |
| EE_GS10_ | MKVLWAALLVTFLAGCQAEYMPMEGSGGSGSGGSKVEQAVETEPEPELRQQTEWQSG |
| APOE_mut_ | QRWELALGRFWDYLRWVQTLSEQVQEELLSSQVTQELRALMDETMKELKAYKSELEEQL |
| R142A_K143S | TPVAEETRARLSKELQAAQARLGADMEDVCGRLVQYRGEVQAMLGQSTEELRVRLASHL |
| | ASLRKRLLRDADDLQKRLAVYQAGAREGAERGLSAIRERLGPLVEQGRVRAATVGSLAG |
| | QPLQERAQAWGERLRARMEEMGSRTRDRLDEVKEQVAEVRAKLEEQAQQIRLQAEAFQ |
| | ARLKSWFEPLVEDMQRQWAGLVEKVQAAVGTSAAPVPSDNH* (SEQ ID NO: 44) |
| EE_LBD_mut1 | ATGAAGGTTCTGTGGGCTGCGTTGCTGGTCACATTCCTGGCAGGATGCCAGGCCgag |
| | tacatgcccatggagggatccggtgggtccggtagtggtggttccAAGGTGGAGCAAGCGGTGGAGA |
| | CAGAGCCGGAGCCCGAGCTGCGCCAGCAGACCGAGTGGCAGAGCGGCCAGCGC |
| | TGGGAACTGGCACTGGGTCGCTTTTGGGATTACCTGCGCTGGGTGCAGACACTGTCT |
| | GAGCAGGTGCAGGAGGAGCTGCTCAGCTCCCAGGTCACCCAGGAACTGAGGGCG |
| | CTGATGGACGAGACCATGAAGGAGTTGAAGGCCTACAAATCGGAACTGGAGGAACA |
| | ACTGACCCCGGTGGCGGAGGAGACGCGGGCACGGCTGTCCAAGGAGCTGCAGG |
| | CGGCGCAGGCCCGGCTGGGCGCGGACATGGAGGACGTGTGCGGCCGCCTGGTG |
| | CAGTACCGCGGCGAGGTGCAGGCCATGCTCGGCCAGAGCACCGAGGAGCTGCG |
| | GGTGCGCCTCGCCTCCCACCTGgccagcCTGCGTAAGCGGCTCCTCCGCGATGCC |
| | GATGACCTGCAGAAGCGCCTGGCAGTGTACCAGGCCGGGGCCCGCGAGGGCGC |
| | CGAGCGCGGCCTCAGCGCCATCCGCGAGCGCCTGGGGCCCCTGGTGGAACAGG |
| | GCCGCGTGCGGGCCGCCACTGTGGGCTCCCTGGCCGGCCAGCCGCTACAGGAG |
| | CGGGCCCAGGCCTGGGGCGAGCGGCTGCGCGCGCGGATGGAGGAGATGGGCAG |
| | CCGGACCCGCGACCGCCTGGACGAGGTGAAGGAGCAGGTGGCGGAGGTGCGCG |
| | CCAAGCTGGAGGAGCAGGCCCAGCAGAGCCGCAGCCAGGCCGAGGCCTTCCAG |
| | GCCCGCCTCAAGAGCTGGTTCGAGCCCCTGGTGGAAGACATGCAGCGCCAGTGG |
| | GCCGGGCTGGTGGAGAAGGTGCAGGCTGCCGTGGGCACCAGCGCCGCCCCTGT |
| | GCCCAGCGACAATCACTAG (SEQ ID NO: 45) |
| EE_LBD_mut1 | MKVLWAALLVTFLAGCQAEYMPMEGSGGSGSGGSKVEQAVETEPEPELRQQTEWQSG |
| | QRWELALGRFWDYLRWVQTLSEQVQEELLSSQVTQELRALMDETMKELKAYKSELEEQL |
| | TPVAEETRARLSKELQAAQARLGADMEDVCGRLVQYRGEVQAMLGQSTEELRVRLASHL |
| | ASLRKRLLRDADDLQKRLAVYQAGAREGAERGLSAIRERLGPLVEQGRVRAATVGSLAG |
| | QPLQERAQAWGERLRARMEEMGSRTRDRLDEVKEQVAEVRAKLEEQAQQSRSQAEAF |
| | QARLKSWFEPLVEDMQRQWAGLVEKVQAAVGTSAAPVPSDNH* (SEQ ID NO: 46) |
| EE_LBD_18A | ATGAAGGTTCTGTGGGCTGCGTTGCTGGTCACATTCCTGGCAGGATGCCAGGCCgag |
| | tacatgcccatggagggatccggtgggtccggtagtggtggttccAAGGTGGAGCAAGCGGTGGAGA |
| | CAGAGCCGGAGCCCGAGCTGCGCCAGCAGACCGAGTGGCAGAGCGGCCAGCGC |
| | TGGGAACTGGCACTGGGTCGCTTTTGGGATTACCTGCGCTGGGTGCAGACACTGTCT |
| | GAGCAGGTGCAGGAGGAGCTGCTCAGCTCCCAGGTCACCCAGGAACTGAGGGCG |
| | CTGATGGACGAGACCATGAAGGAGTTGAAGGCCTACAAATCGGAACTGGAGGAACA |
| | ACTGACCCCGGTGGCGGAGGAGACGCGGGCACGGCTGTCCAAGGAGCTGCAGG |
| | CGGCGCAGGCCCGGCTGGGCGCGGACATGGAGGACGTGTGCGGCCGCCTGGTG |
| | CAGTACCGCGGCGAGGTGCAGGCCATGCTCGGCCAGAGCACCGAGGAGCTGCG |
| | GGTGCGCCTCGCCTCCCACCTGgccagcCTGCGTAAGCGGCTCCTCCGCGATGCC |
| | GATGACCTGCAGAAGCGCCTGGCAGTGTACCAGGCCGGGGCCCGCGAGGGCGC |
| | CGAGCGCGGCCTCAGCGCCATCCGCGAGCGCCTGGGGCCCCTGGTGGAACAGG |
| | GCCGCGTGCGGGCCGCCACTGTGGGCTCCCTGGCCGGCCAGCCGCTACAGGAG |
| | CGGGCCCAGGCCTGGGGCGAGCGGCTGCGCGCGCGGATGGAGGAGATGGGCAG |
| | CCGGACCCGCGACCGCCTGGACGAGGTGAAGGAGCAGGTGGCGGAGGTGCGCG |
| | CCAAGCTGGAGGAGCAGGCCCAGCAGATACGCCTGCAGGCCGAGGCCTTCCAGG |
| | CCCGCCTCAAGAGCTGGTTCGAGCCCCTGGTGGAAGACATGCAGCGCCAGTGGG |
| | CCGGGCTGGTGGAGAAGGTGCAGGCTGCCGTGGGCACCAGCGCCGCCCCTGTG |
| | CCCAGCGACAATCACGACTGGCTGAAGGCCTTCTACGACAAGGTGGCCGAGAAGC |
| | TGAAGGAGGCCTTCTGA (SEQ ID NO: 47) |
| EE_LBD_18A | MKVLWAALLVTFLAGCQAEYMPMEGSGGSGSGGSKVEQAVETEPEPELRQQTEWQSG |
| | QRWELALGRFWDYLRWVQTLSEQVQEELLSSQVTQELRALMDETMKELKAYKSELEEQL |
| | TPVAEETRARLSKELQAAQARLGADMEDVCGRLVQYRGEVQAMLGQSTEELRVRLASHL |
| | ASLRKRLLRDADDLQKRLAVYQAGAREGAERGLSAIRERLGPLVEQGRVRAATVGSLAG |
| | QPLQERAQAWGERLRARMEEMGSRTRDRLDEVKEQVAEVRAKLEEQAQQIRLQAEAFQ |
| | ARLKSWFEPLVEDMQRQWAGLVEKVQAAVGTSAAPVPSDNHDWLKAFYDKVAEKLKEA |
| | F* (SEQ ID NO: 48) |
| EE_LBD_GSG_ | atgaaggttctgtgggctgcgttgctggtcacattcctggcaggatgccaggccgagtacatgcccatggaggg |
| 18A | atccggtgggtccggtagtggtggttccaaggtggagcaagcggtggagacagagccggagcccgagctgcgcc |
| | agcagaccgagtggcagagcggccagcgctgggaactggcactgggtcgcttttgggattacctgcgctgggtg |
| | cagacactgtctgagcaggtgcaggaggagctgctcagctcccaggtcacccaggaactgagggcgctgatgga |
| | cgagaccatgaaggagttgaaggcctacaaatcggaactggaggaacaactgaccccggtggcggaggaga |
| | cgcgggcacggctgtccaaggagctgcaggcggcgcaggcccggctgggcgcggacatggaggacgtgtgcg |
| | gccgcctggtgcagtaccgcggcgaggtgcaggccatgctcggccagagcaccgaggagctgcgggtgcgcc |
| | tcgcctcccacctggccagcctgcgtaagcggctcctccgcgatgccgatgacctgcagaagcgcctggcagt |
| | gtaccaggccggggcccgcgagggcgccgagcgcggcctcagcgccatccgcgagcgcctggggcccctgg |
| | tggaacagggccgcgtgcgggccgccactgtgggctccctggccggccagccgctacaggagcgggcccagg |
| | cctggggcgagcggctgcgcgcgcggatggaggagatgggcagccggacccgcgaccgcctggacgaggtg |
| | aaggagcaggtggcggaggtgcgcgccaagctggaggagcaggcccagcagatacgcctgcaggccgaggc |
| | cttccaggcccgcctcaagagctggttcgagcccctggtggaagacatgcagcgccagtgggccgggctggtg |
| | gagaaggtgcaggctgccgtgggcaccagcgccgcccctgtgcccagcgacaatcacggcagcggcgactg |
| | gctgaaggccttctacgacaaggtggccgagaagctgaaggaggccttctga (SEQ ID NO: 49) |
| EE_LBD_ | MKVLWAALLVTFLAGCQAEYMPMEGSGGSGSGGSKVEQAVETEPEPELRQQTEWQSG |
| G_18A | QRWELALGRFWDYLRWVQTLSEQVQEELLSSQVTQELRALMDETMKELKAYKSELEEQL |
| GS | TPVAEETRARLSKELQAAQARLGADMEDVCGRLVQYRGEVQAMLGQSTEELRVRLASHL |
| | ASLRKRLLRDADDLQKRLAVYQAGAREGAERGLSAIRERLGPLVEQGRVRAATVGSLAG |
| | QPLQERAQAWGERLRARMEEMGSRTRDRLDEVKEQVAEVRAKLEEQAQQIRLQAEAFQ |
| | ARLKSWFEPLVEDMQRQWAGLVEKVQAAVGTSAAPVPSDNHGSGDWLKAFYDKVAEK |
| | LKEAF* (SEQ ID NO: 50) |
| Moon_GS10_ | ATGAAGGTTCTGTGGGCTGCGTTGCTGGTCACATTCCTGGCAGGATGCCAGGCCaag |
| APOE_mut | aacgagcaggagctgctggagctggacaagtgggccagcctgggatccggtgggtccggtagtggtggttccAA |
| | GGTGGAGCAAGCGGTGGAGACAGAGCCGGAGCCCGAGCTGCGCCAGCAGACCG |
| | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGATACGC |
| | CTGCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACTAG (SEQ ID NO: 51) |
| Moon_GS10_ | MKVLWAALLVTFLAGCQAKNEQELLELDKWASLGSGGSGSGGSKVEQAVETEPEPELR |
| APOE_mut | QQTEWQSGQRWELALGRFWDYLRWVQTLSEQVQEELLSSQVTQELRALMDETMKELK |
| | AYKSELEEQLTPVAEETRARLSKELQAAQARLGADMEDVCGRLVQYRGEVQAMLGQSTE |
| | ELRVRLASHLASLRKRLLRDADDLQKRLAVYQAGAREGAERGLSAIRERLGPLVEQGRVR |
| | AATVGSLAGQPLQERAQAWGERLRARMEEMGSRTRDRLDEVKEQVAEVRAKLEEQAQ |
| | QIRLQAEAFQARLKSWFEPLVEDMQRQWAGLVEKVQAAVGTSAAPVPSDNH* (SEQ ID |
| | NO: 52) |
| Moon_LBD_ | ATGAAGGTTCTGTGGGCTGCGTTGCTGGTCACATTCCTGGCAGGATGCCAGGCCaag |
| mut1 | aacgagcaggagctgctggagctggacaagtgggccagcctgggatccggtgggtccggtagtggtggttccAA |
| | GGTGGAGCAAGCGGTGGAGACAGAGCCGGAGCCCGAGCTGCGCCAGCAGACCG |
| | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGAGCCGC |
| | AGCCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACTAG (SEQ ID NO: 53) |
| Moon_LBD_ | MKVLWAALLVTFLAGCQAKNEQELLELDKWASLGSGGSGSGGSKVEQAVETEPEPELR |
| mut1 | QQTEWQSGQRWELALGRFWDYLRWVQTLSEQVQEELLSSQVTQELRALMDETMKELK |
| | AYKSELEEQLTPVAEETRARLSKELQAAQARLGADMEDVCGRLVQYRGEVQAMLGQSTE |
| | ELRVRLASHLASLRKRLLRDADDLQKRLAVYQAGAREGAERGLSAIRERLGPLVEQGRVR |
| | AATVGSLAGQPLQERAQAWGERLRARMEEMGSRTRDRLDEVKEQVAEVRAKLEEQAQ |
| | QSRSQAEAFQARLKSWFEPLVEDMQRQWAGLVEKVQAAVGTSAAPVPSDNH* (SEQ |
| | ID NO: 54) |
| Moon_LBD_ | ATGAAGGTTCTGTGGGCTGCGTTGCTGGTCACATTCCTGGCAGGATGCCAGGCCaag |
| 18A | aacgagcaggagctgctggagctggacaagtgggccagcctgggatccggtgggtccggtagtggtggttccAA |
| | GGTGGAGCAAGCGGTGGAGACAGAGCCGGAGCCCGAGCTGCGCCAGCAGACCG |
| | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGATACGC |
| | CTGCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACGACTGGCTGAAGGCCTTCT |
| | ACGACAAGGTGGCCGAGAAGCTGAAGGAGGCCTTCTGA (SEQ ID NO: 55) |
| Moon_LBD_ | MKVLWAALLVTFLAGCQAKNEQELLELDKWASLGSGGSGSGGSKVEQAVETEPEPELR |
| 18A | QQTEWQSGQRWELALGRFWDYLRWVQTLSEQVQEELLSSQVTQELRALMDETMKELK |
| | AYKSELEEQLTPVAEETRARLSKELQAAQARLGADMEDVCGRLVQYRGEVQAMLGQSTE |
| | ELRVRLASHLASLRKRLLRDADDLQKRLAVYQAGAREGAERGLSAIRERLGPLVEQGRVR |
| | AATVGSLAGQPLQERAQAWGERLRARMEEMGSRTRDRLDEVKEQVAEVRAKLEEQAQ |
| | QIRLQAEAFQARLKSWFEPLVEDMQRQWAGLVEKVQAAVGTSAAPVPSDNHDWLKAFY |
| | DKVAEKLKEAF* (SEQ ID NO: 56) |
| Moon_LBD_ | ATGAAGGTTCTGTGGGCTGCGTTGCTGGTCACATTCCTGGCAGGATGCCAGGCCaag |
| GSG_18A | aacgagcaggagctgctggagctggacaagtgggccagcctgggatccggtgggtccggtagtggtggttccAA |
| | GGTGGAGCAAGCGGTGGAGACAGAGCCGGAGCCCGAGCTGCGCCAGCAGACCG |
| | AGTGGCAGAGCGGCCAGCGCTGGGAACTGGCACTGGGTCGCTTTTGGGATTACCT |
| | GCGCTGGGTGCAGACACTGTCTGAGCAGGTGCAGGAGGAGCTGCTCAGCTCCCAG |
| | GTCACCCAGGAACTGAGGGCGCTGATGGACGAGACCATGAAGGAGTTGAAGGCCT |
| | ACAAATCGGAACTGGAGGAACAACTGACCCCGGTGGCGGAGGAGACGCGGGCAC |
| | GGCTGTCCAAGGAGCTGCAGGCGGCGCAGGCCCGGCTGGGCGCGGACATGGAG |
| | GACGTGTGCGGCCGCCTGGTGCAGTACCGCGGCGAGGTGCAGGCCATGCTCGGC |
| | CAGAGCACCGAGGAGCTGCGGGTGCGCCTCGCCTCCCACCTGgccagcCTGCGTA |
| | AGCGGCTCCTCCGCGATGCCGATGACCTGCAGAAGCGCCTGGCAGTGTACCAGG |
| | CCGGGGCCCGCGAGGGCGCCGAGCGCGGCCTCAGCGCCATCCGCGAGCGCCT |
| | GGGGCCCCTGGTGGAACAGGGCCGCGTGCGGGCCGCCACTGTGGGCTCCCTGG |
| | CCGGCCAGCCGCTACAGGAGCGGGCCCAGGCCTGGGGCGAGCGGCTGCGCGC |
| | GCGGATGGAGGAGATGGGCAGCCGGACCCGCGACCGCCTGGACGAGGTGAAGG |
| | AGCAGGTGGCGGAGGTGCGCGCCAAGCTGGAGGAGCAGGCCCAGCAGATACGC |
| | CTGCAGGCCGAGGCCTTCCAGGCCCGCCTCAAGAGCTGGTTCGAGCCCCTGGTG |
| | GAAGACATGCAGCGCCAGTGGGCCGGGCTGGTGGAGAAGGTGCAGGCTGCCGTG |
| | GGCACCAGCGCCGCCCCTGTGCCCAGCGACAATCACggcagcggcGACTGGCTGAA |
| | GGCCTTCTACGACAAGGTGGCCGAGAAGCTGAAGGAGGCCTTCTGA (SEQ ID NO: |
| | 57) |
| Moon_LBD_ | MKVLWAALLVTFLAGCQAKNEQELLELDKWASLGSGGSGSGGSKVEQAVETEPEPELR |
| GSG_18A | QQTEWQSGQRWELALGRFWDYLRWVQTLSEQVQEELLSSQVTQELRALMDETMKELK |
| | AYKSELEEQLTPVAEETRARLSKELQAAQARLGADMEDVCGRLVQYRGEVQAMLGQSTE |
| | ELRVRLASHLASLRKRLLRDADDLQKRLAVYQAGAREGAERGLSAIRERLGPLVEQGRVR |
| | AATVGSLAGQPLQERAQAWGERLRARMEEMGSRTRDRLDEVKEQVAEVRAKLEEQAQ |
| | QIRLQAEAFQARLKSWFEPLVEDMQRQWAGLVEKVQAAVGTSAAPVPSDNHGSGDWL |
| | KAFYDKVAEKLKEAF* (SEQ ID NO: 58) |
| CD19_GS10_ | ATGAAGGTTCTGTGGGCTGCGTTGCTGGTCACATTCCTGGCAGGATGCCAGGCCgac |
| APOE_mut | atccagatgacacagactacatcctccctgtctgcctctctgggagacagagtcaccatcagttgcagggcaag |
| | tcaggacatctctaagtatttgaattggtatcagcagaaaccagatggaactgttaaactcctgatctaccata |
| | catcaagattacactcaggagtcccatcaaggttcagtggcagtgggtctggaacagattattctctcaccatt |
| | agcaacctggagcaagaagatattgccacttacttttgccaacagggtaatacgcttccgtacacgttcggagg |
| | ggggactaagttggaaataacaggctccacctctggatccggcaagcccggatctggcgagggatccaccaagg |
| | gcgaggtgaaactgcaggagtcaggacctggcctggtggcgccctcacagagcctgtccgtcacatgcactgtc |
| | tcaggggtctcattacccgactatggtgtaagctggattcgccagcctccacgaaagggtctggagtggctggg |
| | agtaatatggggtagtgaaaccacatactataattcagctctcaaatccagactgaccatcatcaaggacaact |
| | ccaagagccaagttttcttaaaaatgaacagtctgcaaactgatgacacagccatttactactgtgccaaacat |
| | tattactacggtggtagctatgctatggactactggggtcaaggaacctcagtcaccgtctcctcaggatccgg |
| | tgggtccggtagtggtggttccAAGGTGGAGCAAGCGGTGGAGACAGAGCCGGAGCCCGAGCTGC |
| | GCCAGCAGACCGAGTGGCAGAGCGGCCAGCGCTGGGAACTGGCACTGGGTCGCT |
| | TTTGGGATTACCTGCGCTGGGTGCAGACACTGTCTGAGCAGGTGCAGGAGGAGCTG |
| | CTCAGCTCCCAGGTCACCCAGGAACTGAGGGCGCTGATGGACGAGACCATGAAGG |
| | AGTTGAAGGCCTACAAATCGGAACTGGAGGAACAACTGACCCCGGTGGCGGAGGA |
| | GACGCGGGCACGGCTGTCCAAGGAGCTGCAGGCGGCGCAGGCCCGGCTGGGCG |
| | CGGACATGGAGGACGTGTGCGGCCGCCTGGTGCAGTACCGCGGCGAGGTGCAGG |
| | CCATGCTCGGCCAGAGCACCGAGGAGCTGCGGGTGCGCCTCGCCTCCCACCTGg |
| | ccagcCTGCGTAAGCGGCTCCTCCGCGATGCCGATGACCTGCAGAAGCGCCTGGCA |
| | GTGTACCAGGCCGGGGCCCGCGAGGGCGCCGAGCGCGGCCTCAGCGCCATCCG |
| | CGAGCGCCTGGGGCCCCTGGTGGAACAGGGCCGCGTGCGGGCCGCCACTGTGG |
| | GCTCCCTGGCCGGCCAGCCGCTACAGGAGCGGGCCCAGGCCTGGGGCGAGCG |
| | GCTGCGCGCGCGGATGGAGGAGATGGGCAGCCGGACCCGCGACCGCCTGGACG |
| | AGGTGAAGGAGCAGGTGGCGGAGGTGCGCGCCAAGCTGGAGGAGCAGGCCCAG |
| | CAGATACGCCTGCAGGCCGAGGCCTTCCAGGCCCGCCTCAAGAGCTGGTTCGAG |
| | CCCCTGGTGGAAGACATGCAGCGCCAGTGGGCCGGGCTGGTGGAGAAGGTGCAG |
| | GCTGCCGTGGGCACCAGCGCCGCCCCTGTGCCCAGCGACAATCACTAG (SEQ ID |
| | NO: 59) |
| CD19_GS10_ | MKVLWAALLVTFLAGCQADIQMTQTTSSLSASLGDRVTISCRASQDISKYLNWYQQKPD |
| APOE_mut | GTVKLLIYHTSRLHSGVPSRFSGSGSGTDYSLTISNLEQEDIATYFCQQGNTLPYTFGGGT |
| | KLEITGSTSGSGKPGSGEGSTKGEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAKH |
| | YYYGGSYAMDYWGQGTSVTVSSGSGGSGSGGSKVEQAVETEPEPELRQQTEWQSGQR |
| | WELALGRFWDYLRWVQTLSEQVQEELLSSQVTQELRALMDETMKELKAYKSELEEQLTP |
| | VAEETRARLSKELQAAQARLGADMEDVCGRLVQYRGEVQAMLGQSTEELRVRLASHLAS |
| | LRKRLLRDADDLQKRLAVYQAGAREGAERGLSAIRERLGPLVEQGRVRAATVGSLAGQP |
| | LQERAQAWGERLRARMEEMGSRTRDRLDEVKEQVAEVRAKLEEQAQQIRLQAEAFQAR |
| | LKSWFEPLVEDMQRQWAGLVEKVQAAVGTSAAPVPSDNH* (SEQ ID NO: 60) |
| CD19_18A | ATGAAGGTTCTGTGGGCTGCGTTGCTGGTCACATTCCTGGCAGGATGCCAGGCCgac |
| | atccagatgacacagactacatcctccctgtctgcctctctgggagacagagtcaccatcagttgcagggcaag |
| | tcaggacatctctaagtatttgaattggtatcagcagaaaccagatggaactgttaaactcctgatctaccataca |
| | tcaagattacactcaggagtcccatcaaggttcagtggcagtgggtctggaacagattattctctcaccattagca |
| | acctggagcaagaagatattgccacttacttttgccaacagggtaatacgcttccgtacacgttcggagggggga |
| | ctaagttggaaataacaggctccacctctggatccggcaagcccggatctggcgagggatccaccaagggcg |
| | aggtgaaactgcaggagtcaggacctggcctggtggcgccctcacagagcctgtccgtcacatgcactgtctca |
| | ggggtctcattacccgactatggtgtaagctggattcgccagcctccacgaaagggtctggagtggctgggagta |
| | atatggggtagtgaaaccacatactataattcagctctcaaatccagactgaccatcatcaaggacaactccaa |
| | gagccaagttttcttaaaaatgaacagtctgcaaactgatgacacagccatttactactgtgccaaacattatta |
| | ctacggtggtagctatgctatggactactggggtcaaggaacctcagtcaccgtctcctcaggatccggtgggtc |
| | cggtagtggtggttccAAGGTGGAGCAAGCGGTGGAGACAGAGCCGGAGCCCGAGCTGC |
| | GCCAGCAGACCGAGTGGCAGAGCGGCCAGCGCTGGGAACTGGCACTGGGTCGCT |
| | TTTGGGATTACCTGCGCTGGGTGCAGACACTGTCTGAGCAGGTGCAGGAGGAGCTG |
| | CTCAGCTCCCAGGTCACCCAGGAACTGAGGGCGCTGATGGACGAGACCATGAAGG |
| | AGTTGAAGGCCTACAAATCGGAACTGGAGGAACAACTGACCCCGGTGGCGGAGGA |
| | GACGCGGGCACGGCTGTCCAAGGAGCTGCAGGCGGCGCAGGCCCGGCTGGGCG |
| | CGGACATGGAGGACGTGTGCGGCCGCCTGGTGCAGTACCGCGGCGAGGTGCAGG |
| | CCATGCTCGGCCAGAGCACCGAGGAGCTGCGGGTGCGCCTCGCCTCCCACCTGg |
| | ccagcCTGCGTAAGCGGCTCCTCCGCGATGCCGATGACCTGCAGAAGCGCCTGGCA |
| | GTGTACCAGGCCGGGGCCCGCGAGGGCGCCGAGCGCGGCCTCAGCGCCATCCG |
| | CGAGCGCCTGGGGCCCCTGGTGGAACAGGGCCGCGTGCGGGCCGCCACTGTGG |
| | GCTCCCTGGCCGGCCAGCCGCTACAGGAGGACTGGCTGAAGGCCTTCTACGACAA |
| | GGTGGCCGAGAAGCTGAAGGAGGCCTTCTGA (SEQ ID NO: 61) |
| CD19_18A | MKVLWAALLVTFLAGCQADIQMTQTTSSLSASLGDRVTISCRASQDISKYLNWYQQKPD |
| | GTVKLLIYHTSRLHSGVPSRFSGSGSGTDYSLTISNLEQEDIATYFCQQGNTLPYTFGGGT |
| | KLEITGSTSGSGKPGSGEGSTKGEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAKH |
| | YYYGGSYAMDYWGQGTSVTVSSGSGGSGSGGSKVEQAVETEPEPELRQQTEWQSGQR |
| | WELALGRFWDYLRWVQTLSEQVQEELLSSQVTQELRALMDETMKELKAYKSELEEQLTP |
| | VAEETRARLSKELQAAQARLGADMEDVCGRLVQYRGEVQAMLGQSTEELRVRLASHLAS |
| | LRKRLLRDADDLQKRLAVYQAGAREGAERGLSAIRERLGPLVEQGRVRAATVGSLAGQP |
| | LQEDWLKAFYDKVAEKLKEAF* (SEQ ID NO: 62) |
| CD19_LBD_ | ATGAAGGTTCTGTGGGCTGCGTTGCTGGTCACATTCCTGGCAGGATGCCAGGCCgac |
| mut1 | atccagatgacacagactacatcctccctgtctgcctctctgggagacagagtcaccatcagttgcagggcaag |
| | tcaggacatctctaagtatttgaattggtatcagcagaaaccagatggaactgttaaactcctgatctaccataca |
| | tcaagattacactcaggagtcccatcaaggttcagtggcagtgggtctggaacagattattctctcaccattagca |
| | acctggagcaagaagatattgccacttacttttgccaacagggtaatacgcttccgtacacgttcggagggggga |
| | ctaagttggaaataacaggctccacctctggatccggcaagcccggatctggcgagggatccaccaagggcg |
| | aggtgaaactgcaggagtcaggacctggcctggtggcgccctcacagagcctgtccgtcacatgcactgtctca |
| | ggggtctcattacccgactatggtgtaagctggattcgccagcctccacgaaagggtctggagtggctgggagta |
| | atatggggtagtgaaaccacatactataattcagctctcaaatccagactgaccatcatcaaggacaactccaa |
| | gagccaagttttcttaaaaatgaacagtctgcaaactgatgacacagccatttactactgtgccaaacattatta |
| | ctacggtggtagctatgctatggactactggggtcaaggaacctcagtcaccgtctcctcaggatccggtgggtc |
| | cggtagtggtggttccAAGGTGGAGCAAGCGGTGGAGACAGAGCCGGAGCCCGAGCTGC |
| | GCCAGCAGACCGAGTGGCAGAGCGGCCAGCGCTGGGAACTGGCACTGGGTCGCT |
| | TTTGGGATTACCTGCGCTGGGTGCAGACACTGTCTGAGCAGGTGCAGGAGGAGCTG |
| | CTCAGCTCCCAGGTCACCCAGGAACTGAGGGCGCTGATGGACGAGACCATGAAGG |
| | AGTTGAAGGCCTACAAATCGGAACTGGAGGAACAACTGACCCCGGTGGCGGAGGA |
| | GACGCGGGCACGGCTGTCCAAGGAGCTGCAGGCGGCGCAGGCCCGGCTGGGCG |
| | CGGACATGGAGGACGTGTGCGGCCGCCTGGTGCAGTACCGCGGCGAGGTGCAGG |
| | CCATGCTCGGCCAGAGCACCGAGGAGCTGCGGGTGCGCCTCGCCTCCCACCTGg |
| | ccagcCTGCGTAAGCGGCTCCTCCGCGATGCCGATGACCTGCAGAAGCGCCTGGCA |
| | GTGTACCAGGCCGGGGCCCGCGAGGGCGCCGAGCGCGGCCTCAGCGCCATCCG |
| | CGAGCGCCTGGGGCCCCTGGTGGAACAGGGCCGCGTGCGGGCCGCCACTGTGG |
| | GCTCCCTGGCCGGCCAGCCGCTACAGGAGCGGGCCCAGGCCTGGGGCGAGCG |
| | GCTGCGCGCGCGGATGGAGGAGATGGGCAGCCGGACCCGCGACCGCCTGGACG |
| | AGGTGAAGGAGCAGGTGGCGGAGGTGCGCGCCAAGCTGGAGGAGCAGGCCCAG |
| | CAGAGCCGCAGCCAGGCCGAGGCCTTCCAGGCCCGCCTCAAGAGCTGGTTCGAG |
| | CCCCTGGTGGAAGACATGCAGCGCCAGTGGGCCGGGCTGGTGGAGAAGGTGCAG |
| | GCTGCCGTGGGCACCAGCGCCGCCCCTGTGCCCAGCGACAATCACTAG (SEQ ID |
| | NO: 63) |
| CD19_LBD_ | MKVLWAALLVTFLAGCQADIQMTQTTSSLSASLGDRVTISCRASQDISKYLNWYQQKPD |
| mut1 | GTVKLLIYHTSRLHSGVPSRFSGSGSGTDYSLTISNLEQEDIATYFCQQGNTLPYTFGGGT |
| | KLEITGSTSGSGKPGSGEGSTKGEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAKH |
| | YYYGGSYAMDYWGQGTSVTVSSGSGGSGSGGSKVEQAVETEPEPELRQQTEWQSGQR |
| | WELALGRFWDYLRWVQTLSEQVQEELLSSQVTQELRALMDETMKELKAYKSELEEQLTP |
| | VAEETRARLSKELQAAQARLGADMEDVCGRLVQYRGEVQAMLGQSTEELRVRLASHLAS |
| | LRKRLLRDADDLQKRLAVYQAGAREGAERGLSAIRERLGPLVEQGRVRAATVGSLAGQP |
| | LQERAQAWGERLRARMEEMGSRTRDRLDEVKEQVAEVRAKLEEQAQQSRSQAEAFQA |
| | RLKSWFEPLVEDMQRQWAGLVEKVQAAVGTSAAPVPSDNH* (SEQ ID NO: 64) |
| CD19_LBD_ | ATGAAGGTTCTGTGGGCTGCGTTGCTGGTCACATTCCTGGCAGGATGCCAGGCCgac |
| 18A | atccagatgacacagactacatcctccctgtctgcctctctgggagacagagtcaccatcagttgcagggcaag |
| | tcaggacatctctaagtatttgaattggtatcagcagaaaccagatggaactgttaaactcctgatctaccataca |
| | tcaagattacactcaggagtcccatcaaggttcagtggcagtgggtctggaacagattattctctcaccattagca |
| | acctggagcaagaagatattgccacttacttttgccaacagggtaatacgcttccgtacacgttcggagggggga |
| | ctaagttggaaataacaggctccacctctggatccggcaagcccggatctggcgagggatccaccaagggcg |
| | aggtgaaactgcaggagtcaggacctggcctggtggcgccctcacagagcctgtccgtcacatgcactgtctca |
| | ggggtctcattacccgactatggtgtaagctggattcgccagcctccacgaaagggtctggagtggctgggagta |
| | atatggggtagtgaaaccacatactataattcagctctcaaatccagactgaccatcatcaaggacaactccaa |
| | gagccaagttttcttaaaaatgaacagtctgcaaactgatgacacagccatttactactgtgccaaacattatta |
| | ctacggtggtagctatgctatggactactggggtcaaggaacctcagtcaccgtctcctcaggatccggtgggtc |
| | cggtagtggtggttccAAGGTGGAGCAAGCGGTGGAGACAGAGCCGGAGCCCGAGCTGC |
| | GCCAGCAGACCGAGTGGCAGAGCGGCCAGCGCTGGGAACTGGCACTGGGTCGCT |
| | TTTGGGATTACCTGCGCTGGGTGCAGACACTGTCTGAGCAGGTGCAGGAGGAGCTG |
| | CTCAGCTCCCAGGTCACCCAGGAACTGAGGGCGCTGATGGACGAGACCATGAAGG |
| | AGTTGAAGGCCTACAAATCGGAACTGGAGGAACAACTGACCCCGGTGGCGGAGGA |
| | GACGCGGGCACGGCTGTCCAAGGAGCTGCAGGCGGCGCAGGCCCGGCTGGGCG |
| | CGGACATGGAGGACGTGTGCGGCCGCCTGGTGCAGTACCGCGGCGAGGTGCAGG |
| | CCATGCTCGGCCAGAGCACCGAGGAGCTGCGGGTGCGCCTCGCCTCCCACCTGg |
| | ccagcCTGCGTAAGCGGCTCCTCCGCGATGCCGATGACCTGCAGAAGCGCCTGGCA |
| | GTGTACCAGGCCGGGGCCCGCGAGGGCGCCGAGCGCGGCCTCAGCGCCATCCG |
| | CGAGCGCCTGGGGCCCCTGGTGGAACAGGGCCGCGTGCGGGCCGCCACTGTGG |
| | GCTCCCTGGCCGGCCAGCCGCTACAGGAGCGGGCCCAGGCCTGGGGCGAGCG |
| | GCTGCGCGCGCGGATGGAGGAGATGGGCAGCCGGACCCGCGACCGCCTGGACG |
| | AGGTGAAGGAGCAGGTGGCGGAGGTGCGCGCCAAGCTGGAGGAGCAGGCCCAG |
| | CAGATACGCCTGCAGGCCGAGGCCTTCCAGGCCCGCCTCAAGAGCTGGTTCGAG |
| | CCCCTGGTGGAAGACATGCAGCGCCAGTGGGCCGGGCTGGTGGAGAAGGTGCAG |
| | GCTGCCGTGGGCACCAGCGCCGCCCCTGTGCCCAGCGACAATCACGACTGGCTG |
| | AAGGCCTTCTACGACAAGGTGGCCGAGAAGCTGAAGGAGGCCTTCTGA (SEQ ID |
| | NO: 65) |
| CD19_LBD_ | MKVLWAALLVTFLAGCQADIQMTQTTSSLSASLGDRVTISCRASQDISKYLNWYQQKPD |
| 18A | GTVKLLIYHTSRLHSGVPSRFSGSGSGTDYSLTISNLEQEDIATYFCQQGNTLPYTFGGGT |
| | KLEITGSTSGSGKPGSGEGSTKGEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAKH |
| | YYYGGSYAMDYWGQGTSVTVSSGSGGSGSGGSKVEQAVETEPEPELRQQTEWQSGQR |
| | WELALGRFWDYLRWVQTLSEQVQEELLSSQVTQELRALMDETMKELKAYKSELEEQLTP |
| | VAEETRARLSKELQAAQARLGADMEDVCGRLVQYRGEVQAMLGQSTEELRVRLASHLAS |
| | LRKRLLRDADDLQKRLAVYQAGAREGAERGLSAIRERLGPLVEQGRVRAATVGSLAGQP |
| | LQERAQAWGERLRARMEEMGSRTRDRLDEVKEQVAEVRAKLEEQAQQIRLQAEAFQAR |
| | LKSWFEPLVEDMQRQWAGLVEKVQAAVGTSAAPVPSDNHDWLKAFYDKVAEKLKEAF* |
| | (SEQ ID NO: 66) |
| CD19_LBD_ | ATGAAGGTTCTGTGGGCTGCGTTGCTGGTCACATTCCTGGCAGGATGCCAGGCCgac |
| GSG_18A | atccagatgacacagactacatcctccctgtctgcctctctgggagacagagtcaccatcagttgcagggcaag |
| | tcaggacatctctaagtatttgaattggtatcagcagaaaccagatggaactgttaaactcctgatctaccataca |
| | tcaagattacactcaggagtcccatcaaggttcagtggcagtgggtctggaacagattattctctcaccattagca |
| | acctggagcaagaagatattgccacttacttttgccaacagggtaatacgcttccgtacacgttcggagggggga |
| | ctaagttggaaataacaggctccacctctggatccggcaagcccggatctggcgagggatccaccaagggcg |
| | aggtgaaactgcaggagtcaggacctggcctggtggcgccctcacagagcctgtccgtcacatgcactgtctca |
| | ggggtctcattacccgactatggtgtaagctggattcgccagcctccacgaaagggtctggagtggctgggagta |
| | atatggggtagtgaaaccacatactataattcagctctcaaatccagactgaccatcatcaaggacaactccaa |
| | gagccaagttttcttaaaaatgaacagtctgcaaactgatgacacagccatttactactgtgccaaacattatta |
| | ctacggtggtagctatgctatggactactggggtcaaggaacctcagtcaccgtctcctcaggatccggtgggtc |
| | cggtagtggtggttccAAGGTGGAGCAAGCGGTGGAGACAGAGCCGGAGCCCGAGCTGC |
| | GCCAGCAGACCGAGTGGCAGAGCGGCCAGCGCTGGGAACTGGCACTGGGTCGCT |
| | TTTGGGATTACCTGCGCTGGGTGCAGACACTGTCTGAGCAGGTGCAGGAGGAGCTG |
| | CTCAGCTCCCAGGTCACCCAGGAACTGAGGGCGCTGATGGACGAGACCATGAAGG |
| | AGTTGAAGGCCTACAAATCGGAACTGGAGGAACAACTGACCCCGGTGGCGGAGGA |
| | GACGCGGGCACGGCTGTCCAAGGAGCTGCAGGCGGCGCAGGCCCGGCTGGGCG |
| | CGGACATGGAGGACGTGTGCGGCCGCCTGGTGCAGTACCGCGGCGAGGTGCAGG |
| | CCATGCTCGGCCAGAGCACCGAGGAGCTGCGGGTGCGCCTCGCCTCCCACCTGg |
| | ccagcCTGCGTAAGCGGCTCCTCCGCGATGCCGATGACCTGCAGAAGCGCCTGGCA |
| | GTGTACCAGGCCGGGGCCCGCGAGGGCGCCGAGCGCGGCCTCAGCGCCATCCG |
| | CGAGCGCCTGGGGCCCCTGGTGGAACAGGGCCGCGTGCGGGCCGCCACTGTGG |
| | GCTCCCTGGCCGGCCAGCCGCTACAGGAGCGGGCCCAGGCCTGGGGCGAGCG |
| | GCTGCGCGCGCGGATGGAGGAGATGGGCAGCCGGACCCGCGACCGCCTGGACG |
| | AGGTGAAGGAGCAGGTGGCGGAGGTGCGCGCCAAGCTGGAGGAGCAGGCCCAG |
| | CAGATACGCCTGCAGGCCGAGGCCTTCCAGGCCCGCCTCAAGAGCTGGTTCGAG |
| | CCCCTGGTGGAAGACATGCAGCGCCAGTGGGCCGGGCTGGTGGAGAAGGTGCAG |
| | GCTGCCGTGGGCACCAGCGCCGCCCCTGTGCCCAGCGACAATCACggcagcggcGA |
| | CTGGCTGAAGGCCTTCTACGACAAGGTGGCCGAGAAGCTGAAGGAGGCCTTCTGA |
| | (SEQ ID NO: 67) |
| CD19_LBD_ | MKVLWAALLVTFLAGCQADIQMTQTTSSLSASLGDRVTISCRASQDISKYLNWYQQKPD |
| GSG_18A | GTVKLLIYHTSRLHSGVPSRFSGSGSGTDYSLTISNLEQEDIATYFCQQGNTLPYTFGGGT |
| | KLEITGSTSGSGKPGSGEGSTKGEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAKH |
| | YYYGGSYAMDYWGQGTSVTVSSGSGGSGSGGSKVEQAVETEPEPELRQQTEWQSGQR |
| | WELALGRFWDYLRWVQTLSEQVQEELLSSQVTQELRALMDETMKELKAYKSELEEQLTP |
| | VAEETRARLSKELQAAQARLGADMEDVCGRLVQYRGEVQAMLGQSTEELRVRLASHLAS |
| | LRKRLLRDADDLQKRLAVYQAGAREGAERGLSAIRERLGPLVEQGRVRAATVGSLAGQP |
| | LQERAQAWGERLRARMEEMGSRTRDRLDEVKEQVAEVRAKLEEQAQQIRLQAEAFQAR |
| | LKSWFEPLVEDMQRQWAGLVEKVQAAVGTSAAPVPSDNHGSGDWLKAFYDKVAEKLK |
| | EAF* (SEQ ID NO: 68) |
| MT-1 | A237W |
| (Moon-Tag) | |
| MT-2 | V239W |
| MT-3 | A241W |
| MT-4 | L243W |
| MT-5 | A247W |
| MT-6 | I250W |
| MT-7 | L252W |
| MT-8 | A254W |
| MT-9 | A256W |
| MT-10 | F257W |
| MT-11 | A259W |
| MT-12 | L261W |
| MT-13 | F265W |
| MT-14 | L268W |
| MT-15 | V269W |
| MT-16 | M272W |
| MT-17 | A237R |
| MT-18 | V239R |
| MT-19 | A241R |
| MT-20 | L243R |
| MT-21 | A247R |
| MT-22 | I250R |
| MT-23 | L252R |
| MT-24 | A254R |
| MT-25 | A256R |
| MT-26 | F257R |
| MT-27 | A259R |
| MT-28 | L261R |
| MT-29 | F265R |
| MT-30 | L268R |
| MT-31 | V269R |
| MT-32 | M272R |
| MT-33 | A237S |
| MT-34 | V239S |
| MT-35 | A241S |
| MT-36 | L243S |
| MT-37 | A247S |
| MT-38 | I250S |
| MT-39 | L252S |
| MT-40 | A254S |
| MT-41 | A256S |
| MT-42 | F257S |
| MT-43 | A259S |
| MT-44 | L261S |
| MT-45 | F265S |
| MT-46 | L268S |
| MT-47 | V269S |
| MT-48 | M272S |
| EE-1 (Glu- | A237W |
| Glu affinity | |
| tag) | |
| EE-2 | V239W |
| EE-3 | A241W |
| EE-4 | L243W |
| EE-5 | A247W |
| EE-6 | I250W |
| EE-7 | L252W |
| EE-8 | A254W |
| EE-9 | A256W |
| EE-10 | F257W |
| EE-11 | A259W |
| EE-12 | L261W |
| EE-13 | F265W |
| EE-14 | L268W |
| EE-15 | V269W |
| EE-16 | M272W |
| EE-17 | A237R |
| EE-18 | V239R |
| EE-19 | A241R |
| EE-20 | L243R |
| EE-21 | A247R |
| EE-22 | I250R |
| EE-23 | L252R |
| EE-24 | A254R |
| EE-25 | A256R |
| EE-26 | F257R |
| EE-27 | A259R |
| EE-28 | L261R |
| EE-29 | F265R |
| EE-30 | L268R |
| EE-31 | V269R |
| EE-32 | M272R |
| EE-33 | A237S |
| EE-34 | V239S |
| EE-35 | A241S |
| EE-36 | L243S |
| EE-37 | A247S |
| EE-38 | I250S |
| EE-39 | L252S |
| EE-40 | A254S |
| EE-41 | A256S |
| EE-42 | F257S |
| EE-43 | A259S |
| EE-44 | L261S |
| EE-45 | F265S |
| EE-46 | L268S |
| EE-47 | V269S |
| EE-48 | M272S |
| mmApoE- | ATGAAAGCACTTTGGGCCGTGCTGCTAGTTACGCTCCTGACAGGCTGCCTCGCGGA |
| wt | AGGGGAACCGGAGGTGACGGACCAACTTGAGTGGCAGTCTAATCAGCCCTGGGAA |
| | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCCGCAAGATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcCATC |
| | ACCATCACCATCACTGA (SEQ ID NO: 165) |
| mmApoE- | MKALWAVLLVTLLTGCLAEGEPEVTDQLEWQSNQPWEQALNRFWDYLRWVQTLSDQV |
| wt | QEELQSSQVTQELTALMEDTMTEVKAYKKELEEQLGPVAEETRARLGKEVQAAQARLGA |
| | DMEDLRNRLGQYRNEVHTMLGQSTEEIRARLSTHLRKMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGHHHHHH* (SEQ ID NO: 166) |
| mmApoE- | ATGAAAGCACTTTGGGCCGTGCTGCTAGTTACGCTCCTGACAGGCTGCCTCGCGGA |
| mut_R134A_ | AGGGGAACCGGAGGTGACGGACCAACTTGAGTGGCAGTCTAATCAGCCCTGGGAA |
| K135S | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcCATC |
| | ACCATCACCATCACTGA (SEQ ID NO: 167) |
| mmApoE- | MKALWAVLLVTLLTGCLAEGEPEVTDQLEWQSNQPWEQALNRFWDYLRWVQTLSDQV |
| mut_R134A_ | QEELQSSQVTQELTALMEDTMTEVKAYKKELEEQLGPVAEETRARLGKEVQAAQARLGA |
| K135S | DMEDLRNRLGQYRNEVHTMLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGHHHHHH* (SEQ ID NO: 168) |
| mmApoE_GSG_ | ATGAAAGCACTTTGGGCCGTGCTGCTAGTTACGCTCCTGACAGGCTGCCTCGCGGA |
| Dimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| M117 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGggatccggtGGCGAGATAGCCGCCCTTAAACAGGAGATAGCT |
| | GCCTTGAAAAAGGAGAACGCTGCACTGAAGTGGGAGATTGCGGCTCTCAAGCAGGG |
| | CTATTACggttccggaTTAGGTCAGAGCACAGAGGAAATCAGAGCCCGGCTCAGCACA |
| | CACCTCGCAAGTATGCGCAAGCGCCTAATGCGTGACGCCGAGGATCTGCAGAAGC |
| | GCCTGGCGGTGTACAAAGCTGGCGCAAGAGAGGGCGCCGAGAGAGGCGTCTCGG |
| | CCATTAGAGAGAGACTAGGACCCTTGGTCGAGCAGGGGAGGCAAAGAACAGCCAA |
| | CCTGGGAGCCGGCGCCGCACAACCTCTAAGAGACCGCGCTCAGGCCTTTGGTGAT |
| | CGGATAAGAGGAAGGTTGGAGGAGGTGGGCAATCAGGCAAGAGACCGTCTCGAGG |
| | AAGTGCGCGAGCACATGGAGGAGGTGAGAAGTAAAATGGAAGAACAAACCCAGCAG |
| | ATTAGGCTTCAGGCAGAAATATTTCAGGCCCGCTTAAAGGGCTGGTTCGAGCCTATC |
| | GTGGAGGATATGCATAGACAGTGGGCCAACCTGATGGAGAAGATCCAAGCCTCTGTT |
| | GCAACCAACCCCATCATAACTCCAGTGGCTCAGGAAAACCAGggcagcggcCATCACC |
| | ATCACCATCACTGA (SEQ ID NO: 169) |
| mmApoE_GSG_ | MKALWAVLLVTLLTGCLAEGEPEVTDQLEWQSNQPWEQALNRFWDYLRWVQTLSDQV |
| Dimeric_ | QEELQSSQVTQELTALMEDTMTEVKAYKKELEEQLGPVAEETRARLGKEVQAAQARLGA |
| zipper_to_ | DMEDLRNRLGQYRNEVHTMGSGGEIAALKQEIAALKKENAALKWEIAALKQGYYGSGLG |
| M117 | QSTEEIRARLSTHLASMRKRLMRDAEDLQKRLAVYKAGAREGAERGVSAIRERLGPLVEQ |
| | GRQRTANLGAGAAQPLRDRAQAFGDRIRGRLEEVGNQARDRLEEVREHMEEVRSKMEE |
| | QTQQIRLQAEIFQARLKGWFEPIVEDMHRQWANLMEKIQASVATNPIITPVAQENQGSG |
| | HHHHHH* (SEQ ID NO: 170) |
| mmApoE_GSG_ | ATGAAAGCACTTTGGGCCGTGCTGCTAGTTACGCTCCTGACAGGCTGCCTCGCGGA |
| Trimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| M117 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGggatccggtGGCGAAATTGCCGCTATAAAACAGGAAATAGCTG |
| | CCATCAAGAAGGAAATAGCTGCCATTAAGTGGGAGATTGCCGCGATCAAACAGGGCT |
| | ACGGCggttccggaTTAGGTCAGAGCACAGAGGAAATCAGAGCCCGGCTCAGCACAC |
| | ACCTCGCAAGTATGCGCAAGCGCCTAATGCGTGACGCCGAGGATCTGCAGAAGCG |
| | CCTGGCGGTGTACAAAGCTGGCGCAAGAGAGGGCGCCGAGAGAGGCGTCTCGGC |
| | CATTAGAGAGAGACTAGGACCCTTGGTCGAGCAGGGGAGGCAAAGAACAGCCAAC |
| | CTGGGAGCCGGCGCCGCACAACCTCTAAGAGACCGCGCTCAGGCCTTTGGTGATC |
| | GGATAAGAGGAAGGTTGGAGGAGGTGGGCAATCAGGCAAGAGACCGTCTCGAGGA |
| | AGTGCGCGAGCACATGGAGGAGGTGAGAAGTAAAATGGAAGAACAAACCCAGCAGA |
| | TTAGGCTTCAGGCAGAAATATTTCAGGCCCGCTTAAAGGGCTGGTTCGAGCCTATCG |
| | TGGAGGATATGCATAGACAGTGGGCCAACCTGATGGAGAAGATCCAAGCCTCTGTTG |
| | CAACCAACCCCATCATAACTCCAGTGGCTCAGGAAAACCAGggcagcggcCATCACCA |
| | TCACCATCACTGA (SEQ ID NO: 171) |
| mmApoE_GSG_ | MKALWAVLLVTLLTGCLAEGEPEVTDQLEWQSNQPWEQALNRFWDYLRWVQTLSDQV |
| Trimeric_ | QEELQSSQVTQELTALMEDTMTEVKAYKKELEEQLGPVAEETRARLGKEVQAAQARLGA |
| zipper_to_ | DMEDLRNRLGQYRNEVHTMGSGGEIAAIKQEIAAIKKEIAAIKWEIAAIKQGYGGSGLGQS |
| M117 | TEEIRARLSTHLASMRKRLMRDAEDLQKRLAVYKAGAREGAERGVSAIRERLGPLVEQGR |
| | QRTANLGAGAAQPLRDRAQAFGDRIRGRLEEVGNQARDRLEEVREHMEEVRSKMEEQT |
| | QQIRLQAEIFQARLKGWFEPIVEDMHRQWANLMEKIQASVATNPIITPVAQENQGSGHH |
| | HHHH* (SEQ ID NO: 172) |
| mmApoE_GSG_ | ATGAAAGCACTTTGGGCCGTGCTGCTAGTTACGCTCCTGACAGGCTGCCTCGCGGA |
| Tetrameric_ | AGGGGAACCGGAGGTGACGGACCAACTTGAGTGGCAGTCTAATCAGCCCTGGGAA |
| zipper_ | CAGGCACTCAACCGCTTCTGGGATTATCTGCGCTGGGTACAGACCCTGAGCGACCA |
| to_M117 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGggatccggtGGCGAGCTGGCTGCCATTAAGCAGGAGCTCGCT |
| | GCTATCAAGAAAGAGCTTGCAGCAATTAAGTGGGAATTGGCAGCAATCAAACAGGGC |
| | GCTGGGggttccggaTTAGGTCAGAGCACAGAGGAAATCAGAGCCCGGCTCAGCACA |
| | CACCTCGCAAGTATGCGCAAGCGCCTAATGCGTGACGCCGAGGATCTGCAGAAGC |
| | GCCTGGCGGTGTACAAAGCTGGCGCAAGAGAGGGCGCCGAGAGAGGCGTCTCGG |
| | CCATTAGAGAGAGACTAGGACCCTTGGTCGAGCAGGGGAGGCAAAGAACAGCCAA |
| | CCTGGGAGCCGGCGCCGCACAACCTCTAAGAGACCGCGCTCAGGCCTTTGGTGAT |
| | CGGATAAGAGGAAGGTTGGAGGAGGTGGGCAATCAGGCAAGAGACCGTCTCGAGG |
| | AAGTGCGCGAGCACATGGAGGAGGTGAGAAGTAAAATGGAAGAACAAACCCAGCAG |
| | ATTAGGCTTCAGGCAGAAATATTTCAGGCCCGCTTAAAGGGCTGGTTCGAGCCTATC |
| | GTGGAGGATATGCATAGACAGTGGGCCAACCTGATGGAGAAGATCCAAGCCTCTGTT |
| | GCAACCAACCCCATCATAACTCCAGTGGCTCAGGAAAACCAGggcagcggcCATCACC |
| | ATCACCATCACTGA (SEQ ID NO: 173) |
| mmApoE_GSG_ | MKALWAVLLVTLLTGCLAEGEPEVTDQLEWQSNQPWEQALNRFWDYLRWVQTLSDQV |
| Tetrameric_ | QEELQSSQVTQELTALMEDTMTEVKAYKKELEEQLGPVAEETRARLGKEVQAAQARLGA |
| zipper_ | DMEDLRNRLGQYRNEVHTMGSGGELAAIKQELAAIKKELAAIKWELAAIKQGAGGSGLG |
| to_M117 | QSTEEIRARLSTHLASMRKRLMRDAEDLQKRLAVYKAGAREGAERGVSAIRERLGPLVEQ |
| | GRQRTANLGAGAAQPLRDRAQAFGDRIRGRLEEVGNQARDRLEEVREHMEEVRSKMEE |
| | QTQQIRLQAEIFQARLKGWFEPIVEDMHRQWANLMEKIQASVATNPIITPVAQENQGSG |
| | HHHHHH* (SEQ ID NO: 174) |
| mmApoE_ | ATGAAAGCACTTTGGGCCGTGCTGCTAGTTACGCTCCTGACAGGCTGCCTCGCGGA |
| XTEN_Dimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| M117 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGAGCGGCAGCGAGACCCCCGGCACCAGCGAGAGCGCCAC |
| | CCCCGAGAGCGGCGAGATAGCCGCCCTTAAACAGGAGATAGCTGCCTTGAAAAAG |
| | GAGAACGCTGCACTGAAGTGGGAGATTGCGGCTCTCAAGCAGGGCTATTACTCCGG |
| | CTCAGAGACCCCAGGCACCTCTGAGTCTGCAACTCCTGAGTCATTAGGTCAGAGCA |
| | CAGAGGAAATCAGAGCCCGGCTCAGCACACACCTCGCAAGTATGCGCAAGCGCCT |
| | AATGCGTGACGCCGAGGATCTGCAGAAGCGCCTGGCGGTGTACAAAGCTGGCGCA |
| | AGAGAGGGCGCCGAGAGAGGCGTCTCGGCCATTAGAGAGAGACTAGGACCCTTGG |
| | TCGAGCAGGGGAGGCAAAGAACAGCCAACCTGGGAGCCGGCGCCGCACAACCTC |
| | TAAGAGACCGCGCTCAGGCCTTTGGTGATCGGATAAGAGGAAGGTTGGAGGAGGTG |
| | GGCAATCAGGCAAGAGACCGTCTCGAGGAAGTGCGCGAGCACATGGAGGAGGTGA |
| | GAAGTAAAATGGAAGAACAAACCCAGCAGATTAGGCTTCAGGCAGAAATATTTCAGG |
| | CCCGCTTAAAGGGCTGGTTCGAGCCTATCGTGGAGGATATGCATAGACAGTGGGCC |
| | AACCTGATGGAGAAGATCCAAGCCTCTGTTGCAACCAACCCCATCATAACTCCAGTG |
| | GCTCAGGAAAACCAGggcagcggcCATCACCATCACCATCACTGA (SEQ ID NO: 175) |
| mmApoE_XTEN_ | MKALWAVLLVTLLTGCLAEGEPEVTDQLEWQSNQPWEQALNRFWDYLRWVQTLSDQV |
| Dimeric_ | QEELQSSQVTQELTALMEDTMTEVKAYKKELEEQLGPVAEETRARLGKEVQAAQARLGA |
| zipper_to_ | DMEDLRNRLGQYRNEVHTMSGSETPGTSESATPESGEIAALKQEIAALKKENAALKWEIA |
| M117 | ALKQGYYSGSETPGTSESATPESLGQSTEEIRARLSTHLASMRKRLMRDAEDLQKRLAVYK |
| | AGAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGHHHHHH* (SEQ ID NO: 176) |
| mmApoE_XTEN_ | ATGAAAGCACTTTGGGCCGTGCTGCTAGTTACGCTCCTGACAGGCTGCCTCGCGGA |
| Trimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| M117 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGAGCGGCAGCGAGACCCCCGGCACCAGCGAGAGCGCCAC |
| | CCCCGAGAGCGGCGAAATTGCCGCTATAAAACAGGAAATAGCTGCCATCAAGAAGG |
| | AAATAGCTGCCATTAAGTGGGAGATTGCCGCGATCAAACAGGGCTACGGCTCCGGC |
| | TCAGAGACCCCAGGCACCTCTGAGTCTGCAACTCCTGAGTCATTAGGTCAGAGCAC |
| | AGAGGAAATCAGAGCCCGGCTCAGCACACACCTCGCAAGTATGCGCAAGCGCCTA |
| | ATGCGTGACGCCGAGGATCTGCAGAAGCGCCTGGCGGTGTACAAAGCTGGCGCAA |
| | GAGAGGGCGCCGAGAGAGGCGTCTCGGCCATTAGAGAGAGACTAGGACCCTTGGT |
| | CGAGCAGGGGAGGCAAAGAACAGCCAACCTGGGAGCCGGCGCCGCACAACCTCT |
| | AAGAGACCGCGCTCAGGCCTTTGGTGATCGGATAAGAGGAAGGTTGGAGGAGGTGG |
| | GCAATCAGGCAAGAGACCGTCTCGAGGAAGTGCGCGAGCACATGGAGGAGGTGAG |
| | AAGTAAAATGGAAGAACAAACCCAGCAGATTAGGCTTCAGGCAGAAATATTTCAGGC |
| | CCGCTTAAAGGGCTGGTTCGAGCCTATCGTGGAGGATATGCATAGACAGTGGGCCA |
| | ACCTGATGGAGAAGATCCAAGCCTCTGTTGCAACCAACCCCATCATAACTCCAGTGG |
| | CTCAGGAAAACCAGggcagcggcCATCACCATCACCATCACTGA (SEQ ID NO: 177) |
| mmApoE_XTEN_ | MKALWAVLLVTLLTGCLAEGEPEVTDQLEWQSNQPWEQALNRFWDYLRWVQTLSDQV |
| Trimeric_ | QEELQSSQVTQELTALMEDTMTEVKAYKKELEEQLGPVAEETRARLGKEVQAAQARLGA |
| zipper_to_ | DMEDLRNRLGQYRNEVHTMSGSETPGTSESATPESGEIAAIKQEIAAIKKEIAAIKWEIAAIK |
| M117 | QGYGSGSETPGTSESATPESLGQSTEEIRARLSTHLASMRKRLMRDAEDLQKRLAVYKAG |
| | AREGAERGVSAIRERLGPLVEQGRQRTANLGAGAAQPLRDRAQAFGDRIRGRLEEVGN |
| | QARDRLEEVREHMEEVRSKMEEQTQQIRLQAEIFQARLKGWFEPIVEDMHRQWANLME |
| | KIQASVATNPIITPVAQENQGSGHHHHHH* (SEQ ID NO: 178) |
| mmApoE_XTEN_ | ATGAAAGCACTTTGGGCCGTGCTGCTAGTTACGCTCCTGACAGGCTGCCTCGCGGA |
| Tetrameric_ | AGGGGAACCGGAGGTGACGGACCAACTTGAGTGGCAGTCTAATCAGCCCTGGGAA |
| zipper_ | CAGGCACTCAACCGCTTCTGGGATTATCTGCGCTGGGTACAGACCCTGAGCGACCA |
| to_M117 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGAGCGGCAGCGAGACCCCCGGCACCAGCGAGAGCGCCAC |
| | CCCCGAGAGCGGCGAGCTGGCTGCCATTAAGCAGGAGCTCGCTGCTATCAAGAAA |
| | GAGCTTGCAGCAATTAAGTGGGAATTGGCAGCAATCAAACAGGGCGCTGGGTCCGG |
| | CTCAGAGACCCCAGGCACCTCTGAGTCTGCAACTCCTGAGTCATTAGGTCAGAGCA |
| | CAGAGGAAATCAGAGCCCGGCTCAGCACACACCTCGCAAGTATGCGCAAGCGCCT |
| | AATGCGTGACGCCGAGGATCTGCAGAAGCGCCTGGCGGTGTACAAAGCTGGCGCA |
| | AGAGAGGGCGCCGAGAGAGGCGTCTCGGCCATTAGAGAGAGACTAGGACCCTTGG |
| | TCGAGCAGGGGAGGCAAAGAACAGCCAACCTGGGAGCCGGCGCCGCACAACCTC |
| | TAAGAGACCGCGCTCAGGCCTTTGGTGATCGGATAAGAGGAAGGTTGGAGGAGGTG |
| | GGCAATCAGGCAAGAGACCGTCTCGAGGAAGTGCGCGAGCACATGGAGGAGGTGA |
| | GAAGTAAAATGGAAGAACAAACCCAGCAGATTAGGCTTCAGGCAGAAATATTTCAGG |
| | CCCGCTTAAAGGGCTGGTTCGAGCCTATCGTGGAGGATATGCATAGACAGTGGGCC |
| | AACCTGATGGAGAAGATCCAAGCCTCTGTTGCAACCAACCCCATCATAACTCCAGTG |
| | GCTCAGGAAAACCAGggcagcggcCATCACCATCACCATCACTGA (SEQ ID NO: 179) |
| mmApoE_XTEN_ | MKALWAVLLVTLLTGCLAEGEPEVTDQLEWQSNQPWEQALNRFWDYLRWVQTLSDQV |
| Tetrameric_ | QEELQSSQVTQELTALMEDTMTEVKAYKKELEEQLGPVAEETRARLGKEVQAAQARLGA |
| zipper_ | DMEDLRNRLGQYRNEVHTMSGSETPGTSESATPESGELAAIKQELAAIKKELAAIKWELAA |
| to_M117 | IKQGAGSGSETPGTSESATPESLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGHHHHHH* (SEQ ID NO: 180) |
| mmApoE_GSG_ | ATGAAAGCACTTTGGGCCGTGCTGCTAGTTACGCTCCTGACAGGCTGCCTCGCGGA |
| Dimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| L187 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtGGCGAGATAGCCGCCCTTAAACAGGAGATAGCTGCCTTGAAAAA |
| | GGAGAACGCTGCACTGAAGTGGGAGATTGCGGCTCTCAAGCAGGGCTATTACggttcc |
| | ggaGGAGCCGGCGCCGCACAACCTCTAAGAGACCGCGCTCAGGCCTTTGGTGATC |
| | GGATAAGAGGAAGGTTGGAGGAGGTGGGCAATCAGGCAAGAGACCGTCTCGAGGA |
| | AGTGCGCGAGCACATGGAGGAGGTGAGAAGTAAAATGGAAGAACAAACCCAGCAGA |
| | TTAGGCTTCAGGCAGAAATATTTCAGGCCCGCTTAAAGGGCTGGTTCGAGCCTATCG |
| | TGGAGGATATGCATAGACAGTGGGCCAACCTGATGGAGAAGATCCAAGCCTCTGTTG |
| | CAACCAACCCCATCATAACTCCAGTGGCTCAGGAAAACCAGggcagcggcCATCACCA |
| | TCACCATCACTGA (SEQ ID NO: 181) |
| mmApoE_GSG_ | MKALWAVLLVTLLTGCLAEGEPEVTDQLEWQSNQPWEQALNRFWDYLRWVQTLSDQV |
| Dimeric_ | QEELQSSQVTQELTALMEDTMTEVKAYKKELEEQLGPVAEETRARLGKEVQAAQARLGA |
| zipper_to_ | DMEDLRNRLGQYRNEVHTMLGQSTEEIRARLSTHLASMRKRLMRDAEDLQKRLAVYKA |
| L187 | GAREGAERGVSAIRERLGPLVEQGRQRTANLGSGGEIAALKQEIAALKKENAALKWEIAAL |
| | KQGYYGSGGAGAAQPLRDRAQAFGDRIRGRLEEVGNQARDRLEEVREHMEEVRSKME |
| | EQTQQIRLQAEIFQARLKGWFEPIVEDMHRQWANLMEKIQASVATNPIITPVAQENQGS |
| | GHHHHHH* (SEQ ID NO: 182) |
| mmApoE_GSG_ | ATGAAAGCACTTTGGGCCGTGCTGCTAGTTACGCTCCTGACAGGCTGCCTCGCGGA |
| Trimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| L187 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtGGCGAAATTGCCGCTATAAAACAGGAAATAGCTGCCATCAAGAA |
| | GGAAATAGCTGCCATTAAGTGGGAGATTGCCGCGATCAAACAGGGCTACGGCggttcc |
| | ggaGGAGCCGGCGCCGCACAACCTCTAAGAGACCGCGCTCAGGCCTTTGGTGATC |
| | GGATAAGAGGAAGGTTGGAGGAGGTGGGCAATCAGGCAAGAGACCGTCTCGAGGA |
| | AGTGCGCGAGCACATGGAGGAGGTGAGAAGTAAAATGGAAGAACAAACCCAGCAGA |
| | TTAGGCTTCAGGCAGAAATATTTCAGGCCCGCTTAAAGGGCTGGTTCGAGCCTATCG |
| | TGGAGGATATGCATAGACAGTGGGCCAACCTGATGGAGAAGATCCAAGCCTCTGTTG |
| | CAACCAACCCCATCATAACTCCAGTGGCTCAGGAAAACCAGggcagcggcCATCACCA |
| | TCACCATCACTGA (SEQ ID NO: 183) |
| mmApoE_GSG_ | MKALWAVLLVTLLTGCLAEGEPEVTDQLEWQSNQPWEQALNRFWDYLRWVQTLSDQV |
| Trimeric_ | QEELQSSQVTQELTALMEDTMTEVKAYKKELEEQLGPVAEETRARLGKEVQAAQARLGA |
| zipper_to_ | DMEDLRNRLGQYRNEVHTMLGQSTEEIRARLSTHLASMRKRLMRDAEDLQKRLAVYKA |
| L187 | GAREGAERGVSAIRERLGPLVEQGRQRTANLGSGGEIAAIKQEIAAIKKEIAAIKWEIAAIKQ |
| | GYGGSGGAGAAQPLRDRAQAFGDRIRGRLEEVGNQARDRLEEVREHMEEVRSKMEEQ |
| | TQQIRLQAEIFQARLKGWFEPIVEDMHRQWANLMEKIQASVATNPIITPVAQENQGSGH |
| | HHHHH* (SEQ ID NO: 184) |
| mmApoE_GSG_ | ATGAAAGCACTTTGGGCCGTGCTGCTAGTTACGCTCCTGACAGGCTGCCTCGCGGA |
| Tetrameric_ | AGGGGAACCGGAGGTGACGGACCAACTTGAGTGGCAGTCTAATCAGCCCTGGGAA |
| zipper_ | CAGGCACTCAACCGCTTCTGGGATTATCTGCGCTGGGTACAGACCCTGAGCGACCA |
| to_L187 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtGGCGAGCTGGCTGCCATTAAGCAGGAGCTCGCTGCTATCAAGA |
| | AAGAGCTTGCAGCAATTAAGTGGGAATTGGCAGCAATCAAACAGGGCGCTGGGggttc |
| | cggaGGAGCCGGCGCCGCACAACCTCTAAGAGACCGCGCTCAGGCCTTTGGTGATC |
| | GGATAAGAGGAAGGTTGGAGGAGGTGGGCAATCAGGCAAGAGACCGTCTCGAGGA |
| | AGTGCGCGAGCACATGGAGGAGGTGAGAAGTAAAATGGAAGAACAAACCCAGCAGA |
| | TTAGGCTTCAGGCAGAAATATTTCAGGCCCGCTTAAAGGGCTGGTTCGAGCCTATCG |
| | TGGAGGATATGCATAGACAGTGGGCCAACCTGATGGAGAAGATCCAAGCCTCTGTTG |
| | CAACCAACCCCATCATAACTCCAGTGGCTCAGGAAAACCAGggcagcggcCATCACCA |
| | TCACCATCACTGA (SEQ ID NO: 185) |
| mmApoE_GSG_ | MKALWAVLLVTLLTGCLAEGEPEVTDQLEWQSNQPWEQALNRFWDYLRWVQTLSDQV |
| Tetrameric_ | QEELQSSQVTQELTALMEDTMTEVKAYKKELEEQLGPVAEETRARLGKEVQAAQARLGA |
| zipper_ | DMEDLRNRLGQYRNEVHTMLGQSTEEIRARLSTHLASMRKRLMRDAEDLQKRLAVYKA |
| to_L187 | GAREGAERGVSAIRERLGPLVEQGRQRTANLGSGGELAAIKQELAAIKKELAAIKWELAAI |
| | KQGAGGSGGAGAAQPLRDRAQAFGDRIRGRLEEVGNQARDRLEEVREHMEEVRSKME |
| | EQTQQIRLQAEIFQARLKGWFEPIVEDMHRQWANLMEKIQASVATNPIITPVAQENQGS |
| | GHHHHHH* (SEQ ID NO: 186) |
| mmApoE_XTEN_ | ATGAAAGCACTTTGGGCCGTGCTGCTAGTTACGCTCCTGACAGGCTGCCTCGCGGA |
| Dimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| L187 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGAGCGGCAGCGAGACCCCCGGCACCAGCGAGAGCGCCACCCCCGAGAG |
| | CGGCGAGATAGCCGCCCTTAAACAGGAGATAGCTGCCTTGAAAAAGGAGAACGCTG |
| | CACTGAAGTGGGAGATTGCGGCTCTCAAGCAGGGCTATTACTCCGGCTCAGAGACC |
| | CCAGGCACCTCTGAGTCTGCAACTCCTGAGTCAGGAGCCGGCGCCGCACAACCTC |
| | TAAGAGACCGCGCTCAGGCCTTTGGTGATCGGATAAGAGGAAGGTTGGAGGAGGTG |
| | GGCAATCAGGCAAGAGACCGTCTCGAGGAAGTGCGCGAGCACATGGAGGAGGTGA |
| | GAAGTAAAATGGAAGAACAAACCCAGCAGATTAGGCTTCAGGCAGAAATATTTCAGG |
| | CCCGCTTAAAGGGCTGGTTCGAGCCTATCGTGGAGGATATGCATAGACAGTGGGCC |
| | AACCTGATGGAGAAGATCCAAGCCTCTGTTGCAACCAACCCCATCATAACTCCAGTG |
| | GCTCAGGAAAACCAGggcagcggcCATCACCATCACCATCACTGA (SEQ ID NO: 187) |
| mmApoE_XTEN_ | MKALWAVLLVTLLTGCLAEGEPEVTDQLEWQSNQPWEQALNRFWDYLRWVQTLSDQV |
| Dimeric_ | QEELQSSQVTQELTALMEDTMTEVKAYKKELEEQLGPVAEETRARLGKEVQAAQARLGA |
| zipper_to_ | DMEDLRNRLGQYRNEVHTMLGQSTEEIRARLSTHLASMRKRLMRDAEDLQKRLAVYKA |
| L187 | GAREGAERGVSAIRERLGPLVEQGRQRTANLSGSETPGTSESATPESGEIAALKQEIAALK |
| | KENAALKWEIAALKQGYYSGSETPGTSESATPESGAGAAQPLRDRAQAFGDRIRGRLEEV |
| | GNQARDRLEEVREHMEEVRSKMEEQTQQIRLQAEIFQARLKGWFEPIVEDMHRQWANL |
| | MEKIQASVATNPIITPVAQENQGSGHHHHHH* (SEQ ID NO: 188) |
| mmApoE_XTEN_ | ATGAAAGCACTTTGGGCCGTGCTGCTAGTTACGCTCCTGACAGGCTGCCTCGCGGA |
| Trimeric_ | AGGGGAACCGGAGGTGACGGACCAACTTGAGTGGCAGTCTAATCAGCCCTGGGAA |
| zipper_to_ | CAGGCACTCAACCGCTTCTGGGATTATCTGCGCTGGGTACAGACCCTGAGCGACCA |
| L187 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGAGCGGCAGCGAGACCCCCGGCACCAGCGAGAGCGCCACCCCCGAGAG |
| | CGGCGAAATTGCCGCTATAAAACAGGAAATAGCTGCCATCAAGAAGGAAATAGCTGC |
| | CATTAAGTGGGAGATTGCCGCGATCAAACAGGGCTACGGCTCCGGCTCAGAGACC |
| | CCAGGCACCTCTGAGTCTGCAACTCCTGAGTCAGGAGCCGGCGCCGCACAACCTC |
| | TAAGAGACCGCGCTCAGGCCTTTGGTGATCGGATAAGAGGAAGGTTGGAGGAGGTG |
| | GGCAATCAGGCAAGAGACCGTCTCGAGGAAGTGCGCGAGCACATGGAGGAGGTGA |
| | GAAGTAAAATGGAAGAACAAACCCAGCAGATTAGGCTTCAGGCAGAAATATTTCAGG |
| | CCCGCTTAAAGGGCTGGTTCGAGCCTATCGTGGAGGATATGCATAGACAGTGGGCC |
| | AACCTGATGGAGAAGATCCAAGCCTCTGTTGCAACCAACCCCATCATAACTCCAGTG |
| | GCTCAGGAAAACCAGggcagcggcCATCACCATCACCATCACTGA (SEQ ID NO: 189) |
| mmApoE_ | MKALWAVLLVTLLTGCLAEGEPEVTDQLEWQSNQPWEQALNRFWDYLRWVQTLSDQV |
| XTEN_Trimeric_ | QEELQSSQVTQELTALMEDTMTEVKAYKKELEEQLGPVAEETRARLGKEVQAAQARLGA |
| zipper_to_ | DMEDLRNRLGQYRNEVHTMLGQSTEEIRARLSTHLASMRKRLMRDAEDLQKRLAVYKA |
| L187 | GAREGAERGVSAIRERLGPLVEQGRQRTANLSGSETPGTSESATPESGEIAAIKQEIAAIKK |
| | EIAAIKWEIAAIKQGYGSGSETPGTSESATPESGAGAAQPLRDRAQAFGDRIRGRLEEVGN |
| | QARDRLEEVREHMEEVRSKMEEQTQQIRLQAEIFQARLKGWFEPIVEDMHRQWANLME |
| | KIQASVATNPIITPVAQENQGSGHHHHHH* (SEQ ID NO: 200) |
| mmApoE_XTEN_ | ATGAAAGCACTTTGGGCCGTGCTGCTAGTTACGCTCCTGACAGGCTGCCTCGCGGA |
| Tetrameric_ | AGGGGAACCGGAGGTGACGGACCAACTTGAGTGGCAGTCTAATCAGCCCTGGGAA |
| zipper_ | CAGGCACTCAACCGCTTCTGGGATTATCTGCGCTGGGTACAGACCCTGAGCGACCA |
| to_L187 | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGAGCGGCAGCGAGACCCCCGGCACCAGCGAGAGCGCCACCCCCGAGAG |
| | CGGCGAGCTGGCTGCCATTAAGCAGGAGCTCGCTGCTATCAAGAAAGAGCTTGCAG |
| | CAATTAAGTGGGAATTGGCAGCAATCAAACAGGGCGCTGGGTCCGGCTCAGAGACC |
| | CCAGGCACCTCTGAGTCTGCAACTCCTGAGTCAGGAGCCGGCGCCGCACAACCTC |
| | TAAGAGACCGCGCTCAGGCCTTTGGTGATCGGATAAGAGGAAGGTTGGAGGAGGTG |
| | GGCAATCAGGCAAGAGACCGTCTCGAGGAAGTGCGCGAGCACATGGAGGAGGTGA |
| | GAAGTAAAATGGAAGAACAAACCCAGCAGATTAGGCTTCAGGCAGAAATATTTCAGG |
| | CCCGCTTAAAGGGCTGGTTCGAGCCTATCGTGGAGGATATGCATAGACAGTGGGCC |
| | AACCTGATGGAGAAGATCCAAGCCTCTGTTGCAACCAACCCCATCATAACTCCAGTG |
| | GCTCAGGAAAACCAGggcagcggcCATCACCATCACCATCACTGA (SEQ ID NO: 201) |
| mmApoE_XTEN_ | MKALWAVLLVTLLTGCLAEGEPEVTDQLEWQSNQPWEQALNRFWDYLRWVQTLSDQV |
| Tetrameric_ | QEELQSSQVTQELTALMEDTMTEVKAYKKELEEQLGPVAEETRARLGKEVQAAQARLGA |
| zipper_ | DMEDLRNRLGQYRNEVHTMLGQSTEEIRARLSTHLASMRKRLMRDAEDLQKRLAVYKA |
| to_L187 | GAREGAERGVSAIRERLGPLVEQGRQRTANLSGSETPGTSESATPESGELAAIKQELAAIK |
| | KELAAIKWELAAIKQGAGSGSETPGTSESATPESGAGAAQPLRDRAQAFGDRIRGRLEEV |
| | GNQARDRLEEVREHMEEVRSKMEEQTQQIRLQAEIFQARLKGWFEPIVEDMHRQWANL |
| | MEKIQASVATNPIITPVAQENQGSGHHHHHH* (SEQ ID NO: 202) |
| mmApoE_ | ATGAAAGCACTTTGGGCCGTGCTGCTAGTTACGCTCCTGACAGGCTGCCTCGCGGA |
| GSG_18A_to | AGGGGAACCGGAGGTGACGGACCAACTTGAGTGGCAGTCTAATCAGCCCTGGGAA |
| L187_ | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtGACTGGCTGAAGGCCTTCTACGACAAGGTGGCCGAGAAGCTGA |
| | AGGAGGCCTTCggttccggaGGAGCCGGCGCCGCACAACCTCTAAGAGACCGCGCTC |
| | AGGCCTTTGGTGATCGGATAAGAGGAAGGTTGGAGGAGGTGGGCAATCAGGCAAGA |
| | GACCGTCTCGAGGAAGTGCGCGAGCACATGGAGGAGGTGAGAAGTAAAATGGAAG |
| | AACAAACCCAGCAGATTAGGCTTCAGGCAGAAATATTTCAGGCCCGCTTAAAGGGCT |
| | GGTTCGAGCCTATCGTGGAGGATATGCATAGACAGTGGGCCAACCTGATGGAGAAG |
| | ATCCAAGCCTCTGTTGCAACCAACCCCATCATAACTCCAGTGGCTCAGGAAAACCAG |
| | ggcagcggcCATCACCATCACCATCACTGA (SEQ ID NO: 203) |
| mmApoE_ | MKALWAVLLVTLLTGCLAEGEPEVTDQLEWQSNQPWEQALNRFWDYLRWVQTLSDQV |
| GSG_18A_to_ | QEELQSSQVTQELTALMEDTMTEVKAYKKELEEQLGPVAEETRARLGKEVQAAQARLGA |
| L187 | DMEDLRNRLGQYRNEVHTMLGQSTEEIRARLSTHLASMRKRLMRDAEDLQKRLAVYKA |
| | GAREGAERGVSAIRERLGPLVEQGRQRTANLGSGDWLKAFYDKVAEKLKEAFGSGGAG |
| | AAQPLRDRAQAFGDRIRGRLEEVGNQARDRLEEVREHMEEVRSKMEEQTQQIRLQAEIF |
| | QARLKGWFEPIVEDMHRQWANLMEKIQASVATNPIITPVAQENQGSGHHHHHH* (SEQ |
| | ID NO: 204) |
| mmApoE_ | ATGAAAGCACTTTGGGCCGTGCTGCTAGTTACGCTCCTGACAGGCTGCCTCGCGGA |
| GSG_4F_to_ | AGGGGAACCGGAGGTGACGGACCAACTTGAGTGGCAGTCTAATCAGCCCTGGGAA |
| L187 | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtGACTGGTTCAAGGCCTTCTACGACAAGGTGGCCGAGAAGTTCAA |
| | GGAGGCCTTCggttccggaGGAGCCGGCGCCGCACAACCTCTAAGAGACCGCGCTCA |
| | GGCCTTTGGTGATCGGATAAGAGGAAGGTTGGAGGAGGTGGGCAATCAGGCAAGAG |
| | ACCGTCTCGAGGAAGTGCGCGAGCACATGGAGGAGGTGAGAAGTAAAATGGAAGAA |
| | CAAACCCAGCAGATTAGGCTTCAGGCAGAAATATTTCAGGCCCGCTTAAAGGGCTGG |
| | TTCGAGCCTATCGTGGAGGATATGCATAGACAGTGGGCCAACCTGATGGAGAAGATC |
| | CAAGCCTCTGTTGCAACCAACCCCATCATAACTCCAGTGGCTCAGGAAAACCAGggc |
| | agcggcCATCACCATCACCATCACTGA (SEQ ID NO: 205) |
| mmApoE_ | MKALWAVLLVTLLTGCLAEGEPEVTDQLEWQSNQPWEQALNRFWDYLRWVQTLSDQV |
| GSG_4F_to_ | QEELQSSQVTQELTALMEDTMTEVKAYKKELEEQLGPVAEETRARLGKEVQAAQARLGA |
| L187 | DMEDLRNRLGQYRNEVHTMLGQSTEEIRARLSTHLASMRKRLMRDAEDLQKRLAVYKA |
| | GAREGAERGVSAIRERLGPLVEQGRQRTANLGSGDWFKAFYDKVAEKFKEAFGSGGAG |
| | AAQPLRDRAQAFGDRIRGRLEEVGNQARDRLEEVREHMEEVRSKMEEQTQQIRLQAEIF |
| | QARLKGWFEPIVEDMHRQWANLMEKIQASVATNPIITPVAQENQGSGHHHHHH* (SEQ |
| | ID NO: 206) |
| mmApoE_ | ATGAAAGCACTTTGGGCCGTGCTGCTAGTTACGCTCCTGACAGGCTGCCTCGCGGA |
| GSG_CRAC- | AGGGGAACCGGAGGTGACGGACCAACTTGAGTGGCAGTCTAATCAGCCCTGGGAA |
| 336_to_L187 | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtTTTGACAGGGCTCGCATGCTCGAGGAGTACAGTAAAAGGTTCAA |
| | GAAGTTTGGCTATggttccggaGGAGCCGGCGCCGCACAACCTCTAAGAGACCGCGCT |
| | CAGGCCTTTGGTGATCGGATAAGAGGAAGGTTGGAGGAGGTGGGCAATCAGGCAAG |
| | AGACCGTCTCGAGGAAGTGCGCGAGCACATGGAGGAGGTGAGAAGTAAAATGGAA |
| | GAACAAACCCAGCAGATTAGGCTTCAGGCAGAAATATTTCAGGCCCGCTTAAAGGGC |
| | TGGTTCGAGCCTATCGTGGAGGATATGCATAGACAGTGGGCCAACCTGATGGAGAA |
| | GATCCAAGCCTCTGTTGCAACCAACCCCATCATAACTCCAGTGGCTCAGGAAAACCA |
| | GggcagcggcCATCACCATCACCATCACTGA (SEQ ID NO: 207) |
| mmApoE_ | MKALWAVLLVTLLTGCLAEGEPEVTDQLEWQSNQPWEQALNRFWDYLRWVQTLSDQV |
| GSG_CRAC- | QEELQSSQVTQELTALMEDTMTEVKAYKKELEEQLGPVAEETRARLGKEVQAAQARLGA |
| 336_to_L187 | DMEDLRNRLGQYRNEVHTMLGQSTEEIRARLSTHLASMRKRLMRDAEDLQKRLAVYKA |
| | GAREGAERGVSAIRERLGPLVEQGRQRTANLGSGFDRARMLEEYSKRFKKFGYGSGGA |
| | GAAQPLRDRAQAFGDRIRGRLEEVGNQARDRLEEVREHMEEVRSKMEEQTQQIRLQAEI |
| | FQARLKGWFEPIVEDMHRQWANLMEKIQASVATNPIITPVAQENQGSGHHHHHH* |
| | (SEQ ID NO: 208) |
| mmApoE_ | ATGAAAGCACTTTGGGCCGTGCTGCTAGTTACGCTCCTGACAGGCTGCCTCGCGGA |
| GSG_LAH4_to_ | AGGGGAACCGGAGGTGACGGACCAACTTGAGTGGCAGTCTAATCAGCCCTGGGAA |
| L187 | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtAAAAAAGCTCTGCTCGCACTGGCTCTACACCACTTAGCTCACCT |
| | GGCCCTCCACCTGGCCCTGGCACTCAAAAAAGCCggttccggaGGAGCCGGCGCCG |
| | CACAACCTCTAAGAGACCGCGCTCAGGCCTTTGGTGATCGGATAAGAGGAAGGTTG |
| | GAGGAGGTGGGCAATCAGGCAAGAGACCGTCTCGAGGAAGTGCGCGAGCACATGG |
| | AGGAGGTGAGAAGTAAAATGGAAGAACAAACCCAGCAGATTAGGCTTCAGGCAGAAA |
| | TATTTCAGGCCCGCTTAAAGGGCTGGTTCGAGCCTATCGTGGAGGATATGCATAGAC |
| | AGTGGGCCAACCTGATGGAGAAGATCCAAGCCTCTGTTGCAACCAACCCCATCATA |
| | ACTCCAGTGGCTCAGGAAAACCAGggcagcggcCATCACCATCACCATCACTGA (SEQ |
| | ID NO: 209) |
| mmApoE_ | MKALWAVLLVTLLTGCLAEGEPEVTDQLEWQSNQPWEQALNRFWDYLRWVQTLSDQV |
| GSG_LAH4_to_ | QEELQSSQVTQELTALMEDTMTEVKAYKKELEEQLGPVAEETRARLGKEVQAAQARLGA |
| L187 | DMEDLRNRLGQYRNEVHTMLGQSTEEIRARLSTHLASMRKRLMRDAEDLQKRLAVYKA |
| | GAREGAERGVSAIRERLGPLVEQGRQRTANLGSGKKALLALALHHLAHLALHLALALKKA |
| | GSGGAGAAQPLRDRAQAFGDRIRGRLEEVGNQARDRLEEVREHMEEVRSKMEEQTQQI |
| | RLQAEIFQARLKGWFEPIVEDMHRQWANLMEKIQASVATNPIITPVAQENQGSGHHHH |
| | HH* (SEQ ID NO: 210) |
| mmApoE_ | ATGAAAGCACTTTGGGCCGTGCTGCTAGTTACGCTCCTGACAGGCTGCCTCGCGGA |
| GSG_A2- | AGGGGAACCGGAGGTGACGGACCAACTTGAGTGGCAGTCTAATCAGCCCTGGGAA |
| 17_to_L187 | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtCTGCGGAAGCTCAGAAAGCGCCTGCTTAGACTGTGGAAATTACG |
| | GAAAAGAggttccggaGGAGCCGGCGCCGCACAACCTCTAAGAGACCGCGCTCAGGC |
| | CTTTGGTGATCGGATAAGAGGAAGGTTGGAGGAGGTGGGCAATCAGGCAAGAGACC |
| | GTCTCGAGGAAGTGCGCGAGCACATGGAGGAGGTGAGAAGTAAAATGGAAGAACAA |
| | ACCCAGCAGATTAGGCTTCAGGCAGAAATATTTCAGGCCCGCTTAAAGGGCTGGTTC |
| | GAGCCTATCGTGGAGGATATGCATAGACAGTGGGCCAACCTGATGGAGAAGATCCA |
| | AGCCTCTGTTGCAACCAACCCCATCATAACTCCAGTGGCTCAGGAAAACCAGggcag |
| | cggcCATCACCATCACCATCACTGA (SEQ ID NO: 211) |
| mmApoE_ | MKALWAVLLVTLLTGCLAEGEPEVTDQLEWQSNQPWEQALNRFWDYLRWVQTLSDQV |
| GSG_A2- | QEELQSSQVTQELTALMEDTMTEVKAYKKELEEQLGPVAEETRARLGKEVQAAQARLGA |
| 17_to_L187 | DMEDLRNRLGQYRNEVHTMLGQSTEEIRARLSTHLASMRKRLMRDAEDLQKRLAVYKA |
| | GAREGAERGVSAIRERLGPLVEQGRQRTANLGSGLRKLRKRLLRLWKLRKRGSGGAGAA |
| | QPLRDRAQAFGDRIRGRLEEVGNQARDRLEEVREHMEEVRSKMEEQTQQIRLQAEIFQA |
| | RLKGWFEPIVEDMHRQWANLMEKIQASVATNPIITPVAQENQGSGHHHHHH* (SEQ ID |
| | NO: 212) |
| mmApoE_ | ATGAAAGCACTTTGGGCCGTGCTGCTAGTTACGCTCCTGACAGGCTGCCTCGCGGA |
| GSG_22A_to_ | AGGGGAACCGGAGGTGACGGACCAACTTGAGTGGCAGTCTAATCAGCCCTGGGAA |
| L187 | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGggatccggtCCTGTGCTAGACCTGTTCCGGGAACTCCTAAACGAGCTCCTGGA |
| | AGCGCTTAAGCAGAAGCTGAAAggttccggaGGAGCCGGCGCCGCACAACCTCTAAG |
| | AGACCGCGCTCAGGCCTTTGGTGATCGGATAAGAGGAAGGTTGGAGGAGGTGGGC |
| | AATCAGGCAAGAGACCGTCTCGAGGAAGTGCGCGAGCACATGGAGGAGGTGAGAA |
| | GTAAAATGGAAGAACAAACCCAGCAGATTAGGCTTCAGGCAGAAATATTTCAGGCCC |
| | GCTTAAAGGGCTGGTTCGAGCCTATCGTGGAGGATATGCATAGACAGTGGGCCAAC |
| | CTGATGGAGAAGATCCAAGCCTCTGTTGCAACCAACCCCATCATAACTCCAGTGGCT |
| | CAGGAAAACCAGggcagcggcCATCACCATCACCATCACTGA (SEQ ID NO: 213) |
| mmApoE_ | MKALWAVLLVTLLTGCLAEGEPEVTDQLEWQSNQPWEQALNRFWDYLRWVQTLSDQV |
| GSG_22A_to_ | QEELQSSQVTQELTALMEDTMTEVKAYKKELEEQLGPVAEETRARLGKEVQAAQARLGA |
| L187 | DMEDLRNRLGQYRNEVHTMLGQSTEEIRARLSTHLASMRKRLMRDAEDLQKRLAVYKA |
| | GAREGAERGVSAIRERLGPLVEQGRQRTANLGSGPVLDLFRELLNELLEALKQKLKGSGG |
| | AGAAQPLRDRAQAFGDRIRGRLEEVGNQARDRLEEVREHMEEVRSKMEEQTQQIRLQA |
| | EIFQARLKGWFEPIVEDMHRQWANLMEKIQASVATNPIITPVAQENQGSGHHHHHH* |
| | (SEQ ID NO: 214) |
| mmApoE3_ | ATGAAAGCACTTTGGGCCGTGCTGCTAGTTACGCTCCTGACAGGCTGCCTCGCGGA |
| GSG_18A_ | AGGGGAACCGGAGGTGACGGACCAACTTGAGTGGCAGTCTAATCAGCCCTGGGAA |
| GSG_6his | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcGACT |
| | GGCTGAAGGCCTTCTACGACAAGGTGGCCGAGAAGCTGAAGGAGGCCTTCggcagcg |
| | gcCATCACCATCACCATCACTGA (SEQ ID NO: 215) |
| mmApoE3_ | MKALWAVLLVTLLTGCLAEGEPEVTDQLEWQSNQPWEQALNRFWDYLRWVQTLSDQV |
| GSG_18A_ | QEELQSSQVTQELTALMEDTMTEVKAYKKELEEQLGPVAEETRARLGKEVQAAQARLGA |
| GSG_6his | DMEDLRNRLGQYRNEVHTMLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGDWLKAFYDKVAEKLKEAFGSGHHHHHH* (SEQ ID |
| | NO: 216) |
| mmApoE3_ | ATGAAAGCACTTTGGGCCGTGCTGCTAGTTACGCTCCTGACAGGCTGCCTCGCGGA |
| GSG_4F_ | AGGGGAACCGGAGGTGACGGACCAACTTGAGTGGCAGTCTAATCAGCCCTGGGAA |
| GSG_6his | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcGACT |
| | GGTTCAAGGCCTTCTACGACAAGGTGGCCGAGAAGTTCAAGGAGGCCTTCggcagcgg |
| | cCATCACCATCACCATCACTGA (SEQ ID NO: 217) |
| mmApoE3_ | MKALWAVLLVTLLTGCLAEGEPEVTDQLEWQSNQPWEQALNRFWDYLRWVQTLSDQV |
| GSG_4F_ | QEELQSSQVTQELTALMEDTMTEVKAYKKELEEQLGPVAEETRARLGKEVQAAQARLGA |
| GSG_6his | DMEDLRNRLGQYRNEVHTMLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGDWFKAFYDKVAEKFKEAFGSGHHHHHH* (SEQ ID |
| | NO: 218) |
| mmApoE3_ | ATGAAAGCACTTTGGGCCGTGCTGCTAGTTACGCTCCTGACAGGCTGCCTCGCGGA |
| GSG_CRAC- | AGGGGAACCGGAGGTGACGGACCAACTTGAGTGGCAGTCTAATCAGCCCTGGGAA |
| 336_GSG_6his | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcTTTGA |
| | CAGGGCTCGCATGCTCGAGGAGTACAGTAAAAGGTTCAAGAAGTTTGGCTATggcagc |
| | ggcCATCACCATCACCATCACTGA (SEQ ID NO: 219) |
| mmApoE3 | MKALWAVLLVTLLTGCLAEGEPEVTDQLEWQSNQPWEQALNRFWDYLRWVQTLSDQV |
| GSG_CRAC- | QEELQSSQVTQELTALMEDTMTEVKAYKKELEEQLGPVAEETRARLGKEVQAAQARLGA |
| 336_GSG_6his | DMEDLRNRLGQYRNEVHTMLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGFDRARMLEEYSKRFKKFGYGSGHHHHHH* (SEQ ID |
| | NO: 220) |
| mmApoE3_ | ATGAAAGCACTTTGGGCCGTGCTGCTAGTTACGCTCCTGACAGGCTGCCTCGCGGA |
| GSG_LAH4_ | AGGGGAACCGGAGGTGACGGACCAACTTGAGTGGCAGTCTAATCAGCCCTGGGAA |
| GSG_6his | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcAAAA |
| | AAGCTCTGCTCGCACTGGCTCTACACCACTTAGCTCACCTGGCCCTCCACCTGGCC |
| | CTGGCACTCAAAAAAGCCggcagcggcCATCACCATCACCATCACTGA (SEQ ID NO: |
| | 221) |
| mmApoE3_ | MKALWAVLLVTLLTGCLAEGEPEVTDQLEWQSNQPWEQALNRFWDYLRWVQTLSDQV |
| GSG_LAH4_ | QEELQSSQVTQELTALMEDTMTEVKAYKKELEEQLGPVAEETRARLGKEVQAAQARLGA |
| GSG_6his | DMEDLRNRLGQYRNEVHTMLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGKKALLALALHHLAHLALHLALALKKAGSGHHHHHH* |
| | (SEQ ID NO: 222) |
| mmApoE3_ | ATGAAAGCACTTTGGGCCGTGCTGCTAGTTACGCTCCTGACAGGCTGCCTCGCGGA |
| GSG_A2- | AGGGGAACCGGAGGTGACGGACCAACTTGAGTGGCAGTCTAATCAGCCCTGGGAA |
| 17_GSG_6his | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcCTGC |
| | GGAAGCTCAGAAAGCGCCTGCTTAGACTGTGGAAATTACGGAAAAGAggcagcggcCAT |
| | CACCATCACCATCACTGA (SEQ ID NO: 223) |
| mmApoE3_ | MKALWAVLLVTLLTGCLAEGEPEVTDQLEWQSNQPWEQALNRFWDYLRWVQTLSDQV |
| GSG_A2- | QEELQSSQVTQELTALMEDTMTEVKAYKKELEEQLGPVAEETRARLGKEVQAAQARLGA |
| 17_GSG_6his | DMEDLRNRLGQYRNEVHTMLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGLRKLRKRLLRLWKLRKRGSGHHHHHH* (SEQ ID |
| | NO: 224) |
| mmApoE3_ | ATGAAAGCACTTTGGGCCGTGCTGCTAGTTACGCTCCTGACAGGCTGCCTCGCGGA |
| GSG_22A_ | AGGGGAACCGGAGGTGACGGACCAACTTGAGTGGCAGTCTAATCAGCCCTGGGAA |
| GSG_6his1 | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcCCTG |
| | TGCTAGACCTGTTCCGGGAACTCCTAAACGAGCTCCTGGAAGCGCTTAAGCAGAAG |
| | CTGAAAggcagcggcCATCACCATCACCATCACTGA (SEQ ID NO: 225) |
| mmApoE3_ | MKALWAVLLVTLLTGCLAEGEPEVTDQLEWQSNQPWEQALNRFWDYLRWVQTLSDQV |
| GSG_22A_ | QEELQSSQVTQELTALMEDTMTEVKAYKKELEEQLGPVAEETRARLGKEVQAAQARLGA |
| GSG_6his1 | DMEDLRNRLGQYRNEVHTMLGQSTEEIRARLSTHLASMRKRLMRDAEDLQKRLAVYKA |
| | GAREGAERGVSAIRERLGPLVEQGRQRTANLGAGAAQPLRDRAQAFGDRIRGRLEEVG |
| | NQARDRLEEVREHMEEVRSKMEEQTQQIRLQAEIFQARLKGWFEPIVEDMHRQWANLM |
| | EKIQASVATNPIITPVAQENQGSGPVLDLFRELLNELLEALKQKLKGSGHHHHHH* (SEQ |
| | ID NO: 226) |
| Tfrc_ | ATGAAGGTTCTGTGGGCTGCGTTGCTGGTCACATTCCTGGCAGGATGCCAGGCCGA |
| minibinder1_ | CGAGGAGGAGATCCAGAAGGCCATCGAGGAGCTGCTGAGAAAGGGCGTGAGCGA |
| APOE3_R142A_ | GGAGGAGGCCGCCATCATCATCGTGCAGAGATTCAACGTGGCCGTGGTGGTGGTG |
| K143S | GTGCAGGACGAGAGACAGGGCAAGCACATCAGCGAGTACATCAGAAGATACATCC |
| | CCGAGGCCGACGTGATCCTGTTCGCCAACCTGGTGGTGATCAAGGTGGAGACCCA |
| | CGAGCTGAGCACCAGAGTGTGGGAGGCCGCCCAGAAGGCCTACggatccgggggtcc |
| | ggtagtggtggttccAAGGTGGAGCAAGCGGTGGAGACAGAGCCGGAGCCCGAGCTGC |
| | GCCAGCAGACCGAGTGGCAGAGCGGCCAGCGCTGGGAACTGGCACTGGGTCGCT |
| | TTTGGGATTACCTGCGCTGGGTGCAGACACTGTCTGAGCAGGTGCAGGAGGAGCTG |
| | CTCAGCTCCCAGGTCACCCAGGAACTGAGGGCGCTGATGGACGAGACCATGAAGG |
| | AGTTGAAGGCCTACAAATCGGAACTGGAGGAACAACTGACCCCGGTGGCGGAGGA |
| | GACGCGGGCACGGCTGTCCAAGGAGCTGCAGGCGGCGCAGGCCCGGCTGGGCG |
| | CGGACATGGAGGACGTGTGCGGCCGCCTGGTGCAGTACCGCGGCGAGGTGCAGG |
| | CCATGCTCGGCCAGAGCACCGAGGAGCTGCGGGTGCGCCTCGCCTCCCACCTGg |
| | ccagcCTGCGTAAGCGGCTCCTCCGCGATGCCGATGACCTGCAGAAGCGCCTGGCA |
| | GTGTACCAGGCCGGGGCCCGCGAGGGCGCCGAGCGCGGCCTCAGCGCCATCCG |
| | CGAGCGCCTGGGGCCCCTGGTGGAACAGGGCCGCGTGCGGGCCGCCACTGTGG |
| | GCTCCCTGGCCGGCCAGCCGCTACAGGAGCGGGCCCAGGCCTGGGGCGAGCG |
| | GCTGCGCGCGCGGATGGAGGAGATGGGCAGCCGGACCCGCGACCGCCTGGACG |
| | AGGTGAAGGAGCAGGTGGCGGAGGTGCGCGCCAAGCTGGAGGAGCAGGCCCAG |
| | CAGATACGCCTGCAGGCCGAGGCCTTCCAGGCCCGCCTCAAGAGCTGGTTCGAG |
| | CCCCTGGTGGAAGACATGCAGCGCCAGTGGGCCGGGCTGGTGGAGAAGGTGCAG |
| | GCTGCCGTGGGCACCAGCGCCGCCCCTGTGCCCAGCGACAATCACTAG (SEQ ID |
| | NO: 227) |
| Tfrc_ | MKVLWAALLVTFLAGCQADEEEIQKAIEELLRKGVSEEEAAIIIVQRFNVAVVVVVQDERQ |
| minibinder1_ | GKHISEYIRRYIPEADVILFANLVVIKVETHELSTRVWEAAQKAYGSGGSGSGGSKVEQAV |
| APOE3_R142A_ | ETEPEPELRQQTEWQSGQRWELALGRFWDYLRWVQTLSEQVQEELLSSQVTQELRALM |
| K143S | DETMKELKAYKSELEEQLTPVAEETRARLSKELQAAQARLGADMEDVCGRLVQYRGEVQ |
| | AMLGQSTEELRVRLASHLASLRKRLLRDADDLQKRLAVYQAGAREGAERGLSAIRERLGP |
| | LVEQGRVRAATVGSLAGQPLQERAQAWGERLRARMEEMGSRTRDRLDEVKEQVAEVRA |
| | KLEEQAQQIRLQAEAFQARLKSWFEPLVEDMQRQWAGLVEKVQAAVGTSAAPVPSDNH |
| | * (SEQ ID NO: 228) |
| Tfrc_ | ATGAAGGTTCTGTGGGCTGCGTTGCTGGTCACATTCCTGGCAGGATGCCAGGCCGA |
| minibinder2_ | CGAGGAGGAGATCCAGAAGGCCATCGAGGAGCTGCTGAGAAAGGGCGTGAGCGA |
| APOE3_R142A_ | GGAGGAGGCCGCCATCATCATCGTGCAGAGATTCAACGTGGCCGTGGTGGTGGTG |
| K143S | GTGCAGGACGAGAGACAGGGCAAGCACATCAGCGAGTACATCAGAAGATACATCC |
| | CCGAGGCCGACGTGATCCTGTTCGCCAACCTGGTGGTGATCAAGGTGGAGACCCA |
| | CGAGCTGAGCACCAGAGTGTGGGAGGCCGCCCAGAAGGCCTACggatccggtgggtcc |
| | ggtagtggtggttccGAAGGGGAACCGGAGGTGACGGACCAACTTGAGTGGCAGTCTAAT |
| | CAGCCCTGGGAACAGGCACTCAACCGCTTCTGGGATTATCTGCGCTGGGTACAGAC |
| | CCTGAGCGACCAGGTGCAGGAGGAATTACAGAGCTCTCAAGTGACTCAGGAGCTTA |
| | CCGCTCTTATGGAAGACACAATGACTGAGGTTAAGGCATACAAAAAGGAGCTGGAGG |
| | AACAGTTGGGGCCAGTCGCAGAGGAAACTAGGGCACGGCTGGGTAAGGAAGTGCA |
| | GGCCGCTCAGGCCAGGCTTGGGGCCGATATGGAGGATCTGCGCAACCGCCTGGG |
| | ACAGTACCGGAATGAGGTTCACACCATGTTAGGTCAGAGCACAGAGGAAATCAGAG |
| | CCCGGCTCAGCACACACCTCGCAAGTATGCGCAAGCGCCTAATGCGTGACGCCGA |
| | GGATCTGCAGAAGCGCCTGGCGGTGTACAAAGCTGGCGCAAGAGAGGGCGCCGA |
| | GAGAGGCGTCTCGGCCATTAGAGAGAGACTAGGACCCTTGGTCGAGCAGGGGAGG |
| | CAAAGAACAGCCAACCTGGGAGCCGGCGCCGCACAACCTCTAAGAGACCGCGCT |
| | CAGGCCTTTGGTGATCGGATAAGAGGAAGGTTGGAGGAGGTGGGCAATCAGGCAAG |
| | AGACCGTCTCGAGGAAGTGCGCGAGCACATGGAGGAGGTGAGAAGTAAAATGGAA |
| | GAACAAACCCAGCAGATTAGGCTTCAGGCAGAAATATTTCAGGCCCGCTTAAAGGGC |
| | TGGTTCGAGCCTATCGTGGAGGATATGCATAGACAGTGGGCCAACCTGATGGAGAA |
| | GATCCAAGCCTCTGTTGCAACCAACCCCATCATAACTCCAGTGGCTCAGGAAAACCA |
| | GggcagcggcCATCACCATCACCATCACTGA (SEQ ID NO: 229) |
| Tfrc_ | MKVLWAALLVTFLAGCQADEEEIQKAIEELLRKGVSEEEAAIIIVQRFNVAVVVVVQDERQ |
| minibinder1_ | GKHISEYIRRYIPEADVILFANLVVIKVETHELSTRVWEAAQKAYGSGGSGSGGSEGEPEVT |
| APOE3_R142A_ | DQLEWQSNQPWEQALNRFWDYLRWVQTLSDQVQEELQSSQVTQELTALMEDTMTEV |
| K143S | KAYKKELEEQLGPVAEETRARLGKEVQAAQARLGADMEDLRNRLGQYRNEVHTMLGQS |
| | TEEIRARLSTHLASMRKRLMRDAEDLQKRLAVYKAGAREGAERGVSAIRERLGPLVEQGR |
| | QRTANLGAGAAQPLRDRAQAFGDRIRGRLEEVGNQARDRLEEVREHMEEVRSKMEEQT |
| | QQIRLQAEIFQARLKGWFEPIVEDMHRQWANLMEKIQASVATNPIITPVAQENQGSGHH |
| | HHHH* (SEQ ID NO: 230) |
| scFv- | ATGAAGGTTCTGTGGGCTGCGTTGCTGGTCACATTCCTGGCAGGATGCCAGGCCGC |
| CD90_APOE3_ | TTCCGCATCACAAGTACAGCTCGTCCAGTCCGGCGCCGAAGTGAAAAAGCCCGGT |
| R142A_K143S | GCCAGCGTGAAGGTTTCGTGCAAGGCTTCGGGTTACACGTTCACCGGCTACTACGTT |
| | CACTGGGTCCGTCAGGCGCCAGGCCAAGGATTGGAGTGGATGGGATGGGTCAACC |
| | CTAATTCGGGAGACACAAACTATGCGCAGAAATTCCAGGGACGGGTGACGATGACC |
| | AGGGATACATCTATATCGACTGCCTACATGGAACTCTCCGGGCTCCGCTCTGATGAC |
| | ACTGCAGTGTACTACTGTGCAAGGGATGGCGATGAAGACTGGTACTTTGATCTTTGGG |
| | GCCGGGGGACTCCAGTGACGGTTTCATCTGGTATCTTGGGCAGCGGCGGAGGAGG |
| | GTCGGGGGGTGGTGGCTCAGGAGGTGGCGGAAGCGACATCCGCTTGACACAGAG |
| | CCCATCCTCTTTGAGTGCCAGCATTGGCGATAGAGTGACCATCACGTGCCGAGCAT |
| | CTCAAGGCATTTCTCGCAGTCTGGTGTGGTATCAGCAGAAGCCTGGAAAGGCTCCAA |
| | GGTTGCTGATCTATGCCGCTTCTACTCTACAGTCTGGCGTACCCAGTCGCTTCAGTG |
| | GGTCAGGAAGCGGAACAGACTTTACTCTTACCATCTCATCCCTGCAACCAGAAGATTT |
| | TGCCACCTACTACTGTCTGCAGCATAATACATACCCATTCACCTTCGGGCCGGGTAC |
| | CAAGGTCGACATTAAATCTGGCATCCCGGAACAGAAGTTGggatccggtgggtccggtagtgg |
| | tggttccGAAGGGGAACCGGAGGTGACGGACCAACTTGAGTGGCAGTCTAATCAGCC |
| | CTGGGAACAGGCACTCAACCGCTTCTGGGATTATCTGCGCTGGGTACAGACCCTGA |
| | GCGACCAGGTGCAGGAGGAATTACAGAGCTCTCAAGTGACTCAGGAGCTTACCGCT |
| | CTTATGGAAGACACAATGACTGAGGTTAAGGCATACAAAAAGGAGCTGGAGGAACAG |
| | TTGGGGCCAGTCGCAGAGGAAACTAGGGCACGGCTGGGTAAGGAAGTGCAGGCC |
| | GCTCAGGCCAGGCTTGGGGCCGATATGGAGGATCTGCGCAACCGCCTGGGACAGT |
| | ACCGGAATGAGGTTCACACCATGTTAGGTCAGAGCACAGAGGAAATCAGAGCCCGG |
| | CTCAGCACACACCTCGCAAGTATGCGCAAGCGCCTAATGCGTGACGCCGAGGATC |
| | TGCAGAAGCGCCTGGCGGTGTACAAAGCTGGCGCAAGAGAGGGCGCCGAGAGAG |
| | GCGTCTCGGCCATTAGAGAGAGACTAGGACCCTTGGTCGAGCAGGGGAGGCAAAG |
| | AACAGCCAACCTGGGAGCCGGCGCCGCACAACCTCTAAGAGACCGCGCTCAGGC |
| | CTTTGGTGATCGGATAAGAGGAAGGTTGGAGGAGGTGGGCAATCAGGCAAGAGACC |
| | GTCTCGAGGAAGTGCGCGAGCACATGGAGGAGGTGAGAAGTAAAATGGAAGAACAA |
| | ACCCAGCAGATTAGGCTTCAGGCAGAAATATTTCAGGCCCGCTTAAAGGGCTGGTTC |
| | GAGCCTATCGTGGAGGATATGCATAGACAGTGGGCCAACCTGATGGAGAAGATCCA |
| | AGCCTCTGTTGCAACCAACCCCATCATAACTCCAGTGGCTCAGGAAAACCAGggcag |
| | cggcCATCACCATCACCATCACTGA (SEQ ID NO: 231) |
| scFv- | MKVLWAALLVTFLAGCQAASASQVQLVQSGAEVKKPGASVKVSCKASGYTFTGYYVHW |
| CD90_APOE3_ | VRQAPGQGLEWMGWVNPNSGDTNYAQKFQGRVTMTRDTSISTAYMELSGLRSDDTAV |
| R142A_ | YYCARDGDEDWYFDLWGRGTPVTVSSGILGSGGGGSGGGGSGGGGSDIRLTQSPSSL |
| K143S | SASIGDRVTITCRASQGISRSLVWYQQKPGKAPRLLIYAASTLQSGVPSRFSGSGSGTDFT |
| | LTISSLQPEDFATYYCLQHNTYPFTFGPGTKVDIKSGIPEQKLGSGGSGSGGSEGEPEVTD |
| | QLEWQSNQPWEQALNRFWDYLRWVQTLSDQVQEELQSSQVTQELTALMEDTMTEVKA |
| | YKKELEEQLGPVAEETRARLGKEVQAAQARLGADMEDLRNRLGQYRNEVHTMLGQSTE |
| | EIRARLSTHLASMRKRLMRDAEDLQKRLAVYKAGAREGAERGVSAIRERLGPLVEQGRQR |
| | TANLGAGAAQPLRDRAQAFGDRIRGRLEEVGNQARDRLEEVREHMEEVRSKMEEQTQQ |
| | IRLQAEIFQARLKGWFEPIVEDMHRQWANLMEKIQASVATNPIITPVAQENQGSGHHHH |
| | HH* (SEQ ID NO: 232) |
| scFv- | ATGAAGGTTCTGTGGGCTGCGTTGCTGGTCACATTCCTGGCAGGATGCCAGGCCCA |
| CD3_APOE3_ | GGTCCAACTACAGCAGTCCGGAGCCGAACTAGCCCGCCGCGGGGCCAGCGTGAA |
| R142A_K143S | GATGAGCTGCAAGGCTAGCGGTTATACCTTTACTAGATACACAATGCATTGGGTCAAA |
| | CAGCGGCCTGGTCAGGGACTGGAGTGGATTGGCTATATAAATCCAAGTAGAGGGTA |
| | CACTAACTACAATCAAAAGTTCAAAGACAAGGCTACATTGACCACTGATAAGAGTTCTA |
| | GTACCGCTTACATGCAGCTAAGCAGCCTAACATCGGAGGATTCCGCAGTGTATTACT |
| | GTGCTCGCTACTATGACGATCATTATAGCCTTGATTATTGGGGCCAGGGTACAACTCT |
| | AACCGTGTCCTCCGCAAAAACTACCCCCGACATAGTCTTAACCCAGTCTCCAGCAAT |
| | CATGTCCGCTAGCCCTGGGGAGAAAGTAACAATGACGTGTAGTGCCTCTTCATCGGT |
| | CAGTTATATGAATTGGTACCAGCAAAAGTCCGGGACATCCCCAAAGCGGTGGATCTA |
| | TGACACCAGCAAACTGGCCTCTGGGGTGCCAGCCCACTTCCGGGGGTCAGGGTCC |
| | GGTACCTCGTATAGCCTGACGATCTCAGGGATGGAGGCAGAGGACGCAGCCACGT |
| | ACTATTGCCAGCAGTGGTCCAGCAACCCGTTCACTTTTGGGTCAGGAACAAAACTCG |
| | AAATTAATAGAGCCGACACTGCTGCCGCTGGATCAggatccggtgggtccggtagtggtggttcc |
| | AAGGTGGAGCAAGCGGTGGAGACAGAGCCGGAGCCCGAGCTGCGCCAGCAGAC |
| | CGAGTGGCAGAGCGGCCAGCGCTGGGAACTGGCACTGGGTCGCTTTTGGGATTAC |
| | CTGCGCTGGGTGCAGACACTGTCTGAGCAGGTGCAGGAGGAGCTGCTCAGCTCCC |
| | AGGTCACCCAGGAACTGAGGGCGCTGATGGACGAGACCATGAAGGAGTTGAAGGC |
| | CTACAAATCGGAACTGGAGGAACAACTGACCCCGGTGGCGGAGGAGACGCGGGC |
| | ACGGCTGTCCAAGGAGCTGCAGGCGGCGCAGGCCCGGCTGGGCGCGGACATGG |
| | AGGACGTGTGCGGCCGCCTGGTGCAGTACCGCGGCGAGGTGCAGGCCATGCTCG |
| | GCCAGAGCACCGAGGAGCTGCGGGTGCGCCTCGCCTCCCACCTGgccagcCTGCG |
| | TAAGCGGCTCCTCCGCGATGCCGATGACCTGCAGAAGCGCCTGGCAGTGTACCAG |
| | GCCGGGGCCCGCGAGGGCGCCGAGCGCGGCCTCAGCGCCATCCGCGAGCGCC |
| | TGGGGCCCCTGGTGGAACAGGGCCGCGTGCGGGCCGCCACTGTGGGCTCCCTG |
| | GCCGGCCAGCCGCTACAGGAGCGGGCCCAGGCCTGGGGCGAGCGGCTGCGCG |
| | CGCGGATGGAGGAGATGGGCAGCCGGACCCGCGACCGCCTGGACGAGGTGAAG |
| | GAGCAGGTGGCGGAGGTGCGCGCCAAGCTGGAGGAGCAGGCCCAGCAGATACG |
| | CCTGCAGGCCGAGGCCTTCCAGGCCCGCCTCAAGAGCTGGTTCGAGCCCCTGGT |
| | GGAAGACATGCAGCGCCAGTGGGCCGGGCTGGTGGAGAAGGTGCAGGCTGCCGT |
| | GGGCACCAGCGCCGCCCCTGTGCCCAGCGACAATCACTAG (SEQ ID NO: 233) |
| scFv- | MKVLWAALLVTFLAGCQAQVQLQQSGAELARRGASVKMSCKASGYTFTRYTMHWVKQ |
| CD3_APOE3_ | RPGQGLEWIGYINPSRGYTNYNQKFKDKATLTTDKSSSTAYMQLSSLTSEDSAVYYCARY |
| R142A_K143S | YDDHYSLDYWGQGTTLTVSSAKTTPDIVLTQSPAIMSASPGEKVTMTCSASSSVSYMNWY |
| | QQKSGTSPKRWIYDTSKLASGVPAHFRGSGSGTSYSLTISGMEAEDAATYYCQQWSSNP |
| | FTFGSGTKLEINRADTAAAGSGSGGSGSGGSKVEQAVETEPEPELRQQTEWQSGQRWE |
| | LALGRFWDYLRWVQTLSEQVQEELLSSQVTQELRALMDETMKELKAYKSELEEQLTPVAE |
| | ETRARLSKELQAAQARLGADMEDVCGRLVQYRGEVQAMLGQSTEELRVRLASHLASLRK |
| | RLLRDADDLQKRLAVYQAGAREGAERGLSAIRERLGPLVEQGRVRAATVGSLAGQPLQE |
| | RAQAWGERLRARMEEMGSRTRDRLDEVKEQVAEVRAKLEEQAQQIRLQAEAFQARLKS |
| | WFEPLVEDMQRQWAGLVEKVQAAVGTSAAPVPSDNH* (SEQ ID NO: 234) |
| EGFR_ | ATGAAGGTTCTGTGGGCTGCGTTGCTGGTCACATTCCTGGCAGGATGCCAGGCCGA |
| minibinder1_ | CCACTGGGAGGAGGTGTTCAGAAACGCCCTGGAGCACCTGCAGGAGGCCACCCA |
| APOE3_R142A_ | GCAGAACGACCCCCAGAAGGCCAAGAAGATCCTGGAGGAGGCCCACAAGAAGCT |
| K143S | GAGAAGAGAGCTGAGCGAGGAGGAGGCCAGAAGCGTGGTGAGATGGCTGAAGCA |
| | GCTGGTGGACAGAGAGAAGAGCggatccggtgggtccggtagtggtggttccAAGGTGGAGCAA |
| | GCGGTGGAGACAGAGCCGGAGCCCGAGCTGCGCCAGCAGACCGAGTGGCAGAG |
| | CGGCCAGCGCTGGGAACTGGCACTGGGTCGCTTTTGGGATTACCTGCGCTGGGTG |
| | CAGACACTGTCTGAGCAGGTGCAGGAGGAGCTGCTCAGCTCCCAGGTCACCCAGG |
| | AACTGAGGGCGCTGATGGACGAGACCATGAAGGAGTTGAAGGCCTACAAATCGGAA |
| | CTGGAGGAACAACTGACCCCGGTGGCGGAGGAGACGCGGGCACGGCTGTCCAAG |
| | GAGCTGCAGGCGGCGCAGGCCCGGCTGGGCGCGGACATGGAGGACGTGTGCGG |
| | CCGCCTGGTGCAGTACCGCGGCGAGGTGCAGGCCATGCTCGGCCAGAGCACCGA |
| | GGAGCTGCGGGTGCGCCTCGCCTCCCACCTGgccagcCTGCGTAAGCGGCTCCTC |
| | CGCGATGCCGATGACCTGCAGAAGCGCCTGGCAGTGTACCAGGCCGGGGCCCGC |
| | GAGGGCGCCGAGCGCGGCCTCAGCGCCATCCGCGAGCGCCTGGGGCCCCTGGT |
| | GGAACAGGGCCGCGTGCGGGCCGCCACTGTGGGCTCCCTGGCCGGCCAGCCGC |
| | TACAGGAGCGGGCCCAGGCCTGGGGCGAGCGGCTGCGCGCGCGGATGGAGGAG |
| | ATGGGCAGCCGGACCCGCGACCGCCTGGACGAGGTGAAGGAGCAGGTGGCGGA |
| | GGTGCGCGCCAAGCTGGAGGAGCAGGCCCAGCAGATACGCCTGCAGGCCGAGG |
| | CCTTCCAGGCCCGCCTCAAGAGCTGGTTCGAGCCCCTGGTGGAAGACATGCAGCG |
| | CCAGTGGGCCGGGCTGGTGGAGAAGGTGCAGGCTGCCGTGGGCACCAGCGCCG |
| | CCCCTGTGCCCAGCGACAATCACTAG (SEQ ID NO: 235) |
| EGFR_ | MKVLWAALLVTFLAGCQADHWEEVFRNALEHLQEATQQNDPQKAKKILEEAHKKLRREL |
| minibinder1_ | SEEEARSVVRWLKQLVDREKSGSGGSGSGGSKVEQAVETEPEPELRQQTEWQSGQRW |
| APOE3_R142A_ | ELALGRFWDYLRWVQTLSEQVQEELLSSQVTQELRALMDETMKELKAYKSELEEQLTPVA |
| K143S | EETRARLSKELQAAQARLGADMEDVCGRLVQYRGEVQAMLGQSTEELRVRLASHLASLR |
| | KRLLRDADDLQKRLAVYQAGAREGAERGLSAIRERLGPLVEQGRVRAATVGSLAGQPLQ |
| | ERAQAWGERLRARMEEMGSRTRDRLDEVKEQVAEVRAKLEEQAQQIRLQAEAFQARLK |
| | SWFEPLVEDMQRQWAGLVEKVQAAVGTSAAPVPSDNH* (SEQ ID NO: 236) |
| EGFR_ | ATGAAGGTTCTGTGGGCTGCGTTGCTGGTCACATTCCTGGCAGGATGCCAGGCCAG |
| minibinder2_ | CCTGGACGAGGCCAAGAAGCTGCTGCAGGAGGCCGAGAAGCTGGCCAGAAAGCT |
| APOE3_R142A_ | GAACGACAGAACCGAGCTGGCCTACGTGGAGTTCCTGAAGCACAGCCTGGAGACC |
| K143S | GCCAAGAAGCAGAACGACAAGAGAACCATCGAGAGCGTGAGAGACATGGCCAGAG |
| | ACGCCCTGGAGGAGCTGCAGAGCggatccggtgggtccggtagtggtggttccAAGGTGGAGC |
| | AAGCGGTGGAGACAGAGCCGGAGCCCGAGCTGCGCCAGCAGACCGAGTGGCAG |
| | AGCGGCCAGCGCTGGGAACTGGCACTGGGTCGCTTTTGGGATTACCTGCGCTGGG |
| | TGCAGACACTGTCTGAGCAGGTGCAGGAGGAGCTGCTCAGCTCCCAGGTCACCCA |
| | GGAACTGAGGGCGCTGATGGACGAGACCATGAAGGAGTTGAAGGCCTACAAATCG |
| | GAACTGGAGGAACAACTGACCCCGGTGGCGGAGGAGACGCGGGCACGGCTGTCC |
| | AAGGAGCTGCAGGCGGCGCAGGCCCGGCTGGGCGCGGACATGGAGGACGTGTG |
| | CGGCCGCCTGGTGCAGTACCGCGGCGAGGTGCAGGCCATGCTCGGCCAGAGCA |
| | CCGAGGAGCTGCGGGTGCGCCTCGCCTCCCACCTGgccagcCTGCGTAAGCGGCT |
| | CCTCCGCGATGCCGATGACCTGCAGAAGCGCCTGGCAGTGTACCAGGCCGGGGC |
| | CCGCGAGGGCGCCGAGCGCGGCCTCAGCGCCATCCGCGAGCGCCTGGGGCCC |
| | CTGGTGGAACAGGGCCGCGTGCGGGCCGCCACTGTGGGCTCCCTGGCCGGCCA |
| | GCCGCTACAGGAGCGGGCCCAGGCCTGGGGCGAGCGGCTGCGCGCGCGGATG |
| | GAGGAGATGGGCAGCCGGACCCGCGACCGCCTGGACGAGGTGAAGGAGCAGGT |
| | GGCGGAGGTGCGCGCCAAGCTGGAGGAGCAGGCCCAGCAGATACGCCTGCAGG |
| | CCGAGGCCTTCCAGGCCCGCCTCAAGAGCTGGTTCGAGCCCCTGGTGGAAGACAT |
| | GCAGCGCCAGTGGGCCGGGCTGGTGGAGAAGGTGCAGGCTGCCGTGGGCACCA |
| | GCGCCGCCCCTGTGCCCAGCGACAATCACTAG (SEQ ID NO: 237) |
| EGFR_ | MKVLWAALLVTFLAGCQASLDEAKKLLQEAEKLARKLNDRTELAYVEFLKHSLETAKKQN |
| minibinder2_ | DKRTIESVRDMARDALEELQSGSGGSGSGGSKVEQAVETEPEPELRQQTEWQSGQRW |
| APOE3_R142A_ | ELALGRFWDYLRWVQTLSEQVQEELLSSQVTQELRALMDETMKELKAYKSELEEQLTPVA |
| K143S | EETRARLSKELQAAQARLGADMEDVCGRLVQYRGEVQAMLGQSTEELRVRLASHLASLR |
| | KRLLRDADDLQKRLAVYQAGAREGAERGLSAIRERLGPLVEQGRVRAATVGSLAGQPLQ |
| | ERAQAWGERLRARMEEMGSRTRDRLDEVKEQVAEVRAKLEEQAQQIRLQAEAFQARLK |
| | SWFEPLVEDMQRQWAGLVEKVQAAVGTSAAPVPSDNH* (SEQ ID NO: 238) |
| Heart_CRPPR | ATGAAGGTTCTGTGGGCTGCGTTGCTGGTCACATTCCTGGCAGGATGCCAGGCCtgc |
| (SEQ ID | agaccccccagaggatccggtgggtccggtagtggtggttccAAGGTGGAGCAAGCGGTGGAGACA |
| NO: 271) | GAGCCGGAGCCCGAGCTGCGCCAGCAGACCGAGTGGCAGAGCGGCCAGCGCTG |
| | GGAACTGGCACTGGGTCGCTTTTGGGATTACCTGCGCTGGGTGCAGACACTGTCTG |
| | AGCAGGTGCAGGAGGAGCTGCTCAGCTCCCAGGTCACCCAGGAACTGAGGGCGC |
| | TGATGGACGAGACCATGAAGGAGTTGAAGGCCTACAAATCGGAACTGGAGGAACAA |
| | CTGACCCCGGTGGCGGAGGAGACGCGGGCACGGCTGTCCAAGGAGCTGCAGGC |
| | GGCGCAGGCCCGGCTGGGCGCGGACATGGAGGACGTGTGCGGCCGCCTGGTGC |
| | AGTACCGCGGCGAGGTGCAGGCCATGCTCGGCCAGAGCACCGAGGAGCTGCGG |
| | GTGCGCCTCGCCTCCCACCTGgccagcCTGCGTAAGCGGCTCCTCCGCGATGCCGA |
| | TGACCTGCAGAAGCGCCTGGCAGTGTACCAGGCCGGGGCCCGCGAGGGCGCCG |
| | AGCGCGGCCTCAGCGCCATCCGCGAGCGCCTGGGGCCCCTGGTGGAACAGGGC |
| | CGCGTGCGGGCCGCCACTGTGGGCTCCCTGGCCGGCCAGCCGCTACAGGAGCG |
| | GGCCCAGGCCTGGGGCGAGCGGCTGCGCGCGCGGATGGAGGAGATGGGCAGCC |
| | GGACCCGCGACCGCCTGGACGAGGTGAAGGAGCAGGTGGCGGAGGTGCGCGCC |
| | AAGCTGGAGGAGCAGGCCCAGCAGATACGCCTGCAGGCCGAGGCCTTCCAGGCC |
| | CGCCTCAAGAGCTGGTTCGAGCCCCTGGTGGAAGACATGCAGCGCCAGTGGGCC |
| | GGGCTGGTGGAGAAGGTGCAGGCTGCCGTGGGCACCAGCGCCGCCCCTGTGCC |
| | CAGCGACAATCACTAG (SEQ ID NO: 239) |
| Heart_CRPPR | MKVLWAALLVTFLAGCQACRPPRGSGGSGSGGSKVEQAVETEPEPELRQQTEWQSGQ |
| (SEQ ID | RWELALGRFWDYLRWVQTLSEQVQEELLSSQVTQELRALMDETMKELKAYKSELEEQLT |
| NO: 271) | PVAEETRARLSKELQAAQARLGADMEDVCGRLVQYRGEVQAMLGQSTEELRVRLASHLA |
| | SLRKRLLRDADDLQKRLAVYQAGAREGAERGLSAIRERLGPLVEQGRVRAATVGSLAGQ |
| | PLQERAQAWGERLRARMEEMGSRTRDRLDEVKEQVAEVRAKLEEQAQQIRLQAEAFQA |
| | RLKSWFEPLVEDMQRQWAGLVEKVQAAVGTSAAPVPSDNH* (SEQ ID NO: 240) |
| Heart_ | ATGAAGGTTCTGTGGGCTGCGTTGCTGGTCACATTCCTGGCAGGATGCCAGGCCtgc |
| CGRKSKTVC | ggcagaaagagcaagaccgtgtgcggatccggtgggtccggtagtggtggttccAAGGTGGAGCAAGCG |
| (SEQ ID | GTGGAGACAGAGCCGGAGCCCGAGCTGCGCCAGCAGACCGAGTGGCAGAGCGG |
| NO: 272) | CCAGCGCTGGGAACTGGCACTGGGTCGCTTTTGGGATTACCTGCGCTGGGTGCAGA |
| | CACTGTCTGAGCAGGTGCAGGAGGAGCTGCTCAGCTCCCAGGTCACCCAGGAACT |
| | GAGGGCGCTGATGGACGAGACCATGAAGGAGTTGAAGGCCTACAAATCGGAACTG |
| | GAGGAACAACTGACCCCGGTGGCGGAGGAGACGCGGGCACGGCTGTCCAAGGAG |
| | CTGCAGGCGGCGCAGGCCCGGCTGGGCGCGGACATGGAGGACGTGTGCGGCCG |
| | CCTGGTGCAGTACCGCGGCGAGGTGCAGGCCATGCTCGGCCAGAGCACCGAGGA |
| | GCTGCGGGTGCGCCTCGCCTCCCACCTGgccagcCTGCGTAAGCGGCTCCTCCGC |
| | GATGCCGATGACCTGCAGAAGCGCCTGGCAGTGTACCAGGCCGGGGCCCGCGAG |
| | GGCGCCGAGCGCGGCCTCAGCGCCATCCGCGAGCGCCTGGGGCCCCTGGTGGA |
| | ACAGGGCCGCGTGCGGGCCGCCACTGTGGGCTCCCTGGCCGGCCAGCCGCTAC |
| | AGGAGCGGGCCCAGGCCTGGGGCGAGCGGCTGCGCGCGCGGATGGAGGAGATG |
| | GGCAGCCGGACCCGCGACCGCCTGGACGAGGTGAAGGAGCAGGTGGCGGAGGT |
| | GCGCGCCAAGCTGGAGGAGCAGGCCCAGCAGATACGCCTGCAGGCCGAGGCCTT |
| | CCAGGCCCGCCTCAAGAGCTGGTTCGAGCCCCTGGTGGAAGACATGCAGCGCCA |
| | GTGGGCCGGGCTGGTGGAGAAGGTGCAGGCTGCCGTGGGCACCAGCGCCGCCC |
| | CTGTGCCCAGCGACAATCACTAG (SEQ ID NO: 241) |
| Heart_ | MKVLWAALLVTFLAGCQACGRKSKTVCGSGGSGSGGSKVEQAVETEPEPELRQQTEW |
| CGRKSKTVC | QSGQRWELALGRFWDYLRWVQTLSEQVQEELLSSQVTQELRALMDETMKELKAYKSEL |
| (SEQ ID | EEQLTPVAEETRARLSKELQAAQARLGADMEDVCGRLVQYRGEVQAMLGQSTEELRVRL |
| NO: 272) | ASHLASLRKRLLRDADDLQKRLAVYQAGAREGAERGLSAIRERLGPLVEQGRVRAATVGS |
| | LAGQPLQERAQAWGERLRARMEEMGSRTRDRLDEVKEQVAEVRAKLEEQAQQIRLQAE |
| | AFQARLKSWFEPLVEDMQRQWAGLVEKVQAAVGTSAAPVPSDNH* (SEQ ID NO: 242) |
| lung_ | ATGAAGGTTCTGTGGGCTGCGTTGCTGGTCACATTCCTGGCAGGATGCCAGGCCtgc |
| CGFECVRQCPERC | ggcttcgagtgcgtgagacagtgccccgagagatgcggatccggtgggtccggtagtggtggttccAAGGTGG |
| (SEQ ID | AGCAAGCGGTGGAGACAGAGCCGGAGCCCGAGCTGCGCCAGCAGACCGAGTGG |
| NO: 273) | CAGAGCGGCCAGCGCTGGGAACTGGCACTGGGTCGCTTTTGGGATTACCTGCGCT |
| | GGGTGCAGACACTGTCTGAGCAGGTGCAGGAGGAGCTGCTCAGCTCCCAGGTCAC |
| | CCAGGAACTGAGGGCGCTGATGGACGAGACCATGAAGGAGTTGAAGGCCTACAAAT |
| | CGGAACTGGAGGAACAACTGACCCCGGTGGCGGAGGAGACGCGGGCACGGCTGT |
| | CCAAGGAGCTGCAGGCGGCGCAGGCCCGGCTGGGCGCGGACATGGAGGACGTG |
| | TGCGGCCGCCTGGTGCAGTACCGCGGCGAGGTGCAGGCCATGCTCGGCCAGAGC |
| | ACCGAGGAGCTGCGGGTGCGCCTCGCCTCCCACCTGgccagcCTGCGTAAGCGGC |
| | TCCTCCGCGATGCCGATGACCTGCAGAAGCGCCTGGCAGTGTACCAGGCCGGGG |
| | CCCGCGAGGGCGCCGAGCGCGGCCTCAGCGCCATCCGCGAGCGCCTGGGGCC |
| | CCTGGTGGAACAGGGCCGCGTGCGGGCCGCCACTGTGGGCTCCCTGGCCGGCC |
| | AGCCGCTACAGGAGCGGGCCCAGGCCTGGGGCGAGCGGCTGCGCGCGCGGATG |
| | GAGGAGATGGGCAGCCGGACCCGCGACCGCCTGGACGAGGTGAAGGAGCAGGT |
| | GGCGGAGGTGCGCGCCAAGCTGGAGGAGCAGGCCCAGCAGATACGCCTGCAGG |
| | CCGAGGCCTTCCAGGCCCGCCTCAAGAGCTGGTTCGAGCCCCTGGTGGAAGACAT |
| | GCAGCGCCAGTGGGCCGGGCTGGTGGAGAAGGTGCAGGCTGCCGTGGGCACCA |
| | GCGCCGCCCCTGTGCCCAGCGACAATCACTAG (SEQ ID NO: 243) |
| lung_ | MKVLWAALLVTFLAGCQACGFECVRQCPERCGSGGSGSGGSKVEQAVETEPEPELRQ |
| CGFECVRQCPERC | QTEWQSGQRWELALGRFWDYLRWVQTLSEQVQEELLSSQVTQELRALMDETMKELKA |
| (SEQ ID | YKSELEEQLTPVAEETRARLSKELQAAQARLGADMEDVCGRLVQYRGEVQAMLGQSTEE |
| NO: 273) | LRVRLASHLASLRKRLLRDADDLQKRLAVYQAGAREGAERGLSAIRERLGPLVEQGRVRA |
| | ATVGSLAGQPLQERAQAWGERLRARMEEMGSRTRDRLDEVKEQVAEVRAKLEEQAQQI |
| | RLQAEAFQARLKSWFEPLVEDMQRQWAGLVEKVQAAVGTSAAPVPSDNH* (SEQ ID |
| | NO: 244) |
| lung_FDFWITP | ATGAAGGTTCTGTGGGCTGCGTTGCTGGTCACATTCCTGGCAGGATGCCAGGCCttcg |
| (SEQ ID | acttctggatcacccccggatccggtgggtccggtagtggtggttccAAGGTGGAGCAAGCGGTGGAG |
| NO: 274) | ACAGAGCCGGAGCCCGAGCTGCGCCAGCAGACCGAGTGGCAGAGCGGCCAGCG |
| | CTGGGAACTGGCACTGGGTCGCTTTTGGGATTACCTGCGCTGGGTGCAGACACTGT |
| | CTGAGCAGGTGCAGGAGGAGCTGCTCAGCTCCCAGGTCACCCAGGAACTGAGGG |
| | CGCTGATGGACGAGACCATGAAGGAGTTGAAGGCCTACAAATCGGAACTGGAGGAA |
| | CAACTGACCCCGGTGGCGGAGGAGACGCGGGCACGGCTGTCCAAGGAGCTGCAG |
| | GCGGCGCAGGCCCGGCTGGGCGCGGACATGGAGGACGTGTGCGGCCGCCTGGT |
| | GCAGTACCGCGGCGAGGTGCAGGCCATGCTCGGCCAGAGCACCGAGGAGCTGC |
| | GGGTGCGCCTCGCCTCCCACCTGgccagcCTGCGTAAGCGGCTCCTCCGCGATGC |
| | CGATGACCTGCAGAAGCGCCTGGCAGTGTACCAGGCCGGGGCCCGCGAGGGCG |
| | CCGAGCGCGGCCTCAGCGCCATCCGCGAGCGCCTGGGGCCCCTGGTGGAACAG |
| | GGCCGCGTGCGGGCCGCCACTGTGGGCTCCCTGGCCGGCCAGCCGCTACAGGA |
| | GCGGGCCCAGGCCTGGGGCGAGCGGCTGCGCGCGCGGATGGAGGAGATGGGCA |
| | GCCGGACCCGCGACCGCCTGGACGAGGTGAAGGAGCAGGTGGCGGAGGTGCGC |
| | GCCAAGCTGGAGGAGCAGGCCCAGCAGATACGCCTGCAGGCCGAGGCCTTCCAG |
| | GCCCGCCTCAAGAGCTGGTTCGAGCCCCTGGTGGAAGACATGCAGCGCCAGTGG |
| | GCCGGGCTGGTGGAGAAGGTGCAGGCTGCCGTGGGCACCAGCGCCGCCCCTGT |
| | GCCCAGCGACAATCACTAG (SEQ ID NO: 245) |
| lung_FDFWITP | MKVLWAALLVTFLAGCQAFDFWITPGSGGSGSGGSKVEQAVETEPEPELRQQTEWQSG |
| (SEQ ID | QRWELALGRFWDYLRWVQTLSEQVQEELLSSQVTQELRALMDETMKELKAYKSELEEQL |
| NO: 274) | TPVAEETRARLSKELQAAQARLGADMEDVCGRLVQYRGEVQAMLGQSTEELRVRLASHL |
| | ASLRKRLLRDADDLQKRLAVYQAGAREGAERGLSAIRERLGPLVEQGRVRAATVGSLAG |
| | QPLQERAQAWGERLRARMEEMGSRTRDRLDEVKEQVAEVRAKLEEQAQQIRLQAEAFQ |
| | ARLKSWFEPLVEDMQRQWAGLVEKVQAAVGTSAAPVPSDNH* (SEQ ID NO: 246) |
| lung_CTHALWHTC | ATGAAGGTTCTGTGGGCTGCGTTGCTGGTCACATTCCTGGCAGGATGCCAGGCCtgc |
| (SEQ ID | acccacgccctgtggcacacctgcggatccggtgggtccggtagtggtggttccAAGGTGGAGCAAGCG |
| NO: 275) | GTGGAGACAGAGCCGGAGCCCGAGCTGCGCCAGCAGACCGAGTGGCAGAGCGG |
| | CCAGCGCTGGGAACTGGCACTGGGTCGCTTTTGGGATTACCTGCGCTGGGTGCAGA |
| | CACTGTCTGAGCAGGTGCAGGAGGAGCTGCTCAGCTCCCAGGTCACCCAGGAACT |
| | GAGGGCGCTGATGGACGAGACCATGAAGGAGTTGAAGGCCTACAAATCGGAACTG |
| | GAGGAACAACTGACCCCGGTGGCGGAGGAGACGCGGGCACGGCTGTCCAAGGAG |
| | CTGCAGGCGGCGCAGGCCCGGCTGGGCGCGGACATGGAGGACGTGTGCGGCCG |
| | CCTGGTGCAGTACCGCGGCGAGGTGCAGGCCATGCTCGGCCAGAGCACCGAGGA |
| | GCTGCGGGTGCGCCTCGCCTCCCACCTGgccagcCTGCGTAAGCGGCTCCTCCGC |
| | GATGCCGATGACCTGCAGAAGCGCCTGGCAGTGTACCAGGCCGGGGCCCGCGAG |
| | GGCGCCGAGCGCGGCCTCAGCGCCATCCGCGAGCGCCTGGGGCCCCTGGTGGA |
| | ACAGGGCCGCGTGCGGGCCGCCACTGTGGGCTCCCTGGCCGGCCAGCCGCTAC |
| | AGGAGCGGGCCCAGGCCTGGGGCGAGCGGCTGCGCGCGCGGATGGAGGAGATG |
| | GGCAGCCGGACCCGCGACCGCCTGGACGAGGTGAAGGAGCAGGTGGCGGAGGT |
| | GCGCGCCAAGCTGGAGGAGCAGGCCCAGCAGATACGCCTGCAGGCCGAGGCCTT |
| | CCAGGCCCGCCTCAAGAGCTGGTTCGAGCCCCTGGTGGAAGACATGCAGCGCCA |
| | GTGGGCCGGGCTGGTGGAGAAGGTGCAGGCTGCCGTGGGCACCAGCGCCGCCC |
| | CTGTGCCCAGCGACAATCACTAG (SEQ ID NO: 247) |
| lung_CTHALWHTC | MKVLWAALLVTFLAGCQACTHALWHTCGSGGSGSGGSKVEQAVETEPEPELRQQTEW |
| (SEQ ID | QSGQRWELALGRFWDYLRWVQTLSEQVQEELLSSQVTQELRALMDETMKELKAYKSEL |
| NO: 275) | EEQLTPVAEETRARLSKELQAAQARLGADMEDVCGRLVQYRGEVQAMLGQSTEELRVRL |
| | ASHLASLRKRLLRDADDLQKRLAVYQAGAREGAERGLSAIRERLGPLVEQGRVRAATVGS |
| | LAGQPLQERAQAWGERLRARMEEMGSRTRDRLDEVKEQVAEVRAKLEEQAQQIRLQAE |
| | AFQARLKSWFEPLVEDMQRQWAGLVEKVQAAVGTSAAPVPSDNH* (SEQ ID NO: 248) |
| kidney_ | ATGAAGGTTCTGTGGGCTGCGTTGCTGGTCACATTCCTGGCAGGATGCCAGGCCgcc |
| ANTPCGPYTHD | aacaccccctgcggcccctacacccacgactgccccgtgaagagaggatccggtgggtccggtagtggtggtt |
| CPVKR | CCGAAGGGGAACCGGAGGTGACGGACCAACTTGAGTGGCAGTCTAATCAGCCCTGG |
| (SEQ ID | GAACAGGCACTCAACCGCTTCTGGGATTATCTGCGCTGGGTACAGACCCTGAGCGA |
| NO: 276) | CCAGGTGCAGGAGGAATTACAGAGCTCTCAAGTGACTCAGGAGCTTACCGCTCTTAT |
| | GGAAGACACAATGACTGAGGTTAAGGCATACAAAAAGGAGCTGGAGGAACAGTTGG |
| | GGCCAGTCGCAGAGGAAACTAGGGCACGGCTGGGTAAGGAAGTGCAGGCCGCTC |
| | AGGCCAGGCTTGGGGCCGATATGGAGGATCTGCGCAACCGCCTGGGACAGTACCG |
| | GAATGAGGTTCACACCATGTTAGGTCAGAGCACAGAGGAAATCAGAGCCCGGCTCA |
| | GCACACACCTCGCAAGTATGCGCAAGCGCCTAATGCGTGACGCCGAGGATCTGCA |
| | GAAGCGCCTGGCGGTGTACAAAGCTGGCGCAAGAGAGGGCGCCGAGAGAGGCGT |
| | CTCGGCCATTAGAGAGAGACTAGGACCCTTGGTCGAGCAGGGGAGGCAAAGAACA |
| | GCCAACCTGGGAGCCGGCGCCGCACAACCTCTAAGAGACCGCGCTCAGGCCTTT |
| | GGTGATCGGATAAGAGGAAGGTTGGAGGAGGTGGGCAATCAGGCAAGAGACCGTCT |
| | CGAGGAAGTGCGCGAGCACATGGAGGAGGTGAGAAGTAAAATGGAAGAACAAACC |
| | CAGCAGATTAGGCTTCAGGCAGAAATATTTCAGGCCCGCTTAAAGGGCTGGTTCGAG |
| | CCTATCGTGGAGGATATGCATAGACAGTGGGCCAACCTGATGGAGAAGATCCAAGC |
| | CTCTGTTGCAACCAACCCCATCATAACTCCAGTGGCTCAGGAAAACCAGggcagcggc |
| | CATCACCATCACCATCACTGA (SEQ ID NO: 249) |
| kidney_ | MKVLWAALLVTFLAGCQAANTPCGPYTHDCPVKRGSGGSGSGGSEGEPEVTDQLEWQ |
| ANTPCGPYTHD | SNQPWEQALNRFWDYLRWVQTLSDQVQEELQSSQVTQELTALMEDTMTEVKAYKKELE |
| CPVKR | EQLGPVAEETRARLGKEVQAAQARLGADMEDLRNRLGQYRNEVHTMLGQSTEEIRARLS |
| (SEQ ID | THLASMRKRLMRDAEDLQKRLAVYKAGAREGAERGVSAIRERLGPLVEQGRQRTANLGA |
| NO: 276) | GAAQPLRDRAQAFGDRIRGRLEEVGNQARDRLEEVREHMEEVRSKMEEQTQQIRLQAEI |
| | FQARLKGWFEPIVEDMHRQWANLMEKIQASVATNPIITPVAQENQGSGHHHHHH* |
| | (SEQ ID NO: 250) |
| scFv- | ATGAAGGTTCTGTGGGCTGCGTTGCTGGTCACATTCCTGGCAGGATGCCAGGCCCA |
| CD51_1_ | GGTACAGCTGCAGCAGTCTGGAGGAGAGTTAGCGAAGCCAGGCGCCTCAGTGAAG |
| mmAPOE3_ | GTGTCCTGTAAGGCCTCAGGATATACCTTCTCCTCCTTTTGGATGCACTGGGTGCGC |
| R134A_K135S | CAGGCTCCTGGGCAGGGCCTGGAGTGGATAGGGTACATTAACCCTAGGTCCGGCTA |
| | CACTGAATATAATGAGATCTTCCGAGACAAGGCGACCATGACAACCGACACCTCCA |
| | CTTCCACCGCATACATGGAGTTAAGTTCCCTGCGATCAGAGGACACCGCCGTGTACT |
| | ACTGTGCCAGCTTCCTGGGCCGGGGAGCGATGGATTATTGGGGACAGGGCACTAC |
| | GGTGACTGTGAGCAGCGGCGGCGGGGGATCAGGCGGGGGAGGGTCCGGCGGGG |
| | GAGGGTCAGACATTCAGATGACCCAGTCCCCATCATCCCTGTCCGCCAGCGTGGG |
| | GGATAGAGTAACAATCACATGCCGCGCCAGCCAGGACATTTCAAACTACCTGGCCT |
| | GGTACCAGCAGAAACCGGGGAAAGCGCCCAAGCTGCTGATCTACTATACCTCTAAA |
| | ATTCACAGTGGAGTTCCTTCCAGGTTCAGCGGTTCCGGGAGTGGGACAGATTATACA |
| | TTTACGATCAGCAGTCTGCAACCAGAAGACATCGCAACCTACTATTGCCAGCAGGGT |
| | AACACTTTCCCGTATACATTCGGGCAGGGGACAAAGGTCGAGATCAAAggatccggtggg |
| | tccggtagtggtggttccGAAGGGGAACCGGAGGTGACGGACCAACTTGAGTGGCAGTCTA |
| | ATCAGCCCTGGGAACAGGCACTCAACCGCTTCTGGGATTATCTGCGCTGGGTACAG |
| | ACCCTGAGCGACCAGGTGCAGGAGGAATTACAGAGCTCTCAAGTGACTCAGGAGCT |
| | TACCGCTCTTATGGAAGACACAATGACTGAGGTTAAGGCATACAAAAAGGAGCTGGA |
| | GGAACAGTTGGGGCCAGTCGCAGAGGAAACTAGGGCACGGCTGGGTAAGGAAGTG |
| | CAGGCCGCTCAGGCCAGGCTTGGGGCCGATATGGAGGATCTGCGCAACCGCCTG |
| | GGACAGTACCGGAATGAGGTTCACACCATGTTAGGTCAGAGCACAGAGGAAATCAG |
| | AGCCCGGCTCAGCACACACCTCGCAAGTATGCGCAAGCGCCTAATGCGTGACGCC |
| | GAGGATCTGCAGAAGCGCCTGGCGGTGTACAAAGCTGGCGCAAGAGAGGGCGCC |
| | GAGAGAGGCGTCTCGGCCATTAGAGAGAGACTAGGACCCTTGGTCGAGCAGGGGA |
| | GGCAAAGAACAGCCAACCTGGGAGCCGGCGCCGCACAACCTCTAAGAGACCGCG |
| | CTCAGGCCTTTGGTGATCGGATAAGAGGAAGGTTGGAGGAGGTGGGCAATCAGGCA |
| | AGAGACCGTCTCGAGGAAGTGCGCGAGCACATGGAGGAGGTGAGAAGTAAAATGG |
| | AAGAACAAACCCAGCAGATTAGGCTTCAGGCAGAAATATTTCAGGCCCGCTTAAAGG |
| | GCTGGTTCGAGCCTATCGTGGAGGATATGCATAGACAGTGGGCCAACCTGATGGAG |
| | AAGATCCAAGCCTCTGTTGCAACCAACCCCATCATAACTCCAGTGGCTCAGGAAAAC |
| | CAGggcagcggcCATCACCATCACCATCACTGA (SEQ ID NO: 251) |
| scFv- | MKVLWAALLVTFLAGCQAQVQLQQSGGELAKPGASVKVSCKASGYTFSSFWMHWVRQ |
| CD51_1_ | APGQGLEWIGYINPRSGYTEYNEIFRDKATMTTDTSTSTAYMELSSLRSEDTAVYYCASFL |
| mmAPOE3_ | GRGAMDYWGQGTTVTVSSGGGGSGGGGSGGGGSDIQMTQSPSSLSASVGDRVTITCR |
| R134A_K135S | ASQDISNYLAWYQQKPGKAPKLLIYYTSKIHSGVPSRFSGSGSGTDYTFTISSLQPEDIATY |
| | YCQQGNTFPYTFGQGTKVEIKGSGGSGSGGSEGEPEVTDQLEWQSNQPWEQALNRF |
| | WDYLRWVQTLSDQVQEELQSSQVTQELTALMEDTMTEVKAYKKELEEQLGPVAEETRAR |
| | LGKEVQAAQARLGADMEDLRNRLGQYRNEVHTMLGQSTEEIRARLSTHLASMRKRLMR |
| | DAEDLQKRLAVYKAGAREGAERGVSAIRERLGPLVEQGRQRTANLGAGAAQPLRDRAQ |
| | AFGDRIRGRLEEVGNQARDRLEEVREHMEEVRSKMEEQTQQIRLQAEIFQARLKGWFEP |
| | IVEDMHRQWANLMEKIQASVATNPIITPVAQENQGSGHHHHHH* (SEQ ID NO: 252) |
| scFv- | ATGAAGGTTCTGTGGGCTGCGTTGCTGGTCACATTCCTGGCAGGATGCCAGGCCCA |
| CD51_2_APOE3_ | GGTGCAGCTGGTGGAATCCGGGGGGGGGGGGTGCAGCCCGGCCGGAGCAGGC |
| R134A_K135S | GGCTGTCATGTGCCGCATCAGGTTTTACTTTTTCTAGGTATACCATGCATTGGGTCAGA |
| | CAGGCACCAGGCAAAGGTTTGGAGTGGGTTGCTGTGATCAGCTTCGATGGATCTAAT |
| | AAGTATTACGTGGACAGTGTCAAAGGGCGTTTCACCATCTCACGCGATAATTCCGAGA |
| | ACACTCTCTACCTACAGGTGAATATACTGCGCGCCGAAGATACTGCCGTCTATTATTG |
| | CGCAAGGGAAGCCCGGGGATCTTACGCCTTCGACATTTGGGGACAGGGCACAATG |
| | GTTACAGTTTCTTCCGGCGGAGGCGGGAGCGGTGGAGGCGGAAGCGGAGGAGGC |
| | GGCTCTGAGATTGTGCTGACTCAGTCCCCCGCAACTCTCAGCCTCTCACCCGGTGA |
| | ACGAGCGACCCTCTCCTGCCGCGCATCTCAGTCTGTCTCTTCATACCTCGCATGGTA |
| | TCAACAGAAACCCGGACAGGCTCCACGACTGCTCATCTACGATGCATCCAATCGGG |
| | CTACGGGCATCCCAGCAAGATTTAGCGGGTCCGGATCTGGCACCGACTTTACCCTG |
| | ACAATCTCCTCCCTCGAACCTGAAGACTTCGCTGTGTATTATTGCCAGCAGAGGAGC |
| | AACTGGCCACCCTTCACCTTCGGGCCAGGCACAAAAGTTGACATTAAAggatccggtggg |
| | tccggtagtggtggttccGAAGGGGAACCGGAGGTGACGGACCAACTTGAGTGGCAGTCTA |
| | ATCAGCCCTGGGAACAGGCACTCAACCGCTTCTGGGATTATCTGCGCTGGGTACAG |
| | ACCCTGAGCGACCAGGTGCAGGAGGAATTACAGAGCTCTCAAGTGACTCAGGAGCT |
| | TACCGCTCTTATGGAAGACACAATGACTGAGGTTAAGGCATACAAAAAGGAGCTGGA |
| | GGAACAGTTGGGGCCAGTCGCAGAGGAAACTAGGGCACGGCTGGGTAAGGAAGTG |
| | CAGGCCGCTCAGGCCAGGCTTGGGGCCGATATGGAGGATCTGCGCAACCGCCTG |
| | GGACAGTACCGGAATGAGGTTCACACCATGTTAGGTCAGAGCACAGAGGAAATCAG |
| | AGCCCGGCTCAGCACACACCTCGCAAGTATGCGCAAGCGCCTAATGCGTGACGCC |
| | GAGGATCTGCAGAAGCGCCTGGCGGTGTACAAAGCTGGCGCAAGAGAGGGCGCC |
| | GAGAGAGGCGTCTCGGCCATTAGAGAGAGACTAGGACCCTTGGTCGAGCAGGGGA |
| | GGCAAAGAACAGCCAACCTGGGAGCCGGCGCCGCACAACCTCTAAGAGACCGCG |
| | CTCAGGCCTTTGGTGATCGGATAAGAGGAAGGTTGGAGGAGGTGGGCAATCAGGCA |
| | AGAGACCGTCTCGAGGAAGTGCGCGAGCACATGGAGGAGGTGAGAAGTAAAATGG |
| | AAGAACAAACCCAGCAGATTAGGCTTCAGGCAGAAATATTTCAGGCCCGCTTAAAGG |
| | GCTGGTTCGAGCCTATCGTGGAGGATATGCATAGACAGTGGGCCAACCTGATGGAG |
| | AAGATCCAAGCCTCTGTTGCAACCAACCCCATCATAACTCCAGTGGCTCAGGAAAAC |
| | CAGggcagcggcCATCACCATCACCATCACTGA (SEQ ID NO: 253) |
| scFv- | MKVLWAALLVTFLAGCQAQVOLVESGGGVVQPGRSRRLSCAASGFTFSRYTMHWVRQ |
| CD51_2_ | APGKGLEWVAVISFDGSNKYYVDSVKGRFTISRDNSENTLYLQVNILRAEDTAVYYCAREA |
| APOE3_R134A_ | RGSYAFDIWGQGTMVTVSSGGGGSGGGGSGGGGSEIVLTQSPATLSLSPGERATLSCR |
| K135S_ | ASQSVSSYLAWYQQKPGQAPRLLIYDASNRATGIPARFSGSGSGTDFTLTISSLEPEDFAV |
| scFv- | YYCQQRSNWPPFTFGPGTKVDIKGSGGSGSGGSEGEPEVTDQLEWQSNQPWEQALN |
| | RFWDYLRWVQTLSDQVQEELQSSQVTQELTALMEDTMTEVKAYKKELEEQLGPVAEETR |
| | ARLGKEVQAAQARLGADMEDLRNRLGQYRNEVHTMLGQSTEEIRARLSTHLASMRKRL |
| | MRDAEDLQKRLAVYKAGAREGAERGVSAIRERLGPLVEQGRQRTANLGAGAAQPLRDR |
| | AQAFGDRIRGRLEEVGNQARDRLEEVREHMEEVRSKMEEQTQQIRLQAEIFQARLKGWF |
| | EPIVEDMHRQWANLMEKIQASVATNPIITPVAQENQGSGHHHHHH* (SEQ ID NO: |
| | 254) |
| CD146_APOE3_ | ATGAAGGTTCTGTGGGCTGCGTTGCTGGTCACATTCCTGGCAGGATGCCAGGCCCA |
| R134A_K135S | GGTTACCCTGAAGGAGTCCGGGCCAGTTCTGGTGAAGCCTACGGAGACATTGACAC |
| | TGACATGTACAGTTTCCGGGTTTAGCTTGACCAGTAACGCGGTTTCCTGGGTCCGCC |
| | AGCCTCCAGGAAAAGCGCTGGAGTGGATCGCAGCCATCTCCAGCGGAGGCACCA |
| | CCTACTACAACTCAGCATTCAAAAGCAGACTGACCATCTCCCGTGATACCAGTAAGT |
| | CACAGGTGGTTCTGACTATGACAAATATGGACCCCGTCGACACAGCAACATACTATT |
| | GCGCCAGGCGGTACGGGTACGGCTGGTACTTTGACTTCTGGGGACAGGGAACACTT |
| | GTGACAGTAAGTAGCGGGGGCGGCGGGAGCGGGGGCGGGGGCAGCGGCGGCG |
| | GAGGCTCTGACATCCAGATGACTCAGTCTCCGTCAAGCCTGAGCGCTTCCGTCGGA |
| | GATCGCGTGACAATCAACTGTAAAGCCTCGCAGAACATATACAACAGCCTGGCATGG |
| | TACCAGCAGAAGCCTGGCAAGGCTCCCAAGGTGCTGATTTTTTAACGCCAACAGCCT |
| | GCAGACTGGCATTCCTTCTCGGTTTAGTGGCAGTGGCTCTGGAACTGACTTTACCCT |
| | GACGATCTCATCTCTGCAACCAGAGGACTTCGCCACGTACTATTGTCAGCAGTTCTAT |
| | AGTGGGTACACATTCGGTCAGGGAACCAAGCTGGAAATTAAGggatccggtgggtccggtag |
| | tggtggttccGAAGGGGAACCGGAGGTGACGGACCAACTTGAGTGGCAGTCTAATCAGC |
| | CCTGGGAACAGGCACTCAACCGCTTCTGGGATTATCTGCGCTGGGTACAGACCCTG |
| | AGCGACCAGGTGCAGGAGGAATTACAGAGCTCTCAAGTGACTCAGGAGCTTACCGC |
| | TCTTATGGAAGACACAATGACTGAGGTTAAGGCATACAAAAAGGAGCTGGAGGAACA |
| | GTTGGGGCCAGTCGCAGAGGAAACTAGGGCACGGCTGGGTAAGGAAGTGCAGGC |
| | CGCTCAGGCCAGGCTTGGGGCCGATATGGAGGATCTGCGCAACCGCCTGGGACA |
| | GTACCGGAATGAGGTTCACACCATGTTAGGTCAGAGCACAGAGGAAATCAGAGCCC |
| | GGCTCAGCACACACCTCGCAAGTATGCGCAAGCGCCTAATGCGTGACGCCGAGGA |
| | TCTGCAGAAGCGCCTGGCGGTGTACAAAGCTGGCGCAAGAGAGGGCGCCGAGAG |
| | AGGCGTCTCGGCCATTAGAGAGAGACTAGGACCCTTGGTCGAGCAGGGGAGGCAA |
| | AGAACAGCCAACCTGGGAGCCGGCGCCGCACAACCTCTAAGAGACCGCGCTCAG |
| | GCCTTTGGTGATCGGATAAGAGGAAGGTTGGAGGAGGTGGGCAATCAGGCAAGAGA |
| | CCGTCTCGAGGAAGTGCGCGAGCACATGGAGGAGGTGAGAAGTAAAATGGAAGAA |
| | CAAACCCAGCAGATTAGGCTTCAGGCAGAAATATTTCAGGCCCGCTTAAAGGGCTGG |
| | TTCGAGCCTATCGTGGAGGATATGCATAGACAGTGGGCCAACCTGATGGAGAAGATC |
| | CAAGCCTCTGTTGCAACCAACCCCATCATAACTCCAGTGGCTCAGGAAAACCAGggc |
| | agcggcCATCACCATCACCATCACTGA (SEQ ID NO: 255) |
| scFv- | MKVLWAALLVTFLAGCQAQVTLKESGPVLVKPTETLTLTCTVSGFSLTSNAVSWVRQPPG |
| CD146_APOE3_ | KALEWIAAISSGGTTYYNSAFKSRLTISRDTSKSQVVLTMTNMDPVDTATYYCARRYGYGW |
| R134A_K135S | YFDFWGQGTLVTVSSGGGGSGGGGSGGGGSDIQMTQSPSSLSASVGDRVTINCKASQ |
| | NIYNSLAWYQQKPGKAPKVLIFNANSLQTGIPSRFSGSGSGTDFTLTISSLOPEDFATYYC |
| | QQFYSGYTFGQGTKLEIKGSGGSGSGGSEGEPEVTDQLEWQSNQPWEQALNRFWDYL |
| | RWVQTLSDQVQEELQSSQVTQELTALMEDTMTEVKAYKKELEEQLGPVAEETRARLGKE |
| | VQAAQARLGADMEDLRNRLGQYRNEVHTMLGQSTEEIRARLSTHLASMRKRLMRDAED |
| | LQKRLAVYKAGAREGAERGVSAIRERLGPLVEQGRQRTANLGAGAAQPLRDRAQAFGD |
| | RIRGRLEEVGNQARDRLEEVREHMEEVRSKMEEQTQQIRLQAEIFQARLKGWFEPIVED |
| | MHRQWANLMEKIQASVATNPIITPVAQENQGSGHHHHHH* (SEQ ID NO: 256) |
| scFv- | ATGAAGGTTCTGTGGGCTGCGTTGCTGGTCACATTCCTGGCAGGATGCCAGGCCGA |
| CD63_APOE3_ | GGTGAAGCTCGTCGAATCTGGTGGGGGATTGGTCCAGCCGGGTGGCTCCCTGAAG |
| R134A_K135S | CTGTCTTGCGCAACCTCTGGATTCACGTTCAGCGATTACTACATGTCTTGGGTGAGGC |
| | AAACACCCGAAAAGCGCCTGGAGTGGGTTGCCTACATTTCCAGTAGTGGCGGGTCT |
| | ACCTACTACAGCGACACCGTTAAGGGGCAGTTCACAATATCCAGAGACAATGCCAA |
| | GAATACGCTCTACCTCCAGATGTCAAGATTGAAGAGTGAGGATACAGCTATGTACTAC |
| | TGTGCCAGGCGGGAGGACTACGACGGACGCCTGACCTATTGGGGTCAAGGGACAC |
| | TCGTCACTATCTCTGCCGGAGGTTCTGGCGGAGGAGGCTCGGGGGGGGGTGGCTC |
| | AGGTGGGGACATCGTCCTGACACAAAGCCCAGCCAGTCTGGCAGTTTCACTTGGAC |
| | AGCGTGCTACTATTAGTTGTAGGGCATCTAAGTCCGTGAGTACATCAGGCTACAGCTA |
| | TATGAACTGGTACCAGCAGAAGCCCGGACAGCCACCCAAAGTACTTATCTATCTTGC |
| | CTCAAAATTAGAGAGCGGAGTCCCCGCAAGGTTTTCCGGGAGCGGTTCCGGTACTG |
| | ACTTTACCCTCAATATCCACCCAGTGGAGGAAGAGGATGCCGCGACCTACTACTGTC |
| | AACATTCTCGGGAGCTTCCCTACACTTTTGGTGGAGGTACCAAGCTTGAGATTAAGgg |
| | atccggtgggtccggtagtggtggttccGAAGGGGAACCGGAGGTGACGGACCAACTTGAGTG |
| | GCAGTCTAATCAGCCCTGGGAACAGGCACTCAACCGCTTCTGGGATTATCTGCGCT |
| | GGGTACAGACCCTGAGCGACCAGGTGCAGGAGGAATTACAGAGCTCTCAAGTGACT |
| | CAGGAGCTTACCGCTCTTATGGAAGACACAATGACTGAGGTTAAGGCATACAAAAAG |
| | GAGCTGGAGGAACAGTTGGGGCCAGTCGCAGAGGAAACTAGGGCACGGCTGGGTA |
| | AGGAAGTGCAGGCCGCTCAGGCCAGGCTTGGGGCCGATATGGAGGATCTGCGCAA |
| | CCGCCTGGGACAGTACCGGAATGAGGTTCACACCATGTTAGGTCAGAGCACAGAGG |
| | AAATCAGAGCCCGGCTCAGCACACACCTCGCAAGTATGCGCAAGCGCCTAATGCGT |
| | GACGCCGAGGATCTGCAGAAGCGCCTGGCGGTGTACAAAGCTGGCGCAAGAGAG |
| | GGCGCCGAGAGAGGCGTCTCGGCCATTAGAGAGAGACTAGGACCCTTGGTCGAGC |
| | AGGGGAGGCAAAGAACAGCCAACCTGGGAGCCGGCGCCGCACAACCTCTAAGAG |
| | ACCGCGCTCAGGCCTTTGGTGATCGGATAAGAGGAAGGTTGGAGGAGGTGGGCAAT |
| | CAGGCAAGAGACCGTCTCGAGGAAGTGCGCGAGCACATGGAGGAGGTGAGAAGTA |
| | AAATGGAAGAACAAACCCAGCAGATTAGGCTTCAGGCAGAAATATTTCAGGCCCGCT |
| | TAAAGGGCTGGTTCGAGCCTATCGTGGAGGATATGCATAGACAGTGGGCCAACCTG |
| | ATGGAGAAGATCCAAGCCTCTGTTGCAACCAACCCCATCATAACTCCAGTGGCTCAG |
| | GAAAACCAGggcagcggcCATCACCATCACCATCACTGA (SEQ ID NO: 257) |
| scFv- | MKVLWAALLVTFLAGCQAEVKLVESGGGLVQPGGSLKLSCATSGFTFSDYYMSWVRQT |
| CD63_APOE3_ | PEKRLEWVAYISSSGGSTYYSDTVKGQFTISRDNAKNTLYLQMSRLKSEDTAMYYCARRE |
| R134A_ | DYDGRLTYWGQGTLVTISAGGSGGGGSGGGGSGGDIVLTQSPASLAVSLGQRATISCRA |
| K135S | SKSVSTSGYSYMNWYQQKPGQPPKVLIYLASKLESGVPARFSGSGSGTDFTLNIHPVEEE |
| | DAATYYCQHSRELPYTFGGGTKLEIKGSGGSGSGGSEGEPEVTDQLEWQSNQPWEQAL |
| | NRFWDYLRWVQTLSDQVQEELQSSQVTQELTALMEDTMTEVKAYKKELEEQLGPVAEET |
| | RARLGKEVQAAQARLGADMEDLRNRLGQYRNEVHTMLGQSTEEIRARLSTHLASMRKRL |
| | MRDAEDLQKRLAVYKAGAREGAERGVSAIRERLGPLVEQGRQRTANLGAGAAQPLRDR |
| | AQAFGDRIRGRLEEVGNQARDRLEEVREHMEEVRSKMEEQTQQIRLQAEIFQARLKGWF |
| | EPIVEDMHRQWANLMEKIQASVATNPIITPVAQENQGSGHHHHHH* (SEQ ID NO: |
| | 258) |
| scFv- | ATGAAGGTTCTGTGGGCTGCGTTGCTGGTCACATTCCTGGCAGGATGCCAGGCCGA |
| a7b1_APOE3_ | CATTCAGATGACCCAGAGTCCCTCGAGTTTAAGTGCGTCTGTGGGAGATCGTGTGAC |
| R134A_K135S | GATTACATGCCGCGCCTCAGAGTCGGTGGATAATTACGGGATCTCTTTCATGAATTGG |
| | TTTCAGCAAAAACCGGGCAAGGCCCCTAAGCTGCTGATCTACGCAGCCTCTGACTTA |
| | GGATCTGGGGTGCCAAGCCGGTTTTCAGGCTCTGGTTCTGGCACAGACTTCACCCT |
| | GACAATATCGTCTCTCCAGCCAGAAGATTTCGCCACATACTACTGCCACCAGAGTAA |
| | GGAGGTGCCCTACACCTTTGGTCAAGGCACCAAACTCGAAATCAAGGGTGGAGGGG |
| | GTTCTGGGGGCGGAGGCTCTGGGGGAGGCGGCTCCCAGGTTCAGCTAGTTCAATC |
| | TGGGGCTGAGGTCAAGAAACCCGGGGCGTCAGTTAAGGTGAGCTGTAAGGCGAGC |
| | GGATACACATTCACTAAGTACTGGATTAATTGGGTTAGACAGGCCCCGGGCCAAGGA |
| | CTGGAGTGGATGGGGATAATTTATCCTGGCAGCTCAACAAGCAATTACAACGAGAAG |
| | TTTAAAACACGGGTGACGATGACAGTGGATACGAGCACCTCAACAGCATATATGGAA |
| | CTGTCCAGCCTGAGATCTGAAGATACAGCTGTTTACTACTGTGTGAAGAATGATAACT |
| | GGGGCCAGGGTACACTCGTAACAGTATCAAGCggatccggtgggtccggtagtggtggttccGA |
| | AGGGGAACCGGAGGTGACGGACCAACTTGAGTGGCAGTCTAATCAGCCCTGGGAA |
| | CAGGCACTCAACCGCTTCTGGGATTATCTGCGCTGGGTACAGACCCTGAGCGACCA |
| | GGTGCAGGAGGAATTACAGAGCTCTCAAGTGACTCAGGAGCTTACCGCTCTTATGGA |
| | AGACACAATGACTGAGGTTAAGGCATACAAAAAGGAGCTGGAGGAACAGTTGGGGC |
| | CAGTCGCAGAGGAAACTAGGGCACGGCTGGGTAAGGAAGTGCAGGCCGCTCAGG |
| | CCAGGCTTGGGGCCGATATGGAGGATCTGCGCAACCGCCTGGGACAGTACCGGAA |
| | TGAGGTTCACACCATGTTAGGTCAGAGCACAGAGGAAATCAGAGCCCGGCTCAGCA |
| | CACACCTCGCAAGTATGCGCAAGCGCCTAATGCGTGACGCCGAGGATCTGCAGAA |
| | GCGCCTGGCGGTGTACAAAGCTGGCGCAAGAGAGGGCGCCGAGAGAGGCGTCTC |
| | GGCCATTAGAGAGAGACTAGGACCCTTGGTCGAGCAGGGGAGGCAAAGAACAGCC |
| | AACCTGGGAGCCGGCGCCGCACAACCTCTAAGAGACCGCGCTCAGGCCTTTGGTG |
| | ATCGGATAAGAGGAAGGTTGGAGGAGGTGGGCAATCAGGCAAGAGACCGTCTCGA |
| | GGAAGTGCGCGAGCACATGGAGGAGGTGAGAAGTAAAATGGAAGAACAAACCCAG |
| | CAGATTAGGCTTCAGGCAGAAATATTTCAGGCCCGCTTAAAGGGCTGGTTCGAGCCT |
| | ATCGTGGAGGATATGCATAGACAGTGGGCCAACCTGATGGAGAAGATCCAAGCCTCT |
| | GTTGCAACCAACCCCATCATAACTCCAGTGGCTCAGGAAAACCAGggcagcggcCATC |
| | ACCATCACCATCACTGA (SEQ ID NO: 259) |
| scFv- | MKVLWAALLVTFLAGCQADIQMTQSPSSLSASVGDRVTITCRASESVDNYGISFMNWFQ |
| a7b1_APOE3_ | QKPGKAPKLLIYAASDLGSGVPSRFSGSGSGTDFTLTISSLOPEDFATYYCHQSKEVPYTF |
| R134A_K135S | GQGTKLEIKGGGGSGGGGSGGGGSQVQLVQSGAEVKKPGASVKVSCKASGYTFTKYWI |
| | NWVRQAPGQGLEWMGIIYPGSSTSNYNEKFKTRVTMTVDTSTSTAYMELSSLRSEDTAVY |
| | YCVKNDNWGQGTLVTVSSGSGGSGSGGSEGEPEVTDQLEWQSNQPWEQALNRFWD |
| | YLRWVQTLSDQVQEELQSSQVTQELTALMEDTMTEVKAYKKELEEQLGPVAEETRARLGK |
| | EVQAAQARLGADMEDLRNRLGQYRNEVHTMLGQSTEEIRARLSTHLASMRKRLMRDAE |
| | DLQKRLAVYKAGAREGAERGVSAIRERLGPLVEQGRQRTANLGAGAAQPLRDRAQAFG |
| | DRIRGRLEEVGNQARDRLEEVREHMEEVRSKMEEQTQQIRLQAEIFQARLKGWFEPIVE |
| | DMHRQWANLMEKIQASVATNPIITPVAQENQGSGHHHHHH* (SEQ ID NO: 260) |
| scFv- | ATGAAGGTTCTGTGGGCTGCGTTGCTGGTCACATTCCTGGCAGGATGCCAGGCCGA |
| CD117_APOE3_ | TATTCAGATGACACAGTCCCCTTCCTCCCTCTCCGCCTCGGTGGGCGATCGCGTTA |
| R134A_K135S | CAATCACTTGTCGGGCCAGTCAAAGAGGCCTAAGGAATGTGGCAGTTGCGTGGTAC |
| | CAACAGAAGCCCGGTAAAGCCCCCAAACTGCTGATCTATTCTGCATCATCACTCTAT |
| | AGCGGCGTGCCTAGTCGGTTCAGTGGCAGTAGGTCTGGGACAGATTTTACTTTGACC |
| | ATTTCCTCCCTCCAGCCAGAGGACTTCGCGACCTACTATTGCCAACAATGGGCAGTG |
| | CACAGCCTGATCACATTCGGGCAGGGAACGAAAGTCGAAATCAAACGAGGAGGTGG |
| | AGGTTCAGGAGGTGGAGGTAGCGGAGGTGGAGGTAGCGAGGTCCAGTTGGTTGAG |
| | AGTGGCGGCGGGTTGGTACAGCCGGGCGGGTCCCTACGCCTGTCCTGCGCAGCA |
| | TCTGGCTTCAACATATCCGTATATATGATGCACTGGGTCCGCCAAGCCCCCGGGAAG |
| | GGCCTGGAATGGGTGGCAAGCATCTACCCCTATTCGGGATACACCTACTATGCAGA |
| | CAGCGTGAAAGGCCGATTCACGATCTCTGCTGACACCAGTAAGAACACGGCCTATC |
| | TGCAGATGAATTCTTTGAGAGCAGAGGATACAGCCGTTTACTACTGCGCCAGATACGT |
| | ATACCACGCTTTAGACTATTGGGGACAGGGCACCCTGGTAACCGTGTCGTCAggatcc |
| | ggtgggtccggtagtggtggttccGAAGGGGAACCGGAGGTGACGGACCAACTTGAGTGGCA |
| | GTCTAATCAGCCCTGGGAACAGGCACTCAACCGCTTCTGGGATTATCTGCGCTGGGT |
| | ACAGACCCTGAGCGACCAGGTGCAGGAGGAATTACAGAGCTCTCAAGTGACTCAGG |
| | AGCTTACCGCTCTTATGGAAGACACAATGACTGAGGTTAAGGCATACAAAAAGGAGCT |
| | GGAGGAACAGTTGGGGCCAGTCGCAGAGGAAACTAGGGCACGGCTGGGTAAGGAA |
| | GTGCAGGCCGCTCAGGCCAGGCTTGGGGCCGATATGGAGGATCTGCGCAACCGC |
| | CTGGGACAGTACCGGAATGAGGTTCACACCATGTTAGGTCAGAGCACAGAGGAAAT |
| | CAGAGCCCGGCTCAGCACACACCTCGCAAGTATGCGCAAGCGCCTAATGCGTGAC |
| | GCCGAGGATCTGCAGAAGCGCCTGGCGGTGTACAAAGCTGGCGCAAGAGAGGGC |
| | GCCGAGAGAGGCGTCTCGGCCATTAGAGAGAGACTAGGACCCTTGGTCGAGCAGG |
| | GGAGGCAAAGAACAGCCAACCTGGGAGCCGGCGCCGCACAACCTCTAAGAGACC |
| | GCGCTCAGGCCTTTGGTGATCGGATAAGAGGAAGGTTGGAGGAGGTGGGCAATCAG |
| | GCAAGAGACCGTCTCGAGGAAGTGCGCGAGCACATGGAGGAGGTGAGAAGTAAAA |
| | TGGAAGAACAAACCCAGCAGATTAGGCTTCAGGCAGAAATATTTCAGGCCCGCTTAA |
| | AGGGCTGGTTCGAGCCTATCGTGGAGGATATGCATAGACAGTGGGCCAACCTGATG |
| | GAGAAGATCCAAGCCTCTGTTGCAACCAACCCCATCATAACTCCAGTGGCTCAGGA |
| | AAACCAGggcagcggcCATCACCATCACCATCACTGA (SEQ ID NO: 261) |
| scFv- | MKVLWAALLVTFLAGCQADIQMTQSPSSLSASVGDRVTITCRASQRGLRNVAVAWYQQ |
| CD117_APOE3_ | KPGKAPKLLIYSASSLYSGVPSRFSGSRSGTDFTLTISSLQPEDFATYYCQQWAVHSLITFG |
| R134A_K135S | QGTKVEIKRGGGGSGGGGSGGGGSEVQLVESGGGLVQPGGSLRLSCAASGFNISVYM |
| | MHWVRQAPGKGLEWVASIYPYSGYTYYADSVKGRFTISADTSKNTAYLQMNSLRAEDTA |
| | VYYCARYVYHALDYWGQGTLVTVSSGSGGSGSGGSEGEPEVTDQLEWQSNQPWEQAL |
| | NRFWDYLRWVQTLSDQVQEELQSSQVTQELTALMEDTMTEVKAYKKELEEQLGPVAEET |
| | RARLGKEVQAAQARLGADMEDLRNRLGQYRNEVHTMLGQSTEEIRARLSTHLASMRKRL |
| | MRDAEDLQKRLAVYKAGAREGAERGVSAIRERLGPLVEQGRQRTANLGAGAAQPLRDR |
| | AQAFGDRIRGRLEEVGNQARDRLEEVREHMEEVRSKMEEQTQQIRLQAEIFQARLKGWF |
| | EPIVEDMHRQWANLMEKIQASVATNPIITPVAQENQGSGHHHHHH* (SEQ ID NO: |
| | 262) |
| RGD/PK7_ | atgAACGATAAGCTCACCTTATGGACTACACCGGCCCCGAGTCCGAATTGTAGGCTG |
| APOE3_R134A_ | AATGCCGAGAAAGATGCCAAACTGACTCTGGTTTTGACAAAGTGCGGAAGTCAAATAT |
| K135S | TGGCGACTGTAAGCGTGCTGGCCGTGAAAGGGTCCTTGGCCCCGATTTCTGGTACA |
| | GTACAGTCAGCTCATCTGATCATTAGATTTGATGAAAATGGAGTCTTGTTGAACAATTCA |
| | TTTCTGGACCCGGAGTACTGGAATTTCAGGAATGGTGACCTGACGGAAGGGACAGCT |
| | TACACCAACGCTGTGGGCTTCATGCCAAATCTTAGCGCCTACCCCAAATCACATGGA |
| | AAAACTGCAAAATCTAACATCGTGAGCCAGGTCTATTTGAATGGCGACAAAACTAAGC |
| | CTGTCACGCTTACTATTACCTTGAATGGAACCCAAGAGACAGGCGACACGACCGCTT |
| | GTGATTGTCGAGGCGATTGTTTCTGCGGCCCAAGTGCTTATTCCATGTCCTTTTCTTGG |
| | GATTGGAGTGGCCATAACTATATCAACGAGATTTTCGCTACATCCTCTTATACCTTCTC |
| | ATACATCGCCCAGGAAGGTAGCGGAAGCGGCTCCGGGTCGGGAAGCAAGAAGAAG |
| | AAAAAGAAGAAAggatccggtgggtccggtagtggtggttccGAAGGGGAACCGGAGGTGACGG |
| | ACCAACTTGAGTGGCAGTCTAATCAGCCCTGGGAACAGGCACTCAACCGCTTCTGG |
| | GATTATCTGCGCTGGGTACAGACCCTGAGCGACCAGGTGCAGGAGGAATTACAGAG |
| | CTCTCAAGTGACTCAGGAGCTTACCGCTCTTATGGAAGACACAATGACTGAGGTTAAG |
| | GCATACAAAAAGGAGCTGGAGGAACAGTTGGGGCCAGTCGCAGAGGAAACTAGGG |
| | CACGGCTGGGTAAGGAAGTGCAGGCCGCTCAGGCCAGGCTTGGGGCCGATATGGA |
| | GGATCTGCGCAACCGCCTGGGACAGTACCGGAATGAGGTTCACACCATGTTAGGTC |
| | AGAGCACAGAGGAAATCAGAGCCCGGCTCAGCACACACCTCGCAAGTATGCGCAA |
| | GCGCCTAATGCGTGACGCCGAGGATCTGCAGAAGCGCCTGGCGGTGTACAAAGCT |
| | GGCGCAAGAGAGGGCGCCGAGAGAGGCGTCTCGGCCATTAGAGAGAGACTAGGA |
| | CCCTTGGTCGAGCAGGGGAGGCAAAGAACAGCCAACCTGGGAGCCGGCGCCGCA |
| | CAACCTCTAAGAGACCGCGCTCAGGCCTTTGGTGATCGGATAAGAGGAAGGTTGGA |
| | GGAGGTGGGCAATCAGGCAAGAGACCGTCTCGAGGAAGTGCGCGAGCACATGGAG |
| | GAGGTGAGAAGTAAAATGGAAGAACAAACCCAGCAGATTAGGCTTCAGGCAGAAATA |
| | TTTCAGGCCCGCTTAAAGGGCTGGTTCGAGCCTATCGTGGAGGATATGCATAGACAG |
| | TGGGCCAACCTGATGGAGAAGATCCAAGCCTCTGTTGCAACCAACCCCATCATAACT |
| | CCAGTGGCTCAGGAAAACCAGggcagcggcCATCACCATCACCATCACTGA (SEQ ID |
| | NO: 263) |
| RGD/PK7_ | MNDKLTLWTTPAPSPNCRLNAEKDAKLTLVLTKCGSQILATVSVLAVKGSLAPISGTVQSA |
| APOE3_R134A_ | HLIIRFDENGVLLNNSFLDPEYWNFRNGDLTEGTAYTNAVGFMPNLSAYPKSHGKTAKS |
| K135S | NIVSQVYLNGDKTKPVTLTITLNGTQETGDTTACDCRGDCFCGPSAYSMSFSWDWSGH |
| | NYINEIFATSSYTFSYIAQEGSGSGSGSGSKKKKKKKGSGGSGSGGSEGEPEVTDQLEW |
| | QSNQPWEQALNRFWDYLRWVQTLSDQVQEELQSSQVTQELTALMEDTMTEVKAYKKE |
| | LEEQLGPVAEETRARLGKEVQAAQARLGADMEDLRNRLGQYRNEVHTMLGQSTEEIRAR |
| | LSTHLASMRKRLMRDAEDLQKRLAVYKAGAREGAERGVSAIRERLGPLVEQGRQRTANL |
| | GAGAAQPLRDRAQAFGDRIRGRLEEVGNQARDRLEEVREHMEEVRSKMEEQTQQIRLQ |
| | AEIFQARLKGWFEPIVEDMHRQWANLMEKIQASVATNPIITPVAQENQGSGHHHHHH* |
| | (SEQ ID NO: 264) |
| Protein_A/G_ | ATGAAGGTTCTGTGGGCTGCGTTGCTGGTCACATTCCTGGCAGGATGCCAGGCCTC |
| APOE3_ | ATTGAAAGACGATCCAAGCCAGAGCGCGAATTTGTTATCTGAGGCTAAAAAACTGAAC |
| R134A_K135S | GAGAGCCAGGCCCCTAAGGCCGATAACAAATTCAACAAGGAACAACAAAACGCATT |
| | CTATGAAATTCTGCATCTTCCAAATTTAAACGAAGAGCAGCGTAACGGGTTCATTCAGT |
| | CCCTCAAAGACGATCCAAGTCAGTCAGCCAATCTTCTCGCTGAAGCCAAGAAGCTG |
| | AATGACGCTCAAGCTCCTAAGGCTGACAATAAGTTCAACAAGGAGCAGCAAAATGCT |
| | TTCTACGAGATTCTTCACTTGCCAAACCTCACTGAGGAGCAGAGGAATGGATTCATTC |
| | AGAGCTTAAAAGACGATCCAAGCGTGTCGAAGGAGATATTGGCAGAGGCCAAGAAA |
| | CTTAATGATGCCCAGGCCCCTAAAACCACCTATAAGCTCGTGATCAACGGCAAAACT |
| | CTGAAGGGCGAGACAACAACAGAAGCTGTTGACGCGGAAACCGCAGAGCGGCATT |
| | TCAAACAGTACGCCAACGATAATGGTGTTGACGGTGAGTGGACCTACGATGACGCCA |
| | CAAAAACCTTTACTGTCACCGAGAAACCCGAGGTGATCGATGCAAGCGAATTGACAC |
| | CCGCCGTTggatccggtgggtccggtagtggtggttccGAAGGGGAACCGGAGGTGACGGACC |
| | AACTTGAGTGGCAGTCTAATCAGCCCTGGGAACAGGCACTCAACCGCTTCTGGGATT |
| | ATCTGCGCTGGGTACAGACCCTGAGCGACCAGGTGCAGGAGGAATTACAGAGCTCT |
| | CAAGTGACTCAGGAGCTTACCGCTCTTATGGAAGACACAATGACTGAGGTTAAGGCA |
| | TACAAAAAGGAGCTGGAGGAACAGTTGGGGCCAGTCGCAGAGGAAACTAGGGCAC |
| | GGCTGGGTAAGGAAGTGCAGGCCGCTCAGGCCAGGCTTGGGGCCGATATGGAGG |
| | ATCTGCGCAACCGCCTGGGACAGTACCGGAATGAGGTTCACACCATGTTAGGTCAG |
| | AGCACAGAGGAAATCAGAGCCCGGCTCAGCACACACCTCGCAAGTATGCGCAAGC |
| | GCCTAATGCGTGACGCCGAGGATCTGCAGAAGCGCCTGGCGGTGTACAAAGCTGG |
| | CGCAAGAGAGGGCGCCGAGAGAGGCGTCTCGGCCATTAGAGAGAGACTAGGACC |
| | CTTGGTCGAGCAGGGGAGGCAAAGAACAGCCAACCTGGGAGCCGGCGCCGCACA |
| | ACCTCTAAGAGACCGCGCTCAGGCCTTTGGTGATCGGATAAGAGGAAGGTTGGAGG |
| | AGGTGGGCAATCAGGCAAGAGACCGTCTCGAGGAAGTGCGCGAGCACATGGAGGA |
| | GGTGAGAAGTAAAATGGAAGAACAAACCCAGCAGATTAGGCTTCAGGCAGAAATATTT |
| | CAGGCCCGCTTAAAGGGCTGGTTCGAGCCTATCGTGGAGGATATGCATAGACAGTG |
| | GGCCAACCTGATGGAGAAGATCCAAGCCTCTGTTGCAACCAACCCCATCATAACTC |
| | CAGTGGCTCAGGAAAACCAGggcagcggcCATCACCATCACCATCACTGA (SEQ ID |
| | NO: 265) |
| Protein_A/G_ | MKVLWAALLVTFLAGCQASLKDDPSQSANLLSEAKKLNESQAPKADNKFNKEQQNAFY |
| APOE3_ | EILHLPNLNEEQRNGFIQSLKDDPSQSANLLAEAKKLNDAQAPKADNKFNKEQQNAFYEI |
| R134A_K135S | LHLPNLTEEQRNGFIQSLKDDPSVSKEILAEAKKLNDAQAPKTTYKLVINGKTLKGETTTEA |
| | VDAETAERHFKQYANDNGVDGEWTYDDATKTFTVTEKPEVIDASELTPAVGSGGSGSGG |
| | SEGEPEVTDQLEWQSNQPWEQALNRFWDYLRWVQTLSDQVQEELQSSQVTQELTALM |
| | EDTMTEVKAYKKELEEQLGPVAEETRARLGKEVQAAQARLGADMEDLRNRLGQYRNEVH |
| | TMLGQSTEEIRARLSTHLASMRKRLMRDAEDLQKRLAVYKAGAREGAERGVSAIRERLGP |
| | LVEQGRQRTANLGAGAAQPLRDRAQAFGDRIRGRLEEVGNQARDRLEEVREHMEEVRS |
| | KMEEQTQQIRLQAEIFQARLKGWFEPIVEDMHRQWANLMEKIQASVATNPIITPVAQENQ |
| | GSGHHHHHH* (SEQ ID NO: 266) |

### EXAMPLES

The invention now being generally described, it will be more readily understood by reference to the following examples which are included merely for purposes of illustration of certain aspects and embodiments of the present invention, and are not intended to limit the invention.

### Example 1: LNP Production and Characterization

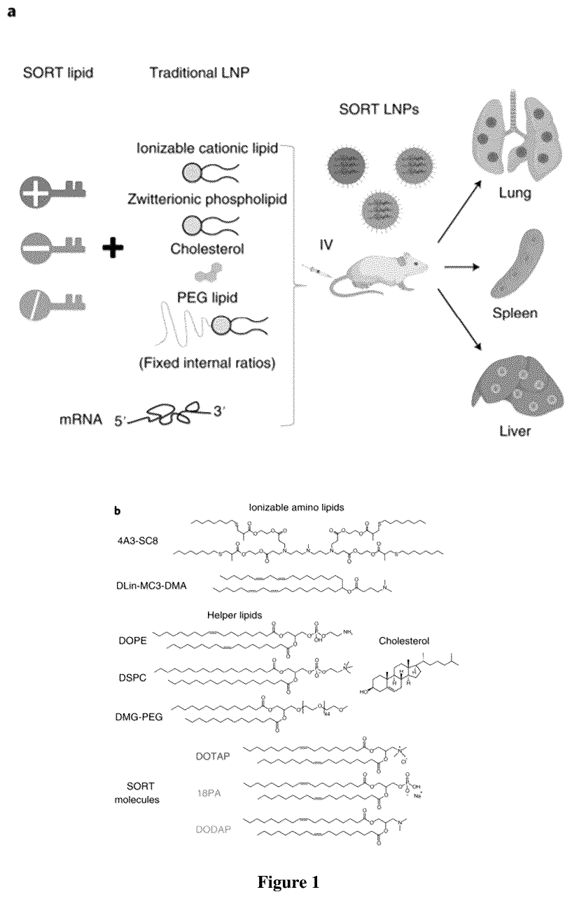

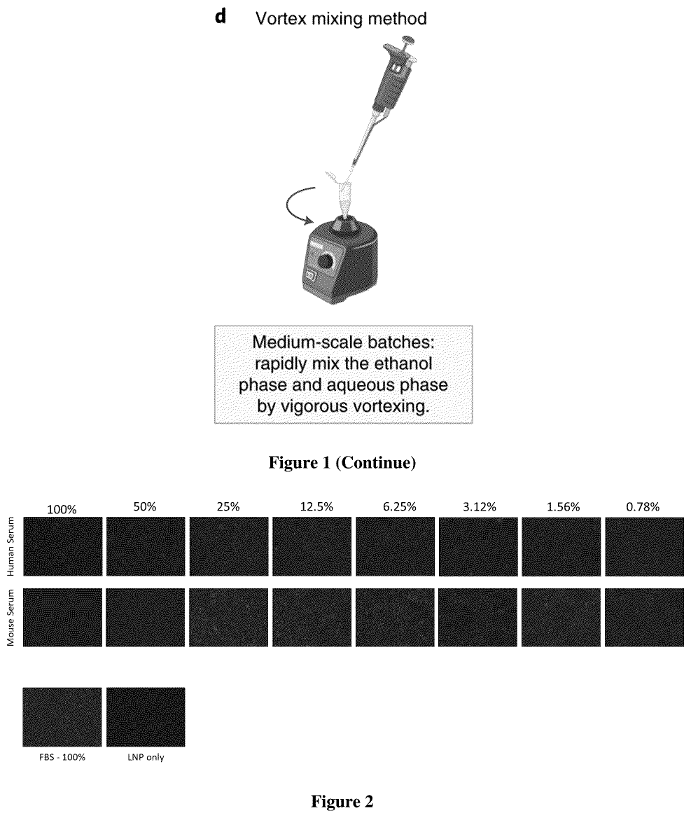

LNPs are synthesized according the protocol outlined in Preparation of selective organ-targeting (SORT) lipid nanoparticles (LNPs) using multiple technical methods for tissue-specific mRNA delivery (Wang X., et al. Nat Protoc. 2023 January; 18 (1): 265-291) (FIG. 1).

| Lipid | [Stock] (mg/ml) | Volume (μL) |
|---|---|---|
| MC3 | 75 | 13.3 |
| DSPC | 10 | 24.6 |
| Cholesterol | 10 | 46.4 |
| DMG-PEG | 10 | 11.7 |

All LNPs used here are made using the MC3 formulation known in the art (from Onpattro). The vortex mixing uses the following aqueous and ethanol phase volumes. mRNAs expressing reporter proteins such as Cre, GFP, or fLuc are encapsulated in LNPs using the following volumes. Final RNA concentration in LNPs are 10 ng/μl.

| | | | | | | | Final volume |
|---|---|---|---|---|---|---|---|
| | Complete | | | | Total | Total | after |
| | lipid mix | | mRNA | Citrate | EtOH | aqueous | compensation |
| MC3 | in EtOH | EtOH | stock | buffer | phase | phase | with PBS |
| formulations | (μL) | (uL) | (uL) | (uL) | (μL) | (μL) | (μL) |
| MC3 LNPs | 21.00 | 9.00 | 10 | 80 | 30 | 90 | 1,000 |
| | (from | | | | | | |
| | Box 2, | | | | | | |
| | step 7) | | | | | | |

Following vortex mixing, a dialysis step is performed using a Pur-A-Lyzer midi (Sigma-Aldrich PURD10005) or similar low MWCO dialysis kit for 2 hours. After dialysis, the total volume is adjusted to 1 mL with PBS.

### General Methods for all Standard Experiments

LNPs are produced according as detailed above. Transductions are performed on HEK-293 FT, HepG2, or primary cell lines, depending on the experiment. Cells are dosed with 15 μL of LNP which equates to 150 ng of RNA.

For experiments testing different protein coronas (the majority listed), LNPs are first synthesized and then mixed in 1:3 volume ratio with media or purified protein (either from Ni-NTA affinity purification, Ni-NTA affinity purification and FPLC/Size exclusion chromatography (SEC) purification, both, or commercial vendor recombinant protein).

In the majority of cases, the protein being tested is in the form of “conditioned media” (CM), which is media collected 48-72h post-transfection from HEK293FT cells transfected with a variety of constructs. As APOE and similar lipoproteins are often secreted, the modified lipoprotein is usually present in the media. CM can be used directly or concentrated using a tool such as an Amicon centrifugal filter (most commonly a 10 kDA MWCO filter).

CM fractions are normally run on a denaturing gel (ex. 4-20% Stain Free gel from BioRad) and imaged to verify expression and secretion of the constructs in question. A BCA assay can also be run on the secreted fractions to measure and normalize protein inputs into the transduction.

LNPs are incubated for 15 minutes at 37 C in an incubator or thermocycler. 16-24h post-transduction, the dosed cells can be imaged by microscope for fluorescence (if LNP cargo is GFP), luminescence via the Brite-Glo assay and a BioTek reader (if cargo is luciferase), or flow using a Cytoflex or similar analysis platform (if cargo is fluorescent protein).

Titering Human and Mouse Serum with LNPs

### Methods

LNPs are produced as detailed above. Transductions are performed on HEK-293 FT. Cells are dosed with 15 μL of LNP which equates to 150 ng of RNA.

LNPs are first synthesized and then mixed in 1:3 volume ratio with media or purified protein. Here, mouse and human serum diluted to different % s with PBS are also used.

LNPs are incubated for 15 minutes at 37 C in an incubator or thermocycler. 16-24h post-transduction, the dosed cells can be imaged by microscope for fluorescence (if LNP cargo is GFP).

### Significance

These images show that varying amounts of mouse or human serum incubated with homemade LNPs can improve their transduction of HEK293FT cells, although at a certain point transduction is negatively impacted, potentially by harming cell health (FIG. 2).

New LNPs with Commercial RNAs

### Methods

LNPs are produced as detailed above. Here, LNPs are made with either unmodified or 5′moU-modified GFP mRNAs. Transductions are performed on HEK-293 FT. Cells are dosed with different RNA amounts, either naked or mixed with wildtype or mutant APOE CM.

LNPs are first synthesized and then mixed in 1:3 volume ratio with media or purified protein. Here, mouse and human serum diluted to different % s with PBS are also used.

LNPs are incubated for 15 minutes at 37 C in an incubator or thermocycler. 16-24h post-transduction, the dosed cells can be imaged by microscope for fluorescence (if LNP cargo is GFP).

### Significance

These images show that our methods work with different types and dosages of commercial RNA (FIG. 3). In additional, they are used to identify a dose used for following experiments. Here, unmodified RNA shows a higher fluorescence than the 5′moU modified RNA. This panel also shows a strong ablation of transduction signal using a mutant-APOE corona.

### Example 2: LNP Production and Characterization: Electron Microscopy and DLC

### Protocol for Dynamic Light Scattering (DLS) and Electron Microscopy (EM)

LNPs are produced by vortex-mixing method as detailed above.

Conditioned media are prepared with the following protocol. Constructs are transfected into HEK293-FT cells, 12-14 hours after transfection, media are changed to Fluorobrite DMEM without FBS. Conditioned media are collected 48-72h post-transfection and filtered through 0.45 μm filters.

LNPs are used for DLS and EM either directly or after incubating with conditioned media or recombinant proteins. DLS measurements are performed using Zetasizer Nano instrument and its software. EM imaging were performed using JEOL 2100F TEM instrument.

### Methods

LNPs are produced as detailed above. Here, LNPs are made with either unmodified or 5′moU-modified GFP mRNAs. Transductions are performed on HEK-293 FT. Cells are dosed with different RNA amounts, either naked or mixed with wildtype or mutant APOE CM.

LNPs are first synthesized and then mixed in 1:3 volume ratio with media or purified protein. Here, mouse and human serum diluted to different % s with PBS are also used.

LNPs are incubated for 15 minutes at 37 C in an incubator or thermocycler. 16-24h post-transduction, the dosed cells can be imaged by microscope for fluorescence (if LNP cargo is GFP).

### Significance

This plot indicates that the average size of particles in the mixtures increases when LNPs are incubated with the conditioned media or purified protein (FIG. 4). This suggests that the LNPs themselves may be getting larger through the formation of a protein corona, or generally through the binding of proteins in the media. This suggests that our strategy is successfully creating a protein corona in vitro.

### Methods

LNPs are produced as detailed above. Here, LNPs are made with either unmodified or 5′moU-modified GFP mRNAs. LNPs are first synthesized and then mixed in 1:3 volume ratio with media or purified protein.

LNPs are incubated for 15 minutes at 37 C in an incubator or thermocycler. These LNPs are then further diluted, and a set of concentrated samples imaged with two different negative staining EM approaches (some information in introduction slides of this section).

### Significance

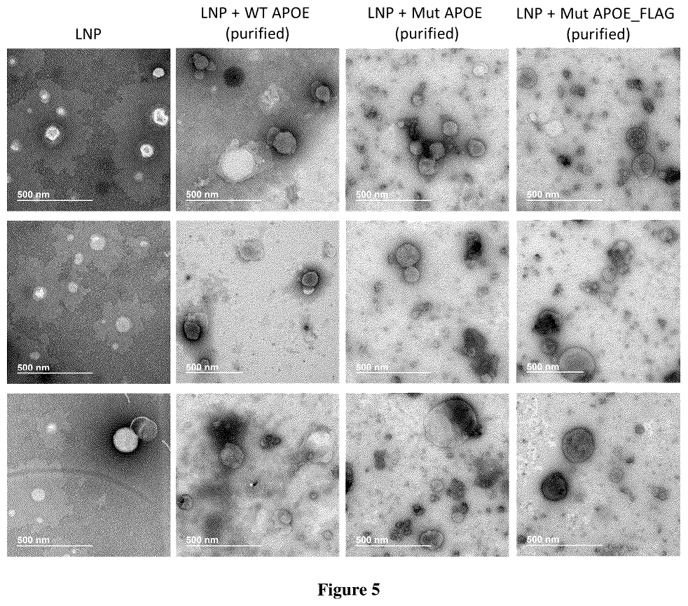

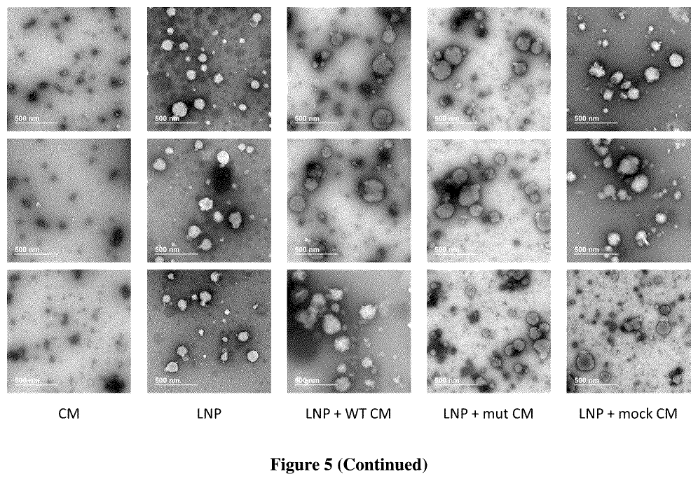

These images back up the idea that the LNPs are expanding when mixed with protein, either from conditioned media or purified material (FIG. 5). This further indicates that a protein corona, or general protein binding, is occurring in our experimental conditions.

Approximate Sizes for LNPs from Different Experimental Conditions, Measured in ImageJ from Representative EM Pictures.

### Methods

LNPs are produced as detailed above. Here, LNPs are made with either unmodified or 5′moU-modified GFP mRNAs. LNPs are first synthesized and then mixed in 1:3 volume ratio with media or purified protein.

LNPs are incubated for 15 minutes at 37 C in an incubator or thermocycler. These LNPs are then further diluted and a set of concentrated samples imaged with two different negative staining EM approaches (some information in introduction slides of this section).

By setting a scale in ImageJ, approximate sizes can be measured with a “ruler” horizontally or vertically across all LNP-morphology objects in each view.

### Significance

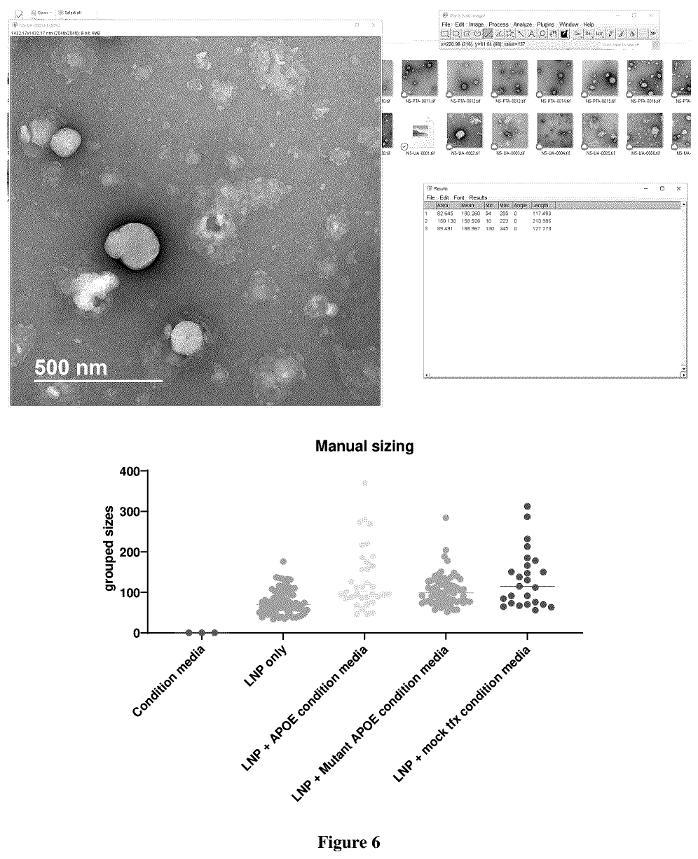

This quantification backs up the observation that the particles appear to be expanding when mixed with a variety of different protein-containing experimental conditions (FIG. 6). Notably, most mixtures cause a slight increase in size relative to the “LNP-only” particles, as well as a wider ranger of sizes (larger standard deviation), suggesting a more heterogenous mixture following incubation.

### Example 3: Protein Production and Characterization

### Straightforward Purification: Using NiNTA and Amicon Filters to Produce More Pure Protein

Initially: Transduction with Conditioned Media

Transfect with PEI into HEK293-FT cells cultured in DMEM+10% FBS at a 24 or 12 well scale. 4-6 hours after transfection, change the media in the flasks to DMEM (Gibco) only. Regular or Fluorobrite DMEM can be used exchangably. Collect media 48-72h post-transfection and either incubate directly with separately produced LNPs, or concentrate and then incubate.

### Protein Purification Via NiNTA

Transfect with PEI into HEK293-FT cells cultured in DMEM+10% FBS at a T75 or T225 (s). 4-6 hours after transfection, change the media in the flasks to DMEM only. Collect media 48-72h post-transfection and concentrate it using a 10 kDa MWCO Amicon Centrifugal Filters. Purify using tabletop Nickel-NTA columns (Thermo Fisher). Optional: SEC FPLC on eluted fractions from NiNTA column.

Purification of a C-Terminally His-Tagged APOE Construct from Either Cell Lysate or Secretion to Media

### Methods

His-tagged APOE transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purify using tabletop Nickel-NTA columns (Thermo Fisher).

Mix fraction for gel with 4× loading Laemmli buffer loading dye (BioRad) with 10% v/v beta-mercaptoethanol and heat in a thermocycler for 10 minutes at 95 C to denature protein. Load a 4-20% TGX mini-PROTEAN gel (BioRad) with the incubated protein samples and a stain free ladder and run at 150-200V in 1× Tris-Glycine-SDS buffer (BioRad) until complete, then image.

### Significance

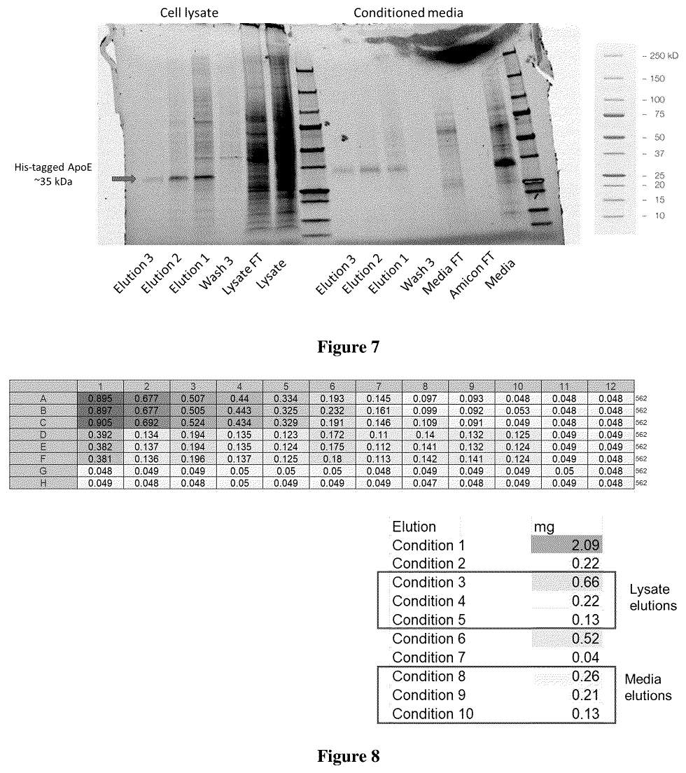

This gel shows that Ni-NTA affinity purification can produce a much purer product than the raw cell lysate or secreted material for use in future transductions (FIG. 7). In general, the secreted material generates an apparent lower yield but higher purity product.

BCA Assay for Protein Purified Via Ni-NTA Purification from Cell Lysate or the Secreted Fraction.

### Methods

His-tagged APOE transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purify using tabletop Nickel-NTA columns (Thermo Fisher). BCA assay is run using a Pierce BCA kit against BSA protein standards.

### Significance

FIG. 8 shows that cell lysate purifications produce a higher total amount of protein. However, the product may be less pure than the purification from secreted fraction (comparing gel images of both).

Transduction Efficiencies Via Flow Cytometry for LNPs Mixed with a Range of Amounts of Purified Protein Purified from Either Cell Lysate or the Secreted Fraction (CM)

### Methods

His-tagged APOE transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purify using tabletop Nickel-NTA columns (Thermo Fisher).

LNPs are produced as detailed above. Transductions are performed on HEK-293FTs in culture. Cells are dosed with 15 μL of LNP which equates to 150 ng of RNA. For experiments testing different protein coronas (the majority listed), LNPs are first synthesized and then mixed in 1:3 volume ratio with media or purified protein. A BCA assay can also be run on the secreted fractions to measure and normalize protein inputs into the transduction. Here, several different amounts of purified protein were used, either 0.5, 1, or 2 μg. LNPs are incubated for 15 minutes at 37 C in an incubator or thermocycler. 16-24h post-transduction, the dosed cells are measured by flow using a Cytoflex.

### Significance

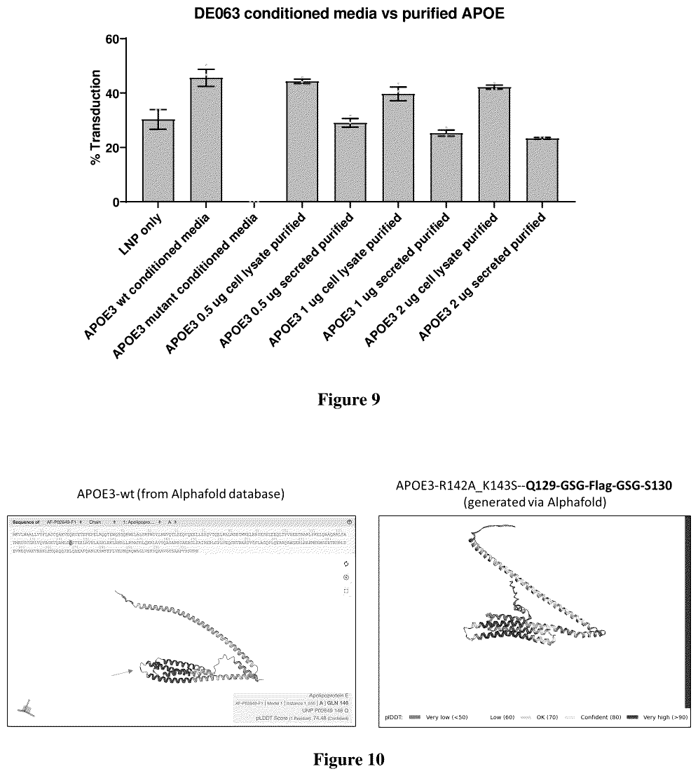

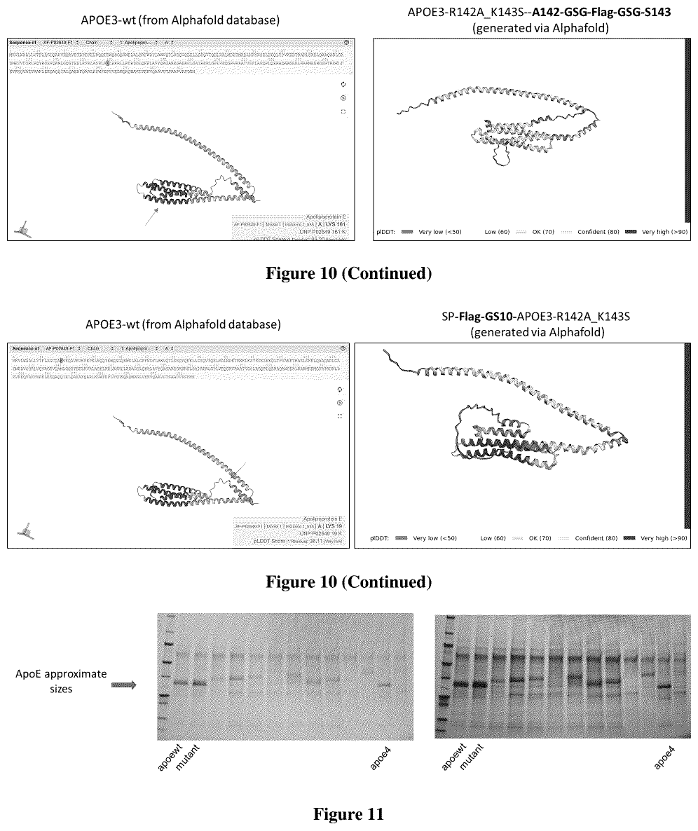

This panel shows that purified protein is still capable of efficient transduction, similar to the conditioned media (FIG. 9). Protein purified from the cell lysate transduces better than protein from purified fractions. Additionally, these results suggest that the inclusion of a C-terminal His tag does not drastically impair LNP binding or another feature of transduction.

AlphaFold Predictions of Structures of Wild-Type and Mutant or Tagged APOE Constructs, Showing a Relatively Conserved Core Structure that Matches the Wildtype Construct.

### Methods

His-tagged APOE transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purify using tabletop Nickel-NTA columns (Thermo Fisher).

Mix fraction for gel with 4× loading Laemmli buffer loading dye (BioRad) with 10% v/v beta-mercaptoethanol and heat in a thermocycler for 10 minutes at 95c to denature protein. Load a 4-20% TGX mini-PROTEAN gel (BioRad) with the incubated protein samples and a stain free ladder and run at 150-200V in 1× Tris-Glycine-SDS buffer (BioRad) until complete, then image.

### Significance

This gel shows the sizes and efficiencies of production of a set of ligand-conjugated APOE constructs (FIG. 11). Importantly, this table suggests that not all are secreted into the media uniformly, indicating that protein purification may be necessary to compare their effects on transduction fairly.

Gels Showing the Effects of Protein Purification on Conditioned Media from Cells Transfected with Either Mouse APOE or Mouse “Dead” Mutant APOE at Each of Several Stages of the Purification Process, from CM to Ni-NTA Fractions to Post-SEC Fractions

### Methods

His-tagged APOE transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purify using tabletop Nickel-NTA columns (Thermo Fisher).

Mix fraction for gel with 4× loading Laemmli buffer loading dye (BioRad) with 10% v/v beta-mercaptoethanol and heat in a thermocycler for 10 minutes at 95c to denature protein. Load a 4-20% TGX mini-PROTEAN gel (BioRad) with the incubated protein samples and a stain free ladder and run at 150-200V in 1× Tris-Glycine-SDS buffer (BioRad) until complete, then image.

### Significance

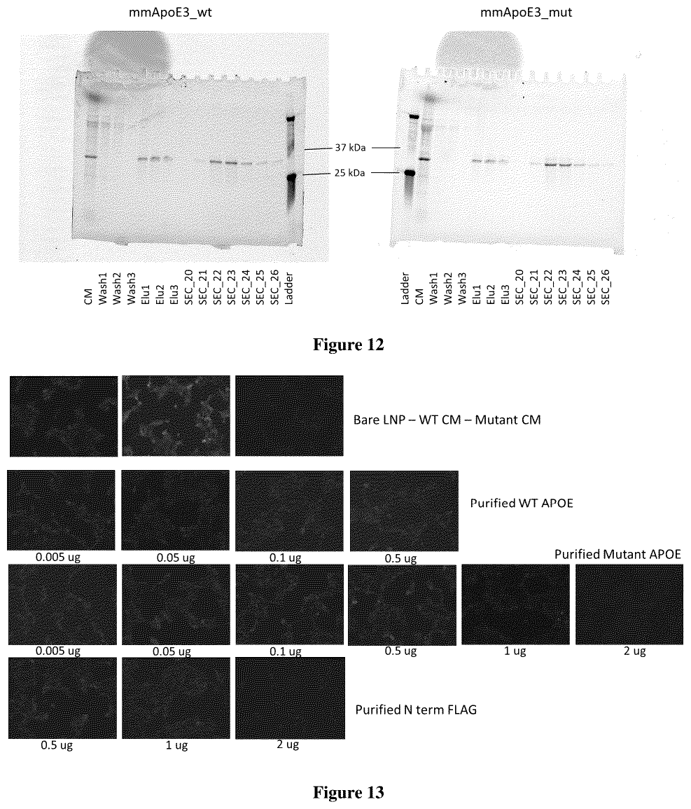

This gel shows the sizes and efficiencies of production of a set of ligand-conjugated APOE constructs (FIG. 12). Importantly, this table suggests that not all are secreted into the media uniformly, indicating that protein purification may be necessary to compare their effects on transduction fairly.

### Methods

His-tagged APOE constructs transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purified using tabletop Nickel-NTA columns (Thermo Fisher).

LNPs are produced as detailed above. Here, LNPs are made with either unmodified or 5′moU-modified GFP mRNAs. Transductions are performed on HEK-293 FT. Cells are dosed with different RNA amounts, either naked or mixed with wildtype or mutant APOE CM. LNPs are first synthesized and then mixed in 1:3 volume ratio with media or purified protein. LNPs are incubated for 15 minutes at 37 C in an incubator or thermocycler. 16-24h post-transduction, the dosed cells can be imaged by microscope for fluorescence (if LNP cargo is GFP).

### Significance

This experiment shows that purified protein is functional at a variety of amounts, including levels much lower than tested previously (FIG. 13). It also reinforces the finding that high protein amounts can be toxic/detrimental to transduction, indicating that dose curves should be done for these experiments.

Homemade LNPs Efficiently Transduced HEK Cells in Culture, Homemade LNPs Transduce Much More Efficiently when Mixed with Media, FBS, or Recombinant Protein

### Methods

LNPs are first synthesized and then mixed in 1:3 volume ratio with media or purified protein. Here, mouse and human serum diluted to different % s with PBS are also used.

LNPs are produced as detailed above. Here, LNPs are made with either unmodified or 5′moU-modified GFP mRNAs. Transductions are performed on HEK-293 FT. Cells are dosed with different RNA amounts, either naked or mixed with wildtype or mutant APOE CM.

LNPs are first synthesized and then mixed in 1:3 volume ratio with media or purified protein. LNPs are incubated for 15 minutes at 37c in an incubator or thermocycler. 16-24h post-transduction, the dosed cells can be imaged by microscope for fluorescence (if LNP cargo is GFP).

### Significance

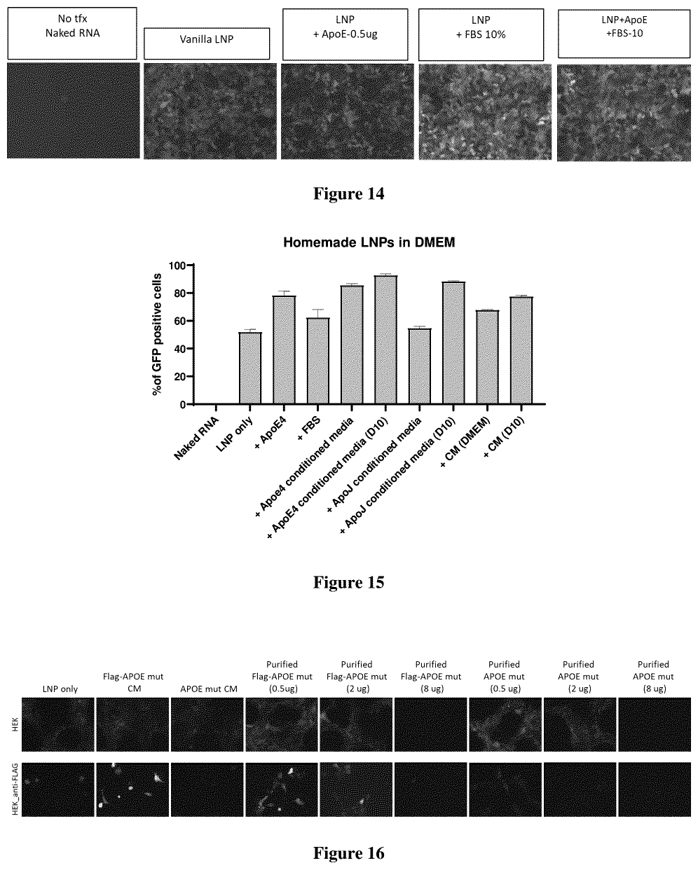

This experiment shows that there is a strong benefit to transduction coming from incubation with serum that can also be recapitulated by incubation with a recombinant lipoprotein (FIG. 14).

### Methods

LNPs are first synthesized and then mixed in 1:3 volume ratio with media or purified protein. Here, mouse and human serum diluted to different % s with PBS are also used.

LNPs are produced as detailed above. Here, LNPs are made with either unmodified or 5′moU-modified GFP mRNAs. Transductions are performed on HEK-293 FT. Cells are dosed with different RNA amounts, either naked or mixed with wildtype or mutant APOE CM.

LNPs are first synthesized and then mixed in 1:3 volume ratio with media or purified protein. LNPs are incubated for 15 minutes at 37 C in an incubator or thermocycler. 16-24h post-transduction, the dosed cells are measured by flow using a Cytoflex.

### Significance

This experiment shows that there is a strong benefit to transduction coming from incubation with serum that can also be recapitulated by incubation with a recombinant lipoprotein (FIG. 15).

### Example 4: Retargeting In Vitro HEK Cell Overexpression

### DE066-Purified FLAG-APOE3

### Methods

His-tagged APOE constructs transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purified using tabletop Nickel-NTA columns (Thermo Fisher).

LNPs are produced according to a protocol known in the art (e.g., Wang X., et al. Nat Protoc. 2023 January; 18 (1): 265-291). Here, LNPs are made with either unmodified or 5′moU-modified GFP mRNAs. Transductions are performed on HEK-293 FT. Cells are dosed with different RNA amounts, either naked or mixed with wildtype or mutant APOE CM.

LNPs are first synthesized and then mixed in 1:3 volume ratio with media or purified protein. LNPs are incubated for 15 minutes at 37 C in an incubator or thermocycler. Here, the LNP constructs are dosed on HEK293FTs and HEK293FTs that were previously uniformly transfected with an anti-FLAG ScFv construct (24 hours previous, using Lipofectamine 3000).

16-24h post-transduction, the dosed cells can be imaged by microscope for fluorescence (if LNP cargo is GFP).

### Significance

This experiment shows how a synthetic ligand-receptor pair (here, a FLAG tag and an anti-FLAG ScFv) can be conjugated with APOE and functionalize the particle through binding. Comparing the transduction efficiencies of the various conditions shows a benefit to transduction when the particle is coated with a FLAG ligand AND the target cell type expresses its “receptor”, while the purified “dead” mutant APOE construct still suppresses transduction at most concentrations tested (FIG. 16).

Transducing HEKs Overexpressing ScFvs Against Our Synthetic Tagged mAPOEs Generates Specific Transduction

### Methods

LNPs are produced as detailed above. Here, LNPs are made with either unmodified or 5′moU-modified GFP mRNAs. Transductions are performed on HEK-293 FT. Cells are dosed with different RNA amounts, either naked or mixed with wildtype or mutant APOE CM.

LNPs are first synthesized and then mixed in 1:3 volume ratio with media or purified protein. LNPs are incubated for 15 minutes at 37 C in an incubator or thermocycler. Here, the LNP constructs are dosed on HEK293FTs and HEK293FTs that were previously uniformly transfected with an anti-EE, moontag, or Suntag ScFv construct (24 hours previous, using Lipofectamine 3000).

16-24h post-transduction, the dosed cells can be imaged by microscope for fluorescence (if LNP cargo is GFP).

### Significance

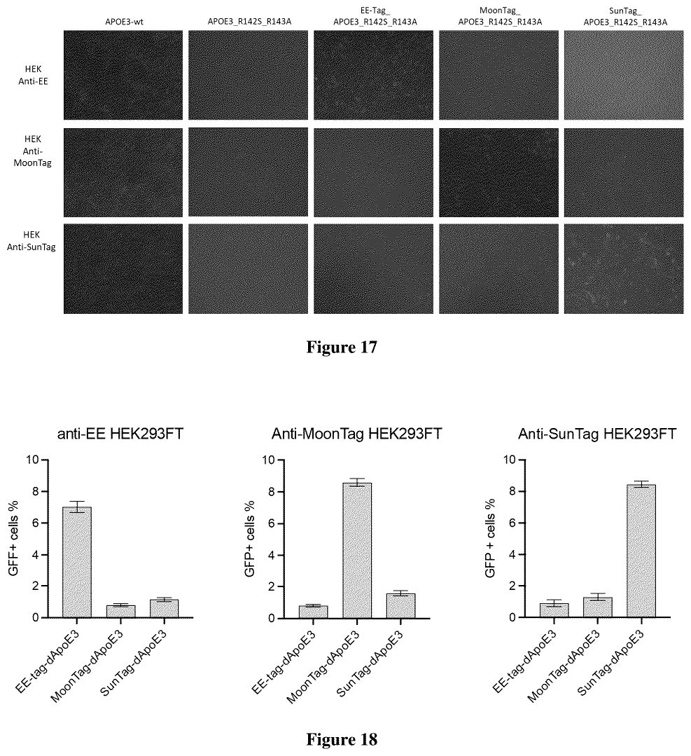

This experiment shows that specific and orthogonal transduction can be set up using APOE constructs conjugated with different ligands. For example, anti-EE tag expressing cells are only transduced well by LNPs coated with EE-conjugated APOE constructs, while anti-Moontag expressing cells are only transduced well by Moontag displaying APOE LNPs (FIG. 17).

### Methods

His-tagged APOE constructs transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purified using tabletop Nickel-NTA columns (Thermo Fisher).

LNPs are produced as detailed above. Here, LNPs are made with either unmodified or 5′moU-modified GFP mRNAs. Transductions are performed on HEK-293 FT. Cells are dosed with different RNA amounts, either naked or mixed with wildtype or mutant APOE CM

LNPs are first synthesized and then mixed in 1:3 volume ratio with media or purified protein. LNPs are incubated for 15 minutes at 37 C in an incubator or thermocycler. Here, the LNP constructs are dosed on HEK293FTs and HEK293FTs that were previously uniformly transfected with an anti-EE, moontag, or Suntag ScFv construct (24 hours previous, using Lipofectamine 3000).

16-24h post-transduction, the dosed cells can be imaged by microscope for fluorescence (if LNP cargo is GFP).

### Significance

This experiment shows that specific and orthogonal transduction can be set up using APOE constructs conjugated with different ligands. For example, anti-EE tag expressing cells are only transduced well by LNPs coated with EE-conjugated APOE constructs, while anti-Moontag expressing cells are only transduced well by Moontag displaying APOE LNPs (FIG. 18).

Conjugation of ScFv to mut-APOE

### Methods

His-tagged APOE constructs transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purified using tabletop Nickel-NTA columns (Thermo Fisher).

LNPs are produced as detailed above. Here, LNPs are made with GFP mRNAs.

LNPs are first synthesized and then mixed in 1:3 volume ratio with media or purified protein. LNPs are incubated for 15 minutes at 37 C in an incubator or thermocycler. Here, the LNP constructs are dosed on HEK293FTs and HEK293FTs that were previously uniformly transfected with CD19 expression construct (24 hours previous, using Lipofectamine 3000).

16-24h post-transduction, the dosed cells can be imaged by microscope for fluorescence (if LNP cargo is GFP). The transduction efficiency was then quantified with a cytoflex analyzer.

### Significance

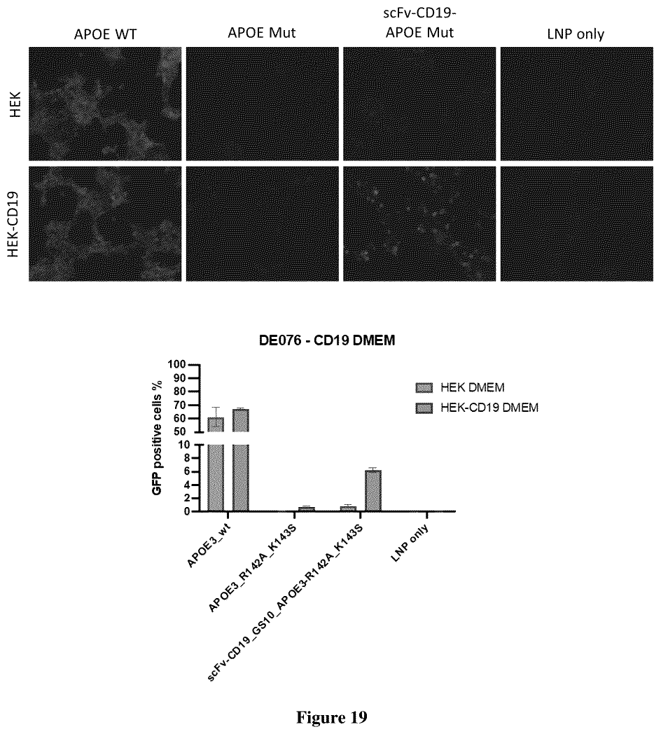

This experiment shows that specific and orthogonal transduction can be set up using APOE constructs conjugated with the CD19 ScFv (and likely other ScFvs for different targets). Here, the CD19-ScFv-APOE enables a boost in transduction efficiency only on the cells expressing the matched receptor target (FIG. 19). This shows that much larger ligands/conjugations than initially tested (relative to the FLAG tags or similar) can be employed.

### Methods

His-tagged APOE constructs transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purified using tabletop Nickel-NTA columns (Thermo Fisher).

LNPs are produced as detailed above. Here, LNPs are made with GFP mRNAs.

LNPs are first synthesized and then mixed in 1:3 volume ratio with media or purified protein. LNPs are incubated for 15 minutes at 37 C in an incubator or thermocycler. Here, the LNP constructs are dosed on HEK293FTs and HEK293FTs that were previously transfected with a CD4 receptor expression construct.

16-24h post-transduction, the transduction efficiency was quantified with a cytoflex analyzer.

### Significance

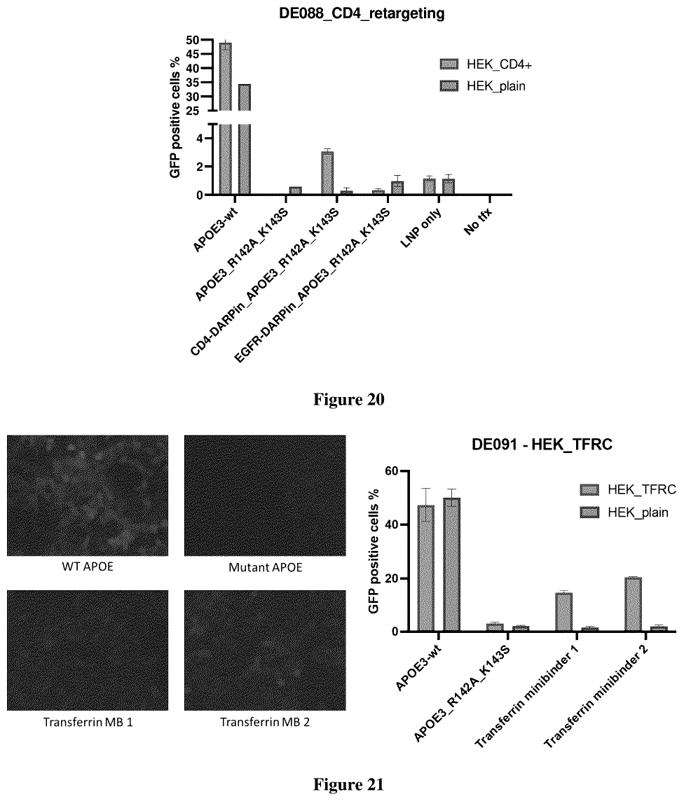

This experiment shows that specific and orthogonal transduction can be set up using APOE constructs conjugated with the CD19 ScFv (and likely other ScFvs for different targets). Here, the CD19-ScFv-APOE enables a boost in transduction efficiency only on the cells expressing the matched receptor target (FIG. 20). This shows that much larger ligands/conjugations than initially tested (relative to the FLAG tags or similar) can be employed.

### Methods

LNPs are produced as detailed above. Here, LNPs are made with GFP mRNAs.

LNPs are first synthesized and then mixed in 1:3 volume ratio with media or purified protein. LNPs are incubated for 15 minutes at 37 C in an incubator or thermocycler. Here, the LNP constructs are dosed on HEK293FTs and HEK293FTs that were previously transfected with a TFRC receptor expression construct.

16-24h post-transduction, the dosed cells can be imaged by microscope for fluorescence (if LNP cargo is GFP). The transduction efficiency was then quantified with a cytoflex analyzer.

### Significance

This experiment shows transduction of cells expressing the TFRC receptor only when the matched anti-TFRC minibinders are presented on the APOE LNP construct (FIG. 21). This demonstrates another form of retargeting/another type of retargeting ligand compatible with our approach.

### Method

LNPs are produced as detailed above. Here, LNPs are made with GFP mRNAs.

LNPs are first synthesized and then mixed in 1:3 volume ratio with media or purified protein. LNPs are incubated for 15 minutes at 37 C in an incubator or thermocycler. Here, the LNP constructs are dosed on HEK293FTs and HEK293FTs that were previously transfected with a CD90 receptor expression construct.

16-24h post-transduction, the dosed cells can be imaged by microscope for fluorescence (if LNP cargo is GFP). The transduction efficiency was then quantified with a cytoflex analyzer.

### Significance

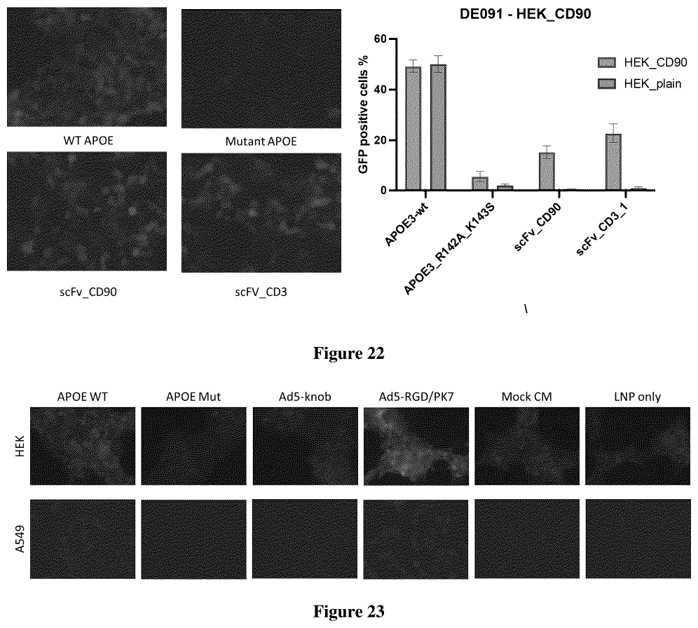

This experiment shows another set of ScFvs capable of enhancing transduction when conjugated to our APOE-LNPs (FIG. 22). Interestingly, both the CD3 and CD90 ScFvs enhance transduction, suggesting either some background transduction (undesirable) or potential homology between the binders/receptors for these combinations.

### Methods

LNPs are produced as detailed above. Here, LNPs are made with GFP mRNAs.

LNPs are first synthesized and then mixed in 1:3 volume ratio with media or purified protein. LNPs are incubated for 15 minutes at 37 C in an incubator or thermocycler. Here, the LNP constructs are dosed on HEK293s and A549s, a model for lung tissue.

16-24h post-transduction, the dosed cells can be imaged by microscope for fluorescence (if LNP cargo is GFP).

### Significance

This experiment tested another type of ligand for compatibility with our approach. The inclusion of the RGD domain from the adenovirus knob in particular caused a boost/rescue of transduction (FIG. 23). By also transducing A549s, which are known to be highly transduced by adenovirus, we show that we can get a corresponding enhancement in another cell type by matching our APOE's conjugated ligand to the target.

### Methods

LNPs are produced as detailed above. Here, LNPs are made with GFP mRNAs.

LNPs are first synthesized and then mixed in 1:3 volume ratio with media or purified protein. LNPs are incubated for 15 minutes at 37c in an incubator or thermocycler. Here, the LNP constructs are dosed on HEK293s and A549s, a model for lung tissue.

16-24h post-transduction, the dosed cells can be imaged by microscope for fluorescence (if LNP cargo is GFP).

### Significance

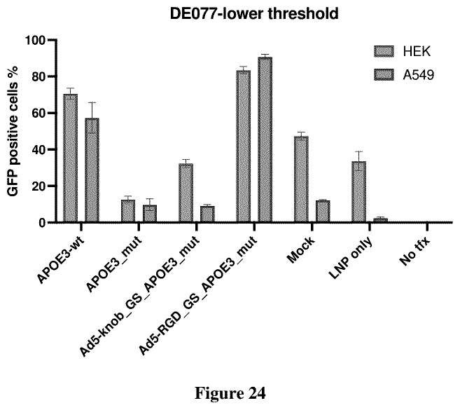

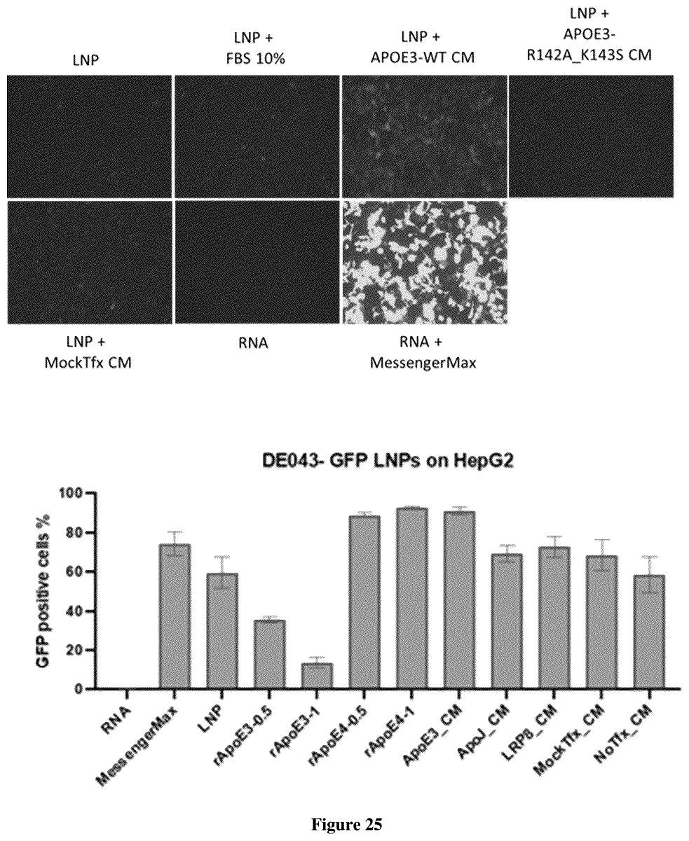

This experiment tested another type of ligand for compatibility with our approach. The inclusion of the RGD domain from the adenovirus knob in particular caused a boost/rescue of transduction (FIG. 24). By also transducing A549s, which are known to be highly transduced by adenovirus, we show that we can get a corresponding enhancement in another cell type by matching our APOE's conjugated ligand to the target.

### Example 5: Retargeting In Vitro Other Cell Types

### GFP LNPs on HepG2

### Methods

LNPs are produced as detailed above. Here, LNPs are made with either unmodified or 5′moU-modified GFP mRNAs. Transductions are performed on HepG2. Cells are dosed with different RNA amounts, either naked or mixed with wildtype or mutant APOE CM

LNPs are first synthesized and then mixed in 1:3 volume ratio with media or purified protein. LNPs are incubated for 15 minutes at 37 C in an incubator or thermocycler.

16-24h post-transduction, the dosed cells can be imaged by microscope for fluorescence (if LNP cargo is GFP).

### Significance

This experiment recapitulates the HEK293 transductions in another common cell line (HepG2s) (FIG. 25). As before, incubation with wildtype conditioned media causes an enhancement to transduction while mutant APOE ablates transduction.

### Example 6: In Vivo Results

Homemade LNPs Transduce Well, have Apparent Tropism to Liver

### Methods

C57/BL6 mice were injected with luciferase RNA LNP formulations prepared as elsewhere in this deck. Approximately 6 μg (higher dose) and 2 μg (lower dose) of RNA was used per mouse. Retro-orbital injections were performed with a total of ˜100 μL of LNP formulation depending on condition. Mixes were diluted up to 100 μL with PBS when lower RNA amounts were injected.

6 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time-course sequence imaging every 5 minutes for 20 minutes. Aura imaging software was used to measure ROIs in photons/s.

### Significance

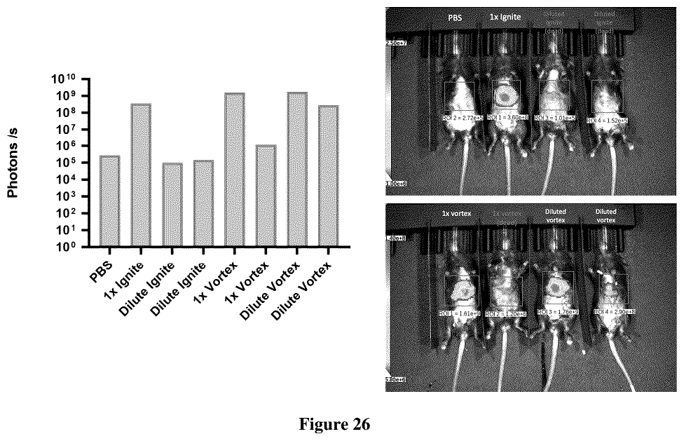

This experiment shows functional transduction with both doses of LNPs tested, using either strategy for production (FIG. 26).

### Last Minute QC: Competition Against Purified Protein Shows Only Modest Transduction Block In Vitro

### Methods

His-tagged APOE constructs transfected with PEI into HEK293-FT cells cultured in DMEM+10% FBS at a T225 scale. 4-6 hours after transfection, change the media in the flasks to DMEM only. Collect media 48-72h post-transfection and concentrate it using a 10 kDa MWCO Amicon Centrifugal Filters. Purified using tabletop Nickel-NTA columns (Thermo Fisher)

LNPs are produced as detailed above. Here, LNPs are made with Luciferase mRNAs.

LNPs are first synthesized and then mixed in 1:3 volume ratio with media or purified protein. LNPs are then mixed in a similar ratio with a second solution, such as 1% serum in PBS.

16-24h post-transduction, the transduction efficiency was quantified by reading out with BriteGlo luciferase assay on a BioTek.

### Significance

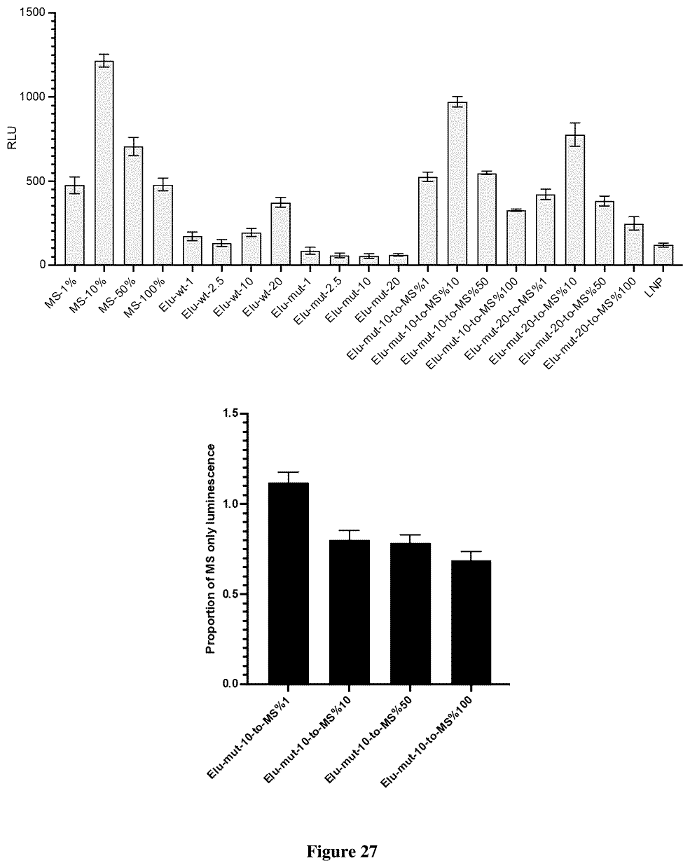

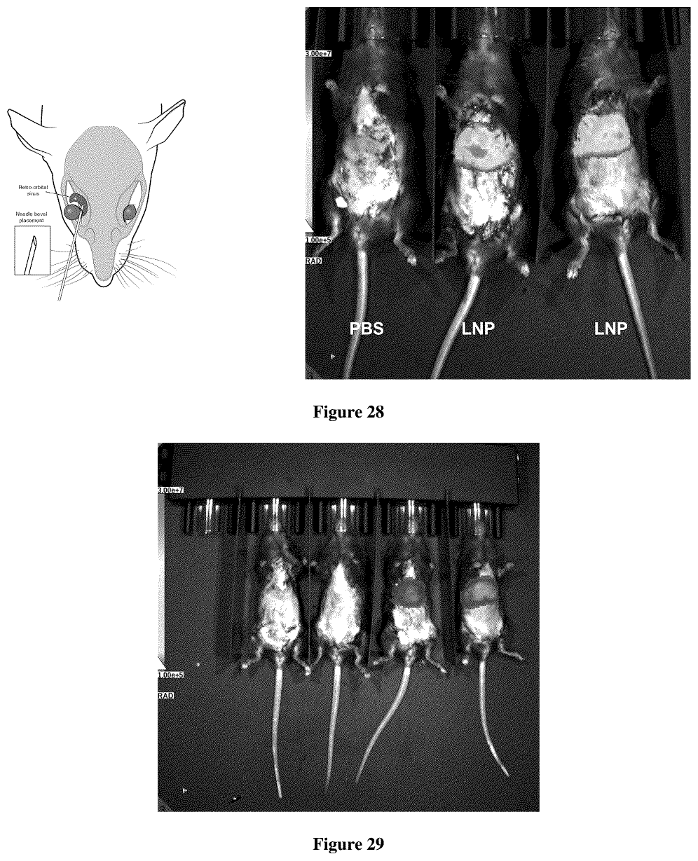

This data represents a “competition assay” to determine the amount of transduction efficiency suppressed by incubation with mutant APOE purified protein. A high level of suppression after a 2^{nd} incubation in serum should indicate that the protein corona is stable, while a return to a similar level of transduction would indicate that the mutant APOE is being competed off of the LNPs (FIG. 27). Here, a moderate 25-35% reduction is noted.

Round 2 LNPs with F.Luc Efficiently Transduce Following Retro-Orbital Injection

### Methods

C57/BL6 mice were injected with luciferase RNA LNP formulations prepared as detailed above. 1.5 μg of RNA was used per mouse. Retro-orbital injections were performed with a total of ˜100 μL of LNP formulation depending on condition.

6 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time-course sequence imaging every 5 minutes for 20 minutes. Aura imaging software was used to measure ROIs in photons/s.

### Significance

These transductions are from a set that includes protein coatings with wildtype and mutant APOE and serve as controls. They show that the LNPs produced in this batch are efficient for luciferase delivery, mainly to the liver/midsection of the animal (FIG. 28).

Wild-Type APOE-Coated LNPs Continue to Transduce (but Weaker) while Mutant APOE-Coated LNPs have No Detectable Signal

### Methods

C57/BL6 mice were injected with luciferase RNA LNP formulations prepared detailed above. 1.5 μg of RNA was used per mouse. Retro-orbital injections were performed with a total of ˜100 μL of LNP formulation depending on condition.

6 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time-course sequence imaging every 5 minutes for 20 minutes. Aura imaging software was used to measure ROIs in photons/s.

### Significance

These transductions are with the same set of LNPs from FIG. 28. They show that incubation of luciferase LNPs with wildtype APOE results in a similar, but less intense transduction of the midsection/liver region of the mouse, while incubation with mutant APOE ablates all transduction, or at least all luciferase signals (FIG. 29). This is significant because it represents a “detargeting” of the liver/other organs with the scaffold molecule for our APOE LNPs (as intended).

Mutant APOE-Coated LNPs have Fluc Signal Equivalent to PBS Injection

### Methods

C57/BL6 mice were injected with luciferase RNA LNP formulations prepared as elsewhere in this deck. 1.5 μg of RNA was used per mouse. Retro-orbital injections were performed with a total of ˜100 μL of LNP formulation depending on condition.

6 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a timecourse sequence imaging every 5 minutes for 20 minutes. Aura imaging software was used to measure ROIs in photons/s.

### Significance

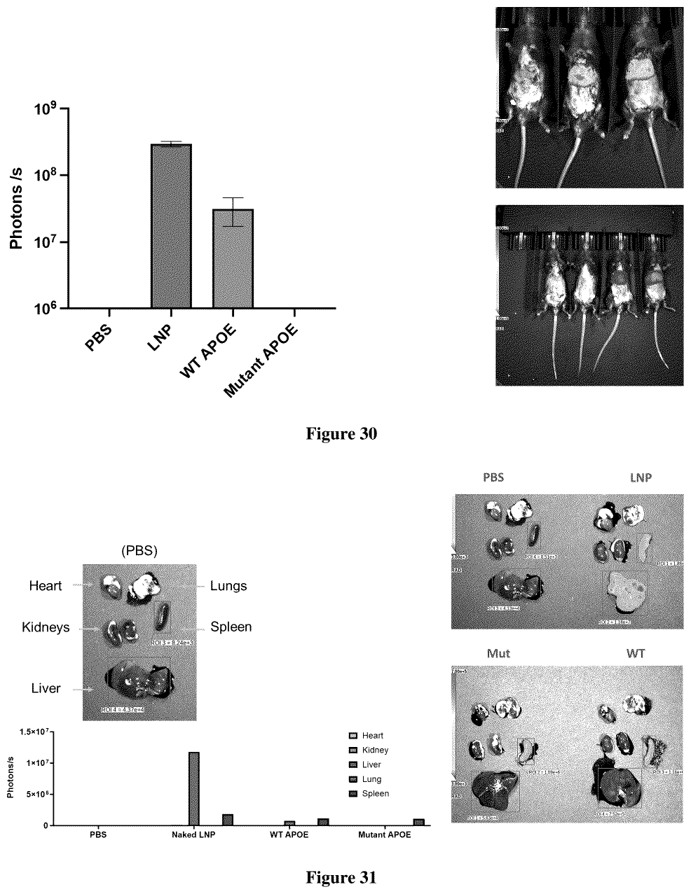

These transductions are with the same set of LNPs from FIG. 28. They show that incubation of luciferase LNPs with wildtype APOE results in a similar, but less intense transduction of the midsection/liver region of the mouse, while incubation with mutant APOE ablates all transduction, or at least all luciferase signals (FIG. 30). This is significant because it represents a “detargeting” of the liver/other organs with the scaffold molecule for our APOE LNPs (as intended). This quantification shows that the signal from LNPs coated with mut APOE is equivalent to the values from PBS injection of the mouse (near-zero).

mmmAPOE Coated Constructs Traffic Only to Spleen

### Methods

C57/BL6 mice were injected with luciferase RNA LNP formulations prepared as elsewhere in this deck. 1.5 μg of RNA was used per mouse. Retro-orbital injections were performed with a total of ˜100 μL of LNP formulation depending on condition.

6 hours post injection, mice were anaesthetized and shaved, then injected with luciferin substrate. IVIS imaging for luciferase signal was performed 15 minutes after luciferin injection. Post injection, mice were sacrificed using CO₂ and organs harvested via dissection. A 60s exposure was used for all images with otherwise default settings. An IVIS Spectrum was used for all imaging, doing a time-course sequence imaging every 5 minutes for 20 minutes. Aura imaging software was used to measure ROIs in photons/s.

### Significance

These images back up the whole-body live imaging of the mice shown in FIGS. 28 and 29. Dissecting out the organs reveals that while the LNPs alone transduce liver and spleen efficiently, the LNPs coated with wildtype APOE show weaker transduction of the liver, and LNPs coated with mutant APOE appear to be completely de-targeted (e.g., no luciferase signal in the liver) (FIG. 31). However, they still show signal in the spleen, potentially a result of being cleared from circulation via phagocytosis or similar process.

### Methods

C57/BL6 mice were injected with luciferase RNA LNP formulations prepared as elsewhere in this deck. 1.5 μg of RNA was used per mouse. Retro-orbital injections were performed with a total of ˜100 μL of LNP formulation depending on condition.

6 hours post injection, mice were anaesthetized and shaved, then injected with luciferin substrate. IVIS imaging for luciferase signal was performed 15 minutes after luciferin injection. Post injection, mice were sacrificed using CO2 and organs harvested via dissection. A 60s exposure was used for all images with otherwise default settings. An IVIS Spectrum was used for all imaging, doing a timecourse sequence imaging every 5 minutes for 20 minutes. Aura imaging software was used to measure ROIs in photons/s.

### Significance

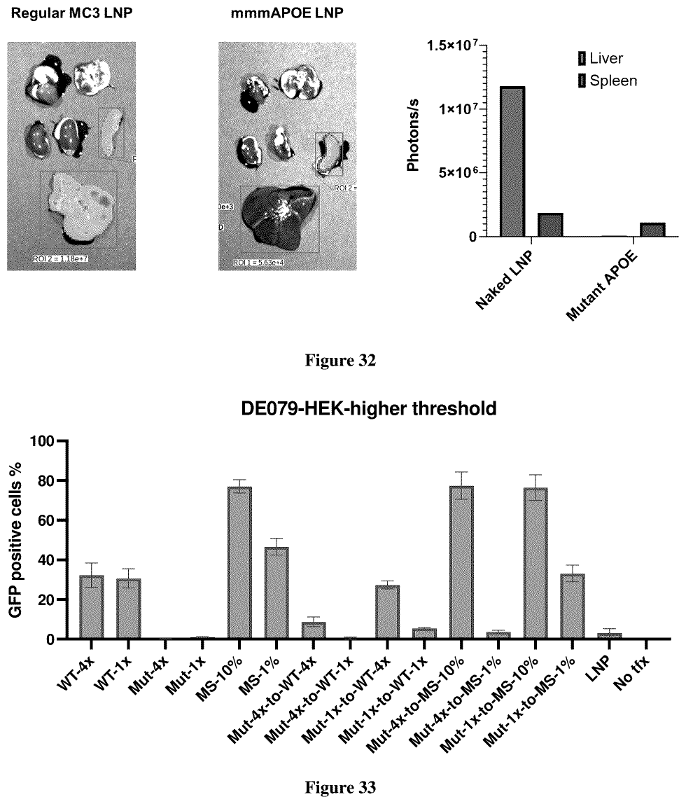

These images back up the whole-body live imaging of the mice shown in preceding slides. Dissecting out the organs reveals that while the LNPs alone transduce liver and spleen efficiently, the LNPs coated with wildtype APOE show weaker transduction of the liver, and LNPs coated with mutant APOE appear to be completely de-targeted (e.g., no luciferase signal in the liver) (FIG. 32). However, they still show signal in the spleen, potentially a result of being cleared from circulation via phagocytosis or similar process.

### Example 7: APOE Competition Assay

HEK293FT cells were transfected with required plasmids, and media was changed to DMEM with no FBS after overnight incubation. Conditioned media were collected after 48h of media change.

LNPs were first synthesized by vortex mixing of lipids and RNA. Then, they were mixed in 1:1 volume ratio with conditioned media of mutant ApoE and incubated for 15 minutes at 37 C. For competition, conditioned media from wild type ApoE or mouse serum (1 or 10%) were added to the already incubated mix. Total volume was completed to 100 ul/well with DMEM. 100 ul from each condition were added to each well of 96-well plate.

16-24h post-transduction, the dosed cells can be imaged by microscope for fluorescence (if LNP cargo is GFP) or plate reader (if LNP cargo is firefly luciferase).

FIG. 33 shows a competition assay for binding of mutant ApoE to LNPs in the presence of wild type ApoE, or mouse serum (MS). 1× or 4× concentrated conditioned media for mutant or wild type ApoE and 1% or 10% of MS were used for this assay. These results show that mutant ApoE can stably bind to LNPs, and when used in high concentration, exchange rate with serum proteins is low. So, they have potential to block non-specific liver accumulation of LNPs in vivo.

### Example 8: APOE Engineering

### Methods

Flag and MoonTag coding sequences were cloned into different positions of ApoE coding sequence in a mammalian expression plasmid.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and RNA. Then, they were mixed in 1:1 volume ratio with conditioned media of mutant ApoE constructs and incubated for 15 minutes at 37 C. Total volume was completed to 100 ul/well with DMEM. 100 ul from each condition were added to each well of 96-well plate.

16-24h post-transduction, the dosed cells can be imaged by microscope for fluorescence and positive cells were quantified by flow cytometry.

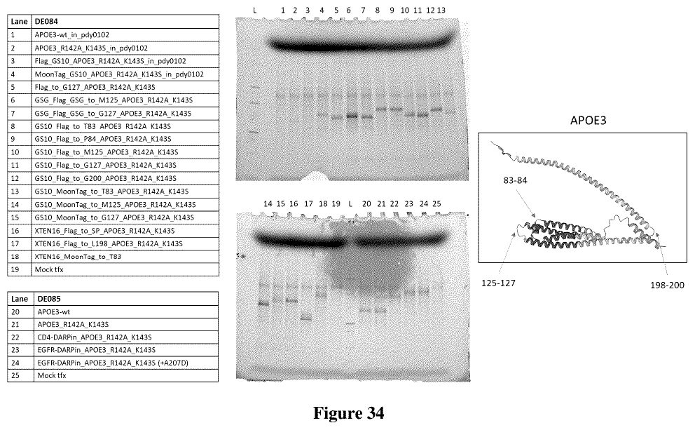

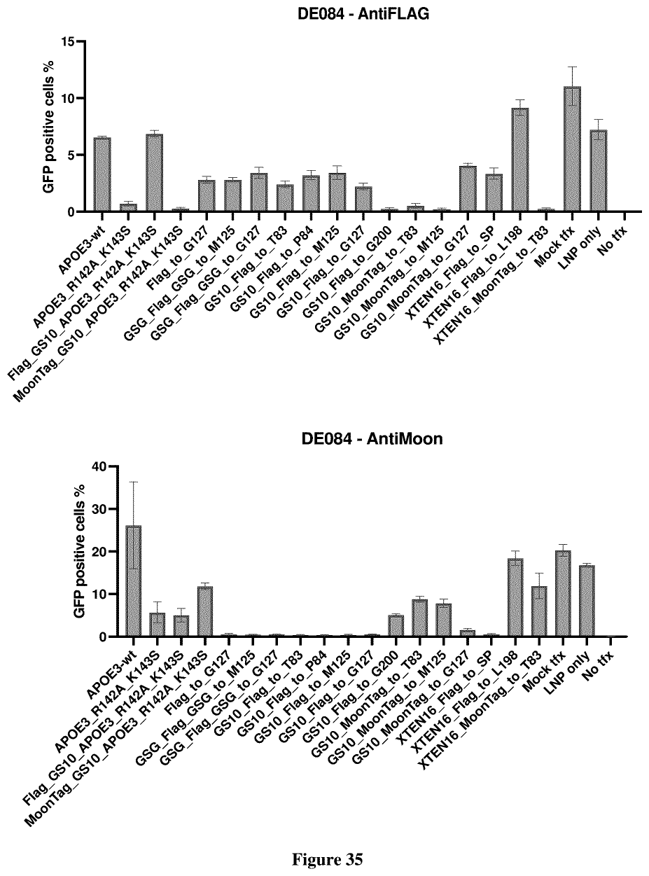

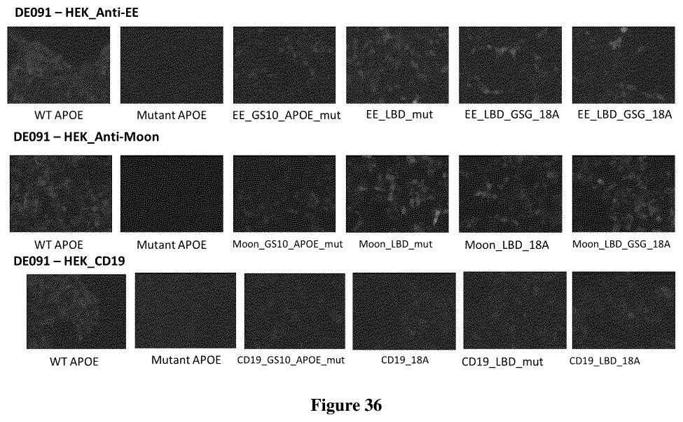

FIG. 34 shows ligand fusion strategy to different position on ApoE protein. The figure shows loop structures on ApoE structure where ligand insertions were planned, and gel images showing expressions of these fused proteins. FIG. 35 shows the results for transduction of HEK cells overexpressing Anti-Flag or Anti-MoonTag receptors, with LNPs coated ApoE fused to Flag or Moon Tag peptides.

These results show that different internal positions on ApoE can be used to fuse targeting peptides, where they can still be functional. But most of these positions were not superior compared to N-terminal fusions.

### Methods

Point mutations were introduced into ApoB LBD, and lipid binding peptide were cloned into C-terminus of ApoE with or without GSG linker.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and RNA. Then, they were mixed in 1:1 volume ratio with conditioned media of mutant ApoE constructs and incubated for 15 minutes at 37 C. For competition, conditioned media from the cross ApoE construct or mouse serum (1 or 10%) were added to the already incubated mix. Total volume was completed to 100 ul/well with DMEM. 100 ul from each condition were added to each well of 96-well plate.

16-24h post-transduction, the dosed cells were imaged by microscope for fluorescence and positive cells were quantified by flow cytometry.

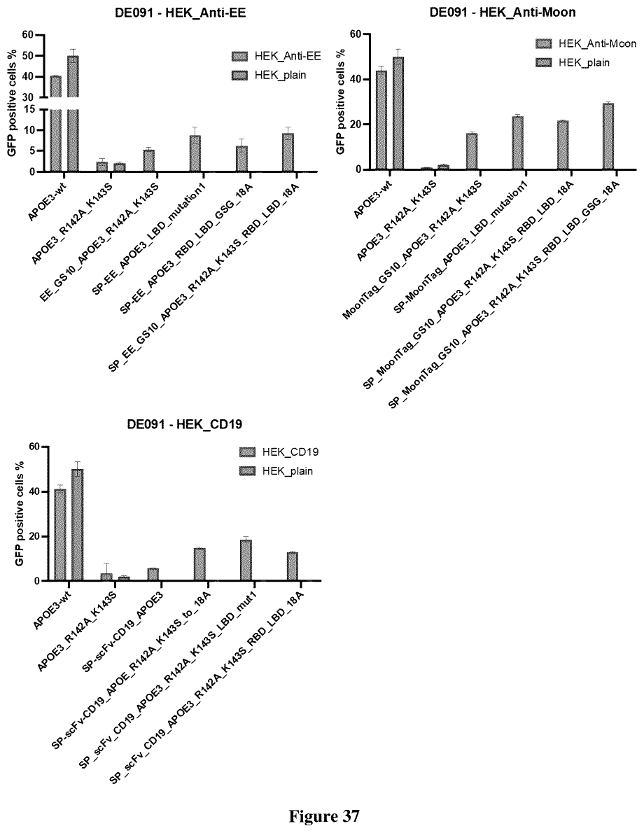

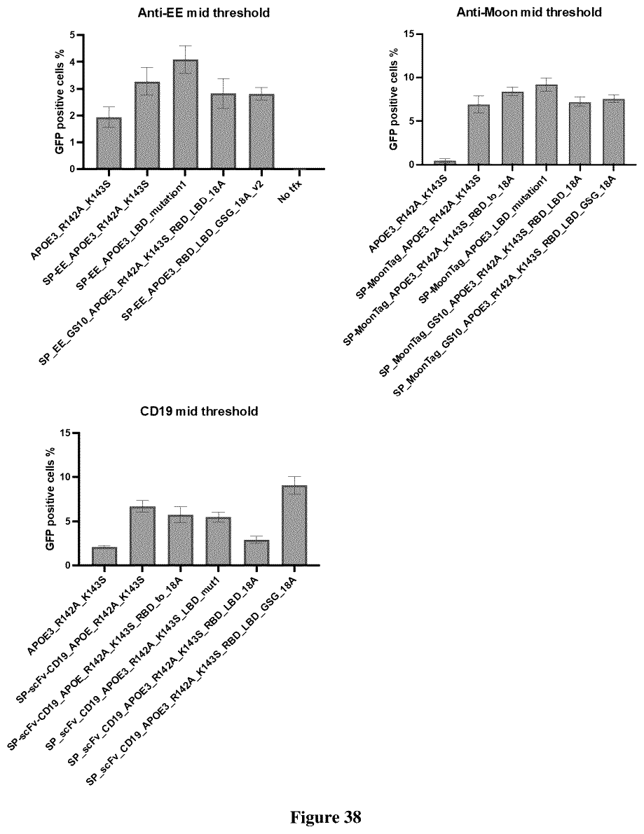

FIG. 36 shows the effects of a double point mutation (1250S_L252S) in lipid binding domain (LBD) of ApoE, or a lipid binding peptide (18A) fused to ApoE, on LNP retargeting efficiency. This figure shows fluorescent microscope images of transductions using LNPs coated with different ApoE constructs. FIGS. 37 and 38 show flow cytometry results for two independent experiments.

Engineering ApoE protein to make it bind to LNPs with higher affinity can both reduce the background and enhance the retargeting efficiency. Here both point mutation and 18A peptide shows promising results to improve retargeting efficiency of ApoE.

### Methods

Point mutations were introduced into ApoE LBD by Gibson cloning.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and RNA. Then, they were mixed in 1:1 volume ratio with conditioned media of mutant ApoE constructs and incubated for 15 minutes at 37 C. Total volume was completed to 100 ul/well with DMEM. 100 ul from each condition were added to each well of 96-well plate.

16-24h post-transduction, the dosed cells were imaged by microscope for fluorescence and positive cells were quantified by flow cytometry.

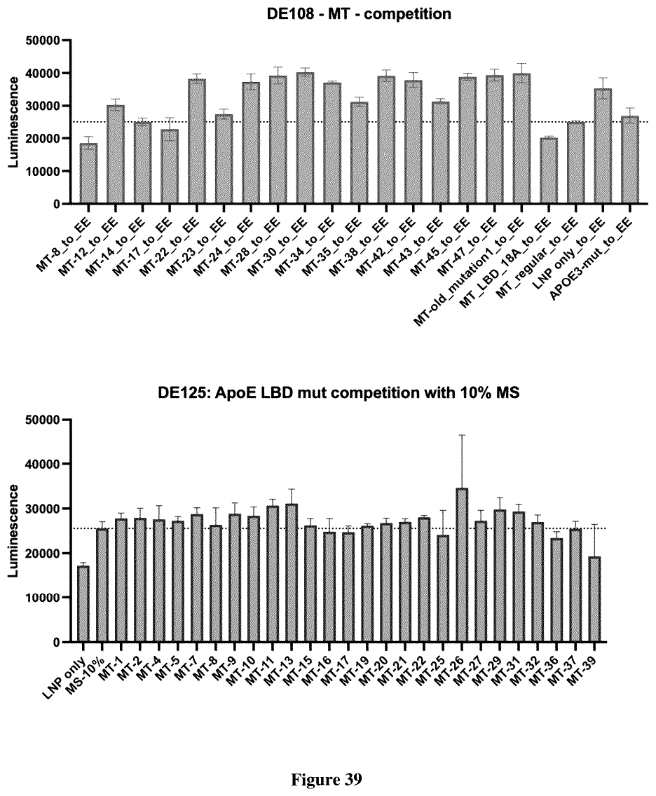

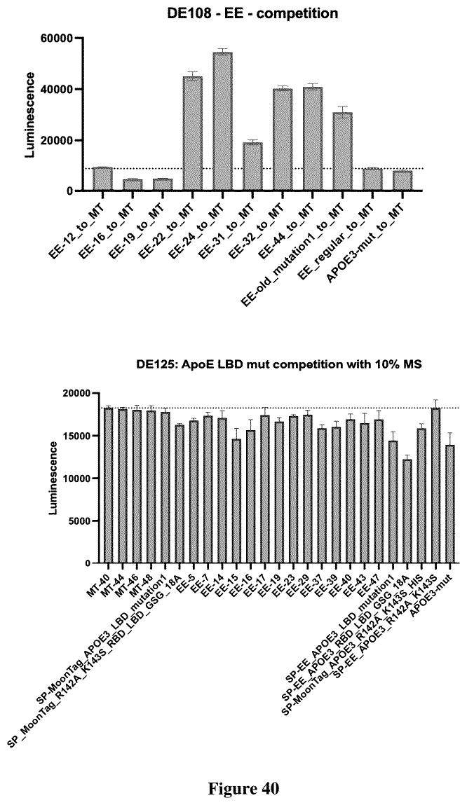

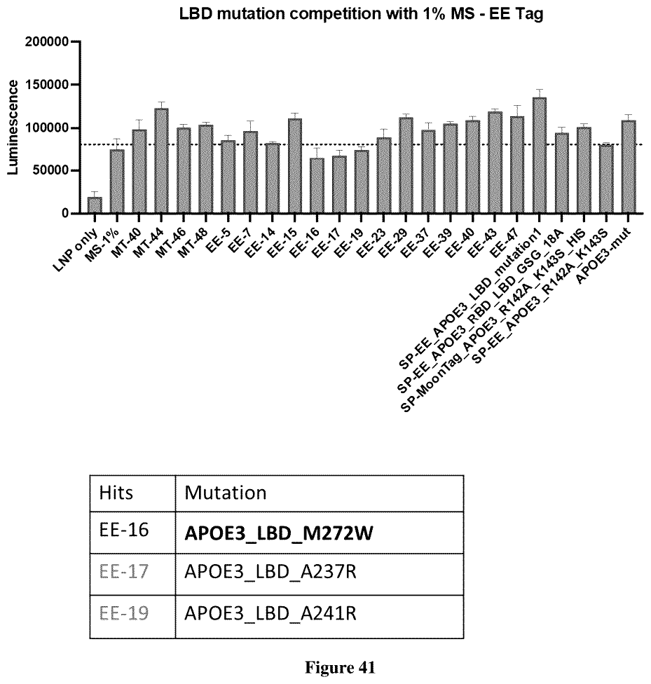

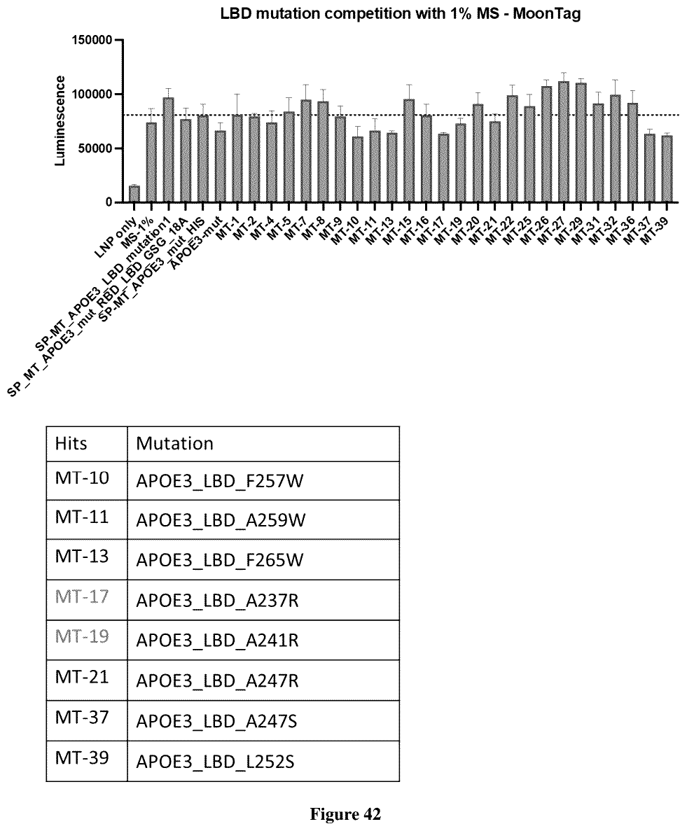

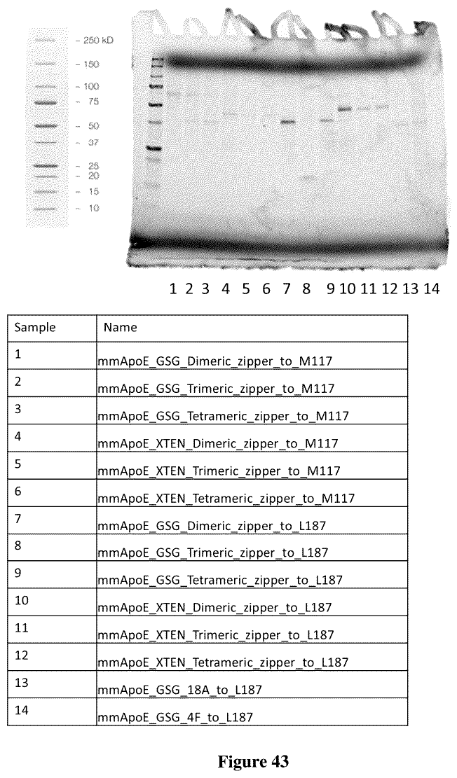

FIG. 39 shows the effects of various point mutations in lipid binding domain (LBD) of ApoE on its competition efficiency for LNP coating. The figure shows competition of MoonTag-ApoE with EE-ApoE or 10% mouse serum (MS). FIG. 40 shows competition of EE-ApoE with MoonTag-ApoE or 10% MS. FIG. 41 shows results of another set of mutations on EE-ApoE. FIG. 42 shows validation experiments for potentially working mutations for EE- and MoonTag-ApoE with 1% MS.

Engineering ApoE protein to make it bind to LNPs with higher affinity can both reduce the background and enhance the retargeting efficiency. By this screen, we found several point mutations (e.g. A237R and A241R) that has potential to improve binding affinity of ApoE, so enhance its blocking activity for non-specific targeting.

### Methods

Leucine zipper and lipid binding peptides were cloned into pre-determined positions of ApoE with GSG or XTEN linkers by Gibson cloning.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and Cy5-labelled RNA. Then, they were mixed in 1:1 volume ratio with conditioned media of mutant ApoE constructs and incubated for 15 minutes at 37 C. Total volume was completed to 100 ul/well with DMEM. 100 ul from each condition were added to each well of 96-well plate.

16-24h post-transduction, the dosed cells were imaged by fluorescence microscope for cellular uptake and payload expression was measured by luciferase assay and quantified by a luminescent plate reader.

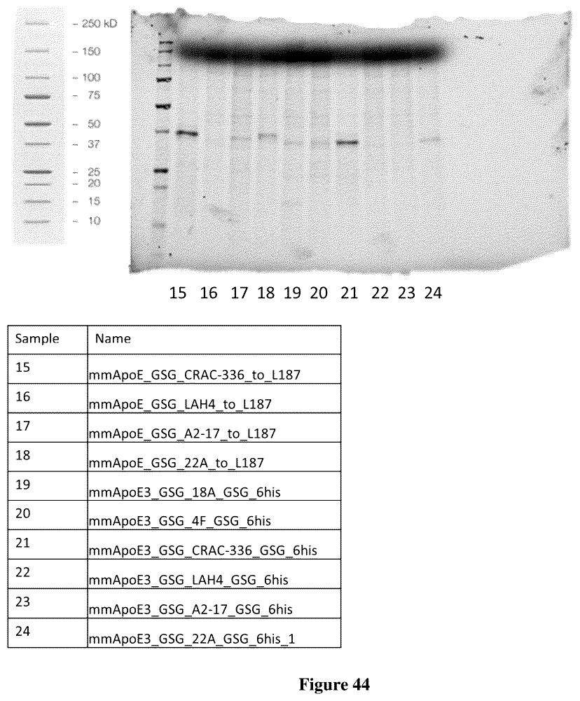

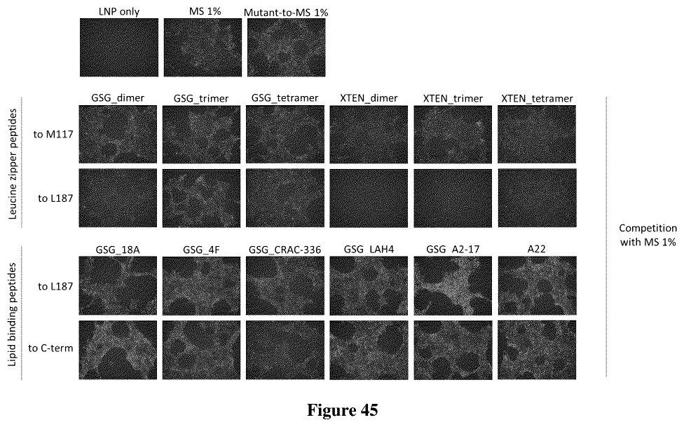

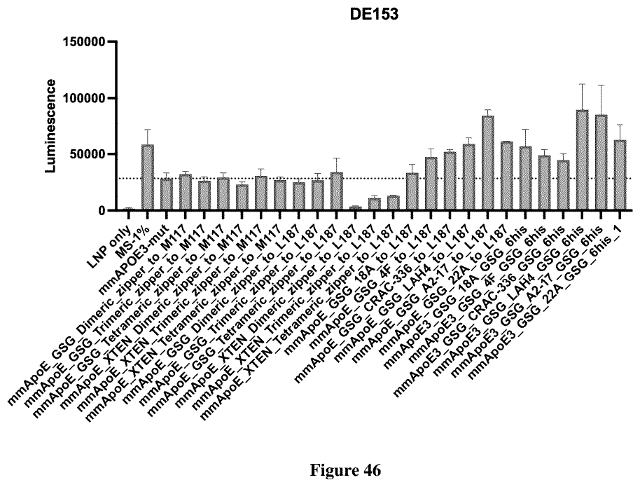

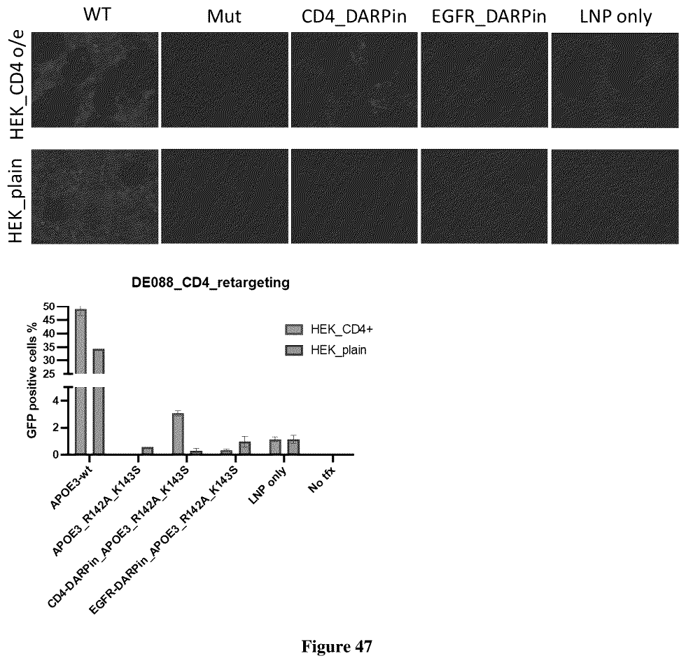

FIG. 43 shows the effects of leucine zipper peptides or several new lipid binding peptides on the competition efficiency of ApoE for LNP coating. This figure shows expression gels for these constructs. FIG. 44 shows expression gels for these constructs. FIG. 45 shows fluorescent images (RNA is labeled with Cy5 dye) of competition assay of these constructs with 1% mouse serum (MS). FIG. 46 shows luminescence readout of the same competition assay.

Engineering ApoE protein to make it bind to LNPs with higher affinity can both reduce the background and enhance the retargeting efficiency. By this screen, we found that oligomerization peptides, especially the dimerization one, cloned with XTEN linker to L187 position has potential to improve binding affinity of ApoE, so enhance its competition with MS and blocking activity for non-specific targeting.

### Example 9: Retargeting In Vitro HEK293FT Cell Overexpression

### Methods

Retargeting ligands were cloned into N-terminus of mutant ApoE with a GS10 linker by Gibson cloning.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and RNA. Then, they were mixed in 1:1 volume ratio with conditioned media of mutant ApoE constructs and incubated for 15 minutes at 37 C. Total volume was completed to 100 ul/well with DMEM. 100 ul from each condition were added to each well of 96-well plate.

16-24h post-transduction, the dosed cells were imaged by fluorescence microscope and positive cells were quantified by flow cytometry.

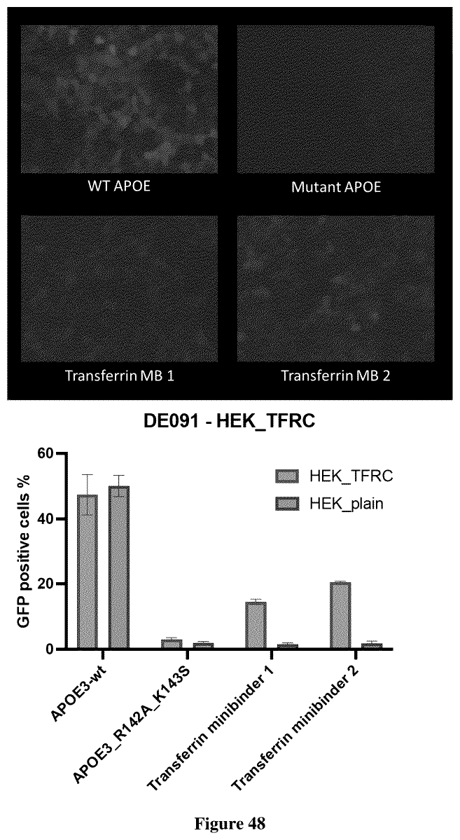

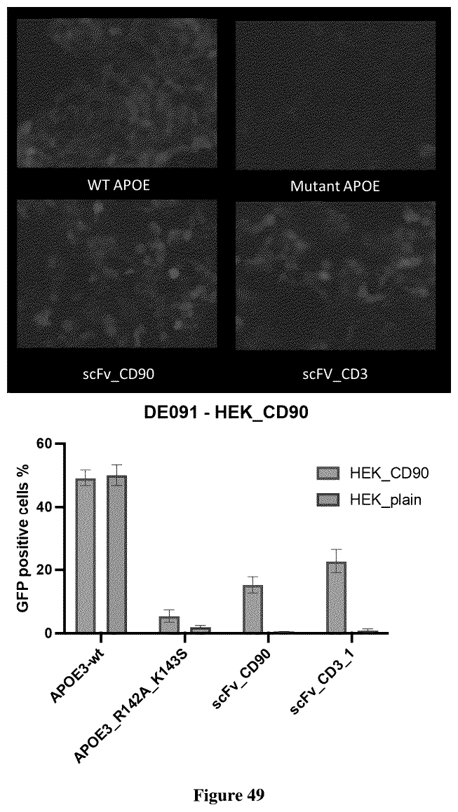

FIG. 47 shows retargeting efficacies with LNPs coated with CD4-binder on either plain HEK293FT cells or expressing corresponding receptors. FIG. 48 shows retargeting efficacies with LNPs coated with TFRC-binder on either plain HEK293FT cells or expressing corresponding receptors. FIG. 49 shows retargeting efficacies with LNPs coated with CD90-binder on either plain HEK293FT cells or expressing corresponding receptors.

Fusion of mutant ApoE protein to a specific receptor-targeting ligand is very important to both prevent non-specific transduction and enable specific retargeting. In these figures, we showed that LNPs coated with retargeting ligand-fused mutant ApoE transduce only cells expressing corresponding receptor, and not plain HEK293FT cells.

### Example 10: Retargeting In Vitro Other Cell Types

### Methods

Retargeting ligands were cloned into N-terminus of mutant ApoE with a GS10 linker by Gibson cloning.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and RNA. Then, they were mixed in 1:1 volume ratio with conditioned media of mutant ApoE constructs and incubated for 15 minutes at 37 C. Total volume was completed to 100 ul/well with DMEM. 100 ul from each condition were added to each well of 96-well plate.

16-24h post-transduction, the dosed cells were imaged by fluorescence microscope and positive cells were quantified by flow cytometry.

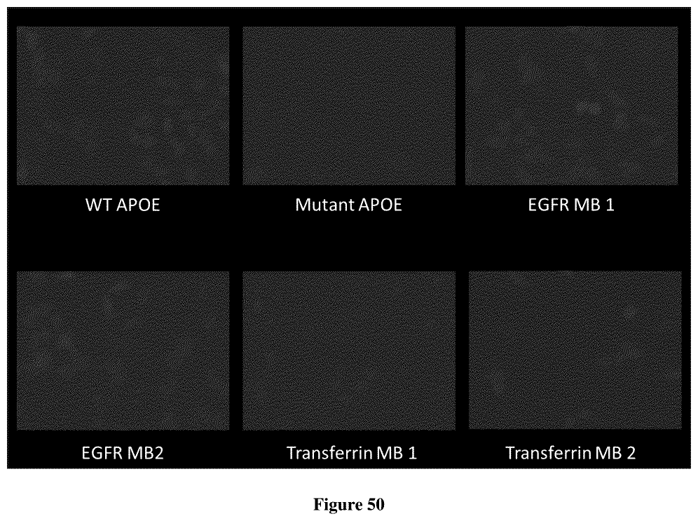

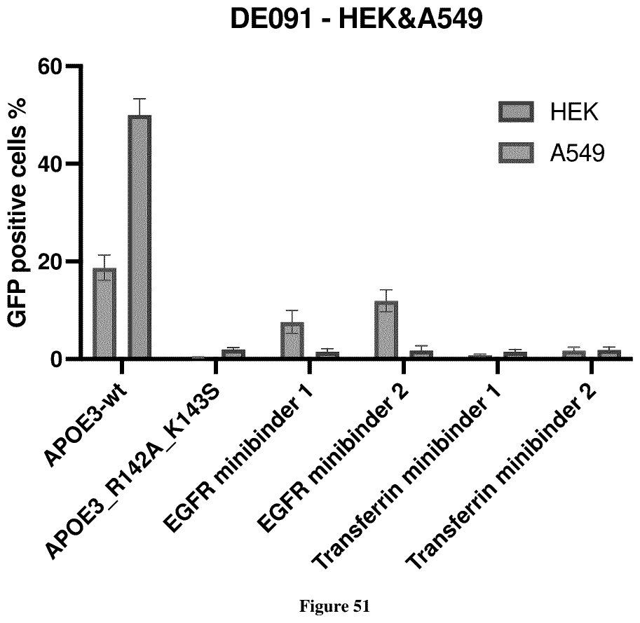

FIGS. 50 and 51 shows transduction level of HEK293T or A549 cells, which is a human lung cancer cell line, with LNPs coated with mutant ApoE fused to EGFR or TFRC minibinders. It is clear that EGFR-minibinders (Cao L. et al. Nature. 2022; 605 (7910): 551-560) induce transduction on A549 cells as they overexpress EGFR naturally, but not on HEK293FT cells. On the other hand, TFRC minibinders didn't induce retargeting in any of these cell lines as they are not overexpressing transferrin receptor (Sahtoe D., et al. Proc Natl Acad Sci USA. 2021 Apr. 27; 118 (17): e2021569118).

Fusion of mutant ApoE protein to a specific receptor-targeting ligand is a promising strategy to both prevent non-specific transduction and enable specific retargeting. To show translational potential of this strategy, it is important to test them on various cell lines which naturally express the targeted receptor.

### Methods

Retargeting ligands were cloned into N-terminus of mutant ApoE with a GS10 linker by Gibson cloning.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and RNA. Then, they were mixed in 1:1 volume ratio with conditioned media of mutant ApoE constructs and incubated for 15 minutes at 37 C. Total volume was completed to 100 ul/well with DMEM, 100 ul from each condition were added to each well of 96-well plate.

16-24h post-transduction, 100 ul of Bright-Glo substrate was added on cells and after 2 min of incubation, the transduction efficiency were quantified by luminescence plate reader.

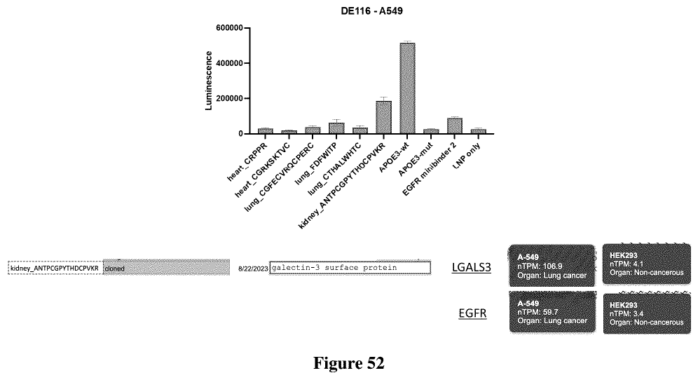

FIG. 52 shows transduction level of A549 cells with LNPs coated with mutant ApoE fused to short peptide binders, shown to target different mouse tissues (e.g. heart, muscle, lung, etc.) in the literature. One of the lung targeting peptides induce slightly higher transduction than the others. Transduction efficiency with a kidney targeting peptide was even higher than the one with EGFR minibinder. TPM values from Protein Atlas database show that A549 cells express higher level of LGALS3 which is the target receptor of this peptide, than EGFR.

Fusion of mutant ApoE protein to a specific receptor-targeting ligand is a promising strategy to both prevent non-specific transduction and enable specific retargeting. To show translational potential of this strategy, it is important to test them on various cell lines which naturally express the targeted receptor.

### Methods

Retargeting ligands were cloned into N-terminus of mutant ApoE with a GS10 linker by Gibson cloning.

HEK293FT cells were transfected with cloned constructs, and media was changed to DMEM with no FBS after overnight incubation. Conditioned media were collected after 48h of media change, and proteins were purified using HisPur Ni-NTA Resin.

LNPs were first synthesized by vortex mixing of lipids and RNA. Then, they were mixed with different protein: RNA ratio with purified mutant ApoE constructs and incubated for 15 minutes at 37 C. For competition, 10% mouse serum was added to the already incubated mix and incubated for another 15 min at 37 C. Total volume was completed to 100 ul/well with DMEM. 100 ul from each condition were added to each well of MLE-12 cells seeded on a 96-well plate.

16-24h post-transduction, 100 ul of Bright-Glo substrate was added on cells and after 2 min of incubation, the transduction efficiency were quantified by luminescence plate reader.

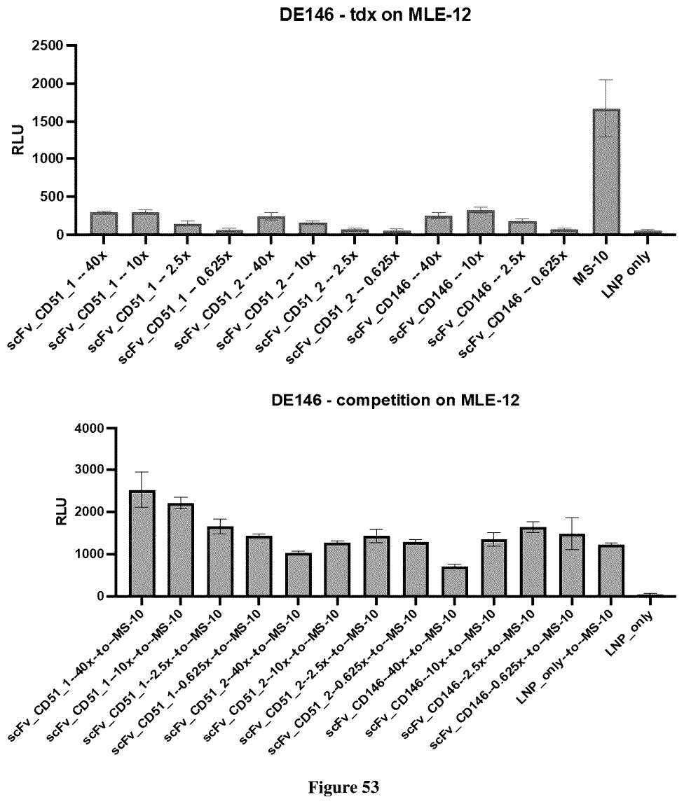

FIG. 53 (top) shows transduction level of MLE-12 cells, which are mouse lung epithelial cells, with LNPs coated with mutant ApoE fused to CD51 or CD146 targeting scFvs. Different protein to RNA (encapsulated in LNP) ratios, 40×, 10×, 2.5×, 0.625× were tested. MS-10 is 10% mouse serum and used as positive control. FIG. 53 (bottom) shows competition assay of proteins with 10% mouse serum for binding to LNPs.

To show translational potential of our retargeting strategy, it is important to test them on various cell lines which naturally express the targeted receptor. Here increasing dose of proteins enabled higher transduction rates on MLE-12 cells. Competition assay with 10% mouse serum showed that 40× ratio of scFv_CD146 inhibit serum-mediated transduction by ˜50%.

### Example 11: In Vivo

### Methods

C57/BL6 mice were injected with luciferase RNA LNP formulations prepared as described above. Approximately 1.5 μg (0.075 mg/kg) of RNA was used per mouse. Retro-orbital injections were performed with a total of ˜100 μL of LNP formulation depending on condition. Mixes were diluted up to 100 uL with PBS when lower RNA amounts were injected.

6 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a timecourse sequence imaging every 5 minutes for 20 minutes. Aura imaging software was used to measure ROIs in photons/s.

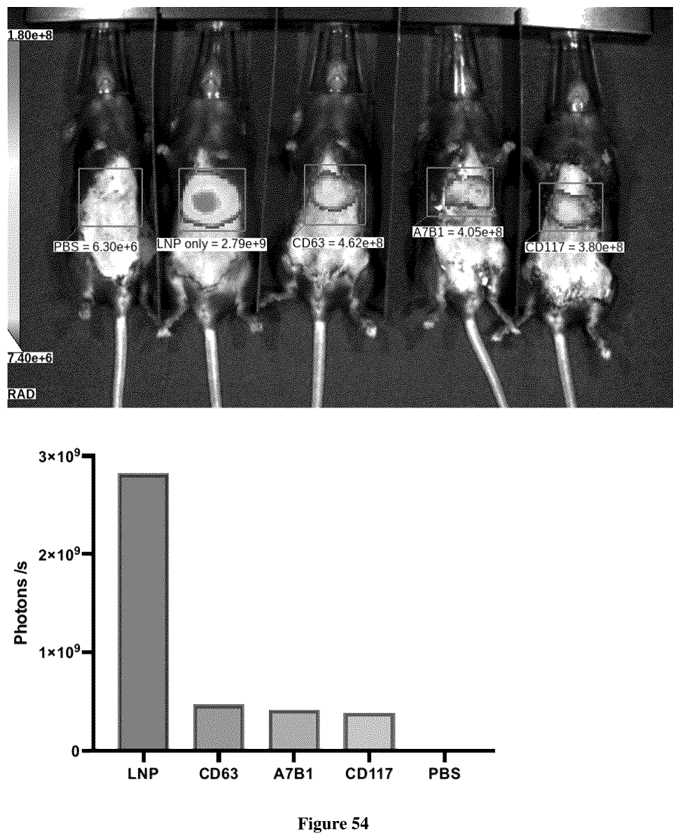

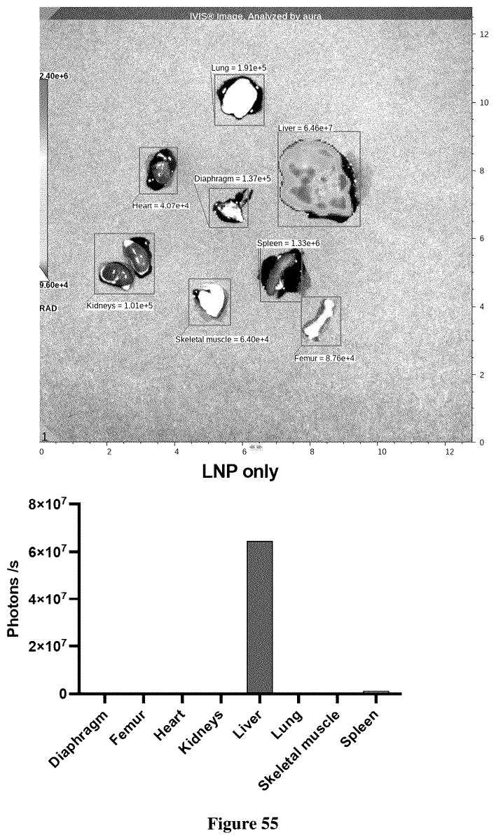

FIG. 54 (top) shows images with IVIS for the mice in question, measuring photons/s. FIG. 54 (bottom) shows quantification of luminescence from a set of LNP or control injections. From left to right; PBS, uncoated LNP, and LNPs coated with mutant ApoE fused to CD63-, A7B1- or CD117-targeting scFvs, respectively.

This experiment shows detargeting from liver with mutant ApoE, but no differential signal was observed in the target organs.

### Methods

C57/BL6 mice were injected with luciferase RNA LNP formulations prepared as elsewhere in this deck. Approximately 1.5 μg (0.075 mg/kg) of RNA was used per mouse. Retro-orbital injections were performed with a total of ˜100 μL of LNP formulation depending on condition. Mixes were diluted up to 100 uL with PBS when lower RNA amounts were injected.

6 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time-course sequence imaging every 5 minutes for 20 minutes. Aura imaging software was used to measure ROIs in photons/s.

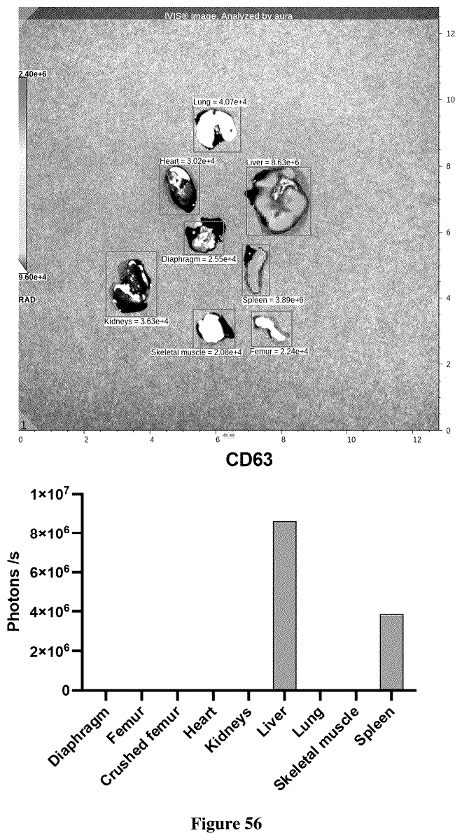

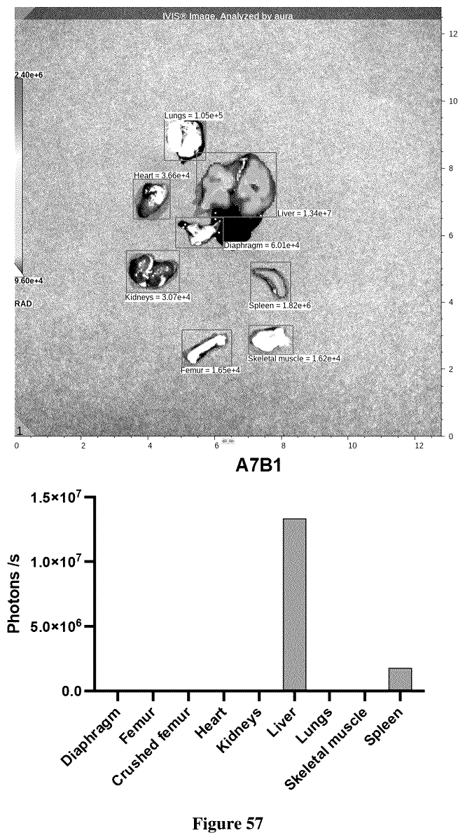

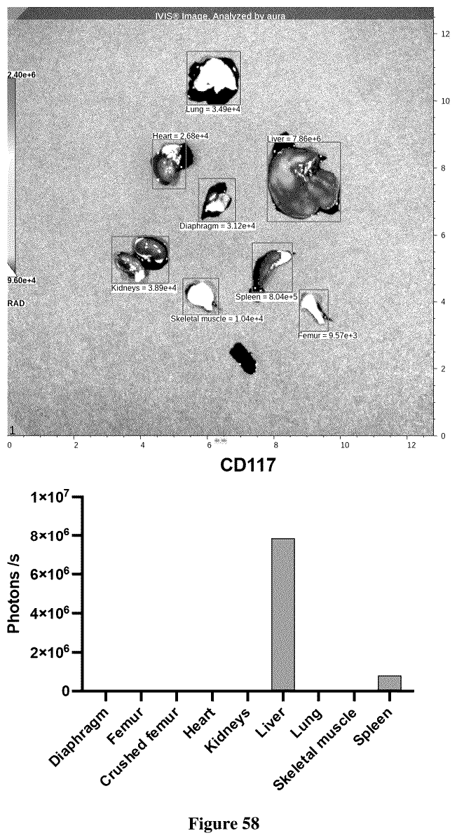

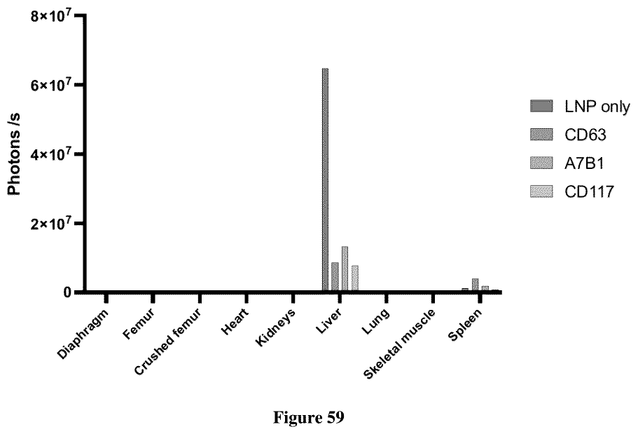

FIG. 55 shows images of various organs ex vivo with IVIS for the mice in question, measuring photons/s and quantification of luminescence from a set of LNP or control injections. This figure shows LNP only, FIG. 56 shows LNP coated with mutant ApoE fused to CD63-targeting scFvs. FIG. 57 shows LNP coated with mutant ApoE fused to A7B1-targeting scFvs. FIG. 58 shows LNP coated with mutant ApoE fused to CD117-targeting scFvs. FIG. 59 shows summary plot showing quantification of organs from mice treated with each particle.

This experiment shows that detargeting from liver with mutant ApoE is still possible in the presence of a targeting ligand, but no differential signal was observed in the target organs,

### Methods

C57/BL6 mice were injected with luciferase expressing mRNA LNP formulations prepared as elsewhere in this deck. Approximately 1.5 μg (0.075 mg/kg) of RNA was used per mouse. Retro-orbital injections were performed with a total of ˜100 μL of LNP formulation depending on condition. Mixes were diluted up to 100 uL with PBS when lower RNA amounts were injected.

6 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time course sequence imaging every 5 minutes for 20 minutes. Aura imaging software was used to measure ROIs in photons/s.

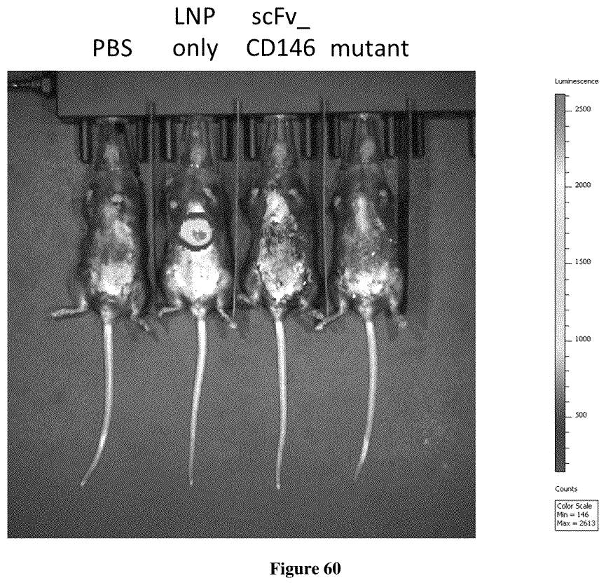

FIG. 60 shows luminescence images with IVIS for the mice in question, measuring photons/s. From left to right; PBS, uncoated LNP, LNP coated with mutant ApoE fused to CD146-targeting scFv, and LNP coated with mutant ApoE, respectively.

This experiment shows that detargeting from liver with mutant ApoE is still possible in the presence of a targeting ligand, but no differential signal was observed in the target organs.

### Methods

Retargeting ligands were cloned into N-terminus of mutant ApoE with a GS10 linker by Gibson cloning.

HEK293FT cells were transfected with cloned constructs, and media was changed to DMEM with no FBS after overnight incubation. Conditioned media were collected after 48h of media change, and proteins were purified using HisPur Ni-NTA Resin.

LNPs were first synthesized by vortex mixing of lipids and RNA. Then, they were mixed with different protein: RNA ratio with purified mutant ApoE constructs and incubated for 15 minutes at 37 C. For competition, 10% mouse serum was added to the already incubated mix and incubated for another 15 min at 37 C. Total volume was completed to 100 ul/well with DMEM. 100 ul from each condition were added to each well of MLE-12 cells seeded on a 96-well plate.

16-24h post-transduction, the dosed cells were imaged by fluorescence microscope and positive cells were quantified by flow cytometry.

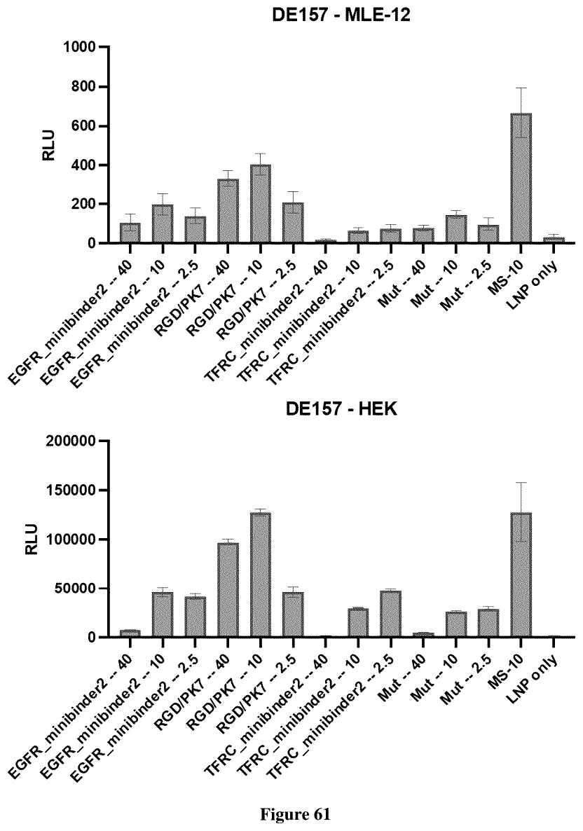

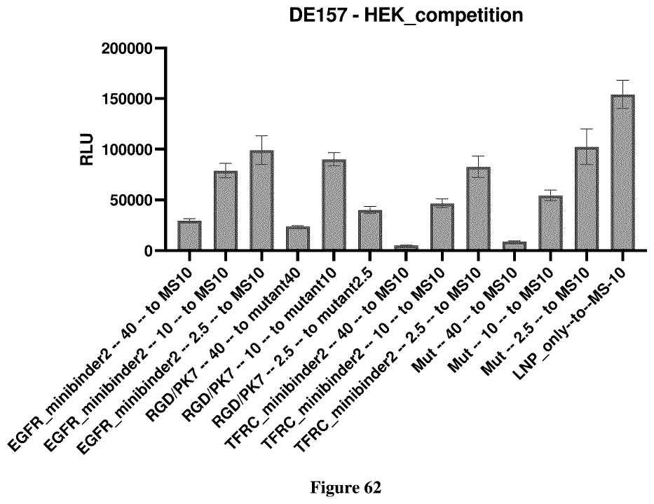

FIG. 61 shows transduction level of MLE-12 and HEK293FT cells with LNPs coated with mutant ApoE either alone or fused to EGFR minibinder, RGD/PK7 peptide, or TFRC minibinder. Different protein to RNA (encapsulated in LNP) ratios, 40×, 10×, 2.5× were tested. MS-10 is 10% mouse serum and used as positive control. FIG. 62 shows competition assay of proteins with 10% mouse serum for binding to LNPs.

To show translational potential of our retargeting strategy, it is important to test them on various cell lines which naturally express the targeted receptor. High dose of proteins, especially RGD/PK7 enabled high transduction rates on both HEK and MLE-12 cells. Competition assay with 10% mouse serum showed that 40× ratio of all 3 proteins almost completely inhibit serum-mediated transduction.

### Methods

FVB.LoxP-Luc mice were injected with Cre expressing mRNA LNP formulations prepared as elsewhere in this deck. Approximately 1.5 μg (0.075 mg/kg) of RNA was used per mouse, Retro-orbital injections were performed with a total of ˜100 μL of LNP formulation depending on condition. Mixes were diluted up to 100 uL with PBS when lower RNA amounts were injected.

24 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time course sequence imaging every 5 minutes for 20 minutes. Aura imaging software was used to measure ROIs in photons/s.

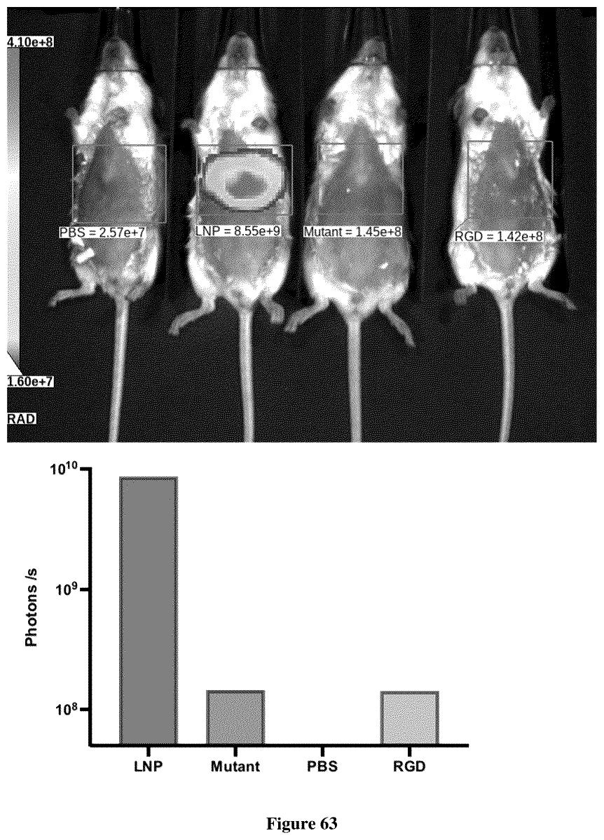

FIG. 63 (top) shows luminescence images with IVIS for the mice in question, measuring photons/s. From left to right; PBS, uncoated LNP, LNP coated with mutant ApoE, and LNP coated with mutant ApoE fused to RGD/PK7 peptide, respectively. FIG. 63 (bottom) shows quantification of luminescence from the whole-body images of the same mice,

This experiment shows that detargeting from liver with mutant ApoE is still possible in the presence of a targeting ligand, but no differential signal was observed in the target organs.

### Methods

FVB.LoxP-Luc mice were injected with Cre expressing mRNA LNP formulations prepared as elsewhere in this deck. Approximately 1.5 μg (0.075 mg/kg) of RNA was used per mouse. Retro-orbital injections were performed with a total of ˜100 μL of LNP formulation depending on condition. Mixes were diluted up to 100 uL with PBS when lower RNA amounts were injected.

24 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time course sequence imaging every 5 minutes for 20 minutes. Aura imaging software was used to measure ROIs in photons/s.

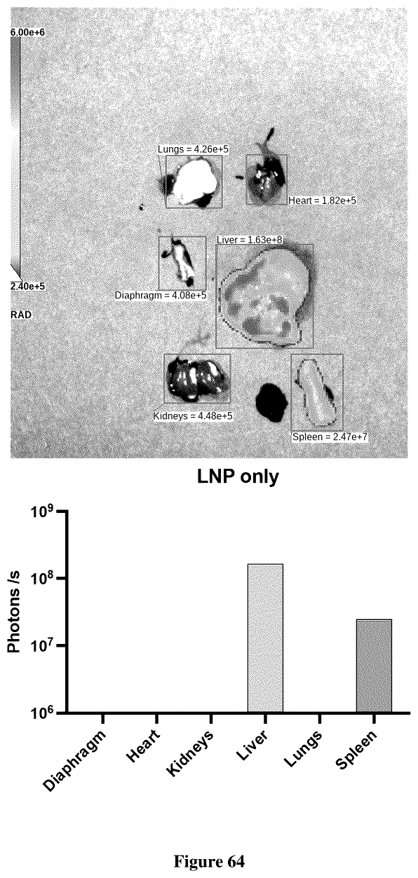

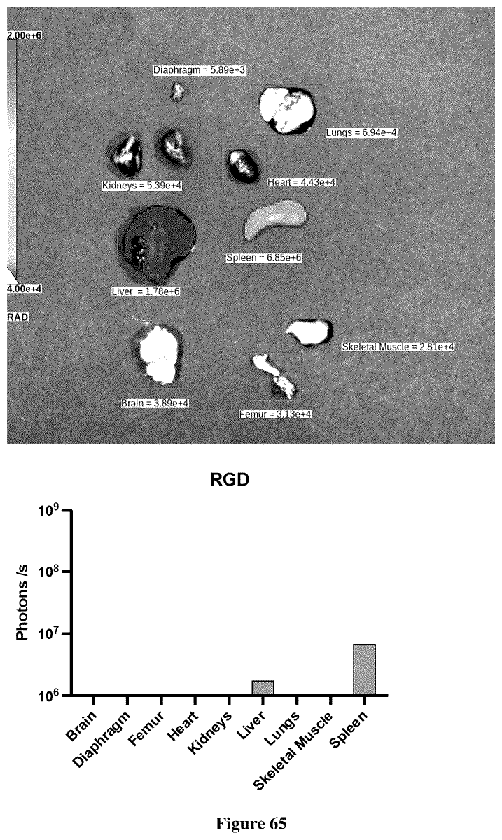

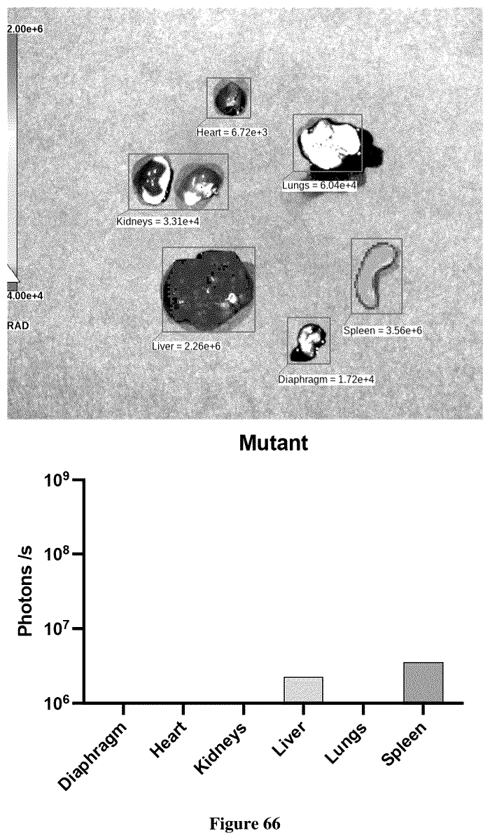

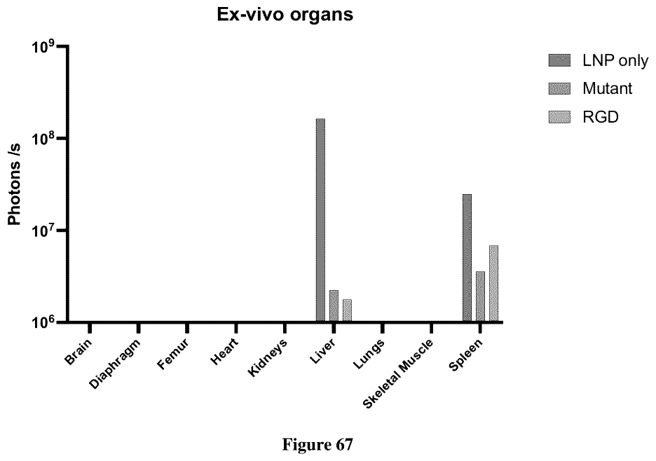

FIG. 64 shows LNP only. FIG. 65 shows LNP coated with mutant ApoE fused to RGD/PK7. FIG. 66 shows LNP coated with mutant ApoE. FIG. 67 shows summary plot showing quantification of organs from mice treated with each particle.

This experiment shows that detargeting from liver with mutant ApoE is still possible in the presence of a targeting ligand, but no differential signal was observed in the target organs.

### Example 12: Retargeting by Antibody Pull Down

### Methods

Protein A/G was cloned into N-terminus of mutant ApoE with a GS10 linker by Gibson cloning.

HEK293FT cells were transfected with cloned constructs, and media was changed to DMEM with no FBS after overnight incubation. Conditioned media were collected after 48h of media change. In parallel, HEK293FT cells were transfected with corresponding receptors for tested constructs.

LNPs were first synthesized by vortex mixing of lipids and RNA. Then, they were mixed in 1:1 volume ratio with conditioned media of mutant ApoE constructs and incubated for 15 minutes at 37 C. Precoated LNPs were incubated with different doses of CD5 (SouthernBiotech, Cat. No. 1547-01) or CD117 (BioLegend Car. No. 105801) antibodies for 15 minutes at 37 C. Total volume was completed to 100 ul/well with DMEM. 100 ul from each condition were added to each well of 96-well plate,

16-24h post-transduction, 100 ul of Bright-Glo substrate was added on cells and after 2 min of incubation, the transduction efficiency were quantified by luminescence plate reader.

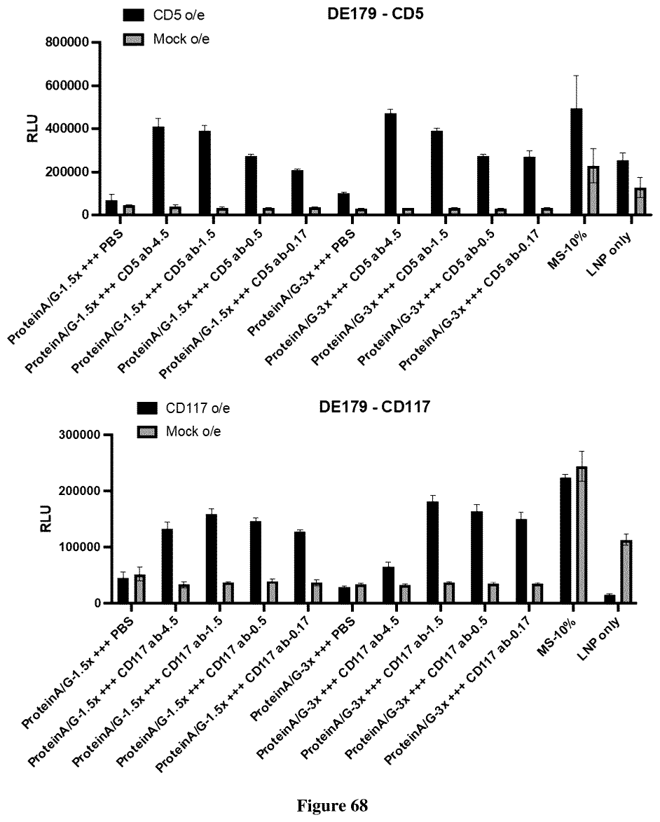

FIG. 68 (top) shows luminescence level on either plain HEK293FT or CD5-overexpressing cells, transduced with different doses of LNPs coated with mutant ApoE fused to Protein A/G and incubated with CD5. FIG. 68 (bottom) shows the results for the same experiment by using plain or CD117-overexpressing cells and LNPs coated with Protein A/G+CD117 antibody.

This experiment tests another strategy for retargeting; Fusing Protein A/G to the mutant ApoE, and incubation first with LNPs, then with receptor targeting antibodies. In these figures, we observed that LNPs coated with Protein A/G-fused mutant ApoE and incubated with CD5 or CD117 antibody transduce cells expressing only the corresponding receptor, and not plain HEK293FT cells. On the other hand, LNPs coated with Protein A/G fused mutant ApoE and incubated with PBS didn't enhance transduction in any cell lines.

### Methods

Protein A/G or Strep coding sequences were cloned into N-terminus of mutant ApoE with a GS10 linker by Gibson cloning.

HEK293FT cells were transfected with cloned constructs, and media was changed to DMEM with no FBS after overnight incubation. Conditioned media were collected after 48h of media change.

LNPs were first synthesized by vortex mixing of lipids and RNA. Then, they were mixed in 1:1 volume ratio with conditioned media of mutant ApoE constructs and incubated for 15 minutes at 37 C. Precoated LNPs were incubated with different doses of antibodies for 15 minutes at 37 C. Total volume was completed to 100 ul/well with RPMI. 100 ul from each condition were added to each well of 96-well plate.

16-24h post-transduction, 100 ul of Bright-Glo substrate was added on cells and after 2 min of incubation, the transduction efficiency were quantified by luminescence plate reader.

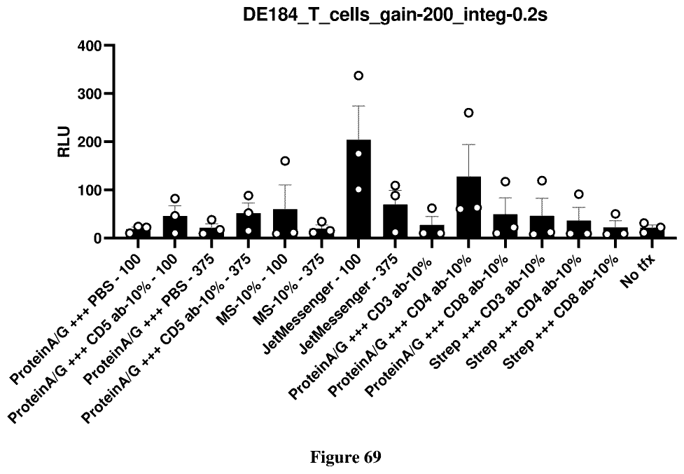

FIG. 69 shows luminescence level on primary mouse T cells cells, transduced with different doses of LNPs (100 or 375 μg of RNA) coated with mutant ApoE fused to Protein A/G or Streptavidin and incubated with CD3, CD4, CD5, or CD8 antibodies either as unconjugated or biotin conjugated.

This experiment tests another strategy for retargeting by pulling down the receptor-targeting antibodies on LNPs. To test the translational potential of this strategy, we tested them on primary T cells isolated from mouse spleen. We observed that LNPs coated with Protein A/G-fused mutant ApoE and incubated with CD5 and CD4 antibodies enable transduction of T cells although the signals were low.

### Methods

Ai14.LoxP-tdTomato mice were injected with Cre expressing mRNA LNP formulations prepared as elsewhere in this deck. Approximately 3 μg (0.15 mg/kg) of RNA was used per mouse. Retro-orbital injections were performed with a total of ˜100 μL of LNP formulation depending on condition. Mixes were diluted up to 100 uL with PBS when lower RNA amounts were injected.

24 hours post injection, mice were anaesthetized and shaved, then injected with luciferin substrate. IVIS imaging for luciferase signal was performed 15 minutes after luciferin injection. A 60s exposure was used for all images with otherwise default settings. An IVIS Spectrum was used for all imaging, doing a time course sequence imaging every 5 minutes for 20 minutes. Aura imaging software was used to measure ROIs in photons/s.

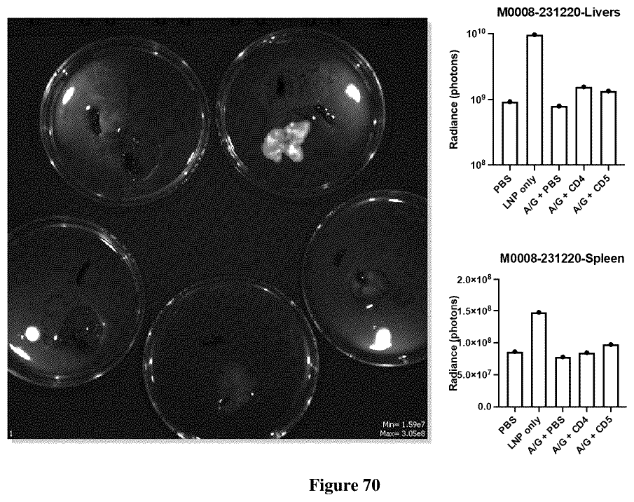

FIG. 70 (left) shows fluorescence images of livers and spleens ex vivo with IVIS for the mice in question, measuring photons/s. FIG. 70 (right) shows quantification of fluorescence from a set of LNP or control injections. Injected samples are PBS (1), LNP only (2), LNPs coated with mutant ApoE fused to Protein A/G and incubated with PBS (3), CD4 antibody (4) or CD5 (5) antibody, respectively.

This experiment tests another strategy for retargeting; Fusing Protein A/G to the mutant ApoE, and incubation first with LNPs, then with receptor targeting antibodies. We observed that detargeting from liver with mutant ApoE is still possible in the presence of protein A/G and pull downed antibody, but very slight increase in signal was observed in the retargeting organs.

### Example 13: Retargeting by Antibody Conjugation

### Methods

LNPs were first synthesized by vortex mixing of lipids and RNA. For conjugation experiments, DSPE-PEG maleimide was introduced to LNP formulation as 0.5% molar ratio, by decreasing DMG-PEG ratio from 1.5 to 1%. So final ratio was 50:10:38.5:1:0.5 for ionizable lipid, DSPC, cholesterol, DMG-PEG, DSPE-PEG maleimide, respectively.

Receptor targeting antibodies were functionalized by incubating with SATA at 10× molar ratio for 30 min at RT. Then, antibodies were purified with desalting columns, and deprotonated by incubation with 1/10 volume of 0.5M sodium deoxycholate for 2 h at RT. After another desalting step, functionalized antibodies were incubated with maleimide-LNPs for 1 h at RT at different doses. Unbound antibodies were eliminated by overnight dialysis using a 300 kDa dialysis tube. Total volume was completed to 100 ul/well with RPMI. 100 ul from each condition were added to each well of 96-well plate.

16-24h post-transduction, 100 ul of Bright-Glo substrate was added on cells and after 2 min of incubation, the transduction efficiency were quantified by luminescence plate reader.

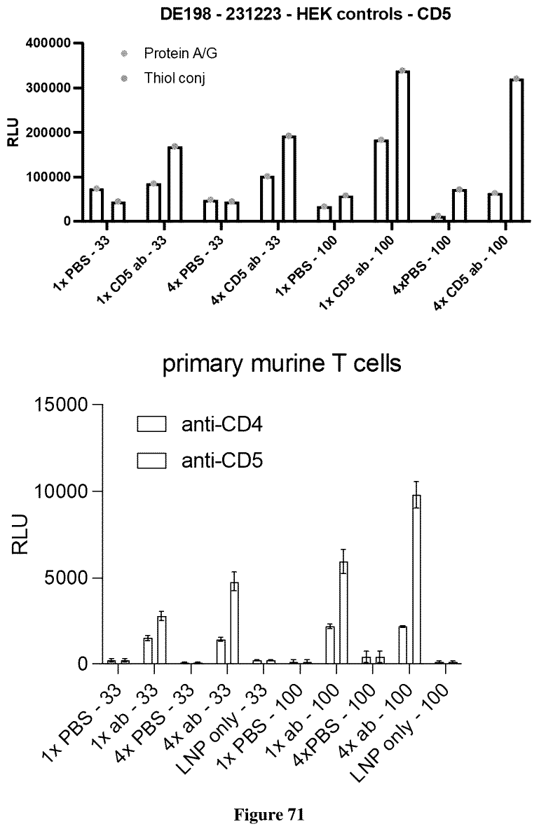

FIG. 71 shows luminescence level on HEK293FT controls and primary mouse T cells, transduced with different doses of LNPs (100 or 375 μg of RNA) conjugated with CD4 or CD5 antibodies.

This experiment tests another strategy for retargeting by covalently attaching the receptor-targeting antibodies on LNPs. To test the translational potential of this strategy, we tested them on primary T cells isolated from mouse spleen. We observed that LNPs conjugated with CD4 and especially CD5 antibodies enable transduction of T cells, and HEK293FT cells overexpressing CD5 with a higher efficiency than the Protein A/G-mediated pull-down strategy.

### Example 14: ApoE Competition Assay

Data using T-cells shows on-target transduction with off-target suppression.

### Methods

C57/BL6 mice injected with luciferase RNA LNP formulations were prepared. LNPs were dosed at 0.075 mg/kg. Retro-orbital injections were performed with a total of ˜100 μL of LNP formulation depending on condition. Mixes were diluted up to 100 uL with PBS when lower RNA amounts were injected.

Mice were injected with luciferin, then sacrificed with CO₂, 6 hours post injection. Spleens were dissected out and imaged using an IVIS, then T cells purified using an EasySep Mouse T Cell Isolation kit (Stem Cell Technology). Cells were counted and normalized, and luminescence was recorded using a Biotek at several gain levels.

### Results

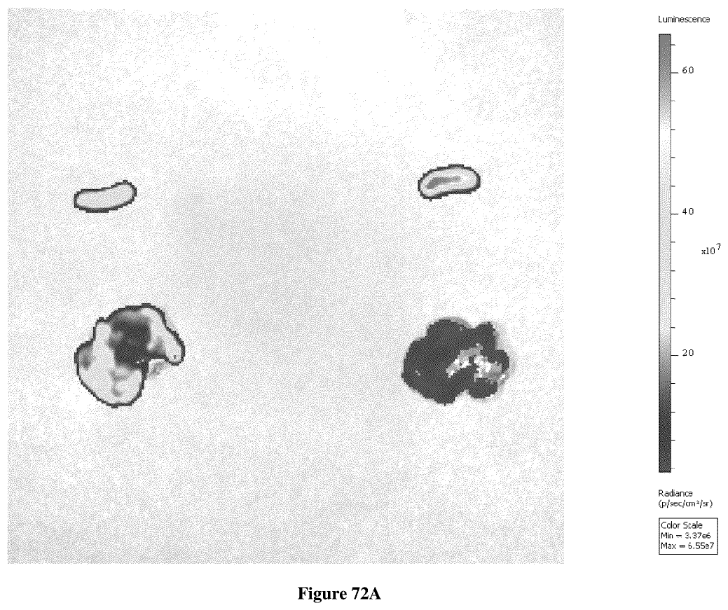

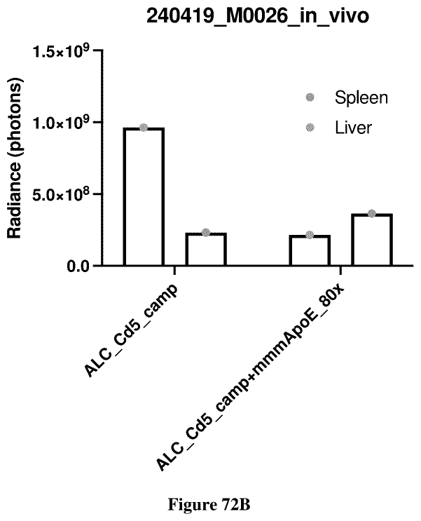

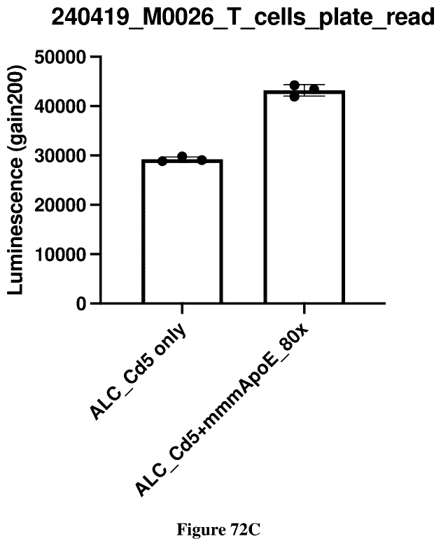

FIG. 72A shows images of organs showing radiance in photons/s. Spleen (top) and liver (bottom) are shown for 2 mice injected with cd5 LNPs alone or LNPs mixed with protein. FIG. 72B shows radiance of spleens and livers in photons/s. FIG. 72C shows luminescence from purified T cells

This experiment shows that compared to LNP only conditions, the addition of the mutant Apo-E protein produces a two-fold effect of reducing (off-target) liver signal and increase on-target T cell signal. Here, the level of liver signal is reduced by ˜75% with a corresponding ˜50% increase of on-target signal.

### Methods

C57/BL6 mice injected with luciferase RNA LNP formulations were prepared. LNPs were dosed at 0.05 mg/kg. Retro-orbital injections were performed with a total of ˜100 μL of LNP formulation depending on condition. Mixes were diluted up to 100 uL with PBS when lower RNA amounts were injected.

Mice were injected with luciferin, then sacrificed with CO₂, 6 hours post injection. Spleens were dissected out and imaged using an IVIS, then T cells purified using an EasySep Mouse T Cell Isolation kit (Stem Cell Technology). Cells were counted and normalized, and luminescence was recorded using a Biotek at several gain levels.

### Results

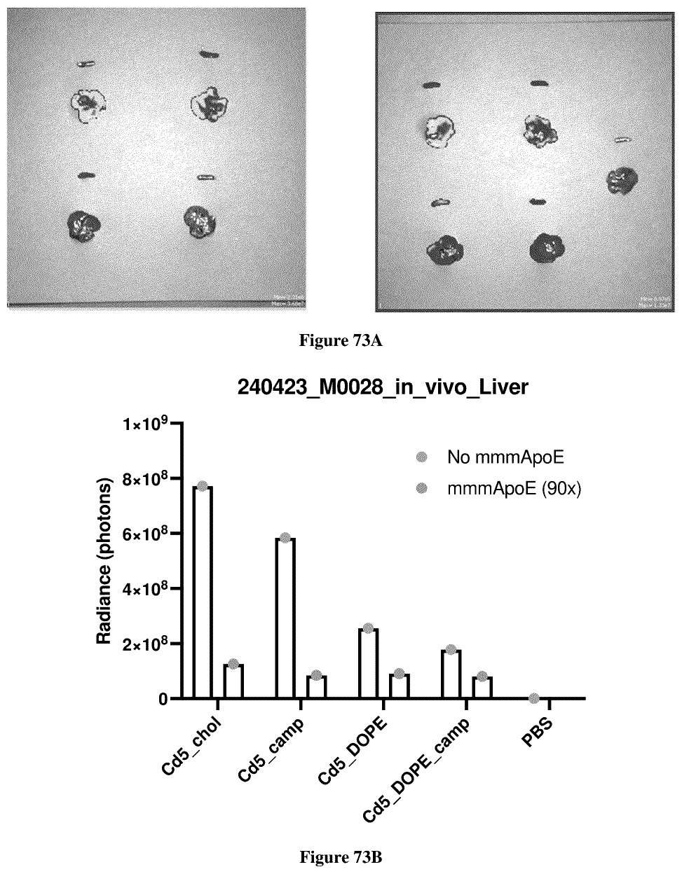

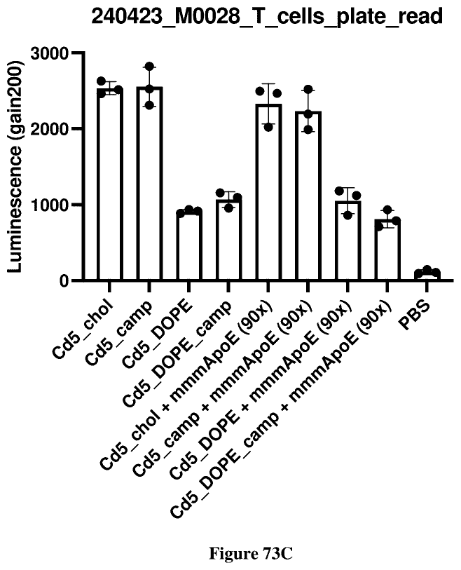

FIG. 73A shows images of organs showing radiance in photons/s. Spleen (top) and liver (bottom) are shown for 9 mice injected with cd5 LNPs alone or LNPs mixed with protein. FIG. 73B shows liver radiance with and without mutant ApoE at 90× dose from all mice. FIG. 73C shows T cell luminescence post purification from each mouse.

This experiment shows that compared to LNP only conditions, the addition of the mutant Apo-E protein reduces liver background by ˜80% without affecting on-target signal. This also shows that the mutant ApoE is compatible with several LNP formulations, including substitution of components from the standard ALC mixture (ALC-0315 from MedChem Express, Cat. No. HY-138170) with campesterol (Millipore Sigma, Cat. No. PHL89514) or DOPE lipids (Millipore Sigma, Cat. No. 850725C).

### Methods

C57/BL6 mice injected with luciferase RNA LNP formulations were prepared. Approximately 0.45 mg/kg of RNA was used per mouse. Retro-orbital injections were performed with a total of ˜100 μL of LNP formulation depending on condition. Mixes were diluted up to 100 uL with PBS when lower RNA amounts were injected.

Mice were injected with luciferin, then sacrificed with CO₂, 6 hours post injection. Spleens were dissected out and T cells purified using an EasySep Mouse T Cell Isolation kit (Stem Cell Technology). Cells were counted and normalized, and luminescence was recorded using a Biotek at several gain levels.

### Results

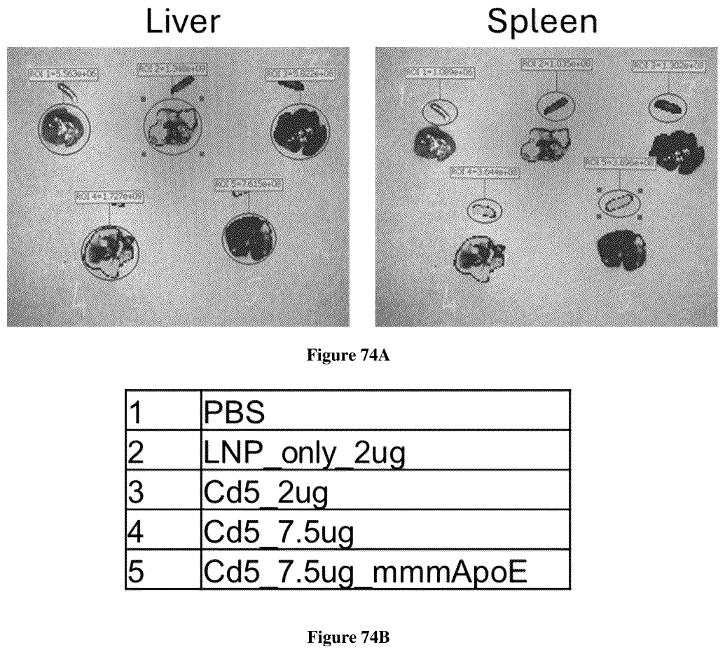

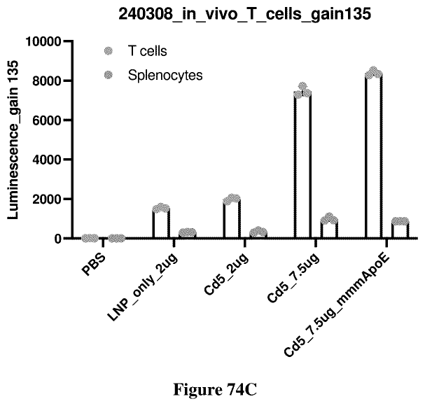

FIG. 74A shows images of various organs ex vivo with IVIS, measuring photons/s or luminescence from a set of LNP or control injections. ROIs calculated per organ to quantify the signal. FIG. 74B shows a list of conditions and doses for LNP injections. FIG. 74C shows quantification of luminescence signals from T cells isolated from mice injected with CD5-conjugated fluc LNPs, unconjugated LNPs or PBS.

This experiment shows that the luminescence signal in the organ images is almost entirely contributed by the T cells, which the CD5 antibody targets. This suggests that conjugate is directing the LNPs to those cells. Additionally, FIGS. 74A-74C show the large difference in signal with higher doses (2 μg vs 7.5 μg) and the increase in T-cell signal comparing unconjugated and conjugated LNPs. Finally, the signal from LNPs mixed with ApoE shows that the off-target liver transduction is reduced by ˜60%, while on-target T cell luminescence is unchanged or improved.

### Methods

C57/BL6 mice injected with luciferase RNA LNP formulations were prepared. Several doses of mg/kg of RNA were used (listed). Retro-orbital injections were performed with a total of ˜100 μL of LNP formulation depending on condition. Mixes were diluted up to 100 uL with PBS when lower RNA amounts were injected.

Mice were injected with luciferin, then sacrificed with CO₂, 6 hours post injection. Spleens were dissected out and imaged using an IVIS, then T cells purified using an EasySep Mouse T Cell Isolation kit (Stem Cell Technology). Cells were counted and normalized, and luminescence was recorded using a Biotek at several gain levels.

Mouse weights: Mouse 1-17.5, Mouse 2-19.4, Mouse 3-20.1, Mouse 4-18.2, and Mouse 5-12.8.

PBS and Cd5_ALC (25 μg=˜2 mg/kg)-Conditions 1 and 5

Cd5 & Cd5 with mono-mmmApoE (mono=monomeric, SEQ ID NO:168) & Cd5 with di-mmmApoE (SEQ ID NO:182)-Conditions 2-3-4

Particles ALC_Cd5=396 ng/ul; Proteins produced in Freestyles (293-F Cells, ThermoFisher, Cat. No. R79007), 2.1 and 2.9 mg/ml (freeze-thawed); Protein: RNA ratio is 180×.

### Results

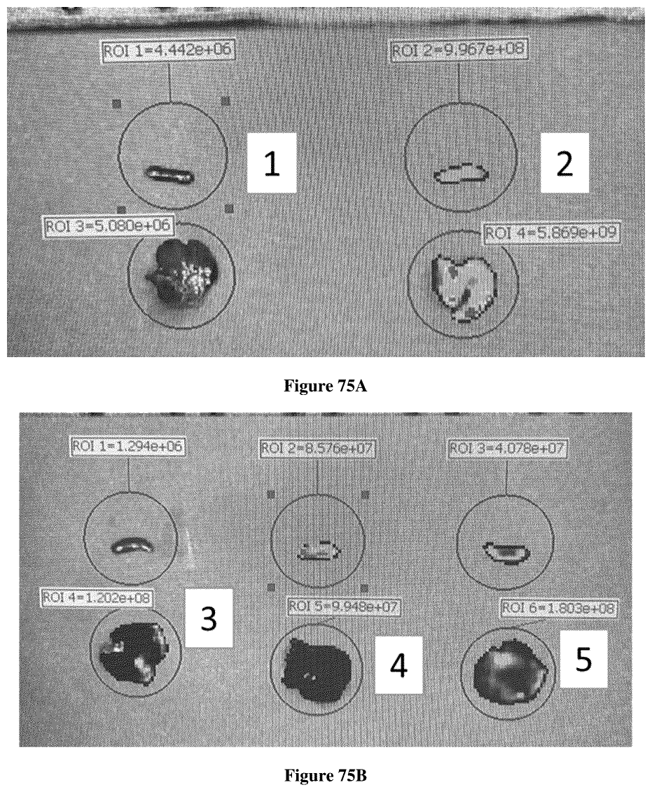

FIG. 75A shows images of organs showing radiance in photons/s. Spleen (top) and liver (bottom) are shown for 5 mice. FIG. 75B shows luminescence of T cells extracted from spleens for PBS and ALC mice.

This experiment shows that the luminescence signal in the organ images is contributed by the T cells, which the CD5 antibody targets. This experiment also shows that the addition of mutant ApoE (mice 4 and 5) increases spleen radiance, potentially due to lower absorbance of LNPs in the liver.

### Methods

C57/BL6 mice injected with luciferase RNA LNP formulations were prepared. Several doses of mg/kg of RNA were used (listed). Retro-orbital injections were performed with a total of ˜100 μL of LNP formulation depending on condition. Mixes were diluted up to 100 uL with PBS when lower RNA amounts were injected.

Mice were injected with luciferin, then sacrificed with CO₂, 6 hours post injection. Spleens were dissected out and imaged using an IVIS, then T cells purified using an EasySep Mouse T Cell Isolation kit (Stem Cell Technology). Cells were counted and normalized, and luminescence was recorded using a Biotek at several gain levels.

Mouse-1: CD5, Mouse-2: CD117, Mouse-3: CD5+mutant, Mouse-4: CD117+mutant, and Mouse-5: PBS. 13 μg for CD5

### Results

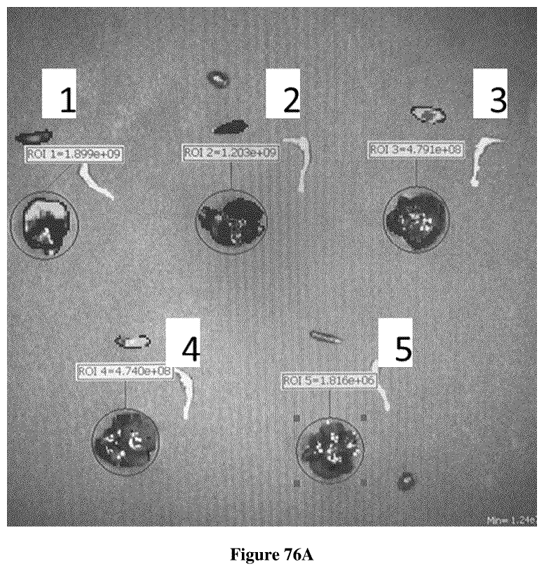

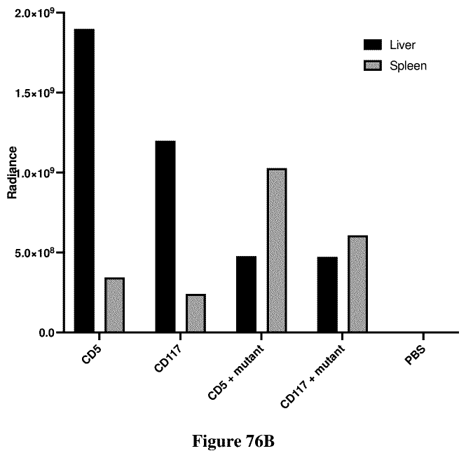

FIG. 76A shows images of organs showing radiance in photons/s. Spleen (top), liver (bottom), and bone marrow (top right). Images are shown for 5 mice. FIG. 76B shows radiance measurements for livers and spleens in pictured organs,

This experiment shows that compared to LNP only conditions, the addition of the mutant Apo-E protein produces a two-fold effect of reducing (off-target) liver signal and increase on-target T cell signal. Here, mutant ApoE reduces liver signal for CD5-conjugated LNPs by 75% and for CD117-conjugated LNPs by 60%. The mutant increases spleen signal by 3×.

FIG. 80 shows luminescence of T cells and HSCs purified from preceding slide mice. This data shows that the addition of mutant ApoE to CD5 conjugated LNPs increases signal while reducing off-target liver transduction.

However, while liver signal goes down for CD117-conjugated LNPs with mutant ApoE, they do not transduce HSCs as efficiently.

Further data shows transduction with distinct LNP formulations used in other experiments, but lacking mutant ApoE.

### Methods

C57/BL6 mice injected with luciferase RNA LNP formulations were prepared. Approximately 0.45 mg/kg of RNA was used per mouse. Retro-orbital injections were performed with a total of ˜100 μL of LNP formulation depending on condition. Mixes were diluted up to 100 uL with PBS when lower RNA amounts were injected.

Mice were injected with luciferin, then sacrificed with CO₂, 6 hours post injection. IVIS imaging for luciferase signal was performed 15 minutes after luciferin injection. A 60s exposure was used for all images with otherwise default settings. An IVIS Spectrum was used for all imaging. Aura imaging software was used to measure ROIs in photons/s.

### Results

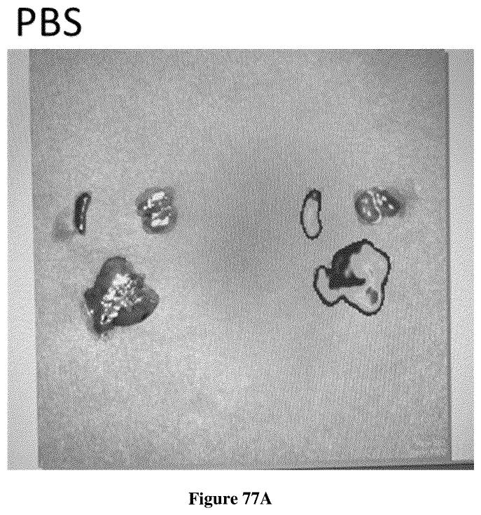

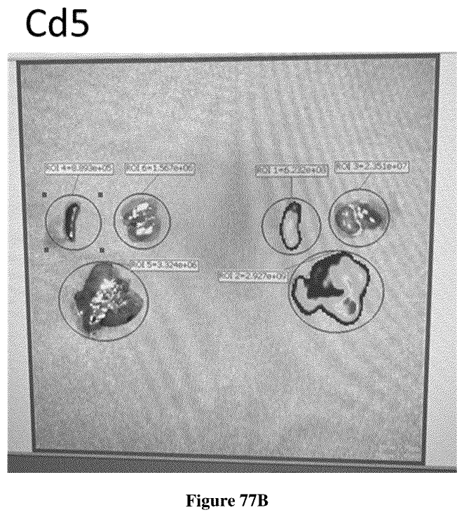

FIG. 77A shows images of various organs ex vivo with IVIS, measuring photons/s or luminescence from a set of LNP or control injections. FIG. 77B shows same image but with ROIs calculated per organ to quantify the signal.

This experiment shows that that antibodies conjugated with CD5 using SATA (ThermoFisher Cat. No. 26102) or TCEP (ThermoFisher 77720) chemistry cause strong luminescence in both the liver (bottom organ in arrangements) and spleen (top left organ). However, mutant apoe is not used here and no-non conjugation control is shown.

### Methods

C57/BL6 mice injected with luciferase RNA LNP formulations were prepared. Approximately 0.45 mg/kg of RNA was used per mouse. Retro-orbital injections were performed with a total of ˜100 μL of LNP formulation depending on condition. Mixes were diluted up to 100 uL with PBS when lower RNA amounts were injected.

Mice were injected with luciferin, then sacrificed with CO₂, 6 hours post injection. Spleens were dissected out and T cells purified using an EasySep Mouse T Cell Isolation kit (Stem Cell Technology). Cells were counted and normalized, and luminescence was recorded using a Biotek at several gain levels.

### Results

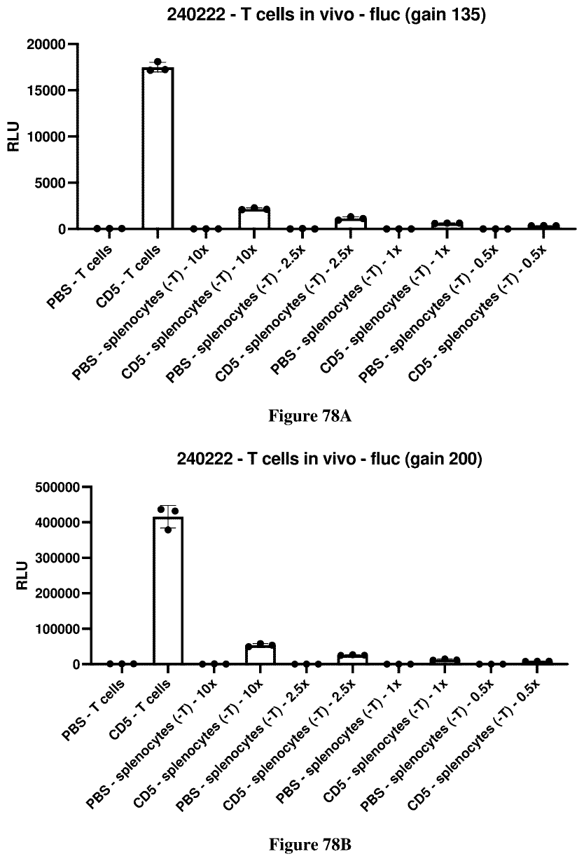

FIG. 78A shows quantification of luminescence signals from T cells isolated from mice injected with CD5-conjugated fluc LNPs or PBS. FIG. 78B shows same data but acquired with higher gain on a Biotek spectrophotometer.

This experiment shows that the luminescence signal in the organ images on the preceding slide is almost entirely contributed by the T cells, which the CD5 antibody targets. This suggests that conjugate is directing the LNPs to those cells.

### Methods

C57/BL6 mice injected with luciferase RNA LNP formulations were prepared. Approximately 0.45 mg/kg of RNA was used per mouse. Retro-orbital injections were performed with a total of ˜100 μL of LNP formulation depending on condition. Mixes were diluted up to 100 uL with PBS when lower RNA amounts were injected.

Mice were sacrificed with CO₂, 6 hours post injection. Spleens were dissected out and T cells purified using an EasySep Mouse T Cell Isolation kit (Stem Cell Technology). Cells were plated and visualized using a Keyence fluorescence microscope.

### Results

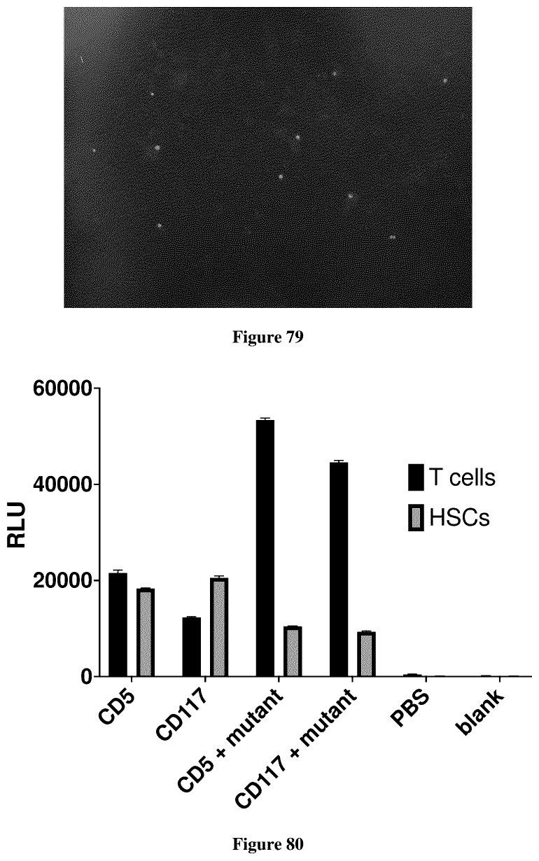

FIG. 79 shows images of T cells isolated from loxP-tdTomato mic injected with CD5-conjugated LNPs carrying Cre mRNA showing positive fluorescence.

This experiment shows that that antibodies conjugated with CD5 using SATA or TCEP chemistry can cause transduction and subsequent expression of Cre.

### INCORPORATION BY REFERENCE

All US and PCT patent application publications and US patents mentioned herein are hereby incorporated by reference in their entirety as if each individual patent application publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition, comprising a lipid nanoparticle and a modified lipid binding protein; wherein the modified lipid binding protein comprises a fused cell-specific binding domain; the modified lipid binding protein has been additionally modified such that it does not bind substantially to its natural receptor; and the fused cell-specific binding domain binds to a target cell.

2. The composition of claim 1, wherein the lipid binding protein is a biomolecular corona protein.

3. The composition of claim 1, wherein lipid binding protein is an Apolipoprotein.

4. The composition of claim 3, wherein the Apolipoprotein is ApoA-I, ApoA-II, ApoA-IV, ApoA-V, ApoB, ApoB-48 and ApoB-100, ApoC-I, ApoC-II, ApoC-III, ApoD, ApoE, ApoF, ApoH (Beta-2-glycoprotein I), ApoJ (Clusterin), ApoL, ApoM, ApoO (Apolipoprotein O), or Apo (a)-component of Lipoprotein (a).

5. (canceled)

6. The composition of claim 1, wherein the lipid binding protein is a Fatty Acid Binding Protein (FABP).

7. The composition of claim 6, wherein the Fatty Acid Binding Protein is L-FABP, I-FABP, or H-FABP.

8. The composition of claim 1, wherein the lipid binding protein is β2-GPI, Vtn5, a cellular retinol-binding protein (CRBP), a cellular retinoic acid-binding protein (CRABP), a sterol carrier protein (SCP), NPC1, NPC2, a StarD protein (like StarD1, StarD2), a phosphatidylserine-binding protein, an oxysterol-binding protein (OSBP), a Acyl-CoA-binding protein (ACBD), a PLIN protein, or perilipin.

9. The composition of claim 1, wherein the lipid binding protein is an engineered lipid binding protein.

10. The composition of claim 1, wherein the lipid binding protein has mutation in its receptor binding domain.

11-16. (canceled)

17. The composition of claim 1, wherein the cell-specific binding domain is fused to the lipid binding protein via a flexible linker.

18. (canceled)

19. The composition of claim 1, wherein the cell-specific binding domain is vitronectin, EGFR DARPin, an scFv, myomixer, an integrin-binding peptide, or (KKEEE) 3K binder (SEQ ID NO: 269).

20. (canceled)

21. The composition of claim 1, wherein the lipid binding protein is further fused to a lipid-binding peptide.

22-25. (canceled)

26. The composition of claim 1, wherein the lipid binding protein comprises a mutation in its lipid-binding domain and/or its amino terminal fragment.

27. The composition of claim 1, wherein the composition further comprises a serum-stabilizing tag.

28. (canceled)

29. The composition of claim 1, wherein the lipid nanoparticle and the modified lipid binding protein are mixed in a ratio of about 1:1, 1:2, 1:3, 1:4, 1:5, 1:6, 1:7, 1:8, 1:9, or 1:10.

30. The composition of claim 1, wherein the modified lipid binding protein is in the form of conditioned media (CM).

31. The composition of claim 1, wherein the composition further comprises a nucleic acid.

32-35. (canceled)

36. A composition, comprising a lipid nanoparticle and a modified lipid binding protein; wherein the modified lipid binding protein comprises a conjugated cell-specific binding domain; the modified lipid binding protein has been additionally modified such that it does not bind substantially to its natural receptor; and the conjugated cell-specific binding domain binds to a target cell.

37. (canceled)

38. A composition, comprising a lipid nanoparticle and a modified lipid binding protein; wherein the lipid nanoparticle comprises a conjugated cell-specific binding domain; the modified lipid binding protein has been additionally modified such that it does not bind substantially to its natural receptor; and the conjugated cell-specific binding domain binds to a target cell.

39-73. (canceled)

74. A method of treating or preventing a disease, comprising administering to a subject in need thereof an effective amount of the composition of claim 1.

75-77. (canceled)


SEQUENCE LISTING (276 sequences)

SEQ ID NO: 1 - DNA, 954 bp, Homo sapiens
  source 1..954: mol_type = unassigned DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 2 - protein, 317 aa, Homo sapiens [sequence omitted]

SEQ ID NO: 3 - DNA, 954 bp, synthetic construct
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 4 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 5 - DNA, 1008 bp, synthetic construct
  source 1..1008: mol_type = other DNA
atgaaggttctgtgggctgcgttgctggtcacattcctggcaggatgccaggccgactacaaggacgacgatgacaagggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 6 - protein, 335 aa, synthetic construct
MKVLWAALLVTFLAGCQADYKDDDDKGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 7 - DNA, 1029 bp, synthetic construct
  source 1..1029: mol_type = other DNA
atgaaggttctgtgggctgcgttgctggtcacattcctggcaggatgccaggccaagaacgagcaggagctgctggagctggacaagtgggccagcctgggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 8 - protein, 342 aa, synthetic construct
MKVLWAALLVTFLAGCQAKNEQELLELDKWASLGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 9 - DNA, 996 bp, synthetic construct
  source 1..996: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggcggatccggtgactacaaggacgacgatgacaagggttccggacagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 10 - protein, 331 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGGSGDYKDDDDKGSGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 11 - DNA, 996 bp, synthetic construct
  source 1..996: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgggatccggtgactacaaggacgacgatgacaagggttccggactcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 12 - protein, 331 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMGSGDYKDDDDKGSGLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 13 - DNA, 1038 bp, synthetic construct
  source 1..1038: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccggatccggtgggtccggtagtggtggttccgactacaaggacgacgatgacaagggatccggagggtcaggatccggcgggagcccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 14 - protein, 345 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTGSGGSGSGGSDYKDDDDKGSGGSGSGGSPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 15 - DNA, 1038 bp, synthetic construct
  source 1..1038: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccgggatccggtgggtccggtagtggtggttccgactacaaggacgacgatgacaagggatccggagggtcaggatccggcgggagcgtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 16 - protein, 345 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPGSGGSGSGGSDYKDDDDKGSGGSGSGGSVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 17 - DNA, 1038 bp, synthetic construct
  source 1..1038: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgggatccggtgggtccggtagtggtggttccgactacaaggacgacgatgacaagggatccggagggtcaggatccggcgggagcctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 18 - protein, 345 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMGSGGSGSGGSDYKDDDDKGSGGSGSGGSLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 19 - DNA, 1038 bp, synthetic construct
  source 1..1038: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggcggatccggtgggtccggtagtggtggttccgactacaaggacgacgatgacaagggatccggagggtcaggatccggcgggagccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 20 - protein, 345 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGGSGGSGSGGSDYKDDDDKGSGGSGSGGSQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 21 - DNA, 1038 bp, synthetic construct
  source 1..1038: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggcggatccggtgggtccggtagtggtggttccgactacaaggacgacgatgacaagggatccggagggtcaggatccggcgggagccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 22 - protein, 345 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGGSGGSGSGGSDYKDDDDKGSGGSGSGGSQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 23 - DNA, 1059 bp, synthetic construct
  source 1..1059: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccggatccggtgggtccggtagtggtggttccaagaacgagcaggagctgctggagctggacaagtgggccagcctgggatccggagggtcaggatccggcgggagcccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 24 - protein, 352 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTGSGGSGSGGSKNEQELLELDKWASLGSGGSGSGGSPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 25 - DNA, 1059 bp, synthetic construct
  source 1..1059: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgggatccggtgggtccggtagtggtggttccaagaacgagcaggagctgctggagctggacaagtgggccagcctgggatccggagggtcaggatccggcgggagcctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 26 - protein, 352 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMGSGGSGSGGSKNEQELLELDKWASLGSGGSGSGGSLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 27 - DNA, 1059 bp, synthetic construct
  source 1..1059: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggcggatccggtgggtccggtagtggtggttccaagaacgagcaggagctgctggagctggacaagtgggccagcctgggatccggagggtcaggatccggcgggagccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 28 - protein, 352 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGGSGGSGSGGSKNEQELLELDKWASLGSGGSGSGGSQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 29 - DNA, 1074 bp, synthetic construct
  source 1..1074: mol_type = other DNA
atgaaggttctgtgggctgcgttgctggtcacattcctggcaggatgccaggccagcggcagcgagacccccggcaccagcgagagcgccacccccgagagcgactacaaggacgacgatgacaagtccggctcagagaccccaggcacctctgagtctgcaactcctgagtcaaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 30 - protein, 357 aa, synthetic construct
MKVLWAALLVTFLAGCQASGSETPGTSESATPESDYKDDDDKSGSETPGTSESATPESKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 31 - DNA, 1074 bp, synthetic construct
  source 1..1074: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccagcggcagcgagacccccggcaccagcgagagcgccacccccgagagcgactacaaggacgacgatgacaagtccggctcagagaccccaggcacctctgagtctgcaactcctgagtcaccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 32 - protein, 357 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTSGSETPGTSESATPESDYKDDDDKSGSETPGTSESATPESPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 33 - DNA, 1095 bp, synthetic construct
  source 1..1095: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccagcggcagcgagacccccggcaccagcgagagcgccacccccgagagcaagaacgagcaggagctgctggagctggacaagtgggccagcctgtccggctcagagaccccaggcacctctgagtctgcaactcctgagtcaccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 34 - protein, 364 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTSGSETPGTSESATPESKNEQELLELDKWASLSGSETPGTSESATPESPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 35 - DNA, 1095 bp, synthetic construct
  source 1..1095: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctgagcggcagcgagacccccggcaccagcgagagcgccacccccgagagcaagaacgagcaggagctgctggagctggacaagtgggccagcctgtccggctcagagaccccaggcacctctgagtctgcaactcctgagtcagccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 36 - protein, 364 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLSGSETPGTSESATPESKNEQELLELDKWASLSGSETPGTSESATPESAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 37 - DNA, 1095 bp, synthetic construct; identical to SEQ ID NO: 35
  source 1..1095: mol_type = other DNA

SEQ ID NO: 38 - protein, 364 aa, synthetic construct; identical to SEQ ID NO: 36

SEQ ID NO: 39 - DNA, 1455 bp, synthetic construct
  source 1..1455: mol_type = other DNA
atgaaggttctgtgggctgcgttgctggtcacattcctggcaggatgccaggccgacctgggcaagaggctgctggaggccgccagggccggccaggacgacgaggtgaggatcctgatggccaacggcgccgacgtgaacgccaaggacgacgagggcaggaccagcctgcacctggccgccagggagggccacctggaaatcgtggaggtgctgctgaaacacggcgccgacgtgaacgcccaggactggtacggtagtactcccctccacctggccgctgcctggggacatctggagattgtggaagtgctgctgaagaacgtggccgacgtgaacgccatggacggcgacggcagcacccccctgcacctggccgcccactacgcccacctggaggtggtggaggtgctgctgaagaacggcgcggacgtcaatgcccaggataagttcggcaagaccgccttcgacatcagcatcgacaacggcaacgaggacctggccgagatcctgcagaagctgaacggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 40 - protein, 484 aa, synthetic construct
MKVLWAALLVTFLAGCQADLGKRLLEAARAGQDDEVRILMANGADVNAKDDEGRTSLHLAAREGHLEIVEVLLKHGADVNAQDWYGSTPLHLAAAWGHLEIVEVLLKNVADVNAMDGDGSTPLHLAAHYAHLEVVEVLLKNGADVNAQDKFGKTAFDISIDNGNEDLAEILQKLNGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 41 - DNA, 1443 bp, synthetic construct
  source 1..1443: mol_type = other DNA
atgaaggttctgtgggctgcgttgctggtcacattcctggcaggatgccaggccgacctgggcaagaagctgctggaggccgccagggccggccaggacgacgaggtgaggatcctgatggccaacggcgccgacgtgaacgccgacgacacctggggctggacccccctgcacctggccgcctaccagggtcatcttgagattgttgaagtgttgttgaaaaatggtgccgatgtgaacgcctacgactacatcgggtggacgccgctacatctagcagctgacggccacctggagatcgtggaggtgctgctgaagaatggcgccgatgttaacgcgagcgactacatcggcgacacccctctgcatctcgcggcacacaacggccaccttgagatcgtcgaggtgctcttaaagcacggcgccgacgtgaacgcccaggacaagttcggcaagaccgccttcgacatcagcatcgacaacggcaacgaggacctggccgagatcctgcagggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 42 - protein, 480 aa, synthetic construct
MKVLWAALLVTFLAGCQADLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 43 - DNA, 1002 bp, synthetic construct
  source 1..1002: mol_type = other DNA
atgaaggttctgtgggctgcgttgctggtcacattcctggcaggatgccaggccgagtacatgcccatggagggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 44 - protein, 333 aa, synthetic construct
MKVLWAALLVTFLAGCQAEYMPMEGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 45 - DNA, 1002 bp, synthetic construct
  source 1..1002: mol_type = other DNA
atgaaggttctgtgggctgcgttgctggtcacattcctggcaggatgccaggccgagtacatgcccatggagggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagagccgcagccaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 46 - protein, 333 aa, synthetic construct
MKVLWAALLVTFLAGCQAEYMPMEGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQSRSQAEAFQARLKSWFEPLVEDMQRQWAGLVEKVQAAVGTSAAPVPSDNH

SEQ ID NO: 47 - DNA, 1056 bp, synthetic construct
  source 1..1056: mol_type = other DNA
atgaaggttctgtgggctgcgttgctggtcacattcctggcaggatgccaggccgagtacatgcccatggagggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacgactggctgaaggccttctacgacaaggtggccgagaagctgaaggaggccttctga

SEQ ID NO: 48 - protein, 351 aa, synthetic construct
MKVLWAALLVTFLAGCQAEYMPMEGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHDWLKAFYDKVAEKLKEAF

SEQ ID NO: 49 - DNA, 1065 bp, synthetic construct
  source 1..1065: mol_type = other DNA
atgaaggttctgtgggctgcgttgctggtcacattcctggcaggatgccaggccgagtacatgcccatggagggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggcagcggcgactggctgaaggccttctacgacaaggtggccgagaagctgaaggaggccttctga

SEQ ID NO: 50 - protein, 354 aa, synthetic construct
MKVLWAALLVTFLAGCQAEYMPMEGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGSGDWLKAFYDKVAEKLKEAF

SEQ ID NO: 51 - DNA, 1029 bp, synthetic construct; identical to SEQ ID NO: 7
  source 1..1029: mol_type = other DNA

SEQ ID NO: 52 - protein, 342 aa, synthetic construct; identical to SEQ ID NO: 8

SEQ ID NO: 53 - DNA, 1029 bp, synthetic construct
  source 1..1029: mol_type = other DNA
atgaaggttctgtgggctgcgttgctggtcacattcctggcaggatgccaggccaagaacgagcaggagctgctggagctggacaagtgggccagcctgggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagagccgcagccaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 54 - protein, 342 aa, synthetic construct
MKVLWAALLVTFLAGCQAKNEQELLELDKWASLGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQSRSQAEAFQARLKSWFEPLVEDMQRQWAGLVEKVQAAVGTSAAPVPSDNH

SEQ ID NO: 55 - DNA, 1083 bp, synthetic construct
  source 1..1083: mol_type = other DNA
atgaaggttctgtgggctgcgttgctggtcacattcctggcaggatgccaggccaagaacgagcaggagctgctggagctggacaagtgggccagcctgggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacgactggctgaaggccttctacgacaaggtggccgagaagctgaaggaggccttctga

SEQ ID NO: 56 - protein, 360 aa, synthetic construct
MKVLWAALLVTFLAGCQAKNEQELLELDKWASLGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHDWLKAFYDKVAEKLKEAF

SEQ ID NO: 57 - DNA, 1092 bp, synthetic construct
  source 1..1092: mol_type = other DNA
atgaaggttctgtgggctgcgttgctggtcacattcctggcaggatgccaggccaagaacgagcaggagctgctggagctggacaagtgggccagcctgggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggcagcggcgactggctgaaggccttctacgacaaggtggccgagaagctgaaggaggccttctga

SEQ ID NO: 58 - protein, 363 aa, synthetic construct
MKVLWAALLVTFLAGCQAKNEQELLELDKWASLGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGSGDWLKAFYDKVAEKLKEAF

SEQ ID NO: 59 - DNA, 1719 bp, synthetic construct
  source 1..1719: mol_type = other DNA
atgaaggttctgtgggctgcgttgctggtcacattcctggcaggatgccaggccgacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 60 - protein, 572 aa, synthetic construct
MKVLWAALLVTFLAGCQADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 61 - DNA, 1491 bp, synthetic construct
  source 1..1491: mol_type = other DNA
atgaaggttctgtgggctgcgttgctggtcacattcctggcaggatgccaggccgacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggaggactggctgaaggccttctacgacaaggtggccgagaagctgaaggaggccttctga

SEQ ID NO: 62 - protein, 496 aa, synthetic construct
MKVLWAALLVTFLAGCQADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQEDWLKAFYDKVAEKLKEAF

SEQ ID NO: 63 - DNA, 1719 bp, synthetic construct
  source 1..1719: mol_type = other DNA
atgaaggttctgtgggctgcgttgctggtcacattcctggcaggatgccaggccgacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagagccgcagccaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 64 - protein, 572 aa, synthetic construct
MKVLWAALLVTFLAGCQADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQSRSQAEAFQARLKSWFEPLVEDMQRQWAGLVEKVQAAVGTSAAPVPSDNH

SEQ ID NO: 65 - DNA, 1773 bp, synthetic construct
  source 1..1773: mol_type = other DNA
atgaaggttctgtgggctgcgttgctggtcacattcctggcaggatgccaggccgacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacgactggctgaaggccttctacgacaaggtggccgagaagctgaaggaggccttctga

SEQ ID NO: 66 - protein, 590 aa, synthetic construct
MKVLWAALLVTFLAGCQADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHDWLKAFYDKVAEKLKEAF

SEQ ID NO: 67 - DNA, 1782 bp, synthetic construct
  source 1..1782: mol_type = other DNA
atgaaggttctgtgggctgcgttgctggtcacattcctggcaggatgccaggccgacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacggcagcggcgactggctgaaggccttctacgacaaggtggccgagaagctgaaggaggccttctga

SEQ ID NO: 68 - protein, 593 aa, synthetic construct
MKVLWAALLVTFLAGCQADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHGSGDWLKAFYDKVAEKLKEAF

SEQ ID NO: 69-164 - skipped (96 entries)

SEQ ID NO: 165 - DNA, 963 bp, synthetic construct
  source 1..963: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctccgcaagatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 166 - protein, 320 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLRKMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 167 - DNA, 963 bp, synthetic construct
  source 1..963: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 168 - protein, 320 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 169 - DNA, 1077 bp, synthetic construct
  source 1..1077: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgggatccggtggcgagatagccgcccttaaacaggagatagctgccttgaaaaaggagaacgctgcactgaagtgggagattgcggctctcaagcagggctattacggttccggattaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 170 - protein, 358 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMGSGGEIAALKQEIAALKKENAALKWEIAALKQGYYGSGLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 171 - DNA, 1077 bp, synthetic construct
  source 1..1077: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgggatccggtggcgaaattgccgctataaaacaggaaatagctgccatcaagaaggaaatagctgccattaagtgggagattgccgcgatcaaacagggctacggcggttccggattaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 172 - protein, 358 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMGSGGEIAAIKQEIAAIKKEIAAIKWEIAAIKQGYGGSGLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 173 - DNA, 1077 bp, synthetic construct
  source 1..1077: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgggatccggtggcgagctggctgccattaagcaggagctcgctgctatcaagaaagagcttgcagcaattaagtgggaattggcagcaatcaaacagggcgctgggggttccggattaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 174 - protein, 358 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMGSGGELAAIKQELAAIKKELAAIKWELAAIKQGAGGSGLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 175 - DNA, 1155 bp, synthetic construct
  source 1..1155: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgagcggcagcgagacccccggcaccagcgagagcgccacccccgagagcggcgagatagccgcccttaaacaggagatagctgccttgaaaaaggagaacgctgcactgaagtgggagattgcggctctcaagcagggctattactccggctcagagaccccaggcacctctgagtctgcaactcctgagtcattaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 176 - protein, 384 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMSGSETPGTSESATPESGEIAALKQEIAALKKENAALKWEIAALKQGYYSGSETPGTSESATPESLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 177 - DNA, 1155 bp, synthetic construct
  source 1..1155: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgagcggcagcgagacccccggcaccagcgagagcgccacccccgagagcggcgaaattgccgctataaaacaggaaatagctgccatcaagaaggaaatagctgccattaagtgggagattgccgcgatcaaacagggctacggctccggctcagagaccccaggcacctctgagtctgcaactcctgagtcattaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 178 - protein, 384 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMSGSETPGTSESATPESGEIAAIKQEIAAIKKEIAAIKWEIAAIKQGYGSGSETPGTSESATPESLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 179 - DNA, 1155 bp, synthetic construct
  source 1..1155: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgagcggcagcgagacccccggcaccagcgagagcgccacccccgagagcggcgagctggctgccattaagcaggagctcgctgctatcaagaaagagcttgcagcaattaagtgggaattggcagcaatcaaacagggcgctgggtccggctcagagaccccaggcacctctgagtctgcaactcctgagtcattaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 180 - protein, 384 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMSGSETPGTSESATPESGELAAIKQELAAIKKELAAIKWELAAIKQGAGSGSETPGTSESATPESLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 181 - DNA, 1077 bp, synthetic construct
  source 1..1077: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtggcgagatagccgcccttaaacaggagatagctgccttgaaaaaggagaacgctgcactgaagtgggagattgcggctctcaagcagggctattacggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 182 - protein, 358 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGGEIAALKQEIAALKKENAALKWEIAALKQGYYGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 183 - DNA, 1077 bp, synthetic construct
  source 1..1077: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtggcgaaattgccgctataaaacaggaaatagctgccatcaagaaggaaatagctgccattaagtgggagattgccgcgatcaaacagggctacggcggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 184 - protein, 358 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGGEIAAIKQEIAAIKKEIAAIKWEIAAIKQGYGGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 185 - DNA, 1077 bp, synthetic construct
  source 1..1077: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtggcgagctggctgccattaagcaggagctcgctgctatcaagaaagagcttgcagcaattaagtgggaattggcagcaatcaaacagggcgctgggggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 186 - protein, 358 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGGELAAIKQELAAIKKELAAIKWELAAIKQGAGGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 187 - DNA, 1155 bp, synthetic construct
  source 1..1155: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgagcggcagcgagacccccggcaccagcgagagcgccacccccgagagcggcgagatagccgcccttaaacaggagatagctgccttgaaaaaggagaacgctgcactgaagtgggagattgcggctctcaagcagggctattactccggctcagagaccccaggcacctctgagtctgcaactcctgagtcaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 188 - protein, 384 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLSGSETPGTSESATPESGEIAALKQEIAALKKENAALKWEIAALKQGYYSGSETPGTSESATPESGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 189 - DNA, 1155 bp, synthetic construct
  source 1..1155: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgagcggcagcgagacccccggcaccagcgagagcgccacccccgagagcggcgaaattgccgctataaaacaggaaatagctgccatcaagaaggaaatagctgccattaagtgggagattgccgcgatcaaacagggctacggctccggctcagagaccccaggcacctctgagtctgcaactcctgagtcaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 190-199 - skipped (10 entries)

SEQ ID NO: 200 - protein, 384 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLSGSETPGTSESATPESGEIAAIKQEIAAIKKEIAAIKWEIAAIKQGYGSGSETPGTSESATPESGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 201 - DNA, 1155 bp, synthetic construct
  source 1..1155: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgagcggcagcgagacccccggcaccagcgagagcgccacccccgagagcggcgagctggctgccattaagcaggagctcgctgctatcaagaaagagcttgcagcaattaagtgggaattggcagcaatcaaacagggcgctgggtccggctcagagaccccaggcacctctgagtctgcaactcctgagtcaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 202 - protein, 384 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLSGSETPGTSESATPESGELAAIKQELAAIKKELAAIKWELAAIKQGAGSGSETPGTSESATPESGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 203 - DNA, 1035 bp, synthetic construct
  source 1..1035: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtgactggctgaaggccttctacgacaaggtggccgagaagctgaaggaggccttcggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 204 - protein, 344 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGDWLKAFYDKVAEKLKEAFGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 205 - DNA, 1035 bp, synthetic construct
  source 1..1035: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtgactggttcaaggccttctacgacaaggtggccgagaagttcaaggaggccttcggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 206 - protein, 344 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGDWFKAFYDKVAEKFKEAFGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 207 - DNA, 1038 bp, synthetic construct
  source 1..1038: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggttttgacagggctcgcatgctcgaggagtacagtaaaaggttcaagaagtttggctatggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 208 - protein, 345 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGFDRARMLEEYSKRFKKFGYGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 209 - DNA, 1059 bp, synthetic construct
  source 1..1059: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtaaaaaagctctgctcgcactggctctacaccacttagctcacctggccctccacctggccctggcactcaaaaaagccggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 210 - protein, 352 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGKKALLALALHHLAHLALHLALALKKAGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 211 - DNA, 1032 bp, synthetic construct
  source 1..1032: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtctgcggaagctcagaaagcgcctgcttagactgtggaaattacggaaaagaggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 212 - protein, 343 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGLRKLRKRLLRLWKLRKRGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 213 - DNA, 1047 bp, synthetic construct
  source 1..1047: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggatccggtcctgtgctagacctgttccgggaactcctaaacgagctcctggaagcgcttaagcagaagctgaaaggttccggaggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 214 - protein, 348 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGSGPVLDLFRELLNELLEALKQKLKGSGGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 215 - DNA, 1026 bp, synthetic construct
  source 1..1026: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggcgactggctgaaggccttctacgacaaggtggccgagaagctgaaggaggccttcggcagcggccatcaccatcaccatcactga

SEQ ID NO: 216 - protein, 341 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGDWLKAFYDKVAEKLKEAFGSGHHHHHH

SEQ ID NO: 217 - DNA, 1026 bp, synthetic construct
  source 1..1026: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggcgactggttcaaggccttctacgacaaggtggccgagaagttcaaggaggccttcggcagcggccatcaccatcaccatcactga

SEQ ID NO: 218 - protein, 341 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGDWFKAFYDKVAEKFKEAFGSGHHHHHH

SEQ ID NO: 219 - DNA, 1029 bp, synthetic construct
  source 1..1029: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggctttgacagggctcgcatgctcgaggagtacagtaaaaggttcaagaagtttggctatggcagcggccatcaccatcaccatcactga

SEQ ID NO: 220 - protein, 342 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGFDRARMLEEYSKRFKKFGYGSGHHHHHH

SEQ ID NO: 221 - DNA, 1050 bp, synthetic construct
  source 1..1050: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggcaaaaaagctctgctcgcactggctctacaccacttagctcacctggccctccacctggccctggcactcaaaaaagccggcagcggccatcaccatcaccatcactga

SEQ ID NO: 222 - protein, 349 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGKKALLALALHHLAHLALHLALALKKAGSGHHHHHH

SEQ ID NO: 223 - DNA, 1023 bp, synthetic construct
  source 1..1023: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggcctgcggaagctcagaaagcgcctgcttagactgtggaaattacggaaaagaggcagcggccatcaccatcaccatcactga

SEQ ID NO: 224 - protein, 340 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGLRKLRKRLLRLWKLRKRGSGHHHHHH

SEQ ID NO: 225 - DNA, 1038 bp, synthetic construct
  source 1..1038: mol_type = other DNA
atgaaagcactttgggccgtgctgctagttacgctcctgacaggctgcctcgcggaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccctgtgctagacctgttccgggaactcctaaacgagctcctggaagcgcttaagcagaagctgaaaggcagcggccatcaccatcaccatcactga

SEQ ID NO: 226 - protein, 345 aa, synthetic construct
MKALWAVLLVTLLTGCLAEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGPVLDLFRELLNELLEALKQKLKGSGHHHHHH

SEQ ID NO: 227 - DNA, 1248 bp, synthetic construct
  source 1..1248: mol_type = other DNA
atgaaggttctgtgggctgcgttgctggtcacattcctggcaggatgccaggccgacgaggaggagatccagaaggccatcgaggagctgctgagaaagggcgtgagcgaggaggaggccgccatcatcatcgtgcagagattcaacgtggccgtggtggtggtggtgcaggacgagagacagggcaagcacatcagcgagtacatcagaagatacatccccgaggccgacgtgatcctgttcgccaacctggtggtgatcaaggtggagacccacgagctgagcaccagagtgtgggaggccgcccagaaggcctacggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 228 - protein, 415 aa, synthetic construct
MKVLWAALLVTFLAGCQADEEEIQKAIEELLRKGVSEEEAAIIIVQRFNVAVVVVVQDERQGKHISEYIRRYIPEADVILFANLVVIKVETHELSTRVWEAAQKAYGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 229 - DNA, 1257 bp, synthetic construct
  source 1..1257: mol_type = other DNA
atgaaggttctgtgggctgcgttgctggtcacattcctggcaggatgccaggccgacgaggaggagatccagaaggccatcgaggagctgctgagaaagggcgtgagcgaggaggaggccgccatcatcatcgtgcagagattcaacgtggccgtggtggtggtggtgcaggacgagagacagggcaagcacatcagcgagtacatcagaagatacatccccgaggccgacgtgatcctgttcgccaacctggtggtgatcaaggtggagacccacgagctgagcaccagagtgtgggaggccgcccagaaggcctacggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 230 - protein, 418 aa, synthetic construct
MKVLWAALLVTFLAGCQADEEEIQKAIEELLRKGVSEEEAAIIIVQRFNVAVVVVVQDERQGKHISEYIRRYIPEADVILFANLVVIKVETHELSTRVWEAAQKAYGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 231 - DNA, 1767 bp, synthetic construct
  source 1..1767: mol_type = other DNA
atgaaggttctgtgggctgcgttgctggtcacattcctggcaggatgccaggccgcttccgcatcacaagtacagctcgtccagtccggcgccgaagtgaaaaagcccggtgccagcgtgaaggtttcgtgcaaggcttcgggttacacgttcaccggctactacgttcactgggtccgtcaggcgccaggccaaggattggagtggatgggatgggtcaaccctaattcgggagacacaaactatgcgcagaaattccagggacgggtgacgatgaccagggatacatctatatcgactgcctacatggaactctccgggctccgctctgatgacactgcagtgtactactgtgcaagggatggcgatgaagactggtactttgatctttggggccgggggactccagtgacggtttcatctggtatcttgggcagcggcggaggagggtcggggggtggtggctcaggaggtggcggaagcgacatccgcttgacacagagcccatcctctttgagtgccagcattggcgatagagtgaccatcacgtgccgagcatctcaaggcatttctcgcagtctggtgtggtatcagcagaagcctggaaaggctccaaggttgctgatctatgccgcttctactctacagtctggcgtacccagtcgcttcagtgggtcaggaagcggaacagactttactcttaccatctcatccctgcaaccagaagattttgccacctactactgtctgcagcataatacatacccattcaccttcgggccgggtaccaaggtcgacattaaatctggcatcccggaacagaagttgggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 232 - protein, 588 aa, synthetic construct
MKVLWAALLVTFLAGCQAASASQVQLVQSGAEVKKPGASVKVSCKASGYTFTGYYVHWVRQAPGQGLEWMGWVNPNSGDTNYAQKFQGRVTMTRDTSISTAYMELSGLRSDDTAVYYCARDGDEDWYFDLWGRGTPVTVSSGILGSGGGGSGGGGSGGGGSDIRLTQSPSSLSASIGDRVTITCRASQGISRSLVWYQQKPGKAPRLLIYAASTLQSGVPSRFSGSGSGTDFTLTISSLQPEDFATYYCLQHNTYPFTFGPGTKVDIKSGIPEQKLGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 233 - DNA, 1701 bp, synthetic construct
  source 1..1701: mol_type = other DNA
atgaaggttctgtgggctgcgttgctggtcacattcctggcaggatgccaggcccaggtccaactacagcagtccggagccgaactagcccgccgcggggccagcgtgaagatgagctgcaaggctagcggttatacctttactagatacacaatgcattgggtcaaacagcggcctggtcagggactggagtggattggctatataaatccaagtagagggtacactaactacaatcaaaagttcaaagacaaggctacattgaccactgataagagttctagtaccgcttacatgcagctaagcagcctaacatcggaggattccgcagtgtattactgtgctcgctactatgacgatcattatagccttgattattggggccagggtacaactctaaccgtgtcctccgcaaaaactacccccgacatagtcttaacccagtctccagcaatcatgtccgctagccctggggagaaagtaacaatgacgtgtagtgcctcttcatcggtcagttatatgaattggtaccagcaaaagtccgggacatccccaaagcggtggatctatgacaccagcaaactggcctctggggtgccagcccacttccgggggtcagggtccggtacctcgtatagcctgacgatctcagggatggaggcagaggacgcagccacgtactattgccagcagtggtccagcaacccgttcacttttgggtcaggaacaaaactcgaaattaatagagccgacactgctgccgctggatcaggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 234 - protein, 566 aa, synthetic construct
MKVLWAALLVTFLAGCQAQVQLQQSGAELARRGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAAAGSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 235 - DNA, 1170 bp, synthetic construct
  source 1..1170: mol_type = other DNA
atgaaggttctgtgggctgcgttgctggtcacattcctggcaggatgccaggccgaccactgggaggaggtgttcagaaacgccctggagcacctgcaggaggccacccagcagaacgacccccagaaggccaagaagatcctggaggaggcccacaagaagctgagaagagagctgagcgaggaggaggccagaagcgtggtgagatggctgaagcagctggtggacagagagaagagcggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 236 - protein, 389 aa, synthetic construct
MKVLWAALLVTFLAGCQADHWEEVFRNALEHLQEATQQNDPQKAKKILEEAHKKLRRELSEEEARSVVRWLKQLVDREKSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 237 - DNA, 1173 bp, synthetic construct
  source 1..1173: mol_type = other DNA
atgaaggttctgtgggctgcgttgctggtcacattcctggcaggatgccaggccagcctggacgaggccaagaagctgctgcaggaggccgagaagctggccagaaagctgaacgacagaaccgagctggcctacgtggagttcctgaagcacagcctggagaccgccaagaagcagaacgacaagagaaccatcgagagcgtgagagacatggccagagacgccctggaggagctgcagagcggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 238 - protein, 390 aa, synthetic construct
MKVLWAALLVTFLAGCQASLDEAKKLLQEAEKLARKLNDRTELAYVEFLKHSLETAKKQNDKRTIESVRDMARDALEELQSGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 239 - DNA, 999 bp, synthetic construct
  source 1..999: mol_type = other DNA
atgaaggttctgtgggctgcgttgctggtcacattcctggcaggatgccaggcctgcagaccccccagaggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 240 - protein, 332 aa, synthetic construct
MKVLWAALLVTFLAGCQACRPPRGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 241 - DNA, 1011 bp, synthetic construct
  source 1..1011: mol_type = other DNA
atgaaggttctgtgggctgcgttgctggtcacattcctggcaggatgccaggcctgcggcagaaagagcaagaccgtgtgcggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 242 - protein, 336 aa, synthetic construct
MKVLWAALLVTFLAGCQACGRKSKTVCGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 243 - DNA, 1023 bp, synthetic construct
  source 1..1023: mol_type = other DNA
atgaaggttctgtgggctgcgttgctggtcacattcctggcaggatgccaggcctgcggcttcgagtgcgtgagacagtgccccgagagatgcggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 244 - protein, 340 aa, synthetic construct
MKVLWAALLVTFLAGCQACGFECVRQCPERCGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 245 - DNA, 1005 bp, synthetic construct
  source 1..1005: mol_type = other DNA
atgaaggttctgtgggctgcgttgctggtcacattcctggcaggatgccaggccttcgacttctggatcacccccggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 246 - protein, 334 aa, synthetic construct
MKVLWAALLVTFLAGCQAFDFWITPGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 247 - DNA, 1011 bp, synthetic construct
  source 1..1011: mol_type = other DNA
atgaaggttctgtgggctgcgttgctggtcacattcctggcaggatgccaggcctgcacccacgccctgtggcacacctgcggatccggtgggtccggtagtggtggttccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctggccagcctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactag

SEQ ID NO: 248 - protein, 336 aa, synthetic construct
MKVLWAALLVTFLAGCQACTHALWHTCGSGGSGSGGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLASLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 249 - DNA, 1041 bp, synthetic construct
  source 1..1041: mol_type = other DNA
atgaaggttctgtgggctgcgttgctggtcacattcctggcaggatgccaggccgccaacaccccctgcggcccctacacccacgactgccccgtgaagagaggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 250 - protein, 346 aa, synthetic construct
MKVLWAALLVTFLAGCQAANTPCGPYTHDCPVKRGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 251 - DNA, 1713 bp, synthetic construct
  source 1..1713: mol_type = other DNA
atgaaggttctgtgggctgcgttgctggtcacattcctggcaggatgccaggcccaggtacagctgcagcagtctggaggagagttagcgaagccaggcgcctcagtgaaggtgtcctgtaaggcctcaggatataccttctcctccttttggatgcactgggtgcgccaggctcctgggcagggcctggagtggatagggtacattaaccctaggtccggctacactgaatataatgagatcttccgagacaaggcgaccatgacaaccgacacctccacttccaccgcatacatggagttaagttccctgcgatcagaggacaccgccgtgtactactgtgccagcttcctgggccggggagcgatggattattggggacagggcactacggtgactgtgagcagcggcggcgggggatcaggcgggggagggtccggcgggggagggtcagacattcagatgacccagtccccatcatccctgtccgccagcgtgggggatagagtaacaatcacatgccgcgccagccaggacatttcaaactacctggcctggtaccagcagaaaccggggaaagcgcccaagctgctgatctactatacctctaaaattcacagtggagttccttccaggttcagcggttccgggagtgggacagattatacatttacgatcagcagtctgcaaccagaagacatcgcaacctactattgccagcagggtaacactttcccgtatacattcgggcaggggacaaaggtcgagatcaaaggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 252 - protein, 570 aa, synthetic construct
MKVLWAALLVTFLAGCQAQVQLQQSGGELAKPGASVKVSCKASGYTFSSFWMHWVRQAPGQGLEWIGYINPRSGYTEYNEIFRDKATMTTDTSTSTAYMELSSLRSEDTAVYYCASFLGRGAMDYWGQGTTVTVSSGGGGSGGGGSGGGGSDIQMTQSPSSLSASVGDRVTITCRASQDISNYLAWYQQKPGKAPKLLIYYTSKIHSGVPSRFSGSGSGTDYTFTISSLQPEDIATYYCQQGNTFPYTFGQGTKVEIKGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 253 - DNA, 1719 bp, synthetic construct
  source 1..1719: mol_type = other DNA
atgaaggttctgtgggctgcgttgctggtcacattcctggcaggatgccaggcccaggtgcagctggtggaatccgggggcggggtggtgcagcccggccggagcaggcggctgtcatgtgccgcatcaggttttactttttctaggtataccatgcattgggtcagacaggcaccaggcaaaggtttggagtgggttgctgtgatcagcttcgatggatctaataagtattacgtggacagtgtcaaagggcgtttcaccatctcacgcgataattccgagaacactctctacctacaggtgaatatactgcgcgccgaagatactgccgtctattattgcgcaagggaagcccggggatcttacgccttcgacatttggggacagggcacaatggttacagtttcttccggcggaggcgggagcggtggaggcggaagcggaggaggcggctctgagattgtgctgactcagtcccccgcaactctcagcctctcacccggtgaacgagcgaccctctcctgccgcgcatctcagtctgtctcttcatacctcgcatggtatcaacagaaacccggacaggctccacgactgctcatctacgatgcatccaatcgggctacgggcatcccagcaagatttagcgggtccggatctggcaccgactttaccctgacaatctcctccctcgaacctgaagacttcgctgtgtattattgccagcagaggagcaactggccacccttcaccttcgggccaggcacaaaagttgacattaaaggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 254 - protein, 572 aa, synthetic construct
MKVLWAALLVTFLAGCQAQVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIKGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 255 - DNA, 1710 bp, synthetic construct
  source 1..1710: mol_type = other DNA
atgaaggttctgtgggctgcgttgctggtcacattcctggcaggatgccaggcccaggttaccctgaaggagtccgggccagttctggtgaagcctacggagacattgacactgacatgtacagtttccgggtttagcttgaccagtaacgcggtttcctgggtccgccagcctccaggaaaagcgctggagtggatcgcagccatctccagcggaggcaccacctactacaactcagcattcaaaagcagactgaccatctcccgtgataccagtaagtcacaggtggttctgactatgacaaatatggaccccgtcgacacagcaacatactattgcgccaggcggtacgggtacggctggtactttgacttctggggacagggaacacttgtgacagtaagtagcgggggcggcgggagcgggggcgggggcagcggcggcggaggctctgacatccagatgactcagtctccgtcaagcctgagcgcttccgtcggagatcgcgtgacaatcaactgtaaagcctcgcagaacatatacaacagcctggcatggtaccagcagaagcctggcaaggctcccaaggtgctgatttttaacgccaacagcctgcagactggcattccttctcggtttagtggcagtggctctggaactgactttaccctgacgatctcatctctgcaaccagaggacttcgccacgtactattgtcagcagttctatagtgggtacacattcggtcagggaaccaagctggaaattaagggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 256 - protein, 569 aa, synthetic construct
MKVLWAALLVTFLAGCQAQVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSGGGGSGGGGSGGGGSDIQMTQSPSSLSASVGDRVTINCKASQNIYNSLAWYQQKPGKAPKVLIFNANSLQTGIPSRFSGSGSGTDFTLTISSLQPEDFATYYCQQFYSGYTFGQGTKLEIKGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 257 - DNA, 1728 bp, synthetic construct
  source 1..1728: mol_type = other DNA
atgaaggttctgtgggctgcgttgctggtcacattcctggcaggatgccaggccgaggtgaagctcgtcgaatctggtgggggattggtccagccgggtggctccctgaagctgtcttgcgcaacctctggattcacgttcagcgattactacatgtcttgggtgaggcaaacacccgaaaagcgcctggagtgggttgcctacatttccagtagtggcgggtctacctactacagcgacaccgttaaggggcagttcacaatatccagagacaatgccaagaatacgctctacctccagatgtcaagattgaagagtgaggatacagctatgtactactgtgccaggcgggaggactacgacggacgcctgacctattggggtcaagggacactcgtcactatctctgccggaggttctggcggaggaggctcgggcgggggtggctcaggtggggacatcgtcctgacacaaagcccagccagtctggcagtttcacttggacagcgtgctactattagttgtagggcatctaagtccgtgagtacatcaggctacagctatatgaactggtaccagcagaagcccggacagccacccaaagtacttatctatcttgcctcaaaattagagagcggagtccccgcaaggttttccgggagcggttccggtactgactttaccctcaatatccacccagtggaggaagaggatgccgcgacctactactgtcaacattctcgggagcttccctacacttttggtggaggtaccaagcttgagattaagggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 258 - protein, 575 aa, synthetic construct
MKVLWAALLVTFLAGCQAEVKLVESGGGLVQPGGSLKLSCATSGFTFSDYYMSWVRQTPEKRLEWVAYISSSGGSTYYSDTVKGQFTISRDNAKNTLYLQMSRLKSEDTAMYYCARREDYDGRLTYWGQGTLVTISAGGSGGGGSGGGGSGGDIVLTQSPASLAVSLGQRATISCRASKSVSTSGYSYMNWYQQKPGQPPKVLIYLASKLESGVPARFSGSGSGTDFTLNIHPVEEEDAATYYCQHSRELPYTFGGGTKLEIKGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 259 - DNA, 1707 bp, synthetic construct
  source 1..1707: mol_type = other DNA
atgaaggttctgtgggctgcgttgctggtcacattcctggcaggatgccaggccgacattcagatgacccagagtccctcgagtttaagtgcgtctgtgggagatcgtgtgacgattacatgccgcgcctcagagtcggtggataattacgggatctctttcatgaattggtttcagcaaaaaccgggcaaggcccctaagctgctgatctacgcagcctctgacttaggatctggggtgccaagccggttttcaggctctggttctggcacagacttcaccctgacaatatcgtctctccagccagaagatttcgccacatactactgccaccagagtaaggaggtgccctacacctttggtcaaggcaccaaactcgaaatcaagggtggagggggttctgggggcggaggctctgggggaggcggctcccaggttcagctagttcaatctggggctgaggtcaagaaacccggggcgtcagttaaggtgagctgtaaggcgagcggatacacattcactaagtactggattaattgggttagacaggccccgggccaaggactggagtggatggggataatttatcctggcagctcaacaagcaattacaacgagaagtttaaaacacgggtgacgatgacagtggatacgagcacctcaacagcatatatggaactgtccagcctgagatctgaagatacagctgtttactactgtgtgaagaatgataactggggccagggtacactcgtaacagtatcaagcggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 260 - protein, 568 aa, synthetic construct
MKVLWAALLVTFLAGCQADIQMTQSPSSLSASVGDRVTITCRASESVDNYGISFMNWFQQKPGKAPKLLIYAASDLGSGVPSRFSGSGSGTDFTLTISSLQPEDFATYYCHQSKEVPYTFGQGTKLEIKGGGGSGGGGSGGGGSQVQLVQSGAEVKKPGASVKVSCKASGYTFTKYWINWVRQAPGQGLEWMGIIYPGSSTSNYNEKFKTRVTMTVDTSTSTAYMELSSLRSEDTAVYYCVKNDNWGQGTLVTVSSGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 261 - DNA, 1722 bp, synthetic construct
  source 1..1722: mol_type = other DNA
atgaaggttctgtgggctgcgttgctggtcacattcctggcaggatgccaggccgatattcagatgacacagtccccttcctccctctccgcctcggtgggcgatcgcgttacaatcacttgtcgggccagtcaaagaggcctaaggaatgtggcagttgcgtggtaccaacagaagcccggtaaagcccccaaactgctgatctattctgcatcatcactctatagcggcgtgcctagtcggttcagtggcagtaggtctgggacagattttactttgaccatttcctccctccagccagaggacttcgcgacctactattgccaacaatgggcagtgcacagcctgatcacattcgggcagggaacgaaagtcgaaatcaaacgaggaggtggaggttcaggaggtggaggtagcggaggtggaggtagcgaggtccagttggttgagagtggcggcgggttggtacagccgggcgggtccctacgcctgtcctgcgcagcatctggcttcaacatatccgtatatatgatgcactgggtccgccaagcccccgggaagggcctggaatgggtggcaagcatctacccctattcgggatacacctactatgcagacagcgtgaaaggccgattcacgatctctgctgacaccagtaagaacacggcctatctgcagatgaattctttgagagcagaggatacagccgtttactactgcgccagatacgtataccacgctttagactattggggacagggcaccctggtaaccgtgtcgtcaggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 262 - protein, 573 aa, synthetic construct
MKVLWAALLVTFLAGCQADIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSSGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 263 - DNA, 1584 bp, synthetic construct
  source 1..1584: mol_type = other DNA
atgaacgataagctcaccttatggactacaccggccccgagtccgaattgtaggctgaatgccgagaaagatgccaaactgactctggttttgacaaagtgcggaagtcaaatattggcgactgtaagcgtgctggccgtgaaagggtccttggccccgatttctggtacagtacagtcagctcatctgatcattagatttgatgaaaatggagtcttgttgaacaattcatttctggacccggagtactggaatttcaggaatggtgacctgacggaagggacagcttacaccaacgctgtgggcttcatgccaaatcttagcgcctaccccaaatcacatggaaaaactgcaaaatctaacatcgtgagccaggtctatttgaatggcgacaaaactaagcctgtcacgcttactattaccttgaatggaacccaagagacaggcgacacgaccgcttgtgattgtcgaggcgattgtttctgcggcccaagtgcttattccatgtccttttcttgggattggagtggccataactatatcaacgagattttcgctacatcctcttataccttctcatacatcgcccaggaaggtagcggaagcggctccgggtcgggaagcaagaagaagaaaaagaagaaaggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 264 - protein, 527 aa, synthetic construct
MNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTACDCRGDCFCGPSAYSMSFSWDWSGHNYINEIFATSSYTFSYIAQEGSGSGSGSGSKKKKKKKGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 265 - DNA, 1629 bp, synthetic construct
  source 1..1629: mol_type = other DNA
atgaaggttctgtgggctgcgttgctggtcacattcctggcaggatgccaggcctcattgaaagacgatccaagccagagcgcgaatttgttatctgaggctaaaaaactgaacgagagccaggcccctaaggccgataacaaattcaacaaggaacaacaaaacgcattctatgaaattctgcatcttccaaatttaaacgaagagcagcgtaacgggttcattcagtccctcaaagacgatccaagtcagtcagccaatcttctcgctgaagccaagaagctgaatgacgctcaagctcctaaggctgacaataagttcaacaaggagcagcaaaatgctttctacgagattcttcacttgccaaacctcactgaggagcagaggaatggattcattcagagcttaaaagacgatccaagcgtgtcgaaggagatattggcagaggccaagaaacttaatgatgcccaggcccctaaaaccacctataagctcgtgatcaacggcaaaactctgaagggcgagacaacaacagaagctgttgacgcggaaaccgcagagcggcatttcaaacagtacgccaacgataatggtgttgacggtgagtggacctacgatgacgccacaaaaacctttactgtcaccgagaaacccgaggtgatcgatgcaagcgaattgacacccgccgttggatccggtgggtccggtagtggtggttccgaaggggaaccggaggtgacggaccaacttgagtggcagtctaatcagccctgggaacaggcactcaaccgcttctgggattatctgcgctgggtacagaccctgagcgaccaggtgcaggaggaattacagagctctcaagtgactcaggagcttaccgctcttatggaagacacaatgactgaggttaaggcatacaaaaaggagctggaggaacagttggggccagtcgcagaggaaactagggcacggctgggtaaggaagtgcaggccgctcaggccaggcttggggccgatatggaggatctgcgcaaccgcctgggacagtaccggaatgaggttcacaccatgttaggtcagagcacagaggaaatcagagcccggctcagcacacacctcgcaagtatgcgcaagcgcctaatgcgtgacgccgaggatctgcagaagcgcctggcggtgtacaaagctggcgcaagagagggcgccgagagaggcgtctcggccattagagagagactaggacccttggtcgagcaggggaggcaaagaacagccaacctgggagccggcgccgcacaacctctaagagaccgcgctcaggcctttggtgatcggataagaggaaggttggaggaggtgggcaatcaggcaagagaccgtctcgaggaagtgcgcgagcacatggaggaggtgagaagtaaaatggaagaacaaacccagcagattaggcttcaggcagaaatatttcaggcccgcttaaagggctggttcgagcctatcgtggaggatatgcatagacagtgggccaacctgatggagaagatccaagcctctgttgcaaccaaccccatcataactccagtggctcaggaaaaccagggcagcggccatcaccatcaccatcactga

SEQ ID NO: 266 - protein, 542 aa, synthetic construct
MKVLWAALLVTFLAGCQASLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKTTYKLVINGKTLKGETTTEAVDAETAERHFKQYANDNGVDGEWTYDDATKTFTVTEKPEVIDASELTPAVGSGGSGSGGSEGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLASMRKRLMRDAEDLQKRLAVYKAGAREGAERGVSAIRERLGPLVEQGRQRTANLGAGAAQPLRDRAQAFGDRIRGRLEEVGNQARDRLEEVREHMEEVRSKMEEQTQQIRLQAEIFQARLKGWFEPIVEDMHRQWANLMEKIQASVATNPIITPVAQENQGSGHHHHHH

SEQ ID NO: 267 - protein, 4 aa, synthetic construct
GGGS

SEQ ID NO: 268 - protein, 15 aa, synthetic construct
GGSGGSGGSGGSGGS

SEQ ID NO: 269 - protein, 16 aa, synthetic construct
KKEEEKKEEEKKEEEK

SEQ ID NO: 270 - protein, 6 aa, synthetic construct
HHHHHH

SEQ ID NO: 271 - protein, 5 aa, synthetic construct
CRPPR

SEQ ID NO: 272 - protein, 9 aa, synthetic construct
CGRKSKTVC

SEQ ID NO: 273 - protein, 13 aa, synthetic construct
CGFECVRQCPERC

SEQ ID NO: 274 - protein, 7 aa, synthetic construct
FDFWITP

SEQ ID NO: 275 - protein, 9 aa, synthetic construct
CTHALWHTC

SEQ ID NO: 276 - protein, 16 aa, synthetic construct
ANTPCGPYTHDCPVKR